(19) United States
(12) Patent Application Publication
(10) Pub. No.: US 2005/0037439 A1
(43) Pub. Date: Feb. 17, 2005

(54) Differentially expressed genes involved in cancer, the polypeptides encoded thereby, and methods of using the same

(76) Inventors: Maureen J. Bourner, O'Fallon, MO (US); Paul J. Hippenmeyer, Chesterfield, MO (US); Richard D. Head, Florissant, MO (US); Richard A. Mazzarella, Webster Groves, MO (US); Nicholas R. Staten, Kirkwood, MO (US); Barbara K. Klein, Town and Country, MO (US)

Correspondence Address: HARNESS, DICKEY & PIERCE, P.L.C., 7700 BONHOMME, SUITE 400, ST LOUIS, MO, 63105, US

(21) Appl. No.: 10/696,639

(22) Filed: Oct. 29, 2003

Related U.S. Application Data
(60) Provisional application No. 60/422,176, filed on Oct. 29, 2002.

(51) Int. Cl. G01N 33/53; G01N 33/567; G01N 33/574; C07K 16/18

(52) U.S. Cl. 435/7.2; 530/388.25

(57) ABSTRACT

The invention relates nucleic acids and their encoded polypeptides, whose expression is modulated in cancer or tumor cells. The invention further relates to methods useful for treating or modulating cancer or tumors in mammals in need of such biological effect. This includes the diagnosis and treatment of oncological disorders. Additionally, the present invention further relates to the use of antibodies against the polypeptides of the present invention as diagnostic probes or as therapeutic agents as well as the use of polynucleotide sequences encoding the polypeptides of the present invention as diagnostic probes or therapeutic agents for the treatment of a broad range of pathological states.

The present application claims priority under Title 35, United States Code, §119 to U.S. Provisional application Ser. No. 60/422,176, filed Oct. 29, 2002, which is incorporated by reference in its entirety as if written herein.

The present application contains a sequence listing in computer readable form under Title 37, § 1.53 (e)(5) entitiled 01040_{—}1.ST25.txt (created Oct. 24, 2003, 2,345 KB), which is incorporated by reference in its entirety as if written herein.

### FIELD OF THE INVENTION

The invention relates generally to the identification of nucleic acids and their encoded polypeptides, whose expression is modulated in cancer or tumor cells. These nucleic acids and proteins may not have previously been identified as having a biological role in cancer. The invention further relates to methods useful for treating or modulating cancer or tumors in mammals in need of such biological effect. This includes the diagnosis and treatment of oncological disorders. Additionally, the present invention further relates to the use of antibodies against the polypeptides of the present invention as diagnostic probes or as therapeutic agents as well as the use of polynucleotide sequences encoding the polypeptides of the present invention as diagnostic probes or therapeutic agents for the treatment of a broad range of pathological states. The present invention also relates to antisense molecules.

### BACKGROUND OF THE INVENTION

Cancer Background

The present invention relates to methods and compositions for the diagnosis, prevention, and treatment of neoplastic cell growth and proliferation, i.e., tumors and cancers (e.g., colon cancer) in mammals, for example, humans. Specifically, genes which are differentially expressed in tumor cells relative to normal cells are identified. Among these are certain Incyte (Palo Alto, Calif.) unique genes.

Malignant tumors, i.e., cancers, are the second leading cause of death in the United States, after heart disease (Boring, et al., CA Cancer J. Clin., 43:7, 1993), and develop in one in three Americans. One of every four Americans dies of cancer. Cancer is characterized primarily by an increase in the number of abnormal, or neoplastic, cells derived from a normal tissue which proliferate to form a tumor mass, the invasion of adjacent tissues by these neoplastic tumor cells, and the generation of malignant cells which spread via the blood or lymphatic system to regional lymph nodes and to distant sites. The latter progression to malignancy is referred to as metastasis.

Cancer can result from a breakdown in the communication between neoplastic cells and their environment, including their normal neighboring cells. Signals, both growth-stimulatory and growth-inhibitory, are routinely exchanged between cells within a tissue. Normally, cells do not divide in the absence of stimulatory signals, and, likewise, will cease dividing in the presence of inhibitory signals. In a cancerous, or neoplastic, state, a cell acquires the ability to “override” these signals and to proliferate under conditions in which normal cells would not grow.

Tumor cells must acquire a number of distinct aberrant traits to proliferate. Reflecting this requirement is the fact that the genomes of certain well-studied tumors carry several different independently altered genes, including activated oncogenes and inactivated tumor suppressor genes. Differential expression of the following suppressor genes has been demonstrated in human cancers: a retinoblastoma gene, RB; the Wilms' tumor gene, WT1 (11p); a gene deleted in colon carcinoma, DCC (18q); the neurofibromatosis type 1 gene, NF1 (17q); and a gene involved in familial adenomatous polyposis coli, APC (5q) (Vogelstein, B. and Kinzler, K. W., Trends Genet., 9:138-141, 1993). Each of these genetic changes appears to be responsible for imparting some of the traits that, in aggregate, represent the full neoplastic phenotype (Hanahan. D. and Weinberg, R. A., Cell, 100:57-70, 2000).

Colon cancer is the second leading cause of cancer-related deaths in the United States. The American Cancer Society estimates that there will be approximately 94,700 new cases of colon cancer in the United States in 1999, and that colon cancer will be responsible for about 47,900 deaths. Colon cancer frequently metastasizes to the liver and the lung.

Unlike lung cancer, in which smoking has been identified as the prime etiologic factor responsible for the disease, the principle mechanisms underlying colon cancer are complex and incompletely understood. Dietary factors are believed to promote carcinogenesis, especially a high fat intake. At the molecular level, a multistep process involving a number of mutations is suspected in the progression of adenomas to colon tumors (Vogelstein et al. (1988) N. Engl. J. Med. 319:525-532). The development and progression of colon cancer is driven by sequential mutations in three gene types: oncogenes, tumor suppressor genes and mismatch repair genes, which control the rate of mutations of other genes, including oncogenes and tumor suppressor genes. These mutations occur as a result of genetic predisposition (germline mutations) or in response to environmental factors (somatic mutations).

Several mutations that are associated with colon cancer have been identified. Germline mutations that have been linked to hereditary, or familial, colon cancer include the tumor suppressor gene adenomatous polyposis coli (APC (Lengauer et al. (1991) Science 253:665-669) and the mismatch-repair genes MutL and MutS (Modrich (1995) Phil. Trans. R. Soc. Lond. B 347:89-95; Kolodner (1996) Genes Dev. 10: 1433-1442). Defective APC has been implicated in familial adenomatous polyposis (FAP) and MutL and MutS in hereditary nonpolyposis colorectal cancer (HNPCC). Somatic mutations identified in association with sporadic colon cancer include the oncogenes K-ras, c-myc, and the tumor suppressor genes p53, APC, neurofibromatosis type I GTPase-activating protein (NF I GAP), deleted in colon cancer (DCC and mutated in colon cancer (MCC) (Midgley et al. (1999) Lancet 353:391-399).

Conventional therapeutic approaches to treat colon cancer include surgical resection, radiation and chemotherapy, including adjuvant therapy. Gene therapeutic approaches include transfer of cytokine or immune antigen genes, transfer of enzyme-prodrug systems (see, e.g., Huber et al. (1993) Cancer Res. 53:4619-4626) and replacement of tumor suppressor genes (see, e.g., Venook et al. (1998) Proc. ASCO 17:43 1 a) using viral vectors (Zwacka et al. (1998) Hematol. Oncol. Chn. North Am. 12:595 615).

While several genes associated with colon cancer have been identified, identification of additional genes linked to development (or inhibition of development) of colon cancer can provide additional diagnostic tools and therapeutic targets. Identification of genes differentially expressed in colon cancer is particularly important in the advancement of drug discovery, diagnostic technologies, and the understanding of the progression and nature of colon cancer. The invention provides for identification of such differentially expressed genes.

Microarrray Background

DNA-based arrays can provide an efficient, high-throughput method to examine gene expression and genetic variability. For example, SNPs, or single nucleotide polymorphisms, are the most common type of human genetic variation. DNA-based arrays can dramatically accelerate the discovery of SNPs in hundreds and even thousands of genes. Likewise, such arrays can be used for SNP genotyping in which DNA samples from individuals or populations are assayed for the presence of selected SNPs. These approaches will ultimately lead to the systematic identification of all genetic variations in the human genome and the correlation of certain genetic variations with disease susceptibility, responsiveness to drug treatments, and other medically relevant information. (See, for example, Wang, D. G. et al. (1998) Science 280:1077-1082.)

DNA-based array technology is especially important for the rapid analysis of global gene expression patterns. For example, genetic predisposition, disease, or therapeutic treatment may directly or indirectly affect the expression of a large number of genes in a given tissue. In this case, it is useful to develop a profile, or transcript image, of all the genes that are expressed and the levels at which they are expressed in that particular tissue. A profile generated from an individual or population affected with a certain disease or undergoing a particular therapy may be compared with a profile likewise generated from a control individual or population. Such analysis does not require knowledge of gene function, as the expression profiles can subjected to mathematical analyses which simply treat each gene as a marker. Furthermore, gene expression profiles may help dissect biological pathways by identifying all the genes expressed, for example, at a certain developmental stage, in a particular tissue, or in response to disease or treatment. (See, for example, Lander, E. S. et al. (1996) Science 274:536-539.)

### SUMMARY OF THE INVENTION

In one aspect, the invention involves a method of assessing the efficacy of an oncological disorder treatment in a subject, wherein the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose cancerous stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian subject. The expression of the nucleic acid sequences may be either increased or decreased in the test cell population as compared to the reference cell population.

In a further aspect, the invention involves a method of diagnosing an oncological disorder, wherein the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose cancerous stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian, subject. The expression of the nucleic acid sequences may be either increased or decreased in the test cell population as compared to the reference cell population.

In another aspect, the invention involves a method of identifying a test therapeutic agent for treating an oncological disorder in a subject involving the steps of providing a test cell population capable of expressing one or more of the nucleic acid sequences of the present invention; contacting the test cell population with the test therapeutic agent; detecting the expression of one or more of these nucleic acid sequences; comparing the expression to that of the nucleic acid sequences in a reference cell population whose cancerous stage is known; and identifying a difference in expression level, if present, between the test cell population and the reference cell population. In different embodiments, the subject may be a mammal or, more preferably, a human. Additionally, the test therapeutic agent may be either a known oncological disorder agent or an unknown oncological disorder agent. The antagonist may be an antibody having selectivity to at least one of the polypeptides of the present invention. The oncological disorder to be treated can be selected from the following diseases or disorders: locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

In a further aspect, the invention involves a method of identifying or determining the susceptibility to, predisposition to, or presence of, an oncological disorder in a subject. In this aspect, the method involves the steps of providing a test cell population capable of expressing one or more of the nucleic acid sequences of the present invention; detecting the expression of one or more of these nucleic acid sequences; comparing the expression to that of the nucleic acid sequences in a reference cell population whose cancerous stage is known; and identifying a difference in expression level, if present, between the test cell population and the reference cell population. The subject may be a mammal, or, more preferably, a human.

In an alternative aspect, the invention involves a method of treating an oncological disorder by administering an agent that modulates the expression or activity of one or more of the nucleic acid sequences of the present invention to a patient suffering from or at risk for developing the oncological disorder. This agent can be one that decreases the expression of one or more of sequences of the present invention that are up regulated in cancerous tissues. Alternatively, it can be one that increases the expression of one or more of sequences of the present invention that are down regulated. Additionally, the agent can be an antibody to a polypeptide encoded by the nucleic acid sequence, an antisense nucleic acid molecule, a peptide, a polypeptide agonist, a polypeptide antagonist, a peptidomimetic, a small molecule, or another drug.

The invention also includes a kit containing one or more reagents for detecting two or more of the nucleic acid sequences of the present invention. Additionally, the invention involves an array of probe nucleic acids capable of detected two or more of the nucleic acids of the present invention.

The polypeptides and nucleic acids of the invention can be used to treat an oncological disorder in a subject. Treatment of an oncological disorder may be in a mammal, preferably a human. In various embodiments, therapeutic compositions containing the polypeptides and nucleic acids of the invention can be used to treat oncological disorders. These therapeutic compositions can include a pharmaceutically acceptable carrier and, additionally, an active ingredient such as an anti-oncological agent or an anti-inflammatory agent. Also provided is a kit containing a therapeutic composition for use in the treatment of an oncological disorder along with a pharmaceutically acceptable carrier, wherein the therapeutic composition is a polypeptide of the present invention, an agonist of a polypeptide of the present invention, or an antagonist of a polypeptide of the present invention.

Also included in the invention is an isolated nucleic acid molecule that is at least 80% identical to the nucleic acid encoding the polypeptide of the present invention or the complement of the nucleic acid sequence, as well as vectors and host cells containing this nucleic acid sequence. Also provided is a method for producing a polypeptide by culturing a host cell transformed with one or more vectors described herein under conditions suitable for the expression of the protein encoded by the vector.

In another aspect, there is provided an isolated polypeptide encoded by an isolated nucleic acid sequence or oligonucleotide described herein. In some aspects, the isolated protein, functional variants or fragments thereof. In another embodiment, a variant or fragment of a protein of the present invention retains the respective activity.

In still further aspects, the invention involves pharmaceutical compositions containing either the isolated nucleic acid, isolated polypeptide or antibody. Another aspect involves methods of detecting the presence of the nucleic acid and polypeptide.

An additional aspect of the present invention is markers and methods of prognosticating or detecting oncological disorders based on the presence of nucleic acid or polypeptide of the present invention in a biological sample.

A further embodiment of the present invention is markers and methods for assessing the efficacy of anti cancer treatments based on monitoring the level of a nucleic acid or polypeptide of the present invention in a biological sample.

Other features and advantages of the invention will be apparent from the following detailed description and from the claims.

### BRIEF DESCRIPTION OF THE DRAWINGS

FIGS. 1-31. Analysis of putative signal sequence and transmembrane regions of potentially over-expressed tumor proteins. The first seventy N-terminal amino acids of each protein was analyzed the Hidden Markov Model (HMM) SignalP program for the presence of a signal sequence (left graph). In addition, each entire protein was examined by the TMHMM program for possible transmembrane regions (right graph). From this information, the subcellular location and membrane topology of each protein was predicted. FIGS. 1-31 shows the SignalP and TMHMM analysis of the protein sequences of PCTUC-5 (SEQ ID NO:39), PCTUC-93 (SEQ ID NO:46), PCTUC-190 (SEQ ID NO:57), PCTUC-239 (SEQ ID NO:65), PCTUC-246 (SEQ ID NO:40), PCTUC-360 (SEQ ID NO:53), PCTUC-462 (SEQ ID NO:58), PCTUC-468 (SEQ ID NO:48), PCTUC-536 (SEQ ID NO:3114), PCTUC-582 (SEQ ID NO:64), PCTUC-605 (SEQ ID NO:71), PCTUC-629 (SEQ ID NO:67), PCTUC-722 (SEQ ID NO:61), PCTUC-748 (SEQ ID NO:63), PCTUC-784 (SEQ ID NO:72), PCTUC-812 (SEQ ID NO:66), PCTUC-856 (SEQ ID NO:49), PCTUC-898 (SEQ ID NO:43), PCTUC-935 (SEQ ID NO:70), PCTUC-936 (SEQ ID NO:42), PCTUC-986 (SEQ ID NO:47), PCTUC-991 (SEQ ID NO:75), PCTUC-992 (SEQ ID NO:60), PCTUC-1054 (SEQ ID NO:59), PCTUC-1061 (SEQ ID NO:55), PCTUC-1073 (SEQ ID NO:56), PCTUC-1075 (SEQ ID NO:73), PCTUC-1078 (SEQ ID NO:68), PCTUC-1082 (SEQ ID NO:54), PCTUC-1122 (SEQ ID NO:62), and PCTUC-250 (SEQ ID NO:41) respectively.

### DETAILED DESCRIPTION OF THE INVENTION

The invention relates to genes associated with a cancerous state. It has been discovered that the level of expression of individual genes, also referred to as markers, and combinations of these genes, correlates with the presence of cancer in a patient. Methods are provided for detecting the presence of cancer in a sample; the absence of cancer in a sample, the stage of a cancer, and with other characteristics of cancer that are relevant to prevention, diagnosis, characterization, and therapy of cancer in a patient. The invention also relates to small molecule or antibody therapeutics for the treatment of carcinomas.

The present invention is based, in part, on identification of novel markers, which are over-expressed in colon cancer cells as compared to their expression in normal (i.e. non-cancerous) colon cells. The markers of the invention correspond to DNA, RNA, and polypeptide molecules, which can be detected in one or both of normal and cancerous colon cells. The enhanced expression of one or more of these markers in colon cells is herein correlated with the cancerous state of the tissue. The invention thus includes compositions, kits, and methods for assessing the cancerous state of colon cells (e.g. cells obtained from a human, cultured human cells, archived or preserved human cells and in vivo cells).

The present invention relates to:

An isolated polypeptide comprising an amino acid sequence selected from the group consisting of:
(a) a mature form of an amino acid sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114;
(b) a variant of a mature form of an amino acid sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114, wherein one or more amino acid residues in said variant differs from the amino acid sequence of said mature form, provided that said variant differs in no more than 20%, of the amino acid residues from the amino acid sequence of said mature form;
(c) an amino acid sequence comprising a sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114; and
(d) a variant of an amino acid sequence comprising a sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114, wherein one or more amino acid residues in said variant differs from the amino acid sequence, provided that said variant differs in no more than 20% of amino acid residues from said amino acid sequence.

An isolated polypeptide wherein the amino acid sequence is selected from the group consisting of: SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114.

An isolated polypeptide wherein the amino acid sequence is selected from the group consisting of: SEQ ID NO:39; SEQ ID NO:56; SEQ ID NO:59; SEQ ID NO:61; SEQ ID NO:62; SEQ ID NO:65; and SEQ ID NO:68.

An isolated polypeptide wherein the amino acid sequence is selected from the group consisting of: SEQ ID NO:43; SEQ ID NO:46; SEQ ID NO:47; SEQ ID NO:54; SEQ ID NO:55; SEQ ID NO:57; SEQ ID NO:58; SEQ ID NO:60; SEQ ID NO:63; SEQ ID NO:64; SEQ ID NO:66; SEQ ID NO:67; SEQ ID NO:70; SEQ ID NO:73, and SEQ ID NO:3114.

An isolated polypeptide wherein the amino acid sequence is selected from the group consisting of: SEQ ID NO:40; SEQ ID NO:41; SEQ ID NO:42; SEQ ID NO:48; SEQ ID NO:49; SEQ ID NO:53; SEQ ID NO:71; SEQ ID NO:72; and SEQ ID NO:75.

An isolated polypeptide wherein the amino acid sequence is selected from the group consisting of: SEQ ID NO: 39; SEQ ID NO:40; SEQ ID NO:41; SEQ ID NO:42; and SEQ ID NO:43.

An isolated polypeptide wherein said amino acid sequence is encoded by a nucleic acid sequence selected from the group consisting of SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113.

An polypeptide wherein said polypeptide comprises an amino acid sequence of a naturally-occurring allelic variant of an amino acid sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114.

An polypeptide wherein said allelic variant comprises an amino acid sequence that is the translation of a nucleic acid sequence differing by a single nucleotide from a nucleic acid sequence selected from the group consisting of SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113.

A polypeptide variant wherein the amino acid substitution is a conservative amino acid.

A nucleic acid molecule selected from the group consisting of SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:1; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113.

A nucleic acid molecule selected from the group consisting of SEQ ID NO:1; SEQ ID NO:18; SEQ ID NO:21; SEQ ID NO:23; SEQ ID NO:24; SEQ ID NO:27; and SEQ ID NO:30.

A nucleic acid molecule selected from the group consisting of SEQ ID NO:5; SEQ ID NO:8; SEQ ID NO:9; SEQ ID NO:16; SEQ ID NO:17; SEQ ID NO:19; SEQ ID NO:20; SEQ ID NO:22; SEQ ID NO:25; SEQ ID NO:26; SEQ ID NO:28; SEQ ID NO:29; SEQ ID NO:32; SEQ ID NO:35; and SEQ ID NO:3113.

A nucleic acid molecule selected from the group consisting of SEQ ID NO:2; SEQ ID NO:3; SEQ ID NO:4; SEQ ID NO:10; SEQ ID NO:11; SEQ ID NO:15; SEQ ID NO:33; SEQ ID NO:34; SEQ ID NO:37.

A nucleic acid molecule selected from the group consisting of SEQ ID NO:1; SEQ ID NO:2; SEQ ID NO:3; SEQ ID NO:4; and SEQ ID NO:5.

A nucleic acid molecule that differs by a single nucleotide from a nucleic acid sequence selected from the group consisting of: SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113.

A nucleic acid molecule that hybridizes under stringent conditions to a nucleotide sequence selected from the group consisting of: SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID. NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113, or a complement of said nucleotide sequence.

A vector comprising a nucleic acid molecule of the present invention.

A vector comprising a nucleic acid molecule of the present invention oberably linked to a promoter.

A cell transformed or transfected with a vector of vector of the present invention.

A cell transformed or transfected with a nucleic acid sequence selected from the group SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113

A microarray comprising nucleic acid sequences selected from the group consisting of SEQ ID NO.77 through SEQ ID NO:3011.

A microarray comprising nucleic acid sequences selected from the group consisting of SEQ ID NO:77 through SEQ ID NO:1993.

A microarray comprising nucleic acid sequences selected from the group consisting of SEQ ID NO:1994 through SEQ ID NO:3011.

A microarray comprising a nucleic acid sequence selected from the group consisting of SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:11; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113.

A microarray comprising a nucleic acid sequence selected from the group consisting of SEQ ID NO:1; SEQ ID NO:18; SEQ ID NO:21; SEQ ID NO:23; SEQ ID NO:24; SEQ ID NO:27; and SEQ ID NO:30.

A microarray comprising a nucleic acid sequence selected from the group consisting of SEQ ID NO:1; SEQ ID NO:18; SEQ ID NO:21; SEQ ID NO:23; SEQ ID NO:24; SEQ ID NO:27; and SEQ ID NO:30.

A microarray comprising a nucleic acid sequence selected from the group consisting of SEQ ID NO:5; SEQ ID NO:8; SEQ ID NO:9; SEQ ID NO:16; SEQ ID NO:17; SEQ ID NO:19; SEQ ID NO:20; SEQ ID NO:22; SEQ ID NO:25; SEQ ID NO:26; SEQ ID NO:28; SEQ ID NO:29; SEQ ID NO:32; SEQ ID NO:35; and SEQ ID NO:3113.

A microarray comprising a nucleic acid sequence selected from the group consisting of SEQ ID NO:2; SEQ ID NO:3; SEQ ID NO:4; SEQ ID NO:10; SEQ ID NO:11; SEQ ID NO:15; SEQ ID NO:33; SEQ ID NO:34; and SEQ ID NO:37.

A microarray comprising a nucleic acid sequence selected from the group consisting of SEQ ID NO:1; SEQ ID NO:2; SEQ ID NO:3; SEQ ID NO:4; and SEQ ID NO:5.

An antibody that immunospecifically-binds to the polypeptide of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114.

The antibody may be monoclonal antibody, an antibody fragment selected from but not limited to a FV fragment, a Fab fragment, (Fab)₂ fragment, a single chain antibody.

The antibody may be a conjugated with at least one polyethylene glycol moiety.

The antibody may be an antagonist.

The antibody may be a humanized antibody or a human antibody.

A method for determining the presence or amount of the polypeptide of the present invention in a sample, the method comprising
(a) providing the sample;
(b) contacting the sample with an antibody that binds immunospecifically to the polypeptide; and
(c) determining the presence or amount of antibody bound to said polypeptide;
thereby determining the presence or amount of polypeptide in said sample.

A method for determining the presence or amount of the nucleic acid molecule of the present invention in a sample, the method comprising:
(a) providing the sample;
(b) contacting the sample with a probe that binds to said nucleic acid molecule; and
(c) determining the presence or amount of the probe bound to said nucleic acid molecule;
thereby determining the presence or amount of the nucleic acid molecule in said sample.

A method of identifying an agent that binds to a polypeptide of claim 1, the method comprising:
(a) contacting said polypeptide with said agent; and
(b) determining whether said agent binds to said polypeptide.

A method for identifying an agent that modulates the expression or activity of the polypeptide of claim 1, the method comprising:
(a) providing a cell expressing said polypeptide in an operational manner;
(b) contacting the cell with said agent; and
(c) determining whether the agent modulates expression or activity of said polypeptide;
whereby an alteration in expression or activity of said peptide indicates said agent modulates expression or activity of said polypeptide.

A method for modulating the activity of the polypeptide of the present invention, the method comprising contacting a cell sample expressing the polypeptide with a compound that binds to the polypeptide in an amount sufficient to modulate the activity of the polypeptide.

A method of treating or preventing a cancer-associated disorder, said method comprising administering to a subject in which such treatment or prevention is desired the polypeptide of present invention in an amount sufficient to treat or prevent said cancer-associated disorder in said subject.

A method of treating or preventing a cancer-associated disorder, said method comprising administering to a subject in which such treatment or prevention is desired an antibody of the present invention in an amount sufficient to treat or prevent said cancer-associated disorder in said subject.

A pharmaceutical composition comprising a polypeptide of the present invention and at least one pharmaceutically acceptable carrier.

A kit comprising the pharmaceutical composition of the present invention.

A method of detecting differentially expressed genes correlated with a cancerous state of a mammalian cell, the method comprising the step of detecting at least one differentially expressed gene product in a test sample derived from a cell suspected of being cancerous, where the gene product is encoded by a sequence of SEQ ID NO:1; SEQ ID NO:2; SEQ ID NO:3; SEQ ID NO:4; SEQ ID NO:5; SEQ ID NO:8; SEQ ID NO:9; SEQ ID NO:10; SEQ ID NO:1; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:32; SEQ ID NO:33; SEQ ID NO:34; SEQ ID NO:35; SEQ ID NO:37; and SEQ ID NO:3113 wherein detection of differentially expressed product is correlated with a cancerous state of the cell from which the test sample was derived.

A method for detecting the presence of a nucleic acid molecule of the present invention in a sample comprising:
(a) contacting the sample with a nucleic acid probe or primer which selectively hybridizes to the nucleic acid molecule; and
(b) determining whether the nucleic acid probe or primer binds to a nucleic acid molecule in the sample to thereby detect the presence of a nucleic acid molecule in the sample.

A method for detecting the presence of a nucleic acid molecule of the present invention in a sample comprising:
(a) contacting the sample with a nucleic acid probe or primer which selectively hybridizes to the nucleic acid molecule; and
(b) determining whether the nucleic acid probe or primer binds to a nucleic acid molecule in the sample to thereby detect the presence of a nucleic acid molecule in the sample.

A method for detecting the presence of a nucleic acid molecule selected from the group consisting of SEQ ID NO:77 through SEQ ID NO:3011 in a sample comprising:
(a) contacting the sample with a nucleic acid probe, or primer which selectively hybridizes to the nucleic acid molecule; and
(b) determining whether the nucleic acid probe or primer binds to a nucleic acid molecule in the sample to thereby detect the presence of a nucleic acid molecule selected from the group consisting of SEQ ID NO:77 through SEQ ID NO:3011 in the sample.

A method for detecting the presence of a nucleic acid molecule selected from the group consisting of SEQ ID NO:77 through SEQ ID NO:1993 in a sample comprising:
(a) contacting the sample with a nucleic acid probe or primer which selectively hybridizes to the nucleic acid molecule; and
(b) determining whether the nucleic acid probe or primer binds to a nucleic acid molecule in the sample to thereby detect the presence of a nucleic acid molecule selected from the group consisting of SEQ ID NO:77 through SEQ ID NO:1993 in the sample.

A method for detecting the presence of a nucleic acid molecule selected from the group consisting of SEQ ID NO:1994 through SEQ ID NO:3011 in a sample comprising:
(a) contacting the sample with a nucleic acid probe or primer which selectively hybridizes to the nucleic acid molecule; and
(b) determining whether the nucleic acid probe or primer binds to a nucleic acid molecule in the sample to thereby detect the presence of a nucleic acid molecule selected from the group consisting of SEQ ID NO:1994 through SEQ ID NO:3011 in the sample.

A method for monitoring the progression of a cancer in a patient, the method comprising:
(a) detecting in a patient sample at a first point in time, the expression of a marker, wherein the marker a nucleic acid molecule of the present invention;
(b) repeating step a) at a subsequent point in time; and
(c) comparing the level of expression detected in steps a) and b), and therefrom monitoring the progression of the cancer.

A method for monitoring the progression of a cancer in a patient, the method comprising:
(a) detecting in a patient sample at a first point in time, the expression of a marker, wherein the marker a nucleic acid molecule of of the present invention;
(b) repeating step a) at a subsequent point in time; and
(c) comparing the level of expression detected in steps a) and b), and therefrom monitoring the progression of the cancer.

A method for monitoring the progression of a cancer in a patient, the method comprising:
(a) detecting in a patient sample at a first point in time, the expression of a marker, wherein the marker is a nucleic acid molecule selected from the group consisting of SEQ ID NO:77 through SEQ ID NO:3011;
(b) repeating step a) at a subsequent point in time; and
(c) comparing the level of expression detected in steps a) and b), and therefrom monitoring the progression of the cancer.

A method for monitoring the progression of a cancer in a patient, the method comprising:
(a) detecting in a patient sample at a first point in time, the expression of a marker, wherein the marker is a nucleic acid molecule selected from the group consisting of SEQ ID NO:77 through SEQ ID NO:1993;
(b) repeating step a) at a subsequent point in time; and
(c) comparing the level of expression detected in steps a) and b), and therefrom monitoring the progression of the cancer.

A method for monitoring the progression of a cancer in a patient, the method comprising:
(a) detecting in a patient sample at a first point in time, the expression of a marker, wherein the marker is a nucleic acid molecule selected from the group consisting of SEQ ID NO:1994 through SEQ ID NO:3011;
(b) repeating step a) at a subsequent point in time; and
(c) comparing the level of expression detected in steps a) and b), and therefrom monitoring the progression of the cancer.

A method of assessing the efficacy of a test compound for inhibiting a cancer in a patient, the method comprising comparing:
(a) expression of a marker in a first sample obtained from the patient exposed to the test compound, wherein the marker is selected the nucleic acid molecule of the present invention, and
(b) expression of the marker in a second sample obtained from the patient, wherein the sample is not exposed to the test compound, wherein a significantly lower level of expression of the marker in the first sample, relative to the second sample, is an indication that the test compound is efficacious for inhibiting the cancer in the patient.

A method of assessing the efficacy of a test compound for inhibiting a cancer in a patient, the method comprising comparing:
(a) expression of a marker in a first sample obtained from the patient exposed to the test compound, wherein the marker is selected the a nucleic acid molecule selected from the group consisting of SEQ ID NO:77 through SEQ ID NO:1993, and
(b) expression of the marker in a second sample obtained from the patient, wherein the sample is not exposed to the test compound, wherein a significantly lower level of expression of the marker in the first sample, relative to the second sample, is an indication that the test compound is efficacious for inhibiting the cancer in the patient.

A method of assessing the efficacy of a therapy for inhibiting a cancer in a patient, the method comprising comparing:
(a) expression of a marker in the first sample obtained from the patient prior to providing at least a portion of the therapy to the patient, wherein the marker is a nucleic acid sequence selected from the group consisting of SEQ ID NO:1994 through SEQ ID NO:3011, and
(b) expression of the marker in a second sample obtained from the patient following provision of the portion of the therapy, wherein a significantly lower level of expression of the marker in the second sample, relative to the first sample, is an indication that the therapy is efficacious for inhibiting the cancer in the patient.

A method of selecting a composition for inhibiting a cancer in a patient, the method comprising:
(a) obtaining a sample comprising cancer cells from the patient;
(b) separately exposing aliquots of the sample in the presence of a plurality of test compositions;
(c) comparing expression of a marker in each of the aliquots, wherein the marker is the nucleic acid of of the present invention; and
(d) selecting one of the test compositions which alters the level of expression of the marker in the aliquot containing that test composition, relative to other test compositions.

A method of selecting a composition for inhibiting a cancer in a patient, the method comprising:
(a) obtaining a sample comprising cancer cells from the patient;
(b) separately exposing aliquots of the sample in the presence of a plurality of test compositions;
(c) comparing expression of a marker in each of the aliquots, wherein the marker is the nucleic acid of the present invention; and
(d) selecting one of the test compositions which alters the level of expression of the marker in the aliquot containing that test composition, relative to other test compositions.

A method of selecting a composition for inhibiting a cancer in a patient, the method comprising:
(a) obtaining a sample comprising cancer cells from the patient;
(b) separately exposing aliquots of the sample in the presence of a plurality of test compositions;
(c) comparing expression of a marker in each of the aliquots, wherein the marker is a nucleic acid sequence selected from the group consisting of SEQ ID NOs:77-1993; and
(d) selecting one of the test compositions which alters the level of expression of the marker in the aliquot containing that test composition, relative to other test compositions.

A method of selecting a composition for inhibiting a cancer in a patient, the method comprising:
(a) obtaining a sample comprising cancer cells from the patient;
(b) separately exposing aliquots of the sample in the presence of a plurality of test compositions;
(c) comparing expression of a marker in each of the aliquots, wherein the marker is a nucleic acid sequence selected from the group consisting of SEQ ID NOs:1994-3011; and
(d) selecting one of the test compositions which alters the level of expression of the marker in the aliquot containing that test composition, relative to other test compositions.

An antisense compound 8 to 30 nucleobases in length targeted to a nucleic acid molecule of the present invention encoding the polypeptide of the present invention, wherein said antisense compound specifically hybridizes with and inhibits the expression of the polypeptide.

Transcriptional profiling analysis was performed on primary colon tumors. The mRNA was isolated from the colon tumors and mRNA was isolated from normal colons. Transcripts that were differentially expressed greater than 2 fold and in 30% or more of the tumors were further analyzed by Signal P analysis and Trans Membrane Hidden Markov Models (TMHMM) to determine if they were membrane associated or secreted. The transcripts determined to be associated with the membrane were then further validated by Sybrgreen (real time PCR) analysis on an additional set of colon and breast tumors compared to corresponding normal tissue.

These sequences that were differentially expressed can be used to develop new small molecule and antibody therapeutics for the treatment of carcinoma, including colon, lung, breast, and prostate tumors. All of these sequences also provide potential biomarkers and efficacy markers for carcinoma. The sequences can be used to generate antibody reagents, full length clones, and can be evaluated in in vitro and in vivo assays to determine function.

The present invention discloses the nucleic acid sequences SEQ ID NOs:1994-3011, which are down-regulated in human colon tumor cells.

The present invention discloses the nucleic acid sequences SEQ ID NOs:1-11, SEQ ID NOs:13-38, SEQ ID NOs:77-1993, and SEQ ID NO:3113, which are up-regulated in human colon tumor cells.

The present invention discloses 31 transcripts, and the proteins encoded by them that are up-regulated in human colon tumor cells. A summary of these differentially expressed genes is included in Table 1.

TABLE 1

| Transcript | | GenBank | Gene | Protein |
|---|---|---|---|---|
| Designation | Gene Name | acc number | sequence | sequence |
| PCTUC_5 | (p-cadherin) | NM 001793 | SEQ ID NO: 1 | SEQ ID NO: 39 |
| PCTUC_93 | (opiate binding- | NM 002545 | SEQ ID NO: 8 | SEQ ID NO: 46 |
| | cell adhesion molecule) | | | |
| PCTUC_190 | (organic anion transporter | NM016354 | SEQ ID NO: 19 | SEQ ID NO: 57 |
| | polypeptide-related | | | |
| | protein 1, OATPRP1) | | | |
| PCTUC_239 | (carcinoembryonic | NM 004363 | SEQ ID NO: 27 | SEQ ID NO: 65 |
| | antigen-related cel | | | |
| | adhesion molecule 5) | | | |
| | (CEACAM5) | | | |
| PCTUC_246 | (RON kinase) | NM 002447 | SEQ ID NO: 2 | SEQ ID NO: 40 |
| PCTUC_250 | G-protein couple | NM 005682 | SEQ ID NO: 3 | SEQ ID NO: 41 |
| | receptor (GPR56) | | | |
| PCTUC_360 | (STEAP1) | NM 012449 | SEQ ID NO: 15 | SEQ ID NO: 53 |
| PCTUC_462 | (bumetanide-sensitive | NM 001046 | SEQ ID NO: 20 | SEQ ID NO: 58 |
| | Na—K—Cl | | | |
| | cotransporter, NKCC1) | | | |
| PCTUC_468 | (neuroligin_1, NLGN1) | NM 014932 | SEQ ID NO: 10 | SEQ ID NO: 48 |
| PCTUC_536 | (COLNOV1) | NM 033408 | SEQ ID NO: 3113 | SEQ ID NO: 3114 |
| PCTUC_582 | (CEACAM8) | NM 001816 | SEQ ID NO: 26 | SEQ ID NO: 64 |
| PCTUC_605 | (epican, CD44) | NM 000610 | SEQ ID NO: 33 | SEQ ID NO: 71 |
| PCTUC_629 | (monocarboxylate | NM 004207 | SEQ ID NO: 29 | SEQ ID NO: 67 |
| | transporter) (MCT3) | | | |
| PCTUC_722 | (integrin beta(4)subunit) | NM 000213 | SEQ ID NO: 23 | SEQ ID NO: 61 |
| PCTUC_748 | (T245 protein, T245) | NM 003270 | SEQ ID NO: 25 | SEQ ID NO: 63 |
| PCTUC_784 | (membrane cofactor | NM 002389 | SEQ ID NO: 34 | SEQ ID NO: 72 |
| | protein, CD46, MCP) | | | |
| PCTUC_812 | (protocadherin-9) (PCDH9) | NM 020403 | SEQ ID NO: 28 | SEQ ID NO: 66 |
| PCTUC_856 | (extraneuronal | NM 021977 | SEQ ID NO: 11 | SEQ ID NO: 49 |
| | monoamine transporter) | | | |
| PCTUC_898 | (KIAA0792) | NM 014698 | SEQ ID NO: 5 | SEQ ID NO: 43 |
| PCTUC_935 | (voltage gated potassium | NM 000218 | SEQ ID NO: 32 | SEQ ID NO: 70 |
| | channel, KCNQ1) | | | |
| PCTUC_936 | (FGFR3) | NM 000142 | SEQ ID NO: 4 | SEQ ID NO: 42 |
| PCTUC_986 | (senescence-associated | NM 021101 | SEQ ID NO: 9 | SEQ ID NO: 47 |
| | epithelial membrane | | | |
| | protein, SEMP1) | | | |
| PCTUC_991 | (osteoblast specific | NM 006475 | SEQ ID NO: 37 | SEQ ID NO: 75 |
| | factor 2, OSF-2os) | | | |
| PCTUC_992 | (NRAMP2) | NM 000617 | SEQ ID NO: 22 | SEQ ID NO: 60 |
| PCTUC_1054 | (polymorphic epithelial | NM 002456 | SEQ ID NO: 21 | SEQ ID NO: 59 |
| | mucin) (PEM) | | | |
| PCTUC_1061 | (KIAA0779) | AB018322 | SEQ ID NO: 17 | SEQ ID NO: 55 |
| PCTUC_1073 | (decay accelerating | NM 000574 | SEQ ID NO: 18 | SEQ ID NO: 56 |
| | factor for complement, | | | |
| | CD55, DAF) | | | |
| PCTUC_1075 | (solute carrier family 6 | NM 003043 | SEQ ID NO: 35 | SEQ ID NO: 73 |
| | member 6, neurotransmitter | | | |
| | transporter, taurine) | | | |
| PCTUC_1078 | (osteopontin) | NM 000582 | SEQ ID NO: 30 | SEQ ID NO: 68 |
| PCTUC_1082 | (claudin 2) | NM 020384 | SEQ ID NO: 16 | SEQ ID NO: 54 |
| PCTUC_1122 | (hepatoma transmembrane | NM 004093 | SEQ ID NO: 24 | SEQ ID NO: 62 |
| | kinase ligand, HTK ligand) | | | |

The following gene descriptions are shown as representative of the types of genes discovered in the present invention by the process described above. Since some of the genes are currently know to be associated with colon cancer, the identification of these genes by this method serves to validate the approach, such as: SEQ ID NO:1; SEQ ID NO:18; SEQ ID NO:21; SEQ ID NO:23; SEQ ID NO:24; SEQ ID NO:27; SEQ ID NO:30.

Description of Representtive Overexpressed Genes Found in Human Colon Tumors.

Opiate (Opioid) Binding-Cell Adhesion Molecule (OBCAM)

OBCAM as first identified by Cho et al., Proc Natl Acad Sci 80: 5176-80, (1983), was characterized as similar to other cell-adhesion molecules and named OBCAM by Schofield et al., EMBO J. February 8(2): 489-95, (1989). The gene was cloned and sequenced from the human brain by Shark and Lee, Gene 155: 213-17, (1995). It was shown to be a neuron specific protein, presumed to play a role as a cell adhesion/recognition molecule, but its function has not been fully elucidated. Indirect evidence indicates a role for this protein in opioid function. Studies have suggested that the function of OBCAM in the brain may involve axonal outgrowth.

SEMP1 (Claudin 1: CLDN1; Senescence-Associated Epithelial Membrane Protein 1)

Swisshelm et al., Gene 226: 285-95, (1999) isolated cDNAs encoding SEMP1 from human mammary epithelial cells. The mRNA was shown to be expressed in human tissues, including adult and fetal liver, pancreas, placenta, adrenals, prostate and ovary, but at low or undetectable levels in a number of human breast cancer cell lines. It is a member of a superfamily of epithelial membrane proteins (EMPs), which may have multiple potential functions, including maintenance and regulation of cell polarity and permeability, perhaps through mechanisms involving tight junctions, (ibid). First identified by Furuse et al., J Cell Biol 141: 1539-50, (1998), SEMP1 expression in normal human mammary epithelial cells, in contrast to low or undetectable levels of expression in a number of breast tumors and breast cancer cell lines, may indicate this protein as a possible tumor-suppressor gene, (Kramer et al., Hum Genet 107: 249-56, (2000)). Expression of this protein and tight junction morphology were altered in blood vessels of human glioblastoma multiforme, (Liebner et al., Acta Neuropathol 100 (3): 323-31, (2000)).

Neuroligin 1 (NLGN1)

NLGN1 was first identified as a neuronal cell surface protein and characterized as a splice site-specific ligand for beta-neurexins by Ichtchenko et al., Cell 81: 435-43, (1995). Neuroligin 1 binds to beta-neurexins only if they lack an insert in the alternatively spliced sequence of the G domain, not if they contain an insert. Findings support a model whereby alternative splicing of neurexins creates a family of cell surface receptors that confer interactive specificity-on their resident neurons, (ibid). It was also suggested that these proteins are part of the machinery employed during the formation and remodeling of CNS synapses, (Scheiffele et al., Cell 101: 657-69, (2000)). Cloned by Kikuno et al., DNA Res 6: 197-205, (1999), neuroligen 1 expression in mammary carcinomas may reflect deregulated gene expression. As carcinomas regressed in a study by Ariazi et al., J Biol Chem 271: 29286-94, (1996), neuroligen 1 expression was repressed or turned off, which is consistent with regulated gene expression in most normal tissue.

Extraneuronal Monoamine Transporter (EMT)

EMTs, polyspecific organic cation transporters in the liver, kidney, and intestine are critical for elimination of many endogenous amines as well as a wide array of drugs and environmental toxins. This gene was cloned from a human kidney carcinoma cell line by Grundemann et al., Nature Neurosci 1: 349-51, (1998). Northern blot analysis detected high level expression of the gene transcript in first-trimester and term placenta, skeletal muscle, prostate, aorta, liver, fetal lung, salivary gland, and adrenal gland. Moderate to low expression was detected in uterus, ovary, kidney, lymph node, lung, trachea, and fetal liver, (Verhaagh et al., Genomics 55: 209-18, (1999)). Wu et al., J. Biol Chem 273: 32776-86, (1998) suggested that this protein plays a significant role in the disposition of cationic neurotoxins and neurotransmitters in the brain. The first report of the extraneuronal monoamine transporter (uptake 2) was by Streich et al., Naunyn Schmiedebergs Arch Pharmacol 353(3): 328-33, (1996). Streich et al. reported that human glioma cells express this transporter and thus, glial cells in the human CNS endowed with this transporter are likely to contribute to the inactivation of neuronally released noradrenaline.

COLNOV1 (Homo Sapiens Hypothetical Protein MBC3205)

COLNOV1 was identified in The Cancer Genome Anatomy Project Database by the National Cancer Institute. It was cloned and submitted by Robert L. Strausberg to GenBank in September 2001 with no journal publication cited. Its function is not yet elucidated.

STEAP1

STEAP1 is a six transmembrane epithelial antigen of the prostate. The gene is expressed predominately in human prostate tissue and is up regulated in multiple cancer cell lines, including prostate, bladder, colon, ovarian, and Ewing sarcoma Hubert et al., Proc Natl Acad Sci 96: 14523-28, (1999). Study results support STEAP as a cell-surface tumor-antigen target for prostate cancer therapy and diagnostic imaging (ibid). The structure of the protein suggests a potential function as a channel or transporter protein. STEAP1 was first reported and cloned by Hubert et al (1999).

Claudin 2

Claudin 2 was first identified from a chicken liver by Furuse et al., J Cell Biol 141: 1539-50, (1998). It is an integral membrane protein localized at tight junctions. Research has concluded that claudins regulate the intestinal barrier in response to immune mediators, (Kinugasa et al., Gastroenterology 118(6): 1001-11, (2000)). It is not clear, but has been speculated that this protein as well as other tight junction proteins may play a role in the molecular mechanism of brain tumor edema, (Papadopoulos et al., Br J Neurosurg 15(2): 101-8, (2001)). Furose et al., J Cell Biol 153(2): 263-272, (2001) cloned the canine equivalent.

KIAA0779

KIAA0779 was first identified and cloned from human brain by Nagase et al., DNA Res 5(5): 277-86, (1998). It may be functionally related to cell signaling/communication, cell structure/motility and nucleic acid management, (ibid).

Decay Accelerating Factor for Complement (CD55, DAF)

CD55 is a 70-kD glycoprotein that aids host tissues to avoid attack by autologous complement proteins. DAF interrupts the complement sequence at an early step in activation, effectively halts progression of the cascade and prevents consequent cell injury. DAF is expressed on the plasma membrane of all cell types that are in intimate contact with plasma complement proteins, (Koretz et al., Br J Cancer 66: 810-814, (1992). Cloned and characterized by Medof et al., Proc Nat Acad Sci 84: 2007-11, (1987), DAF can also act as a signal-transducing molecule. With monoclonal antibodies directed against DAF, human monocytes can be activated in vitro, (Shibuya et al., J Immunol 149: 1758-62, (1992)). Hensel et al., Laboratory Investigation 81: 1553-63, (2001) have isolated a human antibody SC-1, from a patient with a signet ring cell carcinoma of the stomach which induced apoptosis of gastric cancer cells in vitro and is being used successfully in clinical trials, (Vollmers, et al., Oncol Rep 5: 549-52, (1988)). The target for the antibody was discovered to be a modified DAF protein, (Hensel, et al, (2001)). DAF is over expressed on various tumors such as breast, colon, and stomach carcinoma, (Koretz et al., (1992)). Nowicki et al., Am J Reprod Immunol 46(2): 144-8, (2001), have reported that expression of DAF in endometrial adenocarcinoma is inversely related to the stage of tumor. This is consistent with the hypothesis that early stage endometrial adenocarcinoma that is exposed to complement attack may up-regulate DAF to protect malignant cells from complement lysis.

OATPRP1 (Solute Carrier Family 21 (Organic Anion Transporter), Member 12

OATPRP1 was submitted to GenBank Nov. 16, 1999 by Wu et al., (unpublished) as part of identification and characterization of novel human OATP family members.

NKCC1 (Solute Carrier Family 12, Member 2; SLC12A2)

The Na—K—Cl cotransporter, NKCC1 aids transcellular movement of chloride across both secretory and absorptive epithelia. It is reportedly expressed in many tissues, including the basolateral membrane of secretory epithelia where it mediates active chloride secretion, (Quaggin et al., Mamm Genome 6 (8): 55778, (1995)). Specifically, this protein is a bumetanide-sensitive Na—K—Cl cotransporter first discovered by Xu et al., Proc Natl Acad Sci 91: 2201-05, (1994). Cloned and characterized by Payne et al., J Biol Chem 270: 17977-85, (1995), the authors proposed that the NKCC1 gene is involved in the control of normal cell proliferation, while its overexpression results in apparent cell transformation, in a manner similar to some protooncogenes, (Panet et al., J Cell Phys 182: 109-18, (2000)).

PEM (Polymorphic Epithelial Mucin; Mucin 1, MUC1; Peanut-Reactive Urinary Mucin; PUM Mucin; Tumor-Associated Epithelial PEM)

PEM is a large cell surface mucin glycoprotein expressed by most glandular and ductal epithelial cells and some hematopoietic cell lineages. It has a tandem repeat domain that is highly O-glycosylated and alterations in glycosylation have been shown in epithelial cancer cells. PEM was first identified by Karlsson et al., (Ann Hum Genet 47: 263-9, (1983)) as a polymorphism identified from human urinary mucin by SDS polyacrylamide gel electrophoresis followed by detection with radiolabelled lectins. Peanut agglutinin was the most effective lectin; hence one of the proposed names. It is expressed in other normal and malignant tissues of epithelial origin including the mammary gland. This protein is developmentally regulated and aberrantly expressed in breast cancer, (Gendler et al, J Biol Chem 265: 15286-93, (1990)). Individuals with small PEM alleles/genotypes have an increased risk for development of gastric carcinoma, (Silva, et al., Eur J Hum Genet 9(7): 548-52, (2001)). PEM is activated in B-cell lymphoma by translocation and is rearranged and amplified in B-cell lymphoma subsets, (Dyomin, et al., Blood 95: 2666-71, (2000)). PEM was cloned and characterized by Gendler, et al., (1990).

NRAMP2 (Natural Resistance-Associated Macrophage Protein 2; Divalent Cation Transporter 1; DCT1; Divalent Metal Transporter 1; DMTI; Solute Carrier Family 11 (Proton-Coupled Divalent Metal Ion Transporter), Member 2: SLC11A2

NRAMP2 was first identified in the mouse genome by Gruenheid et al., Genomics 25: 514-25, (1995). Human NRAMP2 was isolated and characterized by Vidal et al., Mammalian Genome 6: 224-30, (1995). Its function is mediation of active transport that is proton-coupled and dependent on the cell membrane potential. It is up regulated by dietary iron deficiency and may represent a key mediator of intestinal iron absorption, (Gunshin et al., Nature 388: 482-88, (1997); Tandy, et al., J Biol Chem 275(2): 1023-29, (2000)).

Integrin, Beta-4 (ITGB4)

Integrin beta 4 was first identified, characterized, and cloned by Suzuki, et al., EMBO J. 9(3):757-63, (1990). Integrins are transmembrane glycoprotein receptors that mediate cell-matrix or cell-cell adhesion, and transduce signals that regulate gene expression and cell growth, (Vidal et al., Nature Genet 10:229-34, (1995)). Integrins are heterodimeric molecules that consist of noncovalently linked alpha and beta subunits. Alpha-6/beta-4 is restricted to the ventral surface opposed to the basal membrane zone, suggestive of its role in cell-matrix adhesion. Consistent with this possibility, alpha-6/beta-4 is found to be associated with the hemidesmosomes in stratified and transitional epithelia, (ibid). The cytoplasmic domain of the integrin beta-4 subunit mediates both association with the hemidesmosomal cytoskeleton and recruitment of the signaling adaptor protein SHC. This integrin is a receptor for the laminins, and likely plays a role in invasive carcinomas. Shaw et al., Cell 91: 949-60, (1997) demonstrated in the MDA-MB435 breast carcinoma cell line, that the alpha-6/beta-4 integrin promotes carcinoma invasion through a preferential and localized targeting of phosphoinositide-30H kinase (P13K) activity. Expression of alpha-6/beta-4 integrin has been shown to increase during malignant conversion of mouse epidermal keratinocytes, (Gomez et al., Exp Cell Res 201: 250-61, (1992)).

HTK Ligand (Hepatoma Transmembrane Kinase Ligand; EPH-Related Receptor Tyrosine Kinase Ligand 5: EPLG5; Ligand of EPH-Related kinase 5; LERK5; HTKL; EPHRIN B2; EFNB2)

EPHRIN B2 was first reported by Carpenter, et al., J Neurosci Res 42(2): 199-206, (1995) and cloned from hematopoietic monocytic lineage by Bennett et al., Proc Natl Acad Sci 92(6): 1866-70, (1995). Studies by Sakano, et al., Oncogene 13(4): 813-22, (1996) suggest the involvement of the HTK-HTKL system in the proliferation of HTK+ hematopoietic progenitor cells in the hematopoietic environment. According to Bennett et al., (1995), this ligand and its receptor are widely expressed and may function in a variety of tissues. HTK ligand is reportedly expressed in adult lung and kidney and in fetal heart, lung, kidney, and brain, (Cerretti et al., Immun 32: 1197-1205, (1995)). Unlike most of its family members, the ligand's receptor doesn't appear to be expressed in the CNS. However, similar to other members, it is expressed in primary epithelia and epithelial cell-derived lines, (Bennett et al., (1995)). The HTK-HTKL interaction may represent an early step in the initiation of synapse formation or maturation and may potentiate the ability of the receptor to respond to activity-dependent signals from the extracellular milieu, (Takasu, et al., Science 295: 491-95, (2002)). It is reported that HTK ligand transcripts are highly expressed in several small lung cell carcinoma cell lines and may modulate the biological behavior of these cells through autocrine and/or juxtacrine activation, (Tang et al., Hum Mol Genet 4: 2033-45, (1995)). Increased HTKL expression possibly reflects or induces an increased potential for growth, tumorigenicity, and metastatic abilities in human melanomas. The HTK-HTKL system could be a potential new source for molecular markers as well as a target for new therapies, (Vogt, et al., Clin Cancer Res 4(3): 791-7, (1998)). Studies by Liu et al., Cancer 94(4): 934-9, (2002) demonstrate that the ligand is expressed differentially in colon carcinoma and normal mucosa specimens and thus may play a role in the progression of colon carcinoma.

T245 Protein (T245: TM4SF6; Transmembrane 4 Superfamily Member 6)

Several TM4SF proteins have been shown to stimulate or modulate cell growth, (Bell et al., J Exp Med 175: 527-536, (1992); Higashiyama et al., J Cell Biol 128: 929-938, (1995); Lebel-Binay et al., J Immunol 155: 101-110, (1995); Maecker et al., FASEB J 11: 428-442, (1997)). Some may associate with integrins and control cell adhesion and movement, (Hadjiargyrou et al., J Neurochem 67: 2505-2513, (1996); Hemler et al., Biochem Biophys Acta 1287: 67-71, (1996); Maecker et al., (1997)). TM4SF6 was identified, cloned, and characterized from a human glioma by Maeda et al., Genomics 52: 240-242, (1998). Gene expression is reportedly widely found in human adult tissues. Among the tissues with high-level expression are the liver, pancreas, kidney, and ovary, while low-level expression was observed in the skeletal muscle, lung, and brain, (Maeda et al., (1998

Carcinoembryonic Antigen-Related Cell Adhesion Molecule 5 (CEACAM 5)

CEACAM5 was first described by Gold and Freedman, J Exp Med 121: 439-62, (1965) as a complex immunoreactive glycoprotein comprised of 60% carbohydrate. It is found in adenocarcinomas of endodermally derived digestive system epithelia and in fetal colon. The CEA immunoassay is useful in the diagnosis and serial monitoring of cancer patients for recurrent disease or response to therapy, particularly in colon cancer. CEACAM5 was isolated and characterized by Zimmermann et al., Proc Nat Acad Sci 84: 2960-64, (1987). The CEA gene was renamed CEACAM5, (Beauchemin et al., Exp Cell Res 252: 243-49, (1999)).

Protocadherin-9 (PCDH9)

Protocadherin-9 is a member of the cadherin superfamily. Protocadherins are a subfamily of calcium-dependent cell adhesion and recognition proteins. PCDH9 was first isolated and identified by Strehl et al., Genomics 53: 81-89, (1998) from a human fetal brain library. It is closely related to PCDH1 and PCDH7. Like other protocadherins, PCDH9 is predominately expressed in fetal and adult brain. They have a developmentally regulated expression pattern in brain that suggests this protein directs various aspects of morphogenesis, (Strehl et al., (1998

Monocarboxylate Transporter (MCT3)

The function of the proton-linked monocarboxylate transporter family is transport of substances across the plasma membrane. For example, lactic acid and pyruvate are transported via members of this family. Each member of the family appears to have slightly different substrate and inhibitor specificities and transport kinetics, which are related to the metabolic requirements of the tissues in which they are found. MCT3 was identified, cloned, and sequenced by Price et al, Biochem J 329: 321-28, (1998). MCT3 appears to be the MCT isoform expressed most in muscle fibers where energy metabolism is mainly glycolytic. It is a major route for lactic acid efflux from skeletal muscle and other cells, (Wilson, et al., J Biol Chem 273: 15920-26, (1998)). MCT3 is also found in retinal pigment epithelia, where it transports lactate between two tissue compartments, the interphotoreceptor matrix and the choriocapillaris, (Yoon et al., Biochem Biophys Res Comm 234: 90-94, (1997

Osteopontin (Secreted Phosphoprotein 1; SPP1: OPN: Bone Sialoprotein: Urinary Stone Protein)

Osteopontin is the principal phosphorylated glycoprotein of bone and is expressed in a limited number of other tissues including dentine, (Crosby et al., Genomics 27: 155-160, (1995)). Osteopontin is produced by osteoblasts under stimulation by calcitrol and binds tightly to hydroxyapatite. It has been shown to be involved in the anchoring of osteoclasts to the mineral of bone matrix, (Reinholt et al., Proc Nat Acad Sci 87: 4473-75, (1990)). Urinary calcium oxalate stones also consist of this protein, (Kohri et al., J Biol Chem 268: 15180-84, (1993)). Osteopontin is a constitutive component of normal elastic fibers in human skin and aorta. It has been suggested that it plays a role in modulating crystal nucleation and growth in mineralizing tissues and, more generally, in conditions in which mineral precipitation should be controlled, (Baccarini-Contri et al., Matrix Biol 14: 553-560, (1994)). This protein has been implicated in a number of pathologies, including but not limited to breast, colon, prostate, and lung cancers. There is evidence that osteopontin enhances malignancy, and that signaling pathways directly induced by this protein, as well as interactions with growth factor receptor pathways, can combine to activate expression of genes and functions that contribute to metastasis, (Furger et al., Curr Mol Med 1(5): 621-32, (2001)). Recent clinical evidence also suggests that osteopontin levels in cancer patients' blood or tumors may help provide prognostic information. The earliest report of this protein was by Oldberg, et al., Proc Natl Acad Sci 83 (23): 8819-23 (1986) where they cloned and sequenced osteopontin from a rat. The human gene was cloned and sequenced by Kiefer et al., Nucleic Acids Res 17: 3306, (1989).

Voltage-Gated Potassium Channel, KQT-Like Subfamily, Member 1 (KCNQ1)

KCNQ1 is a member of the largest and most diversified class of ion channels. Their main functions are associated with the regulation of the resting membrane potential and the control of the shape and frequency of action potentials. These channels are made up of multimeric proteins containing alpha subunits, which are directly responsible for channel activity, and gamma subunits, which modify the basic channel activity. KCNQ1 was first identified, cloned, and characterized by Want et al., Nat Genet 12 (1): 17-23, (1996). This specific protein is essential for the repolarization phase of the cardiac action potential and for K+ homeostasis in the inner ear, (Neyroud et al., Circ Res 84: 290-97, (1999)). Northern blot analyses and in situ hybridization indicate that KCNQ1 is expressed in kidney, pancreas, lung, placenta, inner ear, and in heart with the highest level of expression, (Sanguinetti et al., Nature 384: 80-83, (1996); Wang et al., Nat Genet 12: 17-23, (1996); Neyroud et al., Nat Genet 15: 186-89, (1997)). This gene is located on 11p15.5, in a large domain of contiguous genes abnormally imprinted in cancer and with Beckwith-Wiedemann syndrome, (Lee et al., Nat Genet 15: 181-85, (1997)). Mutations in KCNQ1 are the most frequent cause of the long-QT syndrome (LQTS), which is an inherited cardiac disorder that predisposes individuals to syncope, seizures, and sudden cardiac death from ventricular tachyarrhythmias, (Schwartz, et al., Am Heart J 89: 378-90, (1975)).

Epican

Epican is a heparin sulfate proteoglycan. This protein has a novel 339 amino acid domain inserted into the proximal extracellular domain of the standard, leukocyte form of CD44. It was first identified and characterized as a proteoglycan on human keratinocytes by Haggerty et al., J Invest Dermatol 99 (4): 374-80, (1992) and given the name epican, which stands for epidermal intercellular proteoglycan. Epican is expressed on the outer cell surface of squamous-cell carcinomas and can be a target for other tumors as well, (Van Hal, et al., Int J Cancer 68 (4): 520-27, (1996)).

Membrane Cofactor Protein (CD46; MCP; Measles Virus Receptor)

The level of complement activation on cell membranes is regulated by the expression of membrane-bound complement regulatory proteins, which protect normal and tumor cells from uncontrolled complement-mediated injury, (Gorter and Meri, Immunol Today 20: 576-582, (1999)). Membrane cofactor protein is one of these regulatory proteins. Several studies have shown that in situ tumor cells overexpress CD46, (Koretz et al., Br J Cancer 66: 810-814, (1992); Li et al., Br J Cancer 84: 80-86, (2001); Maenpaa et al., Am J Pathol 148: 1139-52, (1996); Niehans et al., Am J Pathol 149: 129-142, (1996); Yamakawa et al., Cancer 73: 2808-17, (1994)). The overexpression of this and other regulatory proteins of complement on tumor cells may prevent an efficient local immune response. According to Durrant and Spendlove in Curr Opin Investig Drugs 7:959-66, (2001), expression of the complement-regulatory proteins CD55, CD46 and CD59 are deregulated in cancer with tumors showing loss of one or more inhibitors and strong overexpression of others. This results in tumors that are resistant to attack by complement. Studies performed in vitro and in vivo have shown that tumor sensitivity to complement can be restored by co-administration of antibodies that bind to the functional domains of complement-regulatory proteins, (Durrant and Spendlove, (2001)). Sparrow et al., Hum Immunol 13: 83-93, (1985) first reported CD46 as HuLy-m5. Purcell et al., Immunogenetics 33: 335-44, (1991) isolated and cloned this protein.

Solute Carrier Family 6 Member 6 (Neurotransmitter Transporter, Taurine; SLC6A6)

Taurine is a major intracellular amino acid in mammals. It is involved in bile acid conjugation in hepatocytes, modulation of calcium flux and neural excitability, osmoregulation, detoxification, and membrane stabilization. The taurine transporter (SLC6A6) has considerable amino acid sequence similarity to sodium- and chloride-dependent transporters, (Uchida et al., Proc Nat Acad Sci 89: 8230-34, (1992)). Ramamoorthy et al., Biochem J 300: 893-900, (1994) cloned and characterized this gene from human placenta. Northern blot analysis by Ramamoorthy et al., (1994) revealed that the principal transcript is expressed abundantly in placenta and skeletal muscle, at intermediate levels in heart, brain, lung, kidney, and pancreas; while at low levels in liver. Cultured human cell lines from placenta, intestine, cervix, and retinal pigment epithelia also contain the transcript.

Osteoblast Specific Factor 2, OSF-2os: Periostin

This protein shares structural and sequence homology with fasciclin I, which is an insect adhesion molecule, (Takeshita et al., Biochem J 294: 271-278, (1993)). There is substantial evidence to suggest that cell-cell and cell-matrix adhesive interactions play a crucial role in tumorigenesis, tumor progression, and in particular metastasis, (Albeda, Lab Invest 68: 4-17, (1993); Tuszynski, et al., Acta Haematol 97: 29-39, (1997)). It has been observed that periostin was over expressed ten-fold in glioblastoma compared to normal brain tissue and the gene is overexpressed in several human tumors including carcinomas of ovary, breast and brain, (Lal et al., Cancer Res 59: 5403-07, (1999)). This protein is also over expressed in ovarian tumors as well, (Ismail et al., Cancer Res 60: 6744-49, (2000)). Results by Sasaki et al., Cancer Letters 172: 37-42, (2001) show that the periostin gene is highly expressed in the surrounding stromal cells of breast and lung cancer tissue but not within the tumor by in situ RNA hybridization, suggesting that expression of periostin may be involved in tumor invasion. This gene was first cloned and characterized by Takeshita et al., (1993). Periostin is expressed in bone and to a lesser extent in the lung, but not other tissues.

CEACAM8 (CGM6, CD66b, NCA-95, NCA-W272)

CEACAM8 is a member of the CEA gene family of cell adhesion molecules, which is primarily expressed in neutrophils and eosinophils (Eades-Perner et. al., (1998) Blood 91: 663-672). Two groups reported the cloning of the gene for CEACAM8 in 1990. Berling et. al. ((1990) Cancer Res 50:6534-6539) cloned the gene from a library constructed from a CML patient and Arakawa et. al. ((1990) BBRC 166:1063-1071) identified the same gene from a library prepared from normal human peripheral white blood cells.

FGFR3

Fibroblast Growth Factor Receptor 3 (“FGFR3”) is a member of the fibroblast growth factor receptor (“FGFR”) family of the receptor tyrosine kinase proteins (RTK's). Fibroblast growth factor receptors mediate growth, differentiation and cellular migration in a diverse range of cell types. Hart, K. C., et al., Mol. Biol. Cell., 12:931-941, 2001. Fibroblast growth factor (“FGF”) signaling plays a role in mitogenesis, mesoderm induction, neuronal survival and neuritic extension, tumor angiogenesis, and atherosclerosis, Pandit, S. G., et al., Biochem. J., 361:231-241, 2002. Ligand binding to the extracellular domain of the FGFR receptors induces receptor dimerization and transphosphorylation of tyrosine residues in the intracellular domains of the receptors. Links between FGFR3 and cancer has recently been uncovered (Hart, K. C., et al., Mol. Biol. Cell., 12:931-941, 2001; Pandit, S. G., et al., Biochem. J., 361:231-241, 2002). In 1990, FGFR3 was reported by Elena B. Pasquale as cek2, one of two novel kinases present in chicken embryos that were homologous to cek1, a chicken fibroblast growth factor receptor, Pasquale, E. B., Proc. Natl. Acad. Sci. USA, 87:5812-5816, 1990. In 1991, Keegan et al. (Natl. Acad. Sci. USA, 88:1095-1099, 1991) reported the isolation of the gene. The cDNA was sequenced in its entirety and designated as Fibroblast Growth Factor Receptor 3 (FGFR3), Anti-FGFR antibodies were first reported at least as earlier as 1991 (Bellot et al. EMBO J., 10:2849-2854, 1991). In 1991, Keegan et al. Oncogene, 6:2229-2236, 1991) attempted to create an antibody specific for FGFR3 but encountered significant difficulties in obtaining an antibody that did not cross react with other FGFR receptors.

GPR56 (TM7XN1, TM7LN4, EGF TM7-Like cDNA)

GPR56, also known as TM7XN1, TM7LN4 or as EGF TM7-like cDNA, is a novel g-protein coupled receptor (GPR) independently identified by two separate groups of research scientists in 1999 (Liu, M., et al. Genomics, 55:296-305, 1999: Zendman, A. J. W., et al., FEBS Letters, 446:292-298, 1999). GPR56, like other g-protein coupled receptors, is dominated by a seven transmembrane (TM7) domain consisting of seven hydrophobic stretches of amino acids, each forming a transmembrane domain that traverses the plasma membrane of the cell. These domains are highly conserved among the various g-protein coupled receptors. The function of a g-protein coupled orphan receptors remains unidentified. However, the presence of mucin-like and/or EGF-like domains suggests that GPR56 is involved in cell-cell binding. While GPR56 lacks the EGF domains, reports suggest that it may be involved in cell-cell adhesion through glycosylation of its N-terminal domain.

P Cadherin (Placental Cadherin, PCAD, Cadherin-3, CDH3 or CDHP)

Cadherins are a superfamily of transmembrane proteins regulating cell-cell adhesion during development and tissue homeostasis (Gumbiner, B. M., J. Cell. Biol., 148:399-404, 2000; Yagi, T. and Takeichi, M., Genes Dev., 14:1169-1180, 2000). Cadherins have five extracellular Ca2+ binding domains and a small cytoplasmic domain that is highly conserved among the classical cadherins. P-cadherin expression is often altered in only a subset of a given type of tumor. It is known that P-cadherin is upregulated in inflammatory bowel diseases such as Crohn's disease and colitis. Aberrant P-cadherin expression has been associated with cell proliferation and dedifferentiation in breast cancer (Gamallo, C., Modern Pathology, 14:650-654, 2001). Human P-cadherin was reported to be the antigen recognized by the NCC-CAD-299 monoclonal antibody raised against a vulvar epidermoid carcinoma (Shimoyama, Y., et al., Cancer Res., 49-2128-2133, 1989). The human gene was isolated by Shimoyama, Y., et al., J. Cell Biol., 109:1787-1794, 1989).

RON kinase

RON (Recepteur d'Origine Nantais) is a receptor protein tyrosine kinase belonging to the hepatocyte growth factor (“HGF”) receptor family. Protein tyrosine kinases are enzymes that transfer the terminal phosphate of adenosine triphosphate (ATP) to a specific tyrosine residue on a target protein. These enzymes are found in all multicellular organisms and play a central role in the regulation of cellular growth and in the differentiation of complex eukaryotes. There are two major classes of tyrosine kinases: transmembrane receptor tyrosine kinases and non-receptor tyrosine kinases. Transmembrane receptor tyrosine kinases are activated directly by binding of peptide growth factors and cytokines to their extracellular domains. Tyrosine kinases that fall within this class include the receptor for hepatocyte growth factor. The normal function of the receptor is to act as transducer of extracellular signals. The Ron gene encodes a 190 kDa protein, the mature form being a disulfide-linked heterodimer. RON comprises a 40-kD extracellular α chain and a 150-kD β chain having an intracellular protein tyrosine kinase domain, the activity of which is increased by ligand-receptor binding (Leonard, E. J. and Danilkovitch, A., Advances in Cancer Research, 2000, 139-165).

KIAA0792

KIAA0792 is a gene of unknown function, primarily expressed in the kidney, brain, ovary, lung and pancreas. The KIAA0792 transcript has been mapped to human chromosome 1 at position Iq42.13. The KIAA0792 locus contains 25 exons spread over 36,774 base pairs of genomic DNA. KIAA0792 consists of a 4074 nucleotide sequence encoding a protein of 807 amino acids. The sequences are available in Genbank, Accession Number AB018335. The protein encoded by KIAA0792 may contain as many as ten transmembrane domains, positioned at amino acids 67-89, 166-188, 212-234, 446-468, 487-509, 528-550, 583-605, 635-657, 689-711, and 717-739. In addition to these transmembrane domains, at least one other recognized domain, DUF221, is located at position numbers 356-806. This domain is present in a number of other putative transmembrane proteins of unknown function.

The present invention discloses genes that have not previously been reported to be over-expressed in human tumor tissue compared to corresponding normal tissue: SEQ ID NO:5; SEQ ID NO:8; SEQ ID NO:9; SEQ ID NO:16; SEQ ID NO:17; SEQ ID NO:19; SEQ ID NO:20; SEQ ID NO:22; SEQ ID NO:25; SEQ ID NO:26; SEQ ID NO:28; SEQ ID NO:29; SEQ ID NO:32; SEQ ID NO:35; and SEQ ID NO:3113.

The present invention also discloses genes that have previously been reported to be over-expressed in human tumor tissue but not specifically in colon tumors: SEQ ID NO:2; SEQ ID NO:3; SEQ ID NO:4; SEQ ID NO:10; SEQ ID NO:11; SEQ ID NO:15; SEQ ID NO:33; SEQ ID NO:34; SEQ ID NO:37.

Polypeptides

Another embodiment of the invention is an isolated polypeptide comprising an amino acid sequence selected from the group consisting of:
(a) a mature form of an amino acid sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114;
(b) a variant of a mature form of an amino acid sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114, wherein one or more amino acid residues in said variant differs from the amino acid sequence of said mature form, provided that said variant differs in no more than 20%, of the amino acid residues from the amino acid sequence of said mature form;
(c) an amino acid sequence comprising a sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114; and
(d) a variant of an amino acid sequence comprising a sequence selected from the group consisting of SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114, wherein one or more amino acid residues in said variant differs from the amino acid sequence, provided that said variant differs in no more than 20% of amino acid residues from said amino acid sequence.

In one aspect of the invention the SEQ ID NO:39; SEQ ID NO:40; SEQ ID NO:41; SEQ ID NO:42; SEQ ID NO:43; SEQ ID NO:46; SEQ ID NO:47; SEQ ID NO:48; SEQ ID NO:49; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:70; SEQ ID NO:71; SEQ ID NO:72; SEQ ID NO:73; SEQ ID NO:74; SEQ ID NO:75; and SEQ ID NO:3114. SEQ ID NO:39; SEQ ID NO:56; SEQ ID NO:59; SEQ ID NO:61; SEQ ID NO:62; SEQ ID NO:65; and SEQ ID NO:68

Forms of a protein of the present invention can be recovered from culture medium or from host cell lysates. If membrane-bound, it can be released from the membrane using a suitable detergent solution (e.g. Triton-X 100) or by enzymatic cleavage. Cells employed in expression of polypeptide can be disrupted by various physical or chemical means, such as freeze-thaw cycling, sonication, mechanical disruption, or cell lysing agents.

It can be desired to purify a protein of the present invention from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the a protein of the present invention. Additionally, other purification methods known to those skilled in the art can be used. Various methods of protein purification can be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular protein produced.

Polypeptide Variants

In addition to the full-length native sequence polypeptides described herein, variants with functions similar to those of the polypeptides disclosed can be prepared. Variants can be prepared by introducing appropriate nucleotide changes into the DNA, and/or by synthesis of the desired polypeptide. Those skilled in the art will appreciate that amino acid changes can alter post-translational processing of a protein of the present invention, such as changing the number or position of glycosylation sites or altering the membrane anchoring characteristics. Variations in the native full-length sequence or in various domains of the a protein of the present invention described herein, can be made, for example, using any of the techniques and guidelines for conservative and non-conservative mutations set forth, for instance, in U.S. Pat. No. 5,364,934. Variations can include the substitution, deletion or insertion of one or more codons encoding the protein of the present invention that results in a change in the amino acid sequence of the a protein of the present invention as compared with the native sequence. Optionally, the variation is by substitution of at least one amino acid with any other amino acid in one or more of the domains of the polypeptides. Guidance in determining which amino acid residue can be inserted, substituted or deleted without adversely affecting the desired activity can be found by comparing the sequence of the polypeptide with that of homologous known protein derived from other mammals, and minimizing the number of amino acid sequence changes made in regions of high homology. Amino acid substitutions can be the result of replacing one amino acid with another amino acid having similar structural and/or chemical properties, such as the replacement of a leucine with a isoleucine. Such substitutions are known as conservative amino acid replacements. Insertions or deletions are, optionally, in the range of about 1 to 5 amino acids. The variation allowed can be determined by systematically making insertions, deletions or substitutions of amino acids in the sequence and testing the resulting variants for activity exhibited by the full length or mature native sequence.

In particular embodiments, conservative substitutions of interest are shown in Table 2 under the heading of preferred substitutions. If such conservative substitutions result in a change in biological activity, then more substantial changes, denominated exemplary substitutions in Table 2, or as further described below in reference to amino acid classes, are introduced and the products screened for activity.

Substantial modifications in function or immunological identity of the polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues can be divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic: trp, tyr, phe.

Non-conservative substitutions will entail exchanging a member of one of these classes for another class. Such substituted residues also can be introduced into the conservative substitution sites or, more preferably, into the remaining (non-conserved) sites.

The variations can be made using methods known in the art such as oligonucleotide mediated (site-directed) mutagenesis, alanine scanning, and PCR mutagenesis. Site-directed mutagenesis (See Carter et al., Nucl. Acids Res., 13:4331 (1986); Zoller et al., Nucl. Acids Res., 10:6487 (1987)), cassette mutagenesis (See Wells et al., Gene, 34:315 (1985)), restriction selection mutagenesis (See Wells et al., Philos. Trans. R. Soc. London SerA, 317:415 (1986)) or any other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and, thus, is less likely to alter the main-chain conformation of the variant (See Cunningham and Wells, Science, 244: 1081-1085 (1989)). Alanine is also typically preferred because it is the most common amino acid. Further, it is frequently found in both buried and exposed positions (See Creighton, The Proteins, (W. H. Freeman & Co., N.Y.); Chothia, J. Mol. Biol., 150:1 (1976)). However, if alanine substitution does not yield adequate amounts of variant, an isosteric amino acid can be used.

Fragments of the protein of the present invention can be prepared by any of a number of conventional techniques. Desired peptide fragments can also be chemically synthesized. An alternative approach involves generating fragments by enzymatic digestion, e.g., by treating the protein with an enzyme known to cleave proteins at sites defined by particular amino acid residues, or by digesting the DNA with suitable restriction enzymes and isolating the desired fragment. Another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, polypeptide fragments share at least one biological and/or immunological activity with the native polypeptide.

Modifications of Polypeptides

Covalent modifications of a protein of the present invention are included within the scope of this invention. One type of covalent modification includes reacting targeted amino acid residues of a polypeptide with an organic derivatizing agent that is capable of reacting with selected side chains or the N- or C-terminal residues of the a protein of the present invention. Derivatization with bifunctional agents is useful, for instance, for crosslinking protein to a water-insoluble support matrix or surface for use in the method for purifying antibodies, and vice-versa. Commonly used crosslinking agents include e.g. 1,1-bis(diazoacetyl)-2-phenylethane glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(pazidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the—amino groups of lysine, arginine, and histidine side chains (See T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the polypeptide included within the scope of this invention comprises altering the native glycosylation pattern of the polypeptide. “Altering the native glycosylation pattern” is intended for purposes herein to mean deleting one or more carbohydrate moieties found in the native sequence (either by removing the underlying glycosylation site or by deleting the glycosylation by chemical and/or enzymatic means), and/or adding one or more glycosylation sites that are not present in the native sequence. In addition, the phrase includes qualitative changes in the glycosylation of the native proteins, involving a change in the nature and proportions of the various carbohydrate moieties present. Addition of glycosylation sites to the polypeptide can be accomplished by altering the amino acid sequence. The alteration can be made, for example, by the addition of, or substitution by, one or more serine or threonine residues to the native sequence (for 0 linked glycosylation sites). The amino acid sequence can optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the polypeptide can be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys, 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a protein or antibody of the present invention comprises linking the polypeptide or antibody to one of a variety of non-proteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

Functional groups capable of reacting with either the amino terminal α-amino group or ε-amino groups of lysines found on the polypeptide of the present invention, agonist, antagonist, or antibody include: carbonates such as the p-nitrophenyl, or succinimidyl; carbonyl imidazole; azlactones; cyclic imide thiones; isocyanates or isothiocyanates; tresyl chloride (EP 714 402, EP 439 508); and aldehydes. Functional groups capable of reacting with carboxylic acid groups, reactive carbonyl groups and oxidized carbohydrate moieties on the polypeptide of the present invention, agonist, antagonist, or antibody include; primary amines; and hydrazine and hydrazide functional groups such as the acyl hydrazides, carbazates, semicarbamates, thiocarbazates, etc. Mercapto groups, if available on the polypeptide of the present invention, agonist, antagonist, or antibody, can also be used as attachment sites for suitably activated polymers with reactive groups such as thiols; maleimides, sulfones, and phenyl glyoxals; see, for example, U.S. Pat. No. 5,093,531, the disclosure of which is hereby incorporated by reference. Other nucleophiles capable of reacting with an electrophilic center include, but are not limited to, for example, hydroxyl, amino, carboxyl, thiol, active methylene and the like.

In one preferred embodiment of the invention secondary amine or amide linkages are formed using the polypeptide of the present invention, agonist, antagonist, or antibody N-terminal amino groups or ε-amino groups of lysine and the activated PEG. In another preferred aspect of the invention, a secondary amine linkage is formed between the N-terminal primary amino group of polypeptide of the present invention, agonist, antagonist, or antibody and single or branched chain PEG aldehyde by reduction with a suitable reducing agent such as NaCNBH₃, NaBH₃, Pyridine Borane etc. as described in Chamow et al., Bioconjugate Chem. 5: 133-140 (1994) and U.S. Pat. No. 5,824,784.

In another preferred embodiment of the invention, polymers activated with amide-forming linkers such as succinimidyl esters, cyclic imide thiones, or the like are used to effect the linkage between the polypeptide of the present invention, agonist, antagonist, or antibody and polymer, see for example, U.S. Pat. No. 5,349,001; U.S. Pat. No. 5,405,877; and Greenwald, et al., Crit. Rev. Ther. Drug Carrier Syst. 17:101-161, 2000, which are incorporated herein by reference. One preferred activated poly(ethylene glycol), which may be bound to the free amino groups of polypeptide of the present invention, agonist, antagonist, or antibody includes single or branched chain N-hydroxysuccinylimide poly(ethylene glycol) may be prepared by activating succinic acid esters of poly(ethylene glycol) with N-hydroxysuccinylimide.

Other preferred embodiments of the invention include using other activated polymers to form covalent linkages of the polymer with the polypeptide of the present invention, agonist, antagonist, or antibody via ε-amino or other groups. For example, isocyanate or isothiocyanate forms of terminally activated polymers can be used to form urea or thiourea-based linkages with the lysine amino groups.

In another preferred aspect of the invention, carbamate (urethane) linkages are formed with protein amino groups as described in U.S. Pat. Nos. 5,122,614, 5,324,844, and 5,612,640, which are hereby incorporated by reference. Examples include N-succinimidyl carbonate, para-nitrophenyl carbonate, and carbonyl imidazole activated polymers. In another preferred embodiment of this invention, a benzotriazole carbonate derivative of PEG is linked to amino groups on the polypeptide of the present invention, agonist, antagonist, or antibody.

The protein of the present invention can also be modified in a way to form a chimeric molecule comprising a protein of the present invention fused to another, heterologous polypeptide, or amino acid sequence.

In one embodiment, such a chimeric molecule comprises a fusion of a protein of the present invention with a tag polypeptide, which provides an epitope to which an anti-tag antibody can selectively bind. The epitope tag is generally placed at the amino- or carboxyl-terminus of the protein. The presence of such epitope-tagged forms of a protein of the present invention can be detected using an antibody against the tag polypeptide. Also, provision of the epitope tag enables the protein to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:63936397 (1990)).

In an alternative embodiment, the chimeric molecule can comprise a fusion of the polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

In another embodiment, the chimeric molecule includes a fusion of a protein of the present invention with a signal peptide to allow or enhance secretion of the peptide or even to change its localization within the host cell. The signal sequence is generally placed at the amino- or carboxyl-terminus of a protein of the present invention, more usually the N-terminus when secretion or membrane localization is desired. Such fusions are typically intermediate products, since the signal peptide is usually specifically cleaved by enzymes of the host cell. Provision of a signal peptide enables the protein to be readily purified following its secretion to the culture medium. Various signal polypeptides, which allow secretion or targeting to compartments within the cell, are well known in the art and are available for use with numerous host cells, including yeast and mammalian cells.

Polynucleotides of the present invention can also be used for gene therapy. Gene therapy refers to therapy that is performed by the administration of a specific nucleic acid to a subject. Delivery of the therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Gene Therapy of Cancer: Translational Approaches from Preclinical Studies to Clinical Implementation E. C. Lattime & S. L. Gerson, eds Academic Press, 2002.

Cells may also be cultured ex vivo in the presence of therapeutic agents or proteins of the present invention in order to proliferate or to produce a desired effect on or activity in such cells. Treated cells can then be introduced in vivo for therapeutic purposes.

Rational Drug Design

The goal of rational drug design is to produce structural analogs of biologically active polypeptide of interest or of small molecules with which they interact, e.g., agonists, antagonists, or inhibitors. Any of these examples can be used to fashion drugs which are more active or stable forms of the polypeptide or which enhance or interfere with the function of the polypeptide in vivo (cf, Hodgson, Bio/Technology, 9: 19-21 (1991)).

In one approach, the three-dimensional structure of the polypeptide, or of a polypeptide-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of the polypeptide can be gained by modeling based on the structure of homologous proteins. In both cases, relevant structural information is used to design analogous polypeptide-like molecules or to identify efficient inhibitors. Useful examples of rational drug design can include molecules which have improved activity or stability as shown by Braxton and Wells, Biochemistry, 31:7796-7801 (1992) or which act as inhibitors, agonists, or antagonists of native peptides as shown by Athauda et al, J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the polypeptide can be made available to perform such analytical studies as X-ray crystallography. In addition, knowledge of the polypeptide amino acid sequence provided herein will provide guidance to those employing computer modeling techniques in place of or in addition to x-ray crystallography.

Nucleic Acid

The present invention further provides isolated nucleic acid molecules that encode a peptide or protein of the present invention. Nucleic acid molecules will consist of, consist essentially of, or comprise a nucleotide sequence that encodes one of the peptides of the present invention, an allelic variant thereof, or an ortholog or paralog thereof.

As used herein, an “isolated” nucleic acid molecule is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. The important point is that the nucleic acid is isolated from remote and unimportant flanking sequences such that it can be subjected to the specific manipulations described herein such as recombinant expression, preparation of probes and primers, and other uses specific to the nucleic acid sequences.

Moreover, an “isolated” nucleic acid molecule, such as a transcript/cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

Accordingly, the present invention provides nucleic acid molecules that comprises the nucleotide sequence shown in SEQ ID NOs:1-38, or any nucleic acid molecule that encodes the protein provided in SEQ ID NOs:39-76. A nucleic acid molecule comprisise a nucleotide sequence when the nucleotide sequence includes the nucleotide sequence of the nucleic acid molecule

Accordingly, the present invention provides nucleic acid molecules that comprise the nucleotide sequence shown in SEQ ID NOs:1-11, SEQ ID NOs:13-38, and SEQ ID NO:3113, or any nucleic acid molecule that encodes the protein provided in SEQ ID NOs:39-49, SEQ ID NOs:51-76, and SEQ ID NO:3114. A nucleic acid molecule consists of a nucleotide sequence when the nucleotide sequence is the complete nucleotide sequence of the nucleic acid molecule.

Accordingly, the present invention provides nucleic acid molecules that consist of the nucleotide sequence shown in SEQ ID NOs:1-11, SEQ ID NOs:13-38, and SEQ ID NO:3113, or any nucleic acid molecule that encodes the protein provided in SEQ ID NOs:39-49, SEQ ID NOs:51-76, and SEQ ID NO:3114. A nucleic acid molecule consists of a nucleotide sequence when the nucleotide sequence is the complete nucleotide sequence of the nucleic acid molecule.

The present invention further provides nucleic acid molecules that consist essentially of the nucleotide sequence shown in SEQ ID NOs:1-11, SEQ ID NOs:13-38, and SEQ ID NO:3113, or any nucleic acid molecule that encodes the protein provided in SEQ ID NOs:39-49, SEQ ID NOs:51-76, and SEQ ID NO:3114. A nucleic acid molecule consists essentially of a nucleotide sequence when such a nucleotide sequence is present with only a few additional nucleic acid residues in the final nucleic acid molecule.

The isolated nucleic acid molecules can encode the mature protein plus additional amino or carboxyl-terminal amino acids, or amino acids interior to the mature peptide (when the mature form has more than one peptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

As mentioned above, the isolated nucleic acid molecules include, but are not limited to, the sequence encoding the peptide alone, the sequence encoding the mature peptide and additional coding sequences, such as a leader or secretory sequence (e.g., a pre-pro or pro-protein sequence), the sequence encoding the mature peptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the nucleic acid molecule may be fused to a marker sequence encoding, for example, a peptide that facilitates purification.

Isolated nucleic acid molecules can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides nucleic acid molecules that encode fragments of the peptides of the present invention as well as nucleic acid molecules that encode obvious variants of the proteins of the present invention that are described above. Such nucleic acid molecules may be naturally occurring, such as allelic variants (same locus), paralogs (different locus), and orthologs (different organism), or may be constructed by recombinant DNA methods or by chemical synthesis. Such non-naturally occurring variants may be made by mutagenesis techniques, including those applied to nucleic acid molecules, cells, or organisms. Accordingly, as discussed above, the variants can contain nucleotide substitutions, deletions, inversions and insertions. Variation can occur in either or both the coding and non-coding regions. The variations can produce both conservative and non-conservative amino acid substitutions.

A fragment comprises a contiguous nucleotide sequence greater than 12 or more nucleotides. Further, a fragment could be at least 30, 40, 50, 100, 250 or 500 nucleotides in length depending on the length of the original nucleotide sequence. The length of the fragment will be based on its intended use. For example, the fragment can encode epitope bearing regions of the peptide, or can be useful as DNA probes and primers. Such fragments can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of gene.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides.

Orthologs, homologs, and allelic variants can be identified using methods well known in the art. These variants comprise a nucleotide sequence encoding a peptide that is typically 60-70%, 70-80%, 80-90%, and more typically at least about 90-95% or more homologous to the nucleotide sequence. Such nucleic acid molecules can readily be identified as being able to hybridize under moderate to stringent conditions. Allelic variants can readily be determined by genetic locus of the encoding gene

As used herein, the term “hybridizes under stringent conditions” is intended to describe conditions for hybridization and washing under which nucleotide sequences encoding a peptide at least 60-70% homologous to each other typically remain hybridized to each other. The conditions can be such that sequences at least about 60%, at least about 70%, at least about 80%, at least about 90%, at least about 95%, at least about 98%, or at least about 99% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6×SSC (sodium chloride/sodium citrate) at about 45° C., followed by one or more washes in 0.2×SSC, 0.1% SDS at 50-65° C. Examples of moderate to low stringency hybridization conditions are well known in the art.

Nucleic Acid Molecule Uses

The nucleic acid molecules of the present invention are useful for probes, primers, chemical intermediates, and in biological assays. The nucleic acid molecules are useful as a hybridization probe for messenger RNA, transcript/cDNA and genomic DNA to isolate full-length cDNA and genomic clones encoding the peptides shown in SEQ ID NOs:39-49, SEQ ID NOs:51-76, and SEQ ID NO:3114 and to isolate cDNA and genomic clones that correspond to variants (alleles, orthologs, etc.) producing the same or related peptides shown in SEQ ID NOs:39-49, SEQ ID NOs:51-76, and SEQ ID NO:3114.

The probe can correspond to any sequence along the entire length of the nucleic acid molecules. Accordingly, it could be derived from 5′ noncoding regions, the coding region, and 3′ noncoding regions.

The nucleic acid molecules are also useful as primers for PCR to amplify any given region of a nucleic acid molecule and are useful to synthesize antisense molecules of desired length and sequence.

The nucleic acid molecules are also useful for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for expressing antigenic portions of the proteins.

The nucleic acid molecules are also useful as probes for determining the chromosomal positions of the nucleic acid molecules by means of in situ hybridization methods.

The nucleic acid molecules are also useful in making vectors containing the gene regulatory regions of the nucleic acid molecules of the present invention.

The nucleic acid molecules are also useful for designing ribozymes corresponding to all, or a part, of the mRNA produced from the nucleic acid molecules described herein.

The nucleic acid molecules are also useful for making vectors that express part, or all, of the peptides.

The nucleic acid molecules are also useful for constructing host cells expressing a part, or all, of the nucleic acid molecules and peptides.

The nucleic acid molecules are also useful for constructing transgenic animals expressing all, or a part, of the nucleic acid molecules and peptides.

The nucleic acid molecules are also useful as hybridization probes for determining the presence, level, form and distribution of nucleic acid expression. Accordingly, the probes can be used to detect the presence of, or to determine levels of, a specific nucleic acid molecule in cells, tissues, and in organisms. The nucleic acid whose level is determined can be DNA or RNA. Accordingly, probes corresponding to the peptides described herein can be used to assess expression and/or gene copy number in a given cell, tissue, or organism. These uses are relevant for diagnosis of disorders involving an increase or decrease in protein expression relative to normal results.

Probes can be used as a part of a diagnostic test kit for identifying cells or tissues that express a protein.

Nucleic Acid Expression Assays

Nucleic acid expression assays are useful for drug screening to identify compounds that modulate nucleic acid expression.

The compounds can be used to treat a disorder associated with nucleic acid expression of the gene, particularly biological and pathological processes that are mediated by the gene in cells and tissues that express it. The method typically includes assaying the ability of the compound to modulate the expression of the nucleic acid and thus identifying a compound that can be used to treat a disorder characterized by undesired nucleic acid expression. The assays can be performed in cell-based and cell-free systems. Cell-based assays include cells naturally expressing the nucleic acid or recombinant cells genetically engineered to express specific nucleic acid sequences.

The assay for nucleic acid expression can involve direct assay of nucleic acid levels, such as mRNA levels. In this embodiment the regulatory regions of these genes can be operably linked to a reporter gene such as luciferase.

Thus, modulators of gene expression can be identified in a method wherein a cell is contacted with a candidate compound and the expression of mRNA determined. The level of expression of mRNA in the presence of the candidate compound is compared to the level of expression of mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

The invention further provides methods of treatment, with the nucleic acid as a target, using a compound identified through drug screening as a gene modulator to modulate nucleic acid expression in cells and tissues that express the nucleic acid. Modulation includes both up-regulation (i.e. activation or agonization) or down-regulation (suppression or antagonization) or nucleic acid expression.

Alternatively, a modulator for nucleic acid expression can be a small molecule or drug identified using the screening assays described herein as long as the drug or small molecule inhibits the nucleic acid expression in the cells and tissues that express the protein.

The nucleic acid molecules are also useful for monitoring the effectiveness of modulating compounds on the expression or activity of the gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Nucleic Acid Diagnostics

The nucleic acid molecules are also useful in diagnostic assays for qualitative changes in nucleic acid expression, and particularly in qualitative changes that lead to pathology. The nucleic acid molecules can be used to detect mutations in genes and gene expression products such as mRNA. The nucleic acid molecules can be used as hybridization probes to detect naturally occurring genetic mutations in the gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the gene associated with a dysfunction provides a diagnostic tool for an active disease or susceptibility to disease when the disease results from overexpression, underexpression, or altered expression of a protein.

Individuals carrying mutations in the gene can be detected at the nucleic acid level by a variety of techniques. Genomic DNA can be analyzed directly or can be amplified by using PCR prior to analysis. RNA or cDNA can be used in the same way. In some uses, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

Alternatively, mutations in a gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site. Perfectly matched sequences can be distinguished from mismatched sequences by nuclease cleavage digestion assays or by differences in melting temperature.

Sequence changes at specific locations can also be assessed by nuclease protection assays such as RNase and SI protection or the chemical cleavage method. Furthermore, sequence differences between a mutant gene and a wild-type gene can be determined by direct DNA sequencing. A variety of automated sequencing procedures can be utilized when performing the diagnostic assays (Naeve, C. W., (1995) Biotechniques 19:448), including sequencing by mass spectrometry (see, e.g., PCT International Publication No. WO 94/16101; Cohen et al., Adv. Chromatogr. 36:127-162 (1996); and Griffin et al., Appl. Biochem. Biotechnol. 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). Examples of other techniques for detecting point mutations include selective oligonucleotide hybridization, selective amplification, and selective primer extension.

Gene amplification and/or expression can be measured in a sample directly. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against a native sequence polypeptide or against a synthetic peptide based on the DNA sequences provided herein or against exogenous sequence fused to DNA and encoding a specific antibody epitope.

The nucleic acid molecules are also useful for testing an individual for a genotype that while not necessarily causing the disease, nevertheless affects the treatment modality. Thus, the nucleic acid molecules can be used to study the relationship between an individual's genotype and the individual's response to a compound used for treatment (pharmacogenomic relationship). Accordingly, the nucleic acid molecules described herein can be used to assess the mutation content of the gene in an individual in order to select an appropriate compound or dosage regimen for treatment.

Thus nucleic acid molecules displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The present invention is directed to antisense compounds, particularly oligonucleotides, which are targeted to a nucleic acid encoding polypeptide of the present invention, and which modulate the expression of the polypeptide of the present invention. Pharmaceutical and other compositions comprising the antisense compounds of the invention are also provided. Further provided are methods of modulating the expression of the polypeptide of the present invention in cells or tissues comprising contacting said cells or tissues with one or more of the antisense compounds or compositions of the invention. Further provided are methods of treating an animal, particularly a human, suspected of having or being prone to a disease or condition associated with expression of the polypeptide of the present invention by administering a therapeutically or prophylactically effective amount of one or more of the antisense compounds or compositions of the invention.

The nucleic acid molecules are thus useful as antisense constructs to control gene expression in cells, tissues, and organisms. A DNA antisense nucleic acid molecule is designed to be complementary to a region of the gene involved in transcription, preventing transcription and hence production of protein. An antisense RNA or DNA nucleic acid molecule would hybridize to the mRNA and thus block translation of mRNA into protein, if targeted to the initiator AUG codon. Alternatively, a DNA antisense molecule can modulate gene expression by stimulating RNAseH-dependent degradation of the target mRNA. Antisense oligonucleotides are anticipated to also contain modified nucleic acids that impart improved properties such as phosphorothioate, polyamide or morpholino groups.

Alternatively, a class of antisense molecules can be used to inactivate mRNA in order to decrease expression of nucleic acid. Accordingly, these molecules can treat a disorder characterized by abnormal or undesired nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the protein, such as substrate binding.

In addition, nucleic acids can be used to generate small interfering RNAs (siRNA) which, when delivered to a cell expressing the target mRNA, can lead to a decrease in the level of that RNA and thus a decrease in protein expressed by that RNA (Elbashir et al., Genes and Development. 15: 188-200, 2001). Generation of siRNAs can be accomplished using commercially available kits (MEGAscript RNAi Kit, Ambion, Inc. Austin, Tex.)

The nucleic acid molecules also provide vectors for gene therapy in patients containing cells that are aberrant in gene expression. Thus, recombinant cells, which include the patient's cells that have been engineered ex vivo and returned to the patient, are introduced into an individual where the cells produce the desired protein to treat the individual.

The invention also encompasses kits for detecting the presence of a nucleic acid in a biological sample. For example, the kit can comprise reagents such as a labeled or labelable nucleic acid or agent capable of detecting nucleic acid in a biological sample; means for determining the amount of nucleic acid in the sample; and means for comparing the amount of nucleic acid in the sample with a standard.

The compound or agent can be packaged in a suitable container. The kit can further comprise instructions for using the kit to detect protein mRNA or DNA.

Vectors/Host Cells

The invention also provides vectors containing the nucleic acid molecules described herein. The term “vector” refers to a vehicle, preferably a nucleic acid molecule, which can transport the nucleic acid molecules. When the vector is a nucleic acid molecule, the nucleic acid molecules are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules. The vectors can function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a transacting factor interacting with the cis-regulatory control region to allow transcription of the nucleic acid molecules from the vector. Alternatively, a trans-acting factor may be supplied by the host cell. Finally, a trans-acting factor can be produced from the vector itself. It is understood, however, that in some embodiments, transcription and/or translation of the nucleic acid molecules can occur in a cell-free system.

The regulatory sequences to which the nucleic acid molecules described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 3rd. ed., Cold Spring Harbor Laboratory Press, Cold Spring Harbor, N.Y., (2001)

A variety of expression vectors can be used to express a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 3rd. ed., Cold Spring Harbor Laboratory Press, Cold Spring Harbor, N.Y., (2001).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate nucleic acid molecule can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the peptide as a fusion protein. Accordingly, the invention provides fusion vectors that allow for the production of the peptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fission moiety. Proteolytic enzymes include, but are not limited to, factor Xa, thrombin, and enterolipase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET11d (Studier et al., Gene Expression Technology: Methods in Enzymology 185:60-89 (1990)).

Recombinant protein expression can be maximized in host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990)119-128). Alternatively, the sequence of the nucleic acid molecule of interest can be altered to provide preferential codon usage for a specific host cell, for example E. coli. (Wada et al., Nucleic Acids Res. 20:2111-2118 (1992)).

The nucleic acid molecules can also be expressed by expression vectors that are operative in yeast. Examples of vectors for expression in yeast e.g., S. cerevisiae include pYepSec1 (Baldari, et al., EMBO J. 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The nucleic acid molecules can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the nucleic acid molecules described herein are expressed in mammalian cells using mammalian expression vectors. Examples of mammalian expression vectors include pCDM8 (Seed, B. Nature 329:840(1987)) and pMT2PC (Kaufman et al., EMBO J. 6:187-195 (1987)).

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook, J., Fritsh, E. F., and Maniatis, T. Molecular Cloning: A Laboratory Manual. 3rd, ed., Cold Spring Harbor Laboratory, Cold Spring Harbor Laboratory Press, Cold Spring Harbor, N.Y., 2001.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the nucleic acid molecule sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 3rd, ed., Cold Spring Harbor Laboratory, Cold Spring Harbor Laboratory Press, Cold Spring Harbor, N.Y., 2001).

Host cells can contain more than one vector. Thus, different nucleotide sequences can be introduced on different vectors of the same cell. Similarly, the nucleic acid molecules can be introduced either alone or with other nucleic acid molecules that are not related to the nucleic acid molecules such as those providing trans-acting factors for expression vectors. When more than one vector is introduced into a cell, the vectors can be introduced independently, co-introduced or joined to the nucleic acid molecule vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the peptide is desired, which is difficult to achieve with multi-transmembrane domain containing proteins, appropriate secretion signals are incorporated into the vector. The signal sequence can be endogenous to the peptides or heterologous to these peptides.

Where the peptide is not secreted into the medium the protein can be isolated from the host cell by standard disruption procedures, including freeze thaw, sonication, mechanical disruption, use of lysing agents and the like. The peptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the peptides described herein, the peptides can have various glycosylation patterns, depending upon the cell, or maybe non-glycosylated as when produced in bacteria. In addition, the peptides may include an initial modified methionine in some cases as a result of a host-mediated process.

Uses of Vectors and Host Cells

The recombinant host cells expressing the proteins described herein have a variety of uses. First, the cells are useful for producing protein that can be further purified to produce desired amounts of protein. Thus, host cells containing expression vectors are useful for protein production.

Host cells are also useful for conducting cell-based assays involving the protein or protein fragments, such as those described above as well as other formats known in the art. Thus, a recombinant host cell expressing a native protein is useful for assaying compounds that stimulate or inhibit protein function.

Host cells are also useful for identifying protein mutants in which these functions are affected. If the mutants naturally occur and give rise to a pathology, host cells containing the mutations are useful to assay compounds that have a desired effect on the mutant protein (for example, stimulating or inhibiting function) which may not be indicated by their effect on the native protein.

Transgenic Animals

Genetically engineered host cells can be further used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a protein and identifying and evaluating modulators of protein activity. Other examples of transgenic animals include non-human primates, sheep, dogs, cows, goats, chickens, and amphibians.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the protein nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

Transgenic animals containing recombinant cells that express the peptides described herein are useful to conduct the assays described herein in an in vivo context. Accordingly, the various physiological factors that are present in vivo and that could effect substrate binding, and protein activation, may not be evident from in vitro cell-free or cell-based assays. Accordingly, it is useful to provide non-human transgenic animals to assay in vivo protein function, including substrate interaction, the effect of specific mutant proteins on protein function and substrate interaction, and the effect of chimeric proteins. It is also possible to assess the effect of null mutations, that is mutations that substantially or completely eliminate one or more protein functions.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

Microarrays Construction and Element Selection

A chemical coupling procedure and an ink jet device can be used to synthesize array elements on the surface of a substrate. (See, e.g., Gamble et al. U.S. Pat. No. 5,981,733) An array analogous to a dot or slot blot may also be used to arrange and link elements to the surface of a substrate using or thermal, UV, mechanical, or chemical bonding procedures, or a vacuum system. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

In another alternative, full-length cDNAs or Expressed Sequence Tags (ESTs) comprise the elements of the microarray. Full-length cDNAs or ESTs representing a genome scan of thousand of unique nucleotide sequences, or corresponding to one of the nucleotide sequences of the present invention, are arranged on an appropriate substrate, e.g., a glass slide. The cDNA is fixed to the slide using, e.g., U.V. cross-linking followed, by thermal and chemical and subsequent drying. (See, e.g., Schena, M. et al. (1995) Science 270:467-470; and Shalon, D. et al. (1996) Genome Res. 6:639-645.) Fluorescent probes are prepared and used for hybridization to the elements on the substrate.

Probe sequences for microarrays may be selected by screening a large number of clones from a variety of cDNA libraries in order to find sequences with conserved protein motifs common to genes coding for signal sequence containing polypeptides. In one embodiment, sequences identified from cDNA libraries, are analyzed to identify those gene sequences with conserved protein motifs using an appropriate analysis program, e.g., the Block 2 Bioanalysis Program (Incyte, Palo Alto, Calif.). This motif analysis program, based on sequence information contained in the Swiss-Prot Database and PROSITE, is a method of determining the function of uncharacterized proteins translated from genomic or cDNA sequences. (See, e.g., Bairoch, A. et al. (1997) Nucleic Acids Res. 25:217-221; and Attwood, T. K. et al. (1997) J. Chem. Inf. Comput. Sci. 37:417-424.) PROSITE may be used to identify functional or structural domains that cannot be detected using conserved motifs due to extreme sequence divergence. The method is based on weight matrices. Motifs identified by this method are then calibrated against the SWISS-PROT database in order to obtain a measure of the chance distribution of the matches.

In another embodiment, Hidden Markov models (HMMs) may be used to find shared motifs, specifically consensus sequences. (See, e.g., Pearson, W. R. and D. J. Lipman (1988) Proc. Natl. Acad. Sci. USA 85:2444-2448; and Smith, T. F. and M. S. Waterman (1981) J. Mol. Biol. 147:195-197.) HMMs were initially developed to examine speech recognition patterns, but are now being used in a biological context to analyze protein and nucleic acid sequences as well as to model protein structure. (See, e.g., Krogh, A. et al. (1994) J. Mol. Biol. 235:1501-1531; and Collin, M. et al. (1993) Protein Sci. 2:305-314.) HMMs have a formal probabilistic basis and use position-specific scores for amino acids or nucleotides. The algorithm continues to incorporate information from newly identified sequences to increase its motif analysis capabilities.

Microarray Uses

Polynucleotide sequences are particularly useful when they are hybridizable array elements in a microarray. Such a microarray can be employed to monitor the expression of genes of unknown function, but which are differentially expressed in precancerous or cancerous tissue. In addition, the microarray can be used to monitor the expression of genes with a known function in tumor biology.

The microarray can be used for large scale genetic or gene expression analysis of a large number of polynucleotide sequences. The microarray can be used in the diagnosis of diseases, such as in the diagnosis of early stages of ductal carcinoma before other definitive symptoms are evident, and in the differential diagnosis of diseases with similar symptoms. The microarray can also be used in the monitoring and evaluation of treatments where altered expression of genes coding for polypeptides implicated in the control of cell proliferation cause disease, such as cancer. Additionally, the microarray can be used to investigate an individual's predisposition to a disease, such as cancer. Furthermore, the microarray can be employed to investigate cellular responses, such as cell proliferation and the like.

When the polynucleotide sequences are employed as hybridizable array elements in a microarray, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment.

With a large enough number of transcript profiles derived from different biological samples, a statistically significant correlation can emerge between cell and tissue source information, such as disease states, treatment outcomes, exposure to various environmental factors or genotypes, and the expression levels of particular genes or groups of genes. Comparisons between transcript profiles of different cells or tissues or of the same cells or tissues under different conditions can be used to discern differences in transcriptional activities. For example, a transcript profile can show differences occurring between two different tissues, such as liver and prostate; between normal and diseased tissue, such as normal and breast tumor or between untreated and treated tissues, such as prostate tumor and irradiated prostate tumor.

In another embodiment of the present invention, kits can be generated which contain the necessary reagents to carry out the assays of the present invention.

Specifically, the invention provides a compartmentalized kit to receive, in close confinement, one or more containers which comprises: (a) a first container comprising one of the nucleic acid molecules that can bind to a fragment of the human genome disclosed herein; and (b) one or more other containers comprising one or more of the following: wash reagents, reagents capable of detecting presence of a bound nucleic acid.

In detail, a compartmentalized kit includes any kit in which reagents are contained in separate containers. Such containers include small glass containers, plastic containers, strips of plastic, glass or paper, or arraying material such as silica. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the nucleic acid probe, containers which contain wash reagents (such as phosphate buffered saline, Tris-buffers, etc.), and containers which contain the reagents used to detect the bound probe. One skilled in the art will readily recognize that the previously unidentified genes of the present invention can be routinely identified using the sequence information disclosed herein can be readily incorporated into one of the established kit formats which are well known in the art, particularly expression arrays.

RNA Isolation, Amplification, and Labeling for Microarray

RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Targets, Part 1. Theory and Nucleic Acid Preparation, P. Tijssen, ed. Elsevier (1993). When sample polynucleotides are amplified it is desirable to amplify the nucleic acid sample and maintain the relative abundances of the original sample, including low abundance transcripts. Total mRNA can be amplified by reverse transcription using a reverse transcriptase, a primer consisting of oligo d(T), and a sequence encoding the phage T7 promoter to provide a single stranded DNA template. The second DNA strand is polymerized using a DNA polymerase and a RNAse which assists in breaking up the DNA/RNA hybrid. After synthesis of the double stranded DNA, T7 RNA polymerase can be added and RNA transcribed from the second DNA strand template (Van Gelder et al. U.S. Pat. No. 5,545,522). RNA can be amplified in vitro, in situ or in vivo (See Eberwine U.S. Pat. No. 5,514,545).

The polynucleotides may be labeled with one or more labeling moieties to allow for detection of hybridized polynucleotide complexes. The labeling moieties can include compositions that can be detected by spectroscopic, photochemical, biochemical, bioelectronic, immunochemical, electrical, optical or chemical means. The labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Hybridization and Analysis of Microarrays

Hybridization causes a denatured polynucleotide and a denatured sample polynucleotide to form a stable duplex through base pairing. Hybridization methods are well known to those skilled in the art (See, for example, Laboratory Techniques in Biochemistry and Molecular Biology, Vol. 24: Hybridization With Nucleic Acid Targets, P. Tijssen, ed. Elsevier, N.Y. (1993)) Hybridization conditions can be defined by salt concentration, temperature, and other chemicals and conditions well known in the art. In particular, stringency can be increased by reducing the concentration of salt, or raising the hybridization temperature.

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Stringent temperature conditions will ordinarily include temperatures of at least about 30° C., more preferably of at least about 37° C., and most preferably of at least about 60° C. Varying additional parameters, such as hybridization time, the concentration of detergent or solvent, and the inclusion or exclusion of carrier DNA, are well known to those skilled in the art. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York, N.Y.; and Sambrook, J. et al. (2001) Molecular Cloning A Laboratory Manual, Cold Spring Harbor Press, Plainview, N.Y.).

Hybridization reactions can be performed in a differential hybridization format. In a differential hybridization format, the differential expression of a set of genes in two biological samples is analyzed. For differential hybridization, polynucleotides from both biological samples are prepared and labeled with different labeling moieties. A mixture of the two labeled polynucleotides is added to a microarray. The microarray is then examined under conditions in which the emissions from the two different labels are individually detectable. Polynucleotides in the microarray that are hybridized to substantially equal numbers of polynucleotides derived from both biological samples give a distinct combined fluorescence (Shalon et al. PCT publication WO95/35505). In a preferred embodiment, the fluorophores Cy3 and Cy5 (Amersham Pharmacia Biotech, Piscataway N.J.) are employed as labels.

After hybridization, the microarray is washed to remove nonhybridized nucleic acids and complex formation between the hybridizable array elements and the polynucleotides is detected. Methods for detecting complex formation are well known to those skilled in the art.

In a differential hybridization experiment, polynucleotides from two or more different biological samples are labeled with two or more different fluorescent labels with different emission wavelengths. Fluorescent signals are detected separately with different photomultipliers set to detect specific wavelengths. The relative abundances/expression levels of the polynucleotides in two or more samples is obtained.

Typically, microarray fluorescence intensities can be normalized to take into account variations in hybridization intensities when more than one microarray is used under similar test conditions. In a preferred embodiment, individual polynucleotide complex hybridization intensities are normalized using the intensities derived from internal normalization controls contained on each microarray.

The microarray can be used to monitor the expression level of large numbers of genes simultaneously and to identify splice variants, mutations, and polymorphisms. This information may be used to determine gene function, to understand the genetic basis of a disease, to diagnose a disease, and to develop and monitor the activities of therapeutic agents.

mRNA Expression Analysis Using Sybrgreen

Where possible, micro-array results were confirmed using the SYBR green procedure. The SYBR green PCR procedure is a way to perform real-time PCR using the SYBR Green 1 Dye. Direct detection of polymerase chain reaction (PCR) product is monitored by measuring the increase in fluorescence caused by the binding of SYBR green dye to double stranded DNA. Gene specific PCR oligonucleotide primer pairs were designed using the Primer Express 1.5 software. (Applied Biosystems, Foster City, Calif.)

One microgram of each mRNA is added to 100 uL of a reverse transcriptase reaction using the ABI Taqman reverse transcription reagents with random hexamers according to the manufacturers protocol (Applied Biosystems, Foster City, Calif.) The thermal cycling conditions included 1 cycle at 25° C. for 10 minutes, 1 cycle at 48° C. for 30 minutes and 1 cycle at 95° C. for 5 minutes. Four hundred microliters of water is then added to the cDNA reaction. The resulting cDNA (10 uL) is added to a 25 uL SYBR green PCR reaction mixture according to the manufactures protocol. (Applied Biosystems, Foster City, Calif.) The thermal cycling conditions included 1 cycle at 95° C. for ten minutes, 40 cycles at 95° C. for 15 s, annealing at 60° C. for 1 minute. Data are expressed as the fold increase normalized to the same gene using the delta delta CT method for relative quantitation. For comparison data obtained with cDNA from colon and breast tumors were compared to data from pools of normal colon and breast cDNA.

Antibodies

The present invention further provides antibodies to the proteins of the present invention. Exemplary antibodies include polyclonal, monoclonal, humanized, bispecific, and heteroconjugate antibodies.

Polyclonal Antibodies

The antibodies can comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include the polypeptide or a fusion protein thereof. It can be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that can be employed include Freund's complete adjuvant and MPL-TDM adjuvant (monophosphoryl Lipid A, synthetic trehalose dicorynomycolate). Alternatively, genetic immunization may a usefull approach to generating antibodies without having to purify the protein of interest (Kilpatrick et al., 17:569-576, 1998). The immunization protocol can be selected by one skilled in the art without undue experimentation.

Monoclonal Antibodies

The antibodies can, alternatively, be monoclonal antibodies. Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001(1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against a protein of the present invention. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods (Goding, supra). Suitable culture media for this purpose include, for example, Dulbecco's Modified Eagle's Medium and RPMI1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison et al., PNAS, 81:6851-6855 (1984)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody. The antibodies can be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Human and Humanized Antibodies

The antibodies of the invention can further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′), or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat, or rabbit having the desired specificity, affinity and capacity. In some instances, Fv framework residues of the human immunoglobulin are replaced by corresponding nonhuman residues. Humanized antibodies can also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a nonhuman immunoglobulin and all or substantially all of the framework regions (FR) are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323329 (1988); and Presta, Curr. Op. Struct. Biol.,2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Homogenous and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13, 65-93 (1995); Tomizuka et al., PNAS 97, 722-727 (2000).

Bispecific Antibodies

Bispecific antibodies are monoclonal, preferably human or humanized, antibodies that have binding specificities for at least two different antigens. In the present case, one of the binding specificities is for a protein of the present invention; the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are cotransfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers, which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al, Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker, which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991). Exemplary bispecific antibodies can bind to two different epitopes on a given polypeptide herein. Alternatively, an arm can be combined with an arm which binds to a triggering molecule on a leukocyte such as a T-cell receptor molecule (e.g. CD2, CD3, CD28, or B7), or Fc receptors for IgG (FcyR), such as FcyRI (CD64), FcyRII (CD32) and FcyRIII (CD16) so as to focus cellular defense mechanisms to the cell expressing the particular a protein of the present invention. Bispecific antibodies can also be used to localize cytotoxic agents to cells, which express a particular a protein of the present invention. These antibodies possess a binding arm to a protein of the present invention and an arm, which binds a cytotoxic agent or a radionuclide chelator, such as EOTUBE, DPTA, DOTA, or TETA. Another bispecific antibody of interest binds the polypeptide and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate, methyl-4-mercaptobutyrimidate, and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 11911195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, momordica charantia inhibitor, curcin, crotin, sapaonaria officinalis inhibitor, gelonin, mitogellin, restrictocin, phenomycin, maytansinoids, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bisazido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Methods of Detecting Proteins and Nucleic Acids

The invention also provides a method for detecting the presence or absence of a protein of the present invention in a biological sample. The method includes obtaining a biological sample from a test subject and contacting the biological sample with a compound or an agent capable of detecting protein or nucleic acid (e.g., mRNA, genomic DNA) that encodes protein such that the presence of a protein of the present invention is detected in the biological sample. An agent for detecting mRNA or genomic DNA is a labeled nucleic acid probe capable of hybridizing to mRNA or genomic DNA. The nucleic acid probe can be, for example, a full-length nucleic acid, such as the nucleic acid of SEQ ID NOs:1-1, SEQ ID NOs:13-38, SEQ ID NO:3113, or a fragment thereof, such as SEQ ID NOs:77-3011, SEQ ID NOs:3012-3083, or an oligonucleotide of at least 15, 30, 50, 100, 250 or 500 nucleotides in length and sufficient to specifically hybridize under stringent conditions to mRNA or genomic DNA. Other suitable probes for use in the diagnostic assays of the invention are described herein.

An agent for detecting protein is an antibody capable of binding to protein, preferably an antibody with a detectable label. Antibodies can be polyclonal, or more preferably, monoclonal. An intact antibody, or a fragment thereof (e.g. Fab or F(ab′)₂ can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting protein, mRNA, or genomic DNA, such that the presence of protein, mRNA or genomic DNA is detected in the biological sample, and comparing the presence of protein, mRNA or genomic DNA in the control sample with the presence of protein, mRNA or genomic DNA in the test sample.

Uses for Ab in Diagnostics and Affinity Purification

The antibodies to the proteins of the invention have various utilities. For example, antibodies can be used in diagnostic assays for a protein of the present invention, e.g., detecting its expression in specific cells, tissues, or serum. Various diagnostic assay techniques known in the art can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety can be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

A competition assay may be employed wherein antibodies specific to the polypeptide are attached to a solid support and the labeled polypeptide and a sample derived from the host are passed over the solid support and the amount of label detected attached to the solid support can be correlated to a quantity of polypeptide in the sample.

Antibodies to a protein of the present invention also are useful for the affinity purification of a protein of the present invention from recombinant cell culture or natural sources. In this process, the antibodies are immobilized on a suitable support, such a Sephadex resin or filter paper, using methods well known in the art. The immobilized antibody then is contacted with a sample containing a protein of the present invention to be purified, and thereafter the support is washed with a suitable solvent that will remove substantially all the material in the sample except the a protein of the present invention, which is bound to the immobilized antibody. Finally, the support is washed with another suitable solvent that will release the protein of the present invention from the antibody.

Binding Assays

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which can be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevrel and Nathans, Proc. Natl. Acad. Sci. USA, 89: 5789-5793 (1991). Many transcriptional activators, such as yeast GAL4, consist of two physically discrete modular domains, one acting as the DNA-binding domain, and the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL4-lacZ reporter gene under control of a GAL4-activated promoter depends on reconstitution of GALA activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Identification of Receptor/Binding Protein

Antagonists can be detected by combining the polypeptide and a potential antagonist with membrane-bound polypeptide receptors or recombinant receptors or a binding protein under appropriate conditions for a competitive inhibition assay. The polypeptide can be labeled, such as by radioactivity, such that the number of polypeptide molecules bound to the receptor or binding protein can be used to determine the effectiveness of the potential antagonist. The gene encoding the receptor or binding protein can be identified by numerous methods known to those of skill in the art, for example, ligand panning and FACS sorting (Coligan et al., Current Protocols in Immun., 1(2): Chapter 5 (1991)).

Preferably, expression cloning is employed wherein polyadenylated RNA is prepared from a cell responsive to the polypeptide and a cDNA library created from this RNA is divided into pools and used to transfect COS cells or other cells that are not responsive to or do not contain binding protein activity to the polypeptide. Transfected cells that are grown on glass slides are exposed to labeled polypeptide or lysates are prepared for testing binding activity. The polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor or binding protein. As an alternative approach for receptor or binding protein identification, labeled polypeptide can be photoaffinity-linked with cell membrane or extract preparations that express or contain the receptor or binding protein. Cross-linked material is resolved by PAGE and exposed to X-ray film. The labeled complex can be excised, resolved into peptide fragments, and subjected to protein micro sequencing. The amino acid sequence obtained from micro sequencing would be used to design a set of degenerate oligonucleotide probes to screen a cDNA library to identify the gene encoding the putative receptor or binding protein.

More specific examples of potential antagonists include a polypeptide that binds to the fusions of immunoglobulin with polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments or conjugated antibodies. Alternatively, a potential antagonist can be a closely related protein, for example, a mutated form of the polypeptide that recognizes the receptor or binding protein but imparts no effect, thereby competitively inhibiting the action of the polypeptide.

Inhibitors of Binding Interactions

When the coding sequences for a polypeptide encode a protein which binds to another protein, the polypeptide can be used in assays to identify the other proteins or molecules involved in the binding interaction. By such methods, inhibitors of the binding interaction can be identified. Proteins involved in such binding interactions can also be used to screen for peptide or small molecule inhibitors or agonists of the binding interaction. Also, the receptor of a protein of the present invention can be used to isolate correlative ligand(s). Screening assays can be designed to find lead compounds that mimic the biological activity of a native protein or a receptor for the protein. Such screening assays will include assays amenable to high-throughput screening of chemical libraries, making them particularly suitable for identifying small molecule drug candidates. Small molecules contemplated include both synthetic organic or inorganic compounds. The assays can be performed in a variety of formats, including protein-protein binding assays, biochemical screening assays, immunoassays and cell based assays, which are well characterized in the art. Such high- and ultra-high throughput assays can also be used to test antisense molecules.

Methods for Identifying Modulators of Expression or Activity Cell Based Assays

The invention provides a method for identifying modulators, i.e., candidate or test compounds or agents (e.g., antibodies to the polypeptides encoded by nucleic acids of SEQ ID NO:77-3011, an antisense nucleic acid molecule, peptides, a polypeptide agonist, a polypeptide antagonist, peptidomimetics, small molecules or other drugs) that bind to the proteins of the present invention or have a stimulatory or inhibitory effect on, for example, expression or activity.

In one embodiment, the invention provides assays for screening candidate or test compounds, which bind to or modulate the activity of the membrane-bound form of a protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carrell et al. (1994) Angew Chem Int Ed Engl 3 3:2061; and Gallop et al. (1994) J Med Chem 3 7:123 3.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci USA 87:6378-6382; Felici (1991) J Mol Biol 222:301-310; Ladner above.).

In one embodiment, an assay is a cell-based assay in which a cell that expresses a membrane-bound form of protein, or a biologically active portion thereof, on the cell surface is contacted with a test compound and the ability of the test compound to bind to a protein determined. The cell, for example, can be of mammalian origin or a yeast cell. Determining the ability of the test compound to bind to the protein can be accomplished, for example, by coupling the test compound with a radioisotope or enzymatic label such that binding of the test compound to the protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of protein; or a biologically active portion thereof, on the cell surface with a known compound which binds the polypeptide of the present invention to form an assay mixture, contacting the assay mixture with a test compound, and determining the ability of the test compound to interact with a protein, wherein determining the ability of the test compound to interact with a protein comprises determining the ability of the test compound to preferentially bind to a polypeptide of the preset invention or a biologically active portion thereof as compared to the known compound.

In another embodiment, an assay is a cell-based assay comprising contacting a cell expressing a membrane-bound form of protein, or a biologically active portion thereof, on the cell surface with a test compound and determining the ability of the test compound to modulate (e.g., stimulate or inhibit) the activity of the protein or biologically active portion thereof. Determining the ability of the test compound to modulate the activity of the protein of the present invention or a biologically active portion thereof can be accomplished, for example, by determining the ability of the protein to bind to or interact with a target molecule. As used herein, a “target molecule” is a molecule with which a protein binds or interacts in nature, for example, a molecule on the surface of a cell which expresses a protein that interacts with a protein of the present invention, a molecule on the surface of a second cell, a molecule in the extracellular milieu, a molecule associated with the internal surface of a cell membrane or a cytoplasmic molecule. A target molecule can be a molecule other than of the present invention or a protein or polypeptide of the present invention. In one embodiment, a target molecule is a component of a signal transduction pathway that facilitates transduction of an extracellular signal (e.g. a signal generated by binding of a compound to a membrane-bound molecule) through the cell membrane and into the cell. The target, for example, can be a second intercellular protein that has catalytic activity or a protein that facilitates the association of downstream signaling molecules with a protein of the present invention.

Determining the ability of the protein to bind to or interact with a target molecule can be accomplished by one of the methods described above for determining direct binding. In one embodiment, determining the ability of the protein to bind to or interact with a target molecule can be accomplished by determining the activity of the target molecule. For example, the activity of the target molecule can be determined by detecting induction of a cellular second messenger of the target (i.e. intracellular Ca²⁺, diacylglycerol, IP3, etc.), detecting catalytic/enzymatic activity of the target an appropriate substrate, detecting the induction of a reporter gene (comprising a regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a protein or biologically active portion thereof with a test compound and determining the ability of the test compound to bind to the protein or biologically active portion thereof. Binding of the test compound to the protein can be determined either directly or indirectly as described above. In one embodiment, the assay comprises contacting the protein or biologically active portion thereof with a known compound which binds a protein of the present invention to form an assay mixture, contacting the assay mixture with a test compound, and determining the ability of the test compound to interact with a protein, wherein determining the ability of the test compound to interact with a protein comprises determining the ability of the test compound to preferentially bind to a protein of the present invention or biologically active portion thereof as compared to the known compound.

In another embodiment, an assay is a cell-free assay comprising contacting protein or biologically active portion thereof with a test compound and determining the ability of the test compound to modulate (e.g. stimulate or inhibit) the activity of the protein or biologically active portion thereof. Determining the ability of the test compound to modulate the activity of a protein of the present invention can be accomplished, for example, by determining the ability of the protein to bind to a target molecule by one of the methods described above for determining direct binding. In an alternative embodiment, determining the ability of the test compound to modulate the activity of a protein of the present invention can be accomplished by determining the ability of the protein further modulate a target molecule. For example, the catalytic/enzymatic activity of the target molecule on an appropriate substrate can be determined as previously described.

In yet another embodiment, the cell-free assay comprises contacting the protein or biologically active portion thereof with a known compound which binds a protein of the present invention to form an assay mixture, contacting the assay mixture with a test compound, and determining the ability of the test compound to interact with a protein, wherein determining the ability of the test compound to interact with a protein comprises determining the ability of the protein to preferentially bind to or modulate the activity of a target molecule.

The cell-free assays of the present invention are amenable to use of either the soluble form or the membrane-bound form of a protein of the present invention. In the case of cell-free assays comprising the membrane-bound form of a protein, it may be desirable to utilize a solubilizing agent such that the membrane-bound form of a protein of the present invention is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X100, Triton® X-114, Thesit®, Isotridecypoly (ethylene glycol ether)n, N-dodecyl-N,N-dimethyl-3-ammonio-1-propahe sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either a protein of the present invention or its target molecule to facilitate separation of complexed from uncomplexed forms of one or both of the proteins, as well as to accommodate automation of the assay. Binding of a test compound to a protein of the present invention, or interaction of a protein of the present invention with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-protein of the present invention fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of a protein of the present invention binding or activity determined using standard techniques.

Other techniques for immobilizing proteins on matrices can also be used in the screening assays of the invention. For example, either a protein of the present invention or its target molecule can be immobilized utilizing conjugation of biotin and streptavidin. Biotinylated protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the present invention or target molecules, but which do not interfere with binding of the protein to its target molecule, can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays that rely on detecting an enzymatic activity associated with the protein or target molecule.

In another embodiment, modulators of a protein of the present invention expression are identified in a method wherein a cell is contacted with a candidate compound and the expression of mRNA or protein in the cell is determined. The level of expression of mRNA or protein in the presence of the candidate compound is compared to the level of expression of mRNA or protein in the absence of the candidate compound. The candidate compound can then be identified as a modulator of a protein of the present invention expression based on this comparison. For example, when expression of mRNA or protein is greater (statistically significantly greater) in the presence of the candidate compound than in its absence, the candidate compound is identified as a stimulator of mRNA or protein expression. Alternatively, when expression of mRNA or protein is less (statistically significantly less) in the presence of the candidate compound than in its absence, the candidate compound is identified as an inhibitor of mRNA or protein expression. The level of mRNA or protein expression in the cells can be determined by methods described herein for detecting mRNA or protein.

In yet another aspect of the invention, the a protein of the present invention can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with a protein of the present invention and modulate activity of a protein of the present invention. Such binding proteins are also likely to be involved in the propagation of signals by the proteins as, for example, upstream or downstream elements of the pathway.

The two-hybrid system is based on the modular nature of most transcription factors, which consist of separable DNA-binding and activation domains. Briefly, the assay utilizes two different DNA constructs. In one construct, the gene that codes for a protein of the present invention is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a protein-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein, which interacts with a protein of the present invention.

In Vivo Tumor Models

For cancer, a variety of well-known animal models can be used to further understand the role of the genes identified herein in the development and pathogenesis of tumors, and to test the efficacy of candidate therapeutic agents, including antibodies and other antagonists of the native protein of the present invention, such as small-molecule antagonists. The in vivo nature of such models makes them particularly predictive of responses in human patients. Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models, Such models can be generated by introducing tumor cells which express the genes of the present invention into syngeneic mice using standard techniques, e.g., subcutaneous injection, tail vein injection, spleen implantation, intraperitoneal implantation, implantation under the renal capsule, or orthotopic implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts (which naturally or through recombinant technology express on or more of the genes of the present invention) has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII, and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds. (CRC Press, Inc., 1991).

The cells introduced into such animals can be derived from known tumor/cancer cell lines, for example, the B 104-1-1 cell line (stable NIH-3T3 cell line transfected with the neu protooncogene); ras-transfected NIH-3T3 cells; Caco-2 (ATCC HTB-37); or a moderately well-differentiated grade II human colon adenocarcinoma cell line, HT-29 (ATCC HTB-38); or from tumors and cancers. Samples of tumor or cancer cells can be obtained from patients undergoing surgery, using standard conditions involving freezing and storing in liquid nitrogen (Karmali et al., Br. J. Cancer, 48: 689-696 (1983)).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Animal models of breast cancer can be generated, for example, by implanting rat neuroblastoma cells (from which the neu oncogene was initially isolated), or neu-transformed NIH-3T3 cells into nude mice, essentially as described by Drebin et al., Proc. Nat. Acad. Sci. USA, 83: 9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54: 4726-4728 (1994) and Too et al., Cancer Research 55: 68i-684 (1995). This model is based on the so-called “METAMOUSE”™ sold by AntiCancer, Inc., (San Diego, Calif.).

Syngenic Tumor Models

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, and WEHI-164 are chemically induced fibrosarcomas of BALB/c female mice (DeLeo et al., J. Exp. Med., 146: 720 (1977)), which provide a highly controllable model system for studying the anti-tumor activities of various agents (Palladino et al., J. Immunol., 138: 4023-4032 (1987)). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small-cell carcinoma of the lung (SCCL). This tumor can be introduced in normal mice upon injection of tumor fragments from an affected mouse or of cells maintained in culture (Zupi et al., Br. J. Cancer, 41: suppl. 4, 30 (1980)). Evidence indicates that tumors can be started from injection of even a single cell and that a very high proportion of infected tumor cells survive. For further information about this tumor model see, Zacharski, Haemostasis, 16: 300-320 (1986).

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals Wu and Sheng eds. (Basel, 1989), p. 301. It is noted, however, that necrosis and inflammatory responses following treatment may actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees, and monkeys. Techniques known in the art to introduce a transgene into such animals include pronucleic microinjection (U.S. Pat. No. 4,873,191); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer (Lavitrano et al., Cell, 57: 717-73 (1989)). For a review, see for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89: 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals can be constructed that have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker that can be used to monitor integration. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell, 69: 915 (1992). The selected cells are then injected into a blastocyst of an animal (e.g., a mouse or rat) to form aggregation chimeras. See, e.g., Bradley, in Teratocarcinomas and Embryonic Stem Cells: A Practical Approach, E. J. Robertson, ed. (IRL: Oxford, 1987), pp. 113-152. A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knock-out animals can also be generated, as is well known in the art, by administering an antisense molecule of the invention. Animals comprising such antisense molecules are specifically contemplated as an embodiment of the invention. Knockout animals can be characterized, for instance, by their ability to defend against certain pathological conditions and by their development of pathological conditions due to absence (knock-out) of the polypeptides.

Animal Models (Non-Rodent)

The efficacy of antibodies specifically binding a protein of the present invention polypeptides identified herein, and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis may merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination and biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response, and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this type of tumor in animals.

Other in vitro and in vivo cancer tests known in the art are also suitable herein.

Screening for Agonists or Antagonists

This invention encompasses methods of screening compounds to identify those that mimic the polypeptide (agonists) or prevent the effect of the polypeptide (antagonists).

Screening assays for antagonist drug candidates are designed to identify compounds that bind or complex with the polypeptides encoded by the genes identified herein, or otherwise interfere with the interaction of the encoded polypeptides with other cellular proteins. Such assays include methods identifying compounds that interfere with the interaction of a gene (mRNA or genomic DNA) encoding a polypeptide, such as those described herein. These screening assays will include assays amenable to high- or ultra-high-throughput screening of chemical libraries, making them particularly suitable for identifying antisense and small molecule drug candidates.

The assays can be performed in a variety of formats, including protein-protein binding assays, biochemical screening assays, immunoassays, target nucleic acid binding assays, and cell-based assays, which are well characterized in the art.

Antisense

Another potential polypeptide antagonist is an antisense construct prepared using antisense technology, where, for example, the antisense molecule acts to block directly the translation of mRNA (or transcription) by hybridizing to targeted mRNA (or genomic DNA) and preventing protein translation (or mRNA transcription) of a protein of the present invention. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the polypeptide. The antisense RNA oligonucleotide hybridizes to the mRNA in vivo and blocks translation of the mRNA molecule into the polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA can be expressed in vivo to inhibit production of the polypeptide. When antisense DNA is used, oligodeoxyribonucleotides derived from the translation initiation site, e.g., between about −10 and +10 positions of the target gene nucleotide sequence, are preferred.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The present invention employs oligomeric antisense compounds, particularly oligonucleotides, for use in modulating the function of nucleic acid molecules including SEQ ID NOs:1-11, SEQ ID NOs:13-38, and SEQ ID NO:3113 encoding SEQ ID NOs:39-49, SEQ ID NOs:51-76, and SEQ ID NO: 3114, ultimately modulating the amount of SEQ ID NOs:39-49, SEQ ID NOs:51-76, and SEQ ID NO: 3114 produced. This is accomplished by providing antisense compounds, which specifically hybridize with one or more nucleic acids including SEQ ID NOs:1-11, SEQ ID NOs:13-38, and SEQ ID NO:3113 encoding SEQ ID NOs:39-49, SEQ ID NOs:51-76, and SEQ ID NO: 3114. As used herein, the terms “target nucleic acid” and “nucleic acid including SEQ ID NOs:1-11, SEQ ID NOs:13-38, and SEQ ID NO:3113 encoding SEQ ID NOs:39-49, SEQ ID NOs:51-76, and SEQ ID NO: 3114 encompass DNA encoding SEQ ID NOs:39-49, SEQ ID NOs:51-76, and SEQ ID NO: 3114, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of SEQ ID NOs:39-49, SEQ ID NOs:51-76, and SEQ ID NO: 3114. In the context of the present invention, “modulation” means either an increase (stimulation) or a decrease (inhibition) in the expression of a gene. In the context of the present invention, inhibition is the preferred form of modulation, of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule including SEQ ID NOs:1-11, SEQ ID NOs:13-38, and SEQ ID NO:3113 encoding SEQ ID NOs:39-49, SEQ ID NOs:51-76, and SEQ ID NO: 3114. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding SEQ ID NOs:39-49, SEQ ID NOs:51-76, and SEQ ID NO: 3114, regardless of the sequence(s) of such codons.

It is also known in the art that a translation termination codon (or “stop codon”) of a gene may have one of three sequences, i.e. 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen, or reversed Hoogsteen hydrogen bonding, between complementary nucleoside or nucleotide bases. For example, adenine and thymine are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleo sides). Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal I linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5;541,306; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C, to C₁₀ alkyl or C₂ to C₁₀ alkenyl and alkynyl. Particularly preferred are O[(CH₂)ₙO]ₘCH₃, O(CH₂)ₙ,OCH₃, O(CH₂)ₙNH₂, O(CH₂)ₙCH₃, O(CH₂)ₙONH₂, and O(CH₂ₙON[(CH₂)ₙCH₃)]₂ where n and m are from 1 to about 10. Other preferred oligonucleotides comprise one of the following at the 2′ position: C₁ to C₁₀, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkyl amino, polyalkyl amino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other preferred modifications include 2′-methoxy (2′-O CH₃), 2′-aminopropoxy (2′-O CH₂ CH₂ CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,12′, 5,596,091; 5,614,617; 5,750,692, and 5,681,941, each of which is herein incorporated by reference.

Another modification of the oligonucleotides of the invention involves chemically linking to the oligonucleotide one or more moieties or conjugates, which enhance the activity, cellular distribution, or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds, which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease, which cleaves the RNA strand of RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently, and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the present invention can be utilized for diagnostics, therapeutics, and prophylaxis and as research reagents and kits. For therapeutics, an animal, preferably a human, suspected of having a disease or disorder, which can be treated by modulating the expression of SEQ ID NOs:39-49, SEQ ID NOs:51-76, and SEQ ID NO: 3114, is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation, or tumor formation, for example.

The antisense compounds of the invention are useful for research and diagnostics, because these compounds hybridize to nucleic acids encoding SEQ ID NOs:39-49, SEQ ID NOs:51-76, and SEQ ID NO: 3114, enabling sandwich and other assays to easily be constructed to exploit this fact. Hybridization of the antisense oligonucleotides of the invention with a nucleic acid encoding SEQ ID NOs:39-49, SEQ ID NOs:51-76, and SEQ ID NO: 3114 can be detected by means known in the art. Such means may include conjugation of an enzyme to the oligonucleotide, radiolabelling of the oligonucleotide or any other suitable detection means. Kits using such detection means for detecting the level of SEQ ID NOs:39-49, SEQ ID NOs:51-76, and SEQ ID NO: 3114 in a sample may also be prepared.

Potential antagonists include small molecules that bind to the active site, the protein-binding site, or other relevant binding site (e.g., co-factor binding site, substrate binding site) of the polypeptide, thereby blocking the normal biological activity of the polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endo nucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides, The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra. Such molecules can have backbone bonds not naturally found in DNA or RNA.

These small molecules can be identified by any one or more of the screening assays discussed herein and/or by any other screening techniques well known for those skilled in the art.

All assays for antagonists are common in that they call for contacting the drug candidate with a polypeptide encoded by a nucleic acid identified herein under conditions and for a time sufficient to allow these two components to interact.

Assays for Antagonists

In another assay for antagonists, mammalian cells or a membrane preparation expressing the receptor would be incubated with labeled polypeptide in the presence of the candidate compound. The ability of the compound to enhance or block this interaction could then be measured.

Potential antagonists include small molecules that bind to the active site, the receptor-binding site, or growth factor or other relevant binding site of the polypeptide, thereby blocking the normal biological activity of the polypeptide. Examples of small molecules include, but are not limited to, antibodies, small peptides or peptide-like molecules, preferably soluble peptides, and synthetic non-peptidyl organic or inorganic compounds.

Drug Screening

This invention is particularly useful for screening compounds by using polypeptides or binding fragment thereof in any of a variety of drug screening techniques. The polypeptide or fragment employed in such a test can either be free in solution, affixed to a solid support, borne on a cell surface, or located intracellularly. One method of drug screening utilizes eukaryotic or prokaryotic host cells, which are stably transformed with recombinant nucleic acids expressing the polypeptide or fragment. Drugs are screened against such transformed cells in competitive binding assays. Such cells, either in viable or fixed form, can be used for standard binding assays. One can measure, for example, the formation of complexes between polypeptide or a fragment and the agent being tested. Alternatively, one can examine the diminution in complex formation between the polypeptide and its target cell or target receptors caused by the agent being tested.

Thus, the present invention provides methods of screening for drugs or any other agents, which can affect a disease or disorder associated with a protein of the present invention. These methods comprise contacting such an agent with an polypeptide or fragment thereof and assaying (i) for the presence of a complex between the agent and the polypeptide or fragment, or (ii) for the presence of a complex between the polypeptide or fragment and the cell, by methods well known in the art. In such competitive binding assays, the polypeptide or fragment is typically labeled. After suitable incubation, free polypeptide or fragment is separated from that present in bound form, and the amount of free or uncomplexed label is a measure of the ability of the particular agent to bind to polypeptide or to interfere with the polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a protein of the present invention, the peptide test compounds are reacted with polypeptide and washed. Bound polypeptide is detected by methods well known in the art. Purified polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding polypeptide specifically compete with a test compound for binding to polypeptide or fragments thereof. In this manner, the antibodies can be used to detect the presence of any peptide which shares one or more antigenic determinants with polypeptide.

Disorders to be Treated

Cancerous Disorders

Polypeptides of the invention may be involved in cancer cell generation, proliferation or metastasis. Detection of the presence or amount of polynucleotides or polypeptides of the invention may be useful for the diagnosis and/or prognosis of one or more types of cancer. For example, the presence or increased expression of a polynucleotide/polypeptide of the invention may indicate a hereditary risk of cancer, a precancerous condition, or an ongoing malignancy. Conversely, a defect in the gene or absence of the polypeptide may be associated with a cancer condition. Identification of single nucleotide polymorphisms associated with cancer or a predisposition to cancer may also be useful for diagnosis or prognosis.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness. Therapeutic compositions of the invention may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

Leukemias

Leukemias and related disorders may be treated or prevented by administration of a therapeutic that promotes or inhibits function of the polynucleotides and/or polypeptides of the invention. Such leukemias and related disorders include but are not limited to acute leukemia, acute lymphocytic leukemia, acute myelocytic leukemia, myeloblastic, promyclocytic, myelomonocytic, monocytic, erythroleukemia, chronic leukemia, chronic myelocytic (granulocytic) leukemia and chronic lymphocytic leukemia (for a review of such disorders, see Fishman et al., 1985, Medicine, 2d Ed., J. B. Lippincott Co., Philadelphia).

Other Activities

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, co-factors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Applications, Administration Protocols, Schedules, Doses, and Formulations

The molecules herein and agonists and antagonists thereto are pharmaceutically useful as a prophylactic and therapeutic agent for various disorders and diseases as set forth above.

Therapeutic compositions of the polypeptides or agonists or antagonists are prepared for storage by mixing the desired molecule having the appropriate degree of purity with optional pharmaceutically acceptable carriers, excipients, or stabilizers Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The polypeptides, agonists, or antagonists will typically be formulated in such vehicles at a concentration of about 0.1 mg/ml to 100 mg/ml.

Another formulation comprises incorporating a polypeptide or antagonist thereof into formed articles. Such articles can be used in modulating cancer cell growth. In addition, tumor invasion and metastasis may be modulated with these articles.

Polypeptide or antagonist to be used for in vivo administration must be sterile. This is readily accomplished by filtration through sterile filtration membranes, prior to or following lyophilization and reconstitution. Polypeptide ordinarily will be stored in lyophilized form or in solution if administered systemically. If in lyophilized form, polypeptide or antagonist thereto is typically formulated in combination with other ingredients for reconstitution with an appropriate diluent at the time for use. An example of a liquid formulation of a polypeptide or antagonist is a sterile, clear, colorless unpreserved solution filled in a single-dose vial for subcutaneous injection. Preserved pharmaceutical compositions suitable for repeated use may contain, for example, depending mainly on the indication and type of polypeptide: a) a polypeptide or agonist or antagonist thereto; b) a buffer capable of maintaining the pH in a range of maximum stability of the polypeptide or other molecule in solution, preferably about 4-8; c) a detergent/surfactant primarily to stabilize the polypeptide or molecule against agitation-induced aggregation; d) an isotonifier; e) a preservative selected from the group of phenol, benzyl alcohol and a benzethonium halide, e.g., chloride; and water.

If the detergent employed is non-ionic, it may, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of the polypeptide or antagonist thereto, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Therapeutic polypeptide compositions generally are placed into a container having a sterile access port, for example, an intravenous solution bag, or vial having a stopper pierceable by a hypodermic injection needle. The formulations are preferably administered as repeated intravenous (iv.), subcutaneous (s.c.), or intramuscular (i.m.) injections, or as aerosol formulations suitable for intranasal or intrapulmonary delivery (for intrapulmonary delivery see, e.g., EP 257,956).

The polypeptide can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3hydroxybutyric acid (EP 133 988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release polypeptide compositions also include liposomally entrapped polypeptides. Liposomes containing the polypeptide are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52 322; EP 36 676; EP 88 046; EP 143 949; EP 142,641; Japanese patent application 83-118008; U.S. Pat. Nos. 4,485,045 and 4,544,545; and EP 102 324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of polypeptide or antagonist thereto will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. If the polypeptide has a narrow host range, for the treatment of human patients formulations comprising human polypeptide, more preferably native-sequence human polypeptide, are preferred. The clinician will administer polypeptide until a dosage is reached that achieves the desired effect for treatment of the condition in question. For example, if the objective is the treatment of CHF, the amount would be one that inhibits the progressive cardiac hypertrophy associated with this condition. The progress of this therapy is easily monitored by echocardiography. Similarly, in patients with hypertrophic cardiomyopathy, polypeptide can be administered on an empirical basis.

With the above guidelines, the effective dose generally is within the range of from about 0.001 to about 1.0 mg/kg, more preferably about 0.0°-1.0 mg/kg, most preferably about 0.01-0.1 mg/kg.

For non-oral use in treating human adult hypertension, it is advantageous to administer polypeptide in the form of an injection at about 0.01 to 50 mg, preferably about 0.05 to 20 mg, most preferably 1 to 20 mg, per kg body weight, 1 to 3 times daily by intravenous injection. For oral administration, a molecule based on the polypeptide is preferably administered at about 5 mg to 1 g, preferably about 10 to 100 mg, per kg body weight, 1 to 3 times daily. It should be appreciated that endotoxin contamination should be kept minimally at a safe level, for example, less than 0.5 ng/mg protein. Moreover, for human administration, the formulations preferably meet sterility, pyrogenicity, general safety, and purity as required by FDA Office and Biologics standards.

The dosage regimen of a pharmaceutical composition containing polypeptide to be used in tissue regeneration will be determined by the attending physician considering various factors that modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration, and other clinical factors. The dosage may vary with the type of matrix used in the reconstitution and with inclusion of other proteins in the pharmaceutical composition. For example, the addition of other known growth factors, such as IGF-I, to the final composition may also affect the dosage. Progress can be monitored by periodic assessment of tissue/bone growth and/or repair, for example, X-rays, histomorphometric determinations, and tetracycline labeling.

The route of polypeptide or antagonist or agonist administration is in accord with known methods, e.g., by injection or infusion by intravenous, intramuscular, intracerebral, intraperitoneal, intracerebral spinal, subcutaneous, intraocular, intraarticular, intrasynovial, intrathecal, oral, topical, or inhalation routes, or by sustained-release systems as noted below. The polypeptide or antagonists thereof also are suitably administered by intratumoral, peritumoral, intralesional, or perilesional routes, to exert local as well as systemic therapeutic effects. The intraperitoneal route is expected to be particularly useful, for example, in the treatment of ovarian tumors.

If a peptide or small molecule is employed as an antagonist or agonist, it is preferably administered orally or non-orally in the form of a liquid or solid to mammals.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate). Therapeutic compositions and combination therapy

Polypeptides, polynucleotides, or modulators of polypeptides of the invention (including inhibitors and stimulators of the biological activity of the polypeptide of the invention) may be administered to treat cancer. Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer.

The composition can also be administered in therapeutically effective amounts as a portion of an anti-cancer cocktail. An anti-cancer cocktail is a mixture of the polypeptide or modulator of the invention with one or more anti-cancer drugs in addition to a pharmaceutically acceptable carrier for delivery. The use of anti-cancer cocktails as a cancer treatment is routine. Anti-cancer drugs that are well known in the art and can be used as a treatment in combination with the polypeptide or modulator of the invention include: Actinomycin D, Aminoglutethimide, Asparaginase, Bleomycin, Busulfan. Carboplatin, Carmustine, Chlorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Floxuridine, 5-Fluorouracil (5-Fu), Flutamide, Hydroxyurea (hydroxycarbamide), Ifosfamide, Interferon Alpha-2a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Octreotide, Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate.

In addition, therapeutic compositions of the invention may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the polypeptide of the invention to reduce the risk of developing cancers.

Pharmaceutical Compositions of Antibodies

Antibodies specifically binding a polypeptide identified herein, as well as other molecules identified by the screening assays disclosed herein, can be administered for the treatment of various disorders in the form of pharmaceutical compositions.

If the polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In Vitro Models for Effective Doses

In vitro models can be used to determine the effective doses of the polypeptide of the invention as a potential cancer treatment. These in vitro models include proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella et al., J. Natl. Can. Inst., 52: 921-30 (1974), mobility and invasive potential of tumor cells in Boyden Chamber assays as described in Pilkington et al., Anticancer Res., 17: 4107-9 (1997), and angiogenesis assays such as induction of vascularization of the chick chorioallantoic membrane or induction of vascular endothelial cell migration as described in Ribatta et al., Intl. J. Dev. Biol., 40: 1189-97 (1999) and Li et al., Clin. Exp. Metastasis, 17:423-9 (1999), respectively. Suitable tumor cells lines are available, e.g. from American Type Tissue Culture Collection catalogs.

Articles of Manufacture

An article of manufacture such as a kit containing polypeptide or antagonists thereof useful for the diagnosis or treatment of the disorders described above comprises at least a container and a label. Suitable containers include, for example, bottles, vials, syringes, and test tubes. The containers may be formed from a variety of materials such as glass or plastic. The container holds a composition that is effective for diagnosing or treating the condition and may have a sterile access port (for example, the container may be an intravenous solution bag or a vial having a stopper pierceable by a hypodermic injection needle). The active agent in the composition is the polypeptide or an agonist or antagonist thereto. The label on, or associated with, the container indicates that the composition is used for diagnosing or treating the condition of choice. The article of manufacture may further comprise a second container comprising a pharmaceutically acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a second or third container with another active agent as described above.

Diagnosis, Prognosis, and Management of Cancer

The polynucleotides described herein, as well as their gene products and corresponding genes and gene products, are of particular interest as genetic or biochemical markers (e.g., in blood or tissues) that will detect the earliest changes along the carcinogenesis pathway and/or to monitor the efficacy of various therapies and preventive interventions.

For example, the level of expression of certain polynucleotides can be indicative of a poorer prognosis, and therefore warrant more aggressive chemo- or radio-therapy for a patient or vice versa.

The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy.

Determining expression of certain polynucleotides and comparison of a patients profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides described herein, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for metastatic colon cancer can also have clinical implications for breast cancer or ovarian cancer.

Staging.

Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T and a number which describes the tumor's size; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides and corresponding genes and gene products described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of Cancers.

Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well differentiated or low grade tumors. The following guidelines are generally used for grading tumors: 1) GX Grade cannot be assessed; 2) G1 Well differentiated; G2 Moderately well differentiated; 3) G3 Poorly differentiated; 4) G4 Undifferentiated. The polynucleotides of the SEQ ID NOs:77-3011 and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of Colon Cancer.

The polynucleotides corresponding to genes that exhibit the appropriate expression pattern can be used to detect colon cancer in a subject. Colorectal cancer is one of the most common neoplasms in humans and perhaps the most frequent form of hereditary neoplasia.

Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews.

The expression of appropriate polynucleotides can be used in the diagnosis, prognosis and management of colorectal cancer. Detection of colon cancer can be determined using expression levels of any of these sequences alone or in combination with the levels of expression. Determination of the aggressive nature and/or the metastatic potential of a colon cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon E R, et al, Cell (1990) 61(5):759; Hamilton S R et al., Cancer (1993) 72:957; Bodmer W, et al., Nat Genet. (1994) 4(3):217; Fearon E R, Ann NY Acad Sci. (1995) 768:101).

For example, development of colon cancer can be detected by examining the level of expression of a gene corresponding to a polynucleotides described herein to the levels of oncogenes (e.g. ras) or tumor suppressor genes (e.g. FAP or p53). Thus expression of specific marker polynucleotides can be used to discriminate between normal and cancerous colon tissue, to discriminate between colon cancers with different cells of origin, to discriminate between colon cancers with different potential metastatic rates, etc. For a review of markers of cancer, see, e.g., Hanahan et al. (2000) Cell 100:57-70.

Monitoring Efficacy in Clinical Trials

The invention also features a method of monitoring the efficacy of a compound in clinical trials for inhibition of tumors, e.g., colon tumors, in a patient by obtaining a first sample of tumor tissue cells from the patient; administering the compound to the patient; after a time sufficient for the compound to inhibit the tumor, obtaining a second sample of tumor tissue cells from the patient; and detecting in the first and second samples the level any one or more of SEQ ID NO:77-1993, SEQ ID NOs:3012-3083, or the genes of SEQ ID NO:1-11, SEQ ID NOs:13-38, and SEQ ID NO:3113 wherein a level lower in the second sample than in the first sample indicates that the compound is effective to inhibit a tumor in the patient. Alternatively, the efficacy of a compound in clinical trials for inhibition of tumors, e.g., colon tumors, in a patient can be monitored by obtaining a first sample of tumor tissue cells from the patient; administering the compound to the patient; after a time sufficient for the compound to inhibit the tumor, obtaining a second sample of tumor tissue cells from the patient; and detecting in the first and second samples the level of any one or more of nucleic acid sequences of SEQ ID NOs:1994-3011 wherein a level higher in the second sample than in the first sample indicates that the compound is effective to inhibit a tumor in the patient.

Monitoring the influence of agents (e.g., drugs, compounds) on inhibition of tumor growth (e.g., the ability to modulate aberrant cell proliferation and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels, or protein activity, can be monitored in clinical trials of subjects exhibiting increased gene expression, protein levels, or protein activity. In such clinical trials, expression or activity of the polypeptide and preferably, that of other polypeptides that have been implicated in colon cancer, can be used as markers.

For example, and not by way of limitation, genes, including those of the invention, that are modulated in cells by treatment with an agent (e.g., compound, drug or small molecule), which modulates tumor growth (e.g., as identified in a screening assay described herein) can be identified. Thus, to study the effect of agents on cancer, e.g., colon cancer, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, antibody or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of the polypeptide or nucleic acid of the invention in the post-administration sample; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to reduce expression or activity of the polypeptide, i.e., to increase the effectiveness of the agent.

Definitions

A “native sequence” comprises a polypeptide having the same amino acid sequence as a protein of the present invention derived from nature. Such native sequences can be isolated from nature or can be produced by recombinant and/or synthetic means. The term “native sequence” specifically encompasses naturally occurring truncated or secreted forms (e.g., an extracellular domain sequence), naturally occurring variant forms (e.g., alternatively spliced forms), and naturally occurring allelic variants of the protein. In one embodiment of the invention, the native sequence of a protein of the present invention is a mature or full-length native sequence comprising amino acids encompassing the N-terminus to the C-terminus of the known sequence.

A “variant polypeptide” means an active polypeptide as defined herein having at least about 80% amino acid sequence identity with the amino acid sequence of the protein in Table 1. Such identity can be to the residues of the full-length polypeptide or to a specifically derived fragment of the amino acid sequence of the protein. Such variant polypeptides include, for instance, polypeptides wherein one or more amino acid residues are added, or deleted, at the N- and/or C-terminus, as well as within one or more internal domains of each amino acid sequence. Ordinarily, a variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with either the full-length polypeptide or a specifically derived fragment of the amino acid sequence of the protein shown in Table 1. Variant polypeptides do not encompass the native polypeptide sequence.

As used herein with respect to the polypeptide sequences, “percent (%) amino acid sequence identity” is defined as the percentage of amino acid residues in a candidate sequence that are identical with the amino acid residues in a protein of the present invention sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.01). All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated by one skilled in the art that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described above using the ALIGN-2 sequence comparison computer program. However, % amino acid sequence identity can also be determined using the sequence comparison program NCBI-BLAST2 (Altschul et al., Nucleic Acids Res. 25:3389-3402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=1515, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Variant polynucleotide” or “variant nucleic acid sequence” means a nucleic acid molecule which encodes an active polypeptide, as defined herein, and which has at least about 80% nucleic acid sequence identity with either (a) a nucleic acid sequence which encodes residues comprising the known reading frame of the nucleic acid coding for the polypeptide shown in Table 1, or (b) a nucleic acid sequence which encodes another specifically derived fragment of the amino acid sequence shown in Table 1. Ordinarily, a variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes all residues of the polypeptide shown in Table 1, or (b) a nucleic acid sequence which encodes another specifically derived fragment of the amino acid sequence shown in Table 1. Polynucleotide variants do not encompass the native nucleotide sequence.

“Percent (%) nucleic acid sequence identity” with respect to the polypeptide encoding nucleic acid sequences identified herein is defined as the percentage of nucleotides in a candidate sequence that are identical with the nucleotides in a polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.01). All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

Unless specifically stated otherwise, all % nucleic acid sequence identity values used herein are obtained as described above using the ALIGN-2 sequence comparison computer program. However, % nucleic acid sequence identity can also be determined using the sequence comparison program NCBI-BLAST2 (Altschul et al., Nucleic Acids Res. 25:33893402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program NCBIBLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, variant polynucleotides are nucleic acid molecules that encode an active polypeptide and which are capable of hybridizing, preferably under stringent hybridization and wash conditions, to nucleotide sequences encoding the full-length polypeptide shown in Table 1. Variant polypeptides can be those that are encoded by a variant polynucleotide.

The term “conservative”, in the context of the amino acid sequence identity comparisons performed as described above, includes amino acid residues that have similar properties. Preferred conservative substitutions are shown in Table 2.

TABLE 2

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows: 100 times the fraction VY, where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

The term “isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and can include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the protein natural environment will not be present. Ordinarily, however, isolated polypeptide will be prepared by at least one purification step.

An “isolated” nucleic acid molecule encoding a polypeptide is a nucleic acid molecule that is identified and separated from at least one contaminant nucleic acid molecule with which it is ordinarily associated in the natural source of the nucleic acid. Preferably, the isolated nucleic is free of association with all components with which it is naturally associated. An isolated nucleic acid molecule is one that is other than in the form or setting in which it is found in nature. Isolated nucleic acid molecules therefore are distinguished from nucleic acid molecules as they exist in natural cells. However, an isolated nucleic acid molecule encoding a polypeptide includes nucleic acid molecules contained in cells that ordinarily express polypeptides where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome-binding site. Additionally, eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single monoclonal antibodies (including agonist, antagonist, and neutralizing antibodies), antibody compositions with polyepitopic specificity, single chain antibodies, and fragments of antibodies. The term “monoclonal antibody” as used herein refers to an antibody obtained from a population of substantially homogeneous antibodies, i.e., the individual antibodies comprising the population are identical except for possible naturally occurring mutations that can be present in minor amounts. The “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature, which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Active” or “activity” for the purposes herein refers to form(s) of polypeptide which retain a biological and/or an immunological activity of native or naturally-occurring polypeptide, wherein “biological” activity refers to a biological function (either inhibitory or stimulatory), which includes enzymatic activity, caused by a native or naturally-occurring polypeptide other than the ability to induce the production of an antibody against an antigenic epitope possessed by a native or naturally-occurring polypeptide and an “immunological” activity refers to the ability to induce the production of an antibody against an antigenic epitope possessed by a native or naturally-occurring polypeptide. A preferred biological activity includes, for example, the property of the polypeptide to degrade extracellular matrix as for example in the case of proteases discovered to do so described in this invention.

The term “antagonist” is used in the broadest sense, and includes any molecule that partially or fully blocks, inhibits, or neutralizes a biological activity of a native polypeptide disclosed herein. In a similar manner, the term “agonist” is used in the broadest sense and includes any molecule that mimics a biological activity of a native polypeptide disclosed herein. Suitable agonist or antagonist molecules specifically include agonist or antagonist antibodies or antibody fragments, fragments or amino acid sequence variants of native polypeptides, peptides, antisense molecules, and small organic molecules. Methods for identifying agonists or antagonists of a polypeptide include contacting a polypeptide, mRNA or gene with a candidate agonist or antagonist molecule and measuring a detectable change in one or more biological activities normally associated with the polypeptide.

“Treatment” refers to both therapeutic treatment and prophylactic or preventative measures, wherein the object is to prevent or slow down (lessen) the targeted pathologic condition or disorder. More specifically, “treatment” is an intervention performed with the intention of preventing the development or altering the pathology of a cancerous disorder. The concept of treatment is used in the broadest sense, and specifically includes the prevention (prophylaxis), moderation, reduction, and curing of cancerous disorders of any stage. Accordingly, “treatment” refers to both therapeutic treatment and prophylactic or preventative measures, wherein the object is to prevent or slow down (lessen) a cancerous disorder. The disorder may result from any cause. Subjects in need of treatment include those already with the disorder as well as those susceptible to the disorder or those in whom the disorder needs to be prevented.

“Chronic” administration refers to administration of the agent(s) in a continuous mode as opposed to an acute mode, so as to maintain the initial therapeutic effect (activity) for an extended period of time.

“Intermittent” administration is treatment that is not consecutively done without interruption, but rather is cyclic in nature.

“Microarray” refers to an array of distinct polynucleotides or oligonucleotides arranged on a substrate such as paper, nylon, or other type of membrane, filter, gel, polymer, chip, glass slide, or any other suitable support, including solid supports. The polynucleotides or oligonucleotides (the backbone chemistry can be any available in the art) can be synthesized on a substrate or prepared before application to the substrate.

Administration “in combination with” one or more further therapeutic agents includes both simultaneous (concurrent) and consecutive administration in any order of one or more therapeutic agents.

“Carriers” as used herein include pharmaceutically acceptable carriers, excipients, or stabilizers, which are nontoxic to the cell or mammal being exposed thereto at the dosages and concentrations, employed. Often the physiologically acceptable carrier is an aqueous pH buffered solution. Examples of physiologically acceptable carriers include buffers such as phosphate, citrate, and other organic acids; antioxidants including ascorbic acid; low molecular weight (i.e., less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; lipids such as cationic lipids, salt-forming counterions such as sodium; and/or nonionic surfactants such as TWEEN™. polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see Zapata et al., Protein Eng. 8(10): 1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment that contains a complete antigen-recognition and antigen-binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CHI) of the heavy chain. Fab fragments differ from Fab′ fragments by the addition of a few residues at the carboxy terminus of the heavy chain CH1 domain including one or more cysteines from the antibody hinge region. Fab-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known to one of ordinary skill in the art.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one that has been identified and separated and/or recovered from a component of its natural environment. Contaminant components of its natural environment are materials, which would interfere with diagnostic or therapeutic uses for the antibody, and can include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present, Ordinarily, however, isolated antibody will be prepared by at least one purification step.

The word “label”, when used herein, refers to a detectable compound or composition, which is conjugated directly or indirectly to the antibody so as to generate a “labeled” antibody. The label can be detectable by itself (e.g. radioisotope labels or fluorescent labels) or, in the case of an enzymatic label, can catalyze chemical alteration of a substrate compound or composition, which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

As used herein, the terms “cancer” or “cancerous” refer to or describe the physiological conditions in mammals that are typically characterized by unregulated cell growth. This can include benign growth, pre-malignant growth or malignant growth wherein the cells of the primary growth have spread to other sites. As used herein, the terms include any neoplasia, described as “an abnormal mass of tissue, the growth of which exceeds and is uncoordinated with that of the normal tissues and persists in the same excessive manner after cessation of the stimuli which evoke the change.” (referenced in Robbins, S. L. Pathologic Basis of Disease, W. B. Saunders Co. 1974). Examples of cancers include but are not limited to, carcinomas including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancers such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. The preferred cancers for treatment according to the methods of the invention described herein are breast, colon, lung, melanoma, ovarian, and prostate cancer.

The term “cytotoxic agent” as used herein refers to a substance that inhibits or prevents the function of cells and/or causes destruction of cells. The term is intended to include radioactive isotopes (e.g., ¹³¹I, ¹²⁵I, ⁹⁰Y, and ¹⁸⁶Re), chemotherapeutic agents, and toxins such as enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Specific examples include Adriamycin, Doxorubicin, 5-Fluorouracil, Cytosine arabinoside (“Ara-C”), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vinblastine, Vinorelbine, Carboplatin, Teniposide, Daunomycin, Carminomycin, Aminopterin, Dactinomycin, Mitomycins, Esperamicins (See U.S. Pat. No. 4,675,187), Melphalan, and other related nitrogen mustards. Also included in this definition are hormonal agents that act to regulate or inhibit hormone action on tumors, such as tamoxifen and onapristone.

“Growth-inhibitory agent” when used herein refers to a compound or composition that inhibits growth of a cell, such as a Wnt-overexpressing cancer cell, either in vitro or in vivo. Thus, a growth-inhibitory agent is one that significantly reduces the percentage of malignant cells in S phase. Examples of growth-inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information on growth-inhibitory agents can be found in The Molecular Basis of Cancer, Mendelsohn and Israel, eds., Chapter 1, entitled “Cell cycle regulation, oncogenes, and antineoplastic drugs” by Murakami et al., (VVB Saunders: Philadelphia, 1995), p. 13. Additional examples include tumor necrosis factor (TNF), an antibody capable of inhibiting or neutralizing the angiogenic activity of acidic or basic FGF or hepatocyte growth factor (HGF), an antibody capable of inhibiting or neutralizing the coagulant activities of tissue factor, protein C, or protein S (see, WO 91/01753, published 21 Feb. 1991), or an antibody capable of binding to HER2 receptor (WO 89/06692), such as the 4D5 antibody (and functional equivalents thereof) (e.g., WO 92/22653).

In a pharmacological sense, in the context of the present invention, a “therapeutically effective amount” of an active agent such as a polypeptide or agonist or antagonist thereto or an antibody, refers to an amount effective in the treatment of a cancerous disorder in a mammal and can be determined empirically. Determination of a therapeutically effective amount can be accomplished by any method known to those skilled in the art.

As used herein, an “effective amount” of an active agent such as a polypeptide or agonist or antagonist thereto or an antibody, refers to an amount effective for carrying out a stated purpose, wherein such amounts may be determined empirically for the desired effect.

The herein described nucleic acids, polypeptides, antibodies, agonists, and antagonists, when used therapeutically are referred to herein as “Therapeutics”. Methods of administration of therapeutics include, but are not limited to, intradermal, intramuscular, intraperitoneal, intravenous, subcutaneous, intranasal, epidural, and oral routes. The therapeutics of the present invention may be administered by any convenient route, for example by infusion or bolus injection, by absorption through epithelial or mucocutaneous linings (e.g., oral mucosal, rectal and intestinal mucosal, etc.) and may be administered together with other biologically-active agents. Administration can be systemic or local. In addition, it may be advantageous to administer the therapeutic into the central nervous system by any suitable route, including intraventricular and intrathecal injection.

Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a therapeutic of the present invention including, e.g.: (i) encapsulation in liposomes, microparticles, microcapsules; (ii) recombinant cells capable of expressing the therapeutic; (iii) receptor mediated endocytosis (See, e.g., Wu and Wu, 1987. J Biol Chem 262:4429-4432); (iv) construction of a therapeutic nucleic acid as part of a retroviral or other vector, and the like.

In one embodiment of the present invention, the therapeutic may be delivered in a vesicle, in particular a liposome. In a liposome, the protein of the present invention is combined, in addition to other pharmaceutically acceptable carriers, with amphipathic agents such as lipids, which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference.

In yet another embodiment, the therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the therapeutic is a nucleic acid encoding a protein, the therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the therapeutics of the present invention are generally about 20-500 micrograms (μg) of active compound per kilogram (Kg) body weight. Suitable dosage ranges for intranasal administration are generally about 0.01 μg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Unless otherwise defined, all technical and scientific terms used herein have the same meaning as commonly understood by one of ordinary skill in the art to which this invention pertains. In case of conflict, the present specification, including definitions, will control. In addition, the materials, methods, and examples are illustrative only and are not intended to be limiting. Although methods and materials similar or equivalent to those described herein can be used in the practice or testing of the present invention, suitable methods and materials are described below.

All commercially available reagents referred to in the examples were used according to manufacturer's instructions unless otherwise indicated. The source of those cells identified by ATCC accession numbers in the following examples, and throughout the specification, is the American Type Culture Collection, Manassas, Va.

All publications, patent applications, patents, and other references mentioned herein are incorporated by reference in their entirety.

The invention will be further described in the following examples, which do not limit the scope of the invention described in the claims. The following examples are offered for illustrative purposes only, and are not intended to limit the scope of the present invention in any way.

### EXAMPLES

### Example 1

Sample Preparation

Total RNA was isolated from 10 primary colon tumors and 10 normal colon samples using the Ambion Totally RNA kit for isolation of total cellular RNA, catalog number #1910, 2130 Woodward Street, Austin, Tex. 78744-1832. According to the protocol the samples were lysed in a guanidinium based lysis solution and were then extracted sequentially with a Phenol:Chloroform:IAA and Acid-Phenol:Chloroform. The RNA is then precipitated with isopropanol. Poly A+ RNA was extracted using the Oligotex mRNA midi kit, catalog number #70042, 28159 Avenue Stanford, Valencia, Calif., 91355. Using this kit the poly A+ RNA was purified by hybridizing the poly A+ tail to a dT oligomer coupled to a solid-phase matrix.

### Example 2

Probe Generation

Each polyA⁺ RNA sample was reverse transcribed using MMLV reverse-transcriptase, 0.05 μg/ul oligo-dT primer (21 mer), 1× first strand buffer, 0.03 units/ul RNase inhibitor, 500 uM dATP, 500 uM dGTP, 500 uM dTTP, 40 uM dCTP, 40 uM dCTP-Cy3 (BDS) or dCTP-Cy5 (Amersham Pharmacia Biotech). The reverse transcription reaction was performed in a 25 ml volume containing 200 ng polyA⁺ RNA with GEMBRIGHT kits. Specific control polyA⁺ RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, the control mRNAs (YCFR06, YCFR45, YCFR67, YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA respectively. The control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA differential expression patterns. After incubation at 37° C. for 2 hr, each reaction sample (one with Cy3 and another with Cy5 labeling) was treated with 2.5 ml of 0.5M sodium hydroxide and incubated for 20 minutes at 85° C. to the stop the reaction and degrade the RNA. Probes were purified using two successive CHROMA SPIN 30 gel filtration spin columns (Clontech, Palo Alto, Calif. USA) and after combining, both reaction samples were ethanol precipitated using 1 ml of glycogen (1 mg/ml), 60 ml sodium acetate, and 300 ml of 100% ethanol. The probe was then dried to completion using a SpeedVAC (Savant) and resuspended in 14 ul 5×SSC/0.2% SDS.

### Example 3

Hybridizaton

Hybridization reactions contained 9 μl of probe mixture consisting of 0.2 μg each of both Cy3 and Cy5 labeled cDNA synthesis products in 5×SSC, 0.2% SDS hybridization buffer. The probe mixture was heated to 65° C. for 5 minutes and was aliquoted onto the microarray surface and covered with an 1.8 cm² coverslip. The arrays were transferred to a waterproof chamber having a cavity just slightly larger than a microscope slide. The chamber was kept at 100% humidity internally by the addition of 140 μl of 5×SSC in a corner of the chamber. The chamber containing the arrays was incubated for about 6.5 hours at 60° C. The arrays were washed for 10 min at 45° C. in high stringency wash buffer (1×SSC, 0.1% SDS), three times for 10 minutes each at 45° C. in low stringency wash buffer (0.1×SSC), and then dried.

### Example 4

Detection

The microscope used to detect the reporter-labeled hybridization complexes was equipped with an Innova 70 mixed gas 10 W laser (Coherent Lasers, Santa Clara, Calif.) capable of generating spectral lines at 488 nm for excitation of Cy3, and 632 nm for excitation of Cy5. The excitation laser light was focused on the array using a 20× microscope objective (Nikon). The slide containing the array was placed on a computer-controlled X-Y stage on the microscope and raster-scanned past the objective. The 1.8 cm×1.8 cm array used in the present example was scanned with a resolution of 20 micrometers.

In two separate scans, a mixed gas multiline laser excited the two fluorophores sequentially. Emitted light was split, based on wavelength, into two photomultiplier tube detectors (PMT R1477, Hamamatsu Photonics, San Jose, Calif.) corresponding to the two fluorophores. Appropriate filters positioned between the array and the photomultiplier tubes were used to filter the signals. The emission maxima of the fluorophores used were 565 nm for Cy3 and 650 nm for Cy5. Each array was typically scanned twice, one scan per fluorophore using the appropriate filters at the laser source, although the apparatus was capable of recording the spectra from both fluorophores simultaneously.

The sensitivity of the scans was typically calibrated using the signal intensity generated by a cDNA control species added to the probe mix at a known concentration. A specific location on the array contained a complementary DNA sequence, allowing the intensity of the signal at that location to be correlated with a weight ratio of hybridizing species of 1:100,000. When two probes from different sources (e.g., representing test and control cells), each labeled with a different fluorophore, are hybridized to a single array for the purpose of identifying genes that are differentially expressed, the calibration was done by labeling samples of the calibrating cDNA with the two fluorophores and adding identical amounts of each to the hybridization mixture.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte).

### Example 5

Microarray Analysis

The analysis of hybridized microarrays is a multi-step procedure. Arrays were run through an initial quality control procedure that determines spot quality. Upon completion, spots passing a set of pre-defined standards were used to balance the Cy3 (tumor) and Cy5 (normal pool) signals for each gene using an internal method of background correction and signal normalization. The balanced signals were then used to calculate the balanced differential expression ratio for each transcript represented. The ratio was calculated as:

$$\begin{matrix}Ratio = \frac{Cy3}{BalancedCy5} & if Cy3 >= Cy5 \\ Ratio = - \frac{BalancedCy5}{Cy3} & if Cy3 < Cy5\end{matrix}$$

After normalization the arrays were passed through another series of quality control measures, at the array level, to insure confidence in the differential expression values. For arrays passing all quality control measures, a ratio value of 2 fold or greater was accepted as a significant differential both positive and negative ratios. The value of 2 fold was used as it has consistently demonstrated 90%-95% secondary validation rates. In the context of this experiment, a positive ratio indicates an up-regulation of the transcript in the tumor tissue. Transcripts that demonstrate up-regulation in at least 30% of the tumors profiled were identified for further bioinformatic analysis. The differential expression values were quality controlled internally and are shown in Table 3 (up-regulated transcripts) and Table 4 (down-regulated transcripts).

TABLE 3

| | Colon | Colon | Colon | Colon | Colon |
|---|---|---|---|---|---|
| Internal Id | Tumor 1 | Tumor 2 | Tumor 3 | Tumor 4 | Tumor 5 |
| PCTUC_1 | −1.167605 | 3.33153 | 2.21829 | 1.482095 | 2.386943 |
| PCTUC_2 | 3.010309 | 3.331919 | 1.11844 | 2.034488 | 1.618272 |
| PCTUC_3 | 1.024984 | 1.056008 | −1.133479 | −1.434139 | 4.280367 |
| PCTUC_4 | 2.153777 | 3.903957 | 1.664692 | 1.69709 | 3.688466 |
| PCTUC_5 | 2.057309 | 3.513776 | 2.178211 | 1.767936 | 3.360188 |
| PCTUC_6 | 1.434955 | 2.456625 | 1.647487 | 1.257837 | 2.88564 |
| PCTUC_7 | 1.726415 | 6.022513 | 1.97093 | 1.371469 | 1.267824 |
| PCTUC_8 | 3.44923 | 8.567132 | 3.309122 | 1.418101 | 2.207181 |
| PCTUC_9 | −1.24335 | −1.591661 | −1.413795 | 2.313674 | 1.483923 |
| PCTUC_10 | 1.889236 | 2.209288 | 2.358001 | 2.91982 | 1.86346 |
| PCTUC_11 | 1.825643 | 2.982506 | 2.381234 | 1.8578 | 2.436895 |
| PCTUC_12 | 2.29826 | 3.603747 | 2.093798 | 3.73169 | 2.919872 |
| PCTUC_13 | 1.829951 | 2.163258 | 1.540913 | 1.118439 | 2.699494 |
| PCTUC_14 | 2.409517 | 2.765398 | 1.743494 | 2.035633 | 2.988889 |
| PCTUC_15 | 1.774211 | 4.314919 | 3.921481 | 2.014538 | 4.311935 |
| PCTUC_16 | 2.225139 | 4.326321 | 1.919548 | 1.593435 | 3.5661 |
| PCTUC_17 | 1.499352 | 3.324623 | 2.759565 | 1.119117 | 2.112245 |
| PCTUC_18 | 2.446111 | 2.948642 | 1.698977 | 1.715412 | 3.550059 |
| PCTUC_19 | 2.4004 | 3.573775 | 1.950095 | 1.77725 | 3.201406 |
| PCTUC_20 | 1.648393 | 2.617168 | 1.625779 | 1.529432 | 2.786005 |
| PCTUC_21 | 1.298735 | 2.233336 | 1.202361 | 1.422586 | 2.42449 |
| PCTUC_22 | 1.946348 | 3.223213 | 2.01582 | 1.897335 | 3.349705 |
| PCTUC_23 | 1.966244 | 2.633702 | 1.647908 | 1.584596 | 3.069661 |
| PCTUC_24 | 1.612851 | 1.759476 | 1.227302 | 1.212384 | 2.204627 |
| PCTUC_25 | 1.547841 | 3.747698 | 1.780759 | 1.400986 | 3.299834 |
| PCTUC_27 | 1.696275 | 2.027911 | 1.427261 | 1.591907 | 2.715553 |
| PCTUC_28 | 1.734647 | 2.30457 | 1.349411 | 1.609 | 2.535051 |
| PCTUC_29 | 1.218383 | 2.146439 | 1.305923 | 1.36905 | 1.993406 |
| PCTUC_30 | 2.251479 | 4.269543 | 1.869797 | 1.95957 | 4.609859 |
| PCTUC_31 | 1.273212 | 2.008828 | 1.222321 | 1.216924 | 4.79896 |
| PCTUC_32 | 2.211274 | 2.299184 | 1.538199 | 1.846299 | 2.374974 |
| PCTUC_33 | 1.516001 | 2.527318 | 1.163905 | 1.144805 | 3.056935 |
| PCTUC_34 | 1.738326 | 2.761815 | 1.478411 | 1.590443 | 2.763778 |
| PCTUC_35 | 2.450331 | 3.412452 | 2.060661 | 1.893831 | 3.852642 |
| PCTUC_36 | 1.282602 | 2.816709 | 1.325435 | 1.018326 | 1.998563 |
| PCTUC_37 | 2.157884 | 3.047547 | 1.903155 | 1.270211 | 3.594 |
| PCTUC_38 | 2.032504 | 2.527848 | 1.368973 | 1.775121 | 2.579978 |
| PCTUC_39 | 2.057803 | 2.589417 | 1.472552 | 2.317069 | 8.40739 |
| PCTUC_40 | 1.397248 | 1.999109 | 2.080914 | 1.299083 | 2.359739 |
| PCTUC_41 | 2.106049 | 2.4829 | 1.780815 | 1.566652 | 2.513543 |
| PCTUC_42 | 2.11433 | 2.763852 | 1.680019 | 1.171995 | 3.175251 |
| PCTUC_43 | 1.829138 | 4.590826 | 2.386226 | −1.003516 | 1.231775 |
| PCTUC_44 | 1.995429 | 3.195874 | 1.562907 | 1.796879 | 3.65745 |
| PCTUC_45 | 2.476977 | 3.752093 | 2.011098 | 2.046475 | 4.519679 |
| PCTUC_46 | 1.517254 | 1.824735 | 1.121034 | 1.163115 | 2.756811 |
| PCTUC_47 | 1.971729 | 3.51245 | 2.332144 | 3.026521 | 2.137793 |
| PCTUC_48 | 2.66606 | 5.100231 | 2.502615 | 2.33134 | 3.764472 |
| PCTUC_49 | 2.709622 | 4.09865 | 2.081851 | 2.337645 | 5.108332 |
| PCTUC_50 | 1.657113 | 2.514183 | 1.208705 | 1.449126 | 2.884126 |
| PCTUC_51 | 1.961473 | 2.348377 | 1.406842 | 1.547914 | 2.674841 |
| PCTUC_52 | 1.411542 | 3.194549 | 1.993584 | 1.274938 | 1.806577 |
| PCTUC_53 | 2.388666 | 4.043553 | 1.855829 | 1.771712 | 4.134722 |
| PCTUC_54 | 2.698339 | 2.207234 | 1.160583 | 1.06194 | 2.104804 |
| PCTUC_55 | 1.868779 | 2.478753 | 1.525201 | 1.658344 | 2.55561 |
| PCTUC_56 | 2.17674 | 2.960366 | 1.741503 | 1.720215 | 2.986598 |
| PCTUC_57 | 1.537308 | 2.195412 | 1.311886 | 1.381912 | 1.982858 |
| PCTUC_58 | 1.946348 | 3.042087 | 1.42848 | 1.449061 | 2.855023 |
| PCTUC_59 | 2.022084 | 2.498707 | 1.48394 | 1.429058 | 2.51607 |
| PCTUC_60 | 2.317572 | 2.699497 | 1.835917 | 1.959696 | 3.354239 |
| PCTUC_61 | 2.51545 | 2.741376 | 1.854596 | 1.985688 | 2.947656 |
| PCTUC_62 | 1.618495 | 1.648216 | 1.27043 | 1.286811 | 2.033582 |
| PCTUC_63 | 1.732632 | 2.741961 | 1.420588 | 1.602472 | 3.022122 |
| PCTUC_64 | 1.165401 | 1.982774 | 1.254929 | 1.115399 | 1.751606 |
| PCTUC_65 | 1.336602 | 1.949603 | 1.201375 | 1.276382 | 2.270138 |
| PCTUC_66 | 2.015365 | 2.177838 | 1.630885 | 1.77424 | 2.942512 |
| PCTUC_67 | 2.022251 | 2.405791 | 1.785181 | 1.731284 | 2.81626 |
| PCTUC_68 | 1.317565 | 2.295076 | 1.442385 | 1.217891 | 2.656733 |
| PCTUC_69 | 2.06803 | 2.258815 | 1.488382 | 1.728301 | 2.683501 |
| PCTUC_70 | 2.306625 | 2.912195 | 2.046354 | 1.955802 | 3.254495 |
| PCTUC_71 | 1.905904 | 2.366372 | 1.588232 | 1.677139 | 1.97013 |
| PCTUC_72 | 2.018447 | 3.73358 | 1.421489 | 1.405039 | 2.970055 |
| PCTUC_73 | 1.766525 | 2.412998 | 1.502014 | 1.51544 | 2.075916 |
| PCTUC_74 | 1.691552 | 1.793785 | 1.267592 | 1.185903 | 1.924729 |
| PCTUC_75 | 1.846039 | 3.109019 | 1.663261 | 1.625626 | 2.798696 |
| PCTUC_76 | 1.882731 | 2.066262 | 1.742008 | 2.003987 | 2.153655 |
| PCTUC_77 | 1.369232 | 2.060578 | 1.361874 | 1.062815 | 2.131934 |
| PCTUC_78 | 1.63917 | 2.264273 | 1.436098 | 1.202832 | 2.346673 |
| PCTUC_79 | 2.223718 | 3.031543 | 1.922364 | 1.92482 | 3.363707 |
| PCTUC_80 | 1.467536 | 2.186955 | 1.428232 | 1.371915 | 2.048764 |
| PCTUC_81 | 1.912159 | 2.586055 | 1.59879 | 1.821389 | 2.575871 |
| PCTUC_82 | 1.434465 | 2.289142 | 1.630034 | 1.848266 | 2.4429 |
| PCTUC_83 | 1.891248 | 8.992641 | 1.960748 | 1.456855 | 3.430561 |
| PCTUC_84 | 1.944148 | 2.782467 | 1.558292 | 1.82153 | 7.161571 |
| PCTUC_85 | 1.981983 | 1.957546 | 1.457266 | 1.418137 | 2.307765 |
| PCTUC_86 | 2.379777 | 3.951234 | 2.300241 | 2.094229 | 5.10043 |
| PCTUC_87 | 1.846455 | 2.21849 | 1.671109 | 1.930862 | 2.322542 |
| PCTUC_88 | 2.79995 | 3.726188 | 1.975374 | 2.025747 | 3.535597 |
| PCTUC_89 | 1.382285 | 1.979193 | 1.126635 | 1.163368 | 1.985515 |
| PCTUC_90 | 2.162711 | 2.51384 | 1.80511 | 1.877139 | 2.893159 |
| PCTUC_91 | 1.623899 | 2.305127 | 1.427174 | 1.361678 | 3.340343 |
| PCTUC_92 | 1.828539 | 2.704496 | 1.571857 | 1.476535 | 3.043378 |
| PCTUC_93 | 2.670166 | 6.27784 | 2.639735 | 2.603041 | 5.276127 |
| PCTUC_94 | 1.650576 | 2.347257 | 1.551303 | 1.540996 | 2.319753 |
| PCTUC_95 | 2.15389 | 2.130007 | 1.777067 | 2.088068 | 2.410172 |
| PCTUC_96 | 2.035643 | 2.232943 | 1.849501 | 1.655422 | 3.846957 |
| PCTUC_97 | 2.2532 | 2.703321 | 1.774541 | 1.719427 | 1.937724 |
| PCTUC_98 | 1.823728 | 3.187145 | 1.843798 | 1.578049 | 3.37381 |
| PCTUC_99 | 2.934582 | 3.653785 | 2.223708 | 2.350155 | 3.916004 |
| PCTUC_100 | 2.183041 | 3.85372 | 1.631668 | 1.508529 | 3.716182 |
| PCTUC_101 | 1.998253 | 2.540957 | 1.689489 | 1.447247 | 2.309024 |
| PCTUC_102 | 1.637692 | 2.45107 | 1.566288 | 1.728188 | 2.008309 |
| PCTUC_103 | 2.143254 | 2.904234 | 1.622328 | 1.548801 | 3.37803 |
| PCTUC_104 | 1.696224 | 2.088969 | 1.567019 | 1.532818 | 2.249511 |
| PCTUC_105 | 1.855064 | 2.819359 | 1.567435 | 1.543721 | 2.847837 |
| PCTUC_106 | 2.074095 | 2.583958 | 1.802072 | 2.062965 | 2.517983 |
| PCTUC_107 | 1.436427 | 2.014846 | 1.287173 | 1.42702 | 3.187718 |
| PCTUC_108 | 2.466527 | 2.305113 | 2.542691 | 1.881556 | 2.379215 |
| PCTUC_109 | 2.778593 | 5.835248 | 2.612641 | 2.431126 | 5.114062 |
| PCTUC_110 | 1.765069 | 2.276443 | 1.539449 | 1.588558 | 2.340659 |
| PCTUC_111 | 1.621092 | 2.106955 | 1.258185 | 1.182327 | 2.337887 |
| PCTUC_112 | 1.929109 | 2.886708 | 1.581569 | 1.522645 | 2.817362 |
| PCTUC_113 | 2.183752 | 3.438374 | 1.850835 | 2.011902 | 3.310618 |
| PCTUC_114 | 2.780542 | 6.40439 | 2.686503 | 2.496432 | 4.999533 |
| PCTUC_115 | 2.200367 | 4.069643 | 2.01756 | 2.220671 | 4.177684 |
| PCTUC_116 | 2.287616 | 3.50528 | 2.043065 | 2.038058 | 3.823674 |
| PCTUC_117 | 2.970644 | 6.056041 | 2.398937 | 2.169403 | 2.49033 |
| PCTUC_118 | 2.477434 | 2.172511 | 1.88397 | 1.931524 | 2.371255 |
| PCTUC_119 | 1.776452 | 2.629506 | 1.322001 | 1.291075 | 2.405234 |
| PCTUC_120 | 1.982641 | 4.046946 | 1.732223 | 1.753712 | 4.177029 |
| PCTUC_121 | 2.045416 | 3.467918 | 1.856998 | 1.932082 | 4.08297 |
| PCTUC_122 | 2.082013 | 2.52088 | 1.641259 | 1.772476 | 3.19319 |
| PCTUC_123 | 1.892968 | 2.118932 | 1.447326 | 1.521655 | 2.863929 |
| PCTUC_124 | 2.302006 | 4.194275 | 2.018941 | 2.276732 | 4.738862 |
| PCTUC_125 | 2.642775 | 3.22901 | 2.16624 | 2.453689 | 3.785296 |
| PCTUC_126 | 2.253894 | 3.976677 | 1.994424 | 2.144706 | 4.513967 |
| PCTUC_127 | 2.304186 | 5.744323 | 2.38022 | 2.359522 | 5.583232 |
| PCTUC_128 | 2.553788 | 4.196769 | 2.379538 | 2.46891 | 4.286704 |
| PCTUC_129 | 2.325642 | 2.745098 | 1.756556 | 1.931827 | 3.167695 |
| PCTUC_130 | 1.703418 | 2.36412 | 1.743213 | 1.60323 | 2.148247 |
| PCTUC_131 | 1.724546 | 2.582779 | 1.392246 | 1.469603 | 2.513543 |
| PCTUC_132 | 1.940151 | 2.216878 | 1.970094 | 1.976734 | 2.568789 |
| RCTUC_133 | 2.553717 | 3.466767 | 1.779728 | 1.567245 | 4.051633 |
| PCTUC_134 | 2.376674 | 4.774848 | 2.152457 | 1.8395 | 4.683371 |
| PCTUC_135 | 2.144651 | 2.649289 | 1.787653 | 1.872877 | 3.417997 |
| PCTUC_136 | 2.148641 | 2.10056 | 1.450171 | 1.367047 | 2.250633 |
| PCTUC_137 | 2.130816 | 3.194793 | 1.684232 | 1.613193 | 2.966923 |
| PCTUC_138 | 2.136258 | 3.341462 | 1.642945 | 1.58273 | 3.252918 |
| PCTUC_139 | 1.473054 | 1.985499 | 1.192933 | 1.173492 | 2.241265 |
| PCTUC_140 | 2.382936 | 5.190052 | 2.593684 | 2.288506 | 5.946975 |
| PCTUC_141 | 2.122445 | 3.281591 | 1.948166 | 2.022838 | 3.489888 |
| PCTUC_142 | 2.632055 | 6.609196 | 2.636786 | 2.29089 | 5.705941 |
| PCTUC_143 | 2.046356 | 2.483107 | 1.691251 | 1.867148 | 2.636136 |
| PCTUC_144 | 1.992145 | 2.628616 | 1.684575 | 1.899628 | 2.873412 |
| PCTUC_145 | 2.29488 | 2.37603 | 1.896473 | 1.935271 | 2.790786 |
| PCTUC_146 | 2.349353 | 2.360452 | 1.53855 | 1.534619 | 2.342172 |
| PCTUC_147 | 1.97557 | 1.49492 | 1.63718 | 2.030017 | −1.038416 |
| PCTUC_148 | 2.326489 | 3.609815 | 1.936063 | 1.768154 | 3.691488 |
| PCTUC_149 | 2.223878 | 2.409088 | 1.720194 | 1.945538 | 3.391234 |
| PCTUC_150 | 2.333193 | 4.274464 | 2.124192 | 1.964887 | 4.341965 |
| PCTUC_151 | 2.408074 | 1.836668 | 1.819378 | 1.927453 | 1.641701 |
| PCTUC_152 | 2.381697 | 2.747643 | 1.664237 | 1.933687 | 2.809088 |
| PCTUC_153 | 1.62262 | 2.523015 | 3.05855 | 1.294968 | 2.128255 |
| PCTUC_154 | 1.969294 | 4.241415 | 1.971435 | 1.899327 | 3.933199 |
| PCTUC_155 | 1.909149 | 3.250235 | 1.912516 | 1.746694 | 3.157428 |
| PCTUC_156 | 1.451361 | 2.080686 | 1.431723 | 1.311998 | 2.154091 |
| PCTUC_157 | 2.139517 | 2.237462 | 1.468132 | 1.596464 | 2.680392 |
| PCTUC_158 | 2.340751 | 2.427624 | 1.720458 | 1.846447 | 2.538067 |
| PCTUC_159 | 2.592723 | 2.82934 | 1.762373 | 1.801574 | 3.712104 |
| PCTUC_160 | 1.827864 | 2.666218 | 1.512777 | 1.535254 | 3.099494 |
| PCTUC_161 | 1.970345 | 2.297757 | 1.646088 | 1.982166 | 2.605821 |
| PCTUC_162 | 1.439782 | 2.222644 | 1.286601 | 1.290794 | 2.162714 |
| PCTUC_163 | 2.345697 | 3.837572 | 1.862247 | 1.813233 | 3.450867 |
| PCTUC_164 | 2.004076 | 3.285513 | 1.74896 | 1.715412 | 3.191004 |
| PCTUC_165 | 2.412875 | 3.80279 | 2.015456 | 1.967204 | 4.143846 |
| PCTUC_166 | 2.181621 | 2.471198 | 1.548179 | 1.592577 | 2.671936 |
| PCTUC_167 | 2.096034 | 3.911778 | 2.010473 | 1.741283 | 3.903537 |
| PCTUC_168 | 1.651318 | 2.208828 | 1.587337 | 1.49218 | 2.213346 |
| PCTUC_169 | 1.811134 | 2.690632 | 1.77663 | 1.493697 | 3.244027 |
| PCTUC_170 | 2.528349 | 4.629561 | 2.096909 | 1.838336 | 3.974016 |
| PCTUC_171 | 3.086575 | 5.856294 | 3.12292 | 2.78069 | 5.925259 |
| PCTUC_172 | 2.610924 | 3.449815 | 1.901268 | 1.79184 | 3.248729 |
| PCTUC_173 | 3.236281 | 5.599024 | 2.767506 | 3.135199 | 5.606467 |
| PCTUC_174 | 1.971308 | 3.937824 | 1.747312 | 1.87709 | 3.60039 |
| PCTUC_175 | 2.017454 | 2.898789 | 1.806199 | 1.675621 | 3.033045 |
| PCTUC_176 | 3.282002 | 5.875219 | 2.919173 | 2.342964 | 5.071948 |
| PCTUC_177 | 2.392638 | 2.791999 | 1.879821 | 1.711466 | 2.876557 |
| PCTUC_178 | 2.094212 | 3.932021 | 1.863073 | 1.688921 | 3.519515 |
| PCTUC_179 | 2.173889 | 2.059803 | 1.524988 | 1.567178 | 2.219269 |
| PCTUC_180 | 2.07498 | 4.72869 | 2.369165 | 1.775542 | 3.810833 |
| PCTUC_181 | 2.282053 | 2.447175 | 1.364804 | 1.565102 | 2.661733 |
| PCTUC_182 | 2.141199 | 2.648744 | 1.563208 | 1.846968 | 2.751587 |
| PCTUC_183 | 3.488463 | 3.83314 | 2.357281 | 2.562478 | 3.983159 |
| PCTUC_184 | 2.221076 | 4.22895 | 1.935559 | 1.942214 | 4.078297 |
| PCTUC_185 | 2.449592 | 3.155836 | 1.881579 | 2.034842 | 2.937128 |
| PCTUC_186 | 1.762882 | 2.359142 | 1.321211 | 1.423152 | 2.671936 |
| PCTUC_187 | 1.822807 | 2.58226 | 1.442614 | 1.449022 | 1.951994 |
| PCTUC_188 | 1.923808 | 2.515208 | 2.389021 | 1.911519 | 2.53708 |
| PCTUC_189 | 1.948982 | 2.841221 | 1.626155 | 1.526462 | 3.049721 |
| PCTUC_190 | 1.959037 | 3.718122 | 1.823316 | 3.125287 | 2.35109 |
| PCTUC_191 | 1.410424 | 1.569667 | 1.231558 | 1.251293 | 2.681443 |
| PCTUC_192 | 2.053974 | 2.829744 | 1.849315 | 1.897637 | 2.560419 |
| PCTUC_193 | 2.839539 | 2.441042 | 1.599217 | 1.521155 | 2.689553 |
| PCTUC_194 | 2.15666 | 1.996907 | 1.367373 | 1.717722 | 2.412942 |
| PCTUC_195 | 2.665177 | 2.113436 | 1.762802 | 1.931272 | 2.876521 |
| PCTUC_196 | 2.20503 | 2.648168 | 1.708679 | 1.791299 | 2.873039 |
| PCTUC_197 | 3.055709 | 3.674174 | 2.145274 | 2.452721 | 3.976326 |
| PCTUC_198 | 1.477454 | 1.937081 | 1.136061 | 1.210487 | 1.95604 |
| PCTUC_199 | 1.971808 | 2.898166 | 1.580341 | 1.694481 | 3.270516 |
| PCTUC_200 | 2.122297 | 3.621145 | 1.752643 | 1.561154 | 3.118557 |
| PCTUC_201 | 2.389857 | 3.803328 | 1.984605 | 2.117899 | 4.475106 |
| PCTUC_202 | 2.36793 | 2.626245 | 1.949721 | 2.03951 | 3.720536 |
| PCTUC_203 | 2.429015 | 3.120531 | 1.843767 | 1.782332 | 3.47691 |
| PCTUC_204 | 1.724228 | 3.185202 | 1.524506 | 1.612622 | 3.460114 |
| PCTUC_205 | 2.399812 | 5.050709 | 1.950805 | 2.01819 | 3.854446 |
| PCTUC_206 | 2.016684 | 2.115916 | 1.595259 | 1.762565 | 2.502826 |
| PCTUC_207 | 2.449808 | 3.13117 | 1.808911 | 2.02143 | 3.636165 |
| PCTUC_208 | 1.780541 | 2.820107 | 1.579203 | 1.625143 | 2.405794 |
| PCTUC_209 | 2.801661 | 3.453332 | 2.032379 | 1.982221 | 3.402283 |
| PCTUC_210 | 2.062106 | 2.313428 | 1.465423 | 1.57732 | 2.748908 |
| PCTUC_211 | 3.358549 | 1.721248 | 1.794588 | 3.601527 | 1.869076 |
| PCTUC_212 | 2.351744 | 2.68273 | 1.898117 | 2.273726 | 3.325987 |
| PCTUC_213 | 2.730678 | 3.296502 | 1.68906 | 1.697095 | 2.686597 |
| PCTUC_214 | 2.54486 | 2.329126 | 1.914379 | 2.146495 | 2.587393 |
| PCTUC_215 | 2.029841 | 2.073445 | 1.530253 | 1.860207 | 2.410172 |
| PCTUC_216 | 1.986904 | 3.159026 | 1.655331 | 1.574419 | 2.840889 |
| PCTUC_217 | 1.633083 | 2.003624 | 1.448483 | 1.324029 | 1.629999 |
| PCTUC_218 | 2.244204 | 2.915729 | 2.017862 | 1.920212 | 3.04816 |
| PCTUC_219 | 2.246086 | 2.49719 | 1.829735 | 1.570088 | 2.706755 |
| PCTUC_220 | 3.079557 | 4.331718 | 2.211666 | 2.502485 | 4.280556 |
| PCTUC_221 | 2.239766 | 2.779905 | 1.819913 | 1.950109 | 3.386697 |
| PCTUC_222 | 1.600726 | 3.542366 | 3.206866 | 2.886933 | 1.440514 |
| PCTUC_223 | 2.104468 | 2.600874 | 2.20841 | 1.920786 | 2.423107 |
| PCTUC_224 | 2.151362 | 1.813645 | 3.464472 | 5.390255 | 2.281284 |
| PCTUC_225 | 3.906109 | 5.627452 | 2.692037 | 3.592667 | 19.34424 |
| PCTUC_226 | 3.494833 | 5.578166 | 2.032682 | 1.216262 | 2.716067 |
| PCTUC_227 | 2.810362 | 2.059662 | 1.627353 | 1.244153 | 1.214697 |
| PCTUC_228 | 2.201654 | 2.169393 | 1.981317 | 1.985123 | 2.967201 |
| PCTUC_229 | 11.366358 | 2.173938 | 1.090219 | 1.220566 | −1.039668 |
| PCTUC_230 | 3.375315 | 4.060716 | 2.39143 | 1.668518 | 2.078012 |
| PCTUC_231 | 3.248742 | 3.047224 | 2.090379 | 1.713994 | 1.59874 |
| PCTUC_232 | 1.755365 | 2.104231 | 1.455328 | 1.451029 | 1.513414 |
| PCTUC_233 | 2.583899 | 5.946256 | 1.862887 | 1.460509 | 1.088642 |
| PCTUC_234 | 2.467569 | 1.992606 | 1.732377 | 1.383298 | 1.762978 |
| PCTUC_235 | 2.405652 | 2.480653 | 1.955835 | 1.977734 | 1.048993 |
| PCTUC_236 | 2.641375 | 1.227863 | 2.045434 | 1.940119 | −1.244945 |
| PCTUC_237 | 2.522561 | 2.560848 | 1.990514 | 1.659553 | 1.692141 |
| PCTUC_238 | 2.315423 | 1.905347 | 1.35975 | 1.348598 | 1.412436 |
| PCTUC_239 | 2.102382 | 2.242059 | 1.890734 | 1.040776 | 1.178056 |
| PCTUC_240 | 2.408981 | 2.352341 | 1.59466 | 1.537688 | 1.248939 |
| PCTUC_241 | 1.941355 | 2.154684 | 1.518697 | 1.660347 | 1.22467 |
| PCTUC_242 | 2.47675 | 2.847825 | 1.734511 | 1.592506 | 1.85966 |
| PCTUC_243 | 2.971365 | 2.163603 | 1.472201 | 1.335836 | 1.134337 |
| PCTUC_244 | 4.580625 | 1.684575 | 1.141275 | 1.186582 | 1.499127 |
| PCTUC_245 | 2.870873 | 1.709187 | 1.645507 | 1.94663 | 1.993426 |
| PCTUC_246 | 2.515966 | 1.997775 | 1.543553 | 1.387189 | 2.290185 |
| PCTUC_247 | 7.588651 | 3.970692 | 1.914703 | 1.88352 | 1.212324 |
| PCTUC_248 | 4.41854 | 3.05227 | 1.999376 | 1.683012 | −1.188447 |
| PCTUC_249 | 2.56015 | 2.500697 | 1.469604 | 1.28346 | 1.287332 |
| PCTUC_250 | 2.685042 | 3.859677 | 1.120608 | 1.711187 | 1.100062 |
| PCTUC_251 | 1.91974 | 2.922307 | 1.973029 | 1.510662 | 2.356412 |
| PCTUC_252 | 3.177958 | 1.795341 | 3.212665 | 2.383147 | 1.357193 |
| PCTUC_253 | 3.474582 | 3.422618 | 2.248334 | 2.004326 | 2.14971 |
| PCTUC_254 | 2.120358 | 2.259684 | 1.491716 | 1.528243 | 1.586254 |
| PCTUC_255 | 2.572479 | 2.529292 | 1.830403 | 1.752406 | 1.939718 |
| PCTUC_256 | 3.878601 | 3.361156 | 2.1136 | 1.729907 | 1.614187 |
| PCTUC_257 | 2.163267 | 2.263555 | 1.808706 | 1.676525 | 1.800374 |
| PCTUC_258 | 2.388353 | 2.488551 | 1.975504 | 1.860139 | 1.840538 |
| PCTUC_259 | 3.016547 | 3.151346 | 1.743907 | 2.172231 | 1.840373 |
| PCTUC_260 | 3.98491 | 5.031919 | 2.992211 | 2.621613 | 2.29796 |
| PCTUC_261 | 2.601377 | 1.858821 | 2.109545 | 2.090508 | 1.311203 |
| PCTUC_262 | 3.619794 | 2.708431 | 2.073283 | 2.071872 | 1.774984 |
| PCTUC_263 | 3.303594 | 2.90581 | 2.419173 | 1.984032 | 2.126918 |
| PCTUC_264 | 2.046339 | 2.045633 | 1.52259 | 1.660213 | 1.513248 |
| PCTUC_265 | 2.243312 | 2.335322 | 1.585328 | 1.816227 | 1.789087 |
| PCTUC_266 | 5.015629 | 5.737246 | 2.474179 | 2.203052 | 2.508281 |
| PCTUC_267 | 3.728191 | 3.790784 | 2.335161 | 2.334736 | 2.23775 |
| PCTUC_268 | 3.314795 | 2.694717 | 1.956361 | 1.864354 | 1.89976 |
| PCTUC_269 | 2.84269 | 3.09059 | 2.076964 | 2.083827 | 1.921276 |
| PCTUC_270 | 2.809991 | 3.040862 | 2.439587 | 1.90887 | 2.154098 |
| PCTUC_271 | 2.030727 | 2.214447 | 1.438683 | 1.827851 | 1.479516 |
| PCTUC_272 | 2.689879 | 1.867979 | 2.398205 | 1.676425 | 2.069532 |
| PCTUC_273 | 3.520804 | 2.604538 | 1.902172 | 1.925295 | 1.677577 |
| PCTUC_274 | 2.37426 | 2.281653 | 2.032656 | 2.082908 | 2.151423 |
| PCTUC_275 | 2.22381 | 2.450564 | 1.834214 | 1.665578 | 1.4253 |
| PCTUC_276 | 4.804692 | 7.184418 | 2.741525 | 2.269183 | 1.86227 |
| PCTUC_277 | 2.347694 | 2.035078 | 1.350143 | 1.733654 | 1.361137 |
| PCTUC_278 | 2.294004 | 2.944572 | 2.302722 | 1.926946 | 1.729742 |
| PCTUC_279 | 3.504003 | 3.252621 | 1.94464 | 1.827564 | 1.639722 |
| PCTUC_280 | 2.37598 | 2.145716 | 1.585682 | 1.801991 | 1.743704 |
| PCTUC_281 | 3.037311 | 3.299949 | 2.038734 | 2.113893 | 1.853485 |
| PCTUC_282 | 2.668364 | 2.674275 | 1.758186 | 2.16771 | 1.703983 |
| PCTUC_283 | 2.137884 | 2.072191 | 1.510265 | 2.0916 | 1.643327 |
| PCTUC_284 | 2.53009 | 2.233893 | 1.631901 | 1.9737 | 1.555607 |
| PCTUC_285 | 6.332763 | 6.443246 | 2.791887 | 2.457937 | 2.505342 |
| PCTUC_286 | 5.257563 | 5.886498 | 2.668014 | 2.212737 | 2.399585 |
| PCTUC_287 | 3.52928 | 3.100616 | 1.847619 | 1.494685 | 1.53431 |
| PCTUC_288 | 2.102673 | 2.525493 | 1.98892 | 2.130579 | 1.632277 |
| PCTUC_289 | 3.147058 | 3.372461 | 2.276921 | 2.246916 | 2.168657 |
| PCTUC_290 | 4.222344 | 3.670878 | 2.668959 | 2.422289 | 2.324938 |
| PCTUC_291 | 2.935719 | 2.719058 | 1.784167 | 1.982964 | 1.827909 |
| PCTUC_292 | 2.984468 | 2.835538 | 1.844973 | 1.998731 | 1.794246 |
| PCTUC_293 | 2.489122 | 2.058945 | 1.877674 | 2.014703 | 1.595915 |
| PCTUC_294 | 3.294222 | 3.462772 | 2.499072 | 2.195277 | 2.22694 |
| PCTUC_295 | 3.380114 | 2.602693 | 2.26526 | 1.921976 | 1.776908 |
| PCTUC_296 | 2.686099 | 2.244886 | 1.919786 | 1.873626 | 1.961759 |
| PCTUC_297 | 5.196949 | 5.286231 | 3.698334 | 2.791397 | 2.839808 |
| PCTUC_298 | 5.587264 | 2.298222 | 1.91904 | 2.037368 | 1.683946 |
| PCTUC_299 | 5.689084 | 4.919997 | 2.865614 | 2.004579 | 2.254943 |
| PCTUC_300 | 6.772169 | 3.684442 | 2.120501 | 2.219169 | 1.733218 |
| PCTUC_301 | 2.093773 | 2.044039 | 1.909037 | 1.69156 | 2.246853 |
| PCTUC_302 | 3.991078 | 4.580527 | 3.303583 | 2.55929 | 2.767085 |
| PCTUC_303 | 2.370317 | 2.022451 | 1.91632 | 2.531979 | 2.379047 |
| PCTUC_304 | 4.127187 | 3.126195 | 2.034232 | 2.095927 | 1.715794 |
| PCTUC_305 | 2.935688 | 1.487187 | 2.214246 | 2.058172 | 1.721464 |
| PCTUC_306 | 4.746076 | 3.343698 | 2.685661 | 2.155374 | 2.034321 |
| PCTUC_307 | 3.019703 | 2.24161 | 1.876862 | 2.00637 | 1.467769 |
| PCTUC_308 | 4.673458 | 5.029093 | 2.33969 | 2.097128 | 1.778258 |
| PCTUC_309 | 3.407573 | 3.376861 | 2.267434 | 2.172949 | 1.753296 |
| PCTUC_310 | 2.189645 | 2.488225 | 4.420121 | 2.173265 | 1.631521 |
| PCTUC_311 | 2.658834 | 2.382129 | 4.32649 | 1.990568 | 1.769064 |
| PCTUC_312 | 2.127386 | 1.679055 | 2.66417 | 2.236 | 1.107264 |
| PCTUC_313 | 2.975827 | 3.533522 | 2.131942 | 2.058614 | 1.96968 |
| PCTUC_314 | 2.64863 | 3.640082 | 1.860648 | 2.294988 | 1.724837 |
| PCTUC_315 | 2.214283 | 2.206159 | 1.810798 | 1.912709 | 1.248759 |
| PCTUC_316 | 5.557586 | 6.430025 | 2.668887 | 2.1117 | 2.184844 |
| PCTUC_317 | 3.648384 | 3.385846 | 2.241048 | 2.291818 | 1.939281 |
| PCTUC_318 | 5.378511 | 6.426007 | 2.462233 | 2.000173 | 2.068377 |
| PCTUC_319 | 5.452092 | 7.002919 | 3.448654 | 2.65954 | 2.582983 |
| PCTUC_320 | 3.295201 | 3.224249 | 2.418855 | 2.438641 | 2.368528 |
| PCTUC_321 | 2.895834 | 2.315506 | 1.904166 | 2.068255 | 1.961573 |
| PCTUC_322 | 4.143029 | 5.302269 | 2.638332 | 2.136894 | 2.147911 |
| PCTUC_323 | 2.067646 | 2.595632 | 1.525776 | 1.976109 | 1.566396 |
| PCTUC_324 | 2.691354 | 2.89679 | 2.010389 | 2.294306 | 2.107959 |
| PCTUC_325 | 3.999175 | 3.23015 | 2.139746 | 2.538517 | 1.643733 |
| PCTUC_326 | 2.297636 | 1.943776 | 1.721583 | 2.195745 | 1.830882 |
| PCTUC_327 | 4.567304 | 4.428077 | 2.616247 | 2.32428 | 2.129087 |
| PCTUC_328 | 3.539221 | 4.166342 | 2.380124 | 2.448791 | 2.493793 |
| PCTUC_329 | 1.970259 | 1.817101 | 1.692714 | 1.927202 | 1.728867 |
| PCTUC_330 | 2.135586 | 2.0755 | 1.759994 | 2.094738 | 1.947718 |
| PCTUC_331 | 2.782232 | 2.653003 | 1.978628 | 2.037058 | 2.079432 |
| PCTUC_332 | 3.111092 | 2.687885 | 1.992779 | 1.905652 | 1.79761 |
| PCTUC_333 | 2.318447 | 1.787268 | 1.849104 | 1.798626 | 1.446862 |
| PCTUC_334 | 1.874384 | 1.992494 | 1.40384 | 1.65309 | 1.245036 |
| PCTUC_335 | 2.611389 | 2.576149 | 1.959713 | 1.970436 | 1.718427 |
| PCTUC_336 | 2.330261 | 2.28512 | 1.535005 | 8.98456 | 1.550727 |
| PCTUC_337 | 2.241662 | 2.23638 | 1.488679 | 1.767888 | 1.63189 |
| PCTUC_338 | 2.815969 | 2.403253 | 1.838307 | 2.069623 | 1.498996 |
| PCTUC_339 | 2.58594 | 2.473097 | 1.910527 | 2.242478 | 1.915542 |
| PCTUC_340 | 5.321351 | 3.654036 | 2.543868 | 2.509842 | 1.970748 |
| PCTUC_341 | 2.227542 | 2.025373 | 1.566321 | 1.932986 | 1.604847 |
| PCTUC_342 | 2.521508 | 1.735598 | 1.899227 | 1.924772 | 1.730055 |
| PCTUC_343 | 3.248326 | 2.382129 | 1.834694 | 1.930941 | 1.510622 |
| PCTUC_344 | 3.774367 | 3.566884 | 2.270241 | 2.083904 | 2.056787 |
| PCTUC_345 | 2.884718 | 2.611996 | 1.692929 | 2.084061 | 1.635321 |
| PCTUC_346 | 3.646603 | 3.245387 | 2.435109 | 2.143002 | 1.781983 |
| PCTUC_347 | 4.438542 | 4.959843 | 2.631065 | 2.431357 | 2.271578 |
| PCTUC_348 | 2.75379 | 2.535014 | 1.90771 | 2.465073 | 1.67656 |
| PCTUC_349 | 3.909889 | 4.528754 | 2.668959 | 2.322136 | 2.002069 |
| PCTUC_350 | 2.792134 | 2.57382 | 1.883458 | 2.029199 | 1.951232 |
| PCTUC_351 | 4.496643 | 2.292628 | 1.605078 | 1.953279 | 1.640106 |
| PCTUC_352 | 2.975827 | 3.498726 | 1.761696 | 1.601816 | 1.62745 |
| PCTUC_353 | 5.921041 | 1.897311 | 1.294325 | 1.676672 | 1.453307 |
| PCTUC_354 | 6.059254 | 6.249482 | 3.449207 | 2.921316 | 2.949268 |
| PCTUC_355 | 6.229225 | 5.959078 | 3.044651 | 2.209111 | 2.548369 |
| PCTUC_356 | 3.354931 | 3.866993 | 2.345238 | 2.18334 | 1.88739 |
| PCTUC_357 | 2.751259 | 2.617638 | 2.173257 | 2.049143 | 2.249806 |
| PCTUC_358 | 1.569435 | 1.690702 | 1.300281 | 1.784292 | 1.63359 |
| PCTUC_359 | 2.559835 | 2.73093 | 1.689176 | 1.995529 | 1.681855 |
| PCTUC_360 | 4.107032 | 3.399894 | 2.794858 | 2.497178 | 2.294676 |
| PCTUC_361 | 3.003746 | 2.494177 | 1.995029 | 1.932543 | 1.674815 |
| PCTUC_362 | 2.440848 | 2.140181 | 1.675391 | 1.78602 | 1.55943 |
| PCTUC_363 | 2.743691 | 3.369475 | 2.163374 | 2.025629 | 1.782426 |
| PCTUC_364 | 5.331734 | 6.668924 | 2.512076 | 2.79165 | 2.216435 |
| PCTUC_365 | 2.84881 | 2.181204 | 1.583908 | 1.936329 | 1.921305 |
| PCTUC_366 | 3.93763 | 3.833567 | 2.871508 | 2.51805 | 2.47756 |
| PCTUC_367 | 4.134794 | 3.168116 | 2.510271 | 2.138366 | 1.921304 |
| PCTUC_368 | 3.757106 | 3.676876 | 2.459811 | 2.157374 | 2.073027 |
| PCTUC_369 | 3.708133 | 2.510159 | 2.104059 | 1.597603 | 1.478547 |
| PCTUC_370 | 3.867609 | 3.547782 | 2.471164 | 2.112282 | 2.015814 |
| PCTUC_371 | 2.545983 | 1.902669 | 1.798742 | 2.149657 | 1.362599 |
| PCTUC_372 | 2.120895 | 1.985513 | 1.655881 | 1.857098 | 1.574041 |
| PCTUC_373 | 3.317969 | 3.300145 | 2.590271 | 2.307053 | 2.30217 |
| PCTUC_374 | 1.610954 | 1.528575 | 1.254938 | 1.805304 | 1.661116 |
| PCTUC_375 | 2.64639 | 2.026726 | 2.394302 | 1.965622 | 1.688982 |
| PCTUC_376 | 2.58554 | 2.26397 | 2.149105 | 1.910245 | 1.473766 |
| PCTUC_377 | 1.974547 | 1.843346 | 1.498608 | 1.76156 | 1.304683 |
| PCTUC_378 | 1.799032 | 1.909818 | 1.266351 | 1.805849 | 1.422752 |
| PCTUC_379 | 2.809991 | 3.104309 | 1.618504 | 1.965153 | 1.507427 |
| PCTUC_380 | 2.062649 | 1.906358 | 1.564003 | 2.094136 | 1.68139 |
| PCTUC_381 | 4.934052 | 7.285052 | 2.611982 | 2.38664 | 2.165479 |
| PCTUC_382 | 3.145328 | 3.340626 | 2.167187 | 2.4187 | 1.973113 |
| PCTUC_383 | 3.160824 | 3.060342 | 2.072027 | 2.30552 | 2.01981 |
| PCTUC_384 | 3.120152 | 2.910294 | 1.922039 | 1.802327 | 1.571349 |
| PCTUC_385 | 3.083806 | 3.15393 | 2.544231 | 2.492088 | 2.528433 |
| PCTUC_386 | 3.162037 | 3.094964 | 2.126098 | 2.45989 | 2.204981 |
| PCTUC_387 | 4.007894 | 3.551215 | 3.060653 | 2.760879 | 2.148093 |
| PCTUC_388 | 1.665821 | 1.71606 | 1.326498 | 1.722747 | 1.737454 |
| PCTUC_389 | 1.640459 | 6.711383 | 1.958431 | 2.48378 | 1.404279 |
| PCTUC_390 | 1.75192 | 1.944185 | 1.57942 | 2.008176 | 1.780645 |
| PCTUC_391 | 4.6229 | 4.782019 | 3.078028 | 2.821985 | 2.757624 |
| PCTUC_392 | 3.485953 | 2.236763 | 1.643519 | 2.5109 | 1.94171 |
| PCTUC_393 | 3.429088 | 3.19615 | 2.760777 | 2.330962 | 2.317443 |
| PCTUC_394 | 1.956778 | 2.014125 | 2.407287 | 2.088266 | 1.803808 |
| PCTUC_395 | 2.301491 | 1.941803 | 2.02459 | 2.445048 | 1.522407 |
| PCTUC_396 | 2.572758 | 2.096845 | 1.77806 | 1.988507 | 1.516392 |
| PCTUC_397 | 3.461717 | 3.924062 | 2.397048 | 2.627406 | 2.340878 |
| PCTUC_398 | 2.060445 | 2.129671 | 1.765777 | 2.154751 | 2.0387 |
| PCTUC_399 | 1.784871 | 1.92874 | 1.606977 | 2.111418 | 1.982721 |
| PCTUC_400 | 2.912105 | 2.509347 | 2.208568 | 2.187831 | 1.902238 |
| PCTUC_402 | 2.473379 | 2.035924 | 2.015918 | 2.125032 | 1.846033 |
| PCTUC_403 | 3.044617 | 2.684285 | 2.137905 | 2.04104 | 1.860546 |
| PCTUC_404 | 2.718181 | 2.694169 | 2.300801 | 2.303463 | 2.093521 |
| PCTUC_406 | 3.178427 | 2.74695 | 2.29744 | 2.217696 | 2.166758 |
| PCTUC_407 | 3.056299 | 2.663542 | 2.343237 | 2.35323 | 2.213864 |
| PCTUC_408 | 2.020171 | 2.288822 | 1.580787 | 2.087243 | 2.022461 |
| PCTUC_409 | 2.004701 | 1.91753 | 2.043845 | 2.029877 | 1.374792 |
| PCTUC_410 | 2.823477 | 2.079084 | 1.828117 | 1.971688 | 2.59637 |
| PCTUC_411 | 2.442635 | 2.821673 | 2.1775 | 2.162623 | 2.374948 |
| PCTUC_412 | 2.522494 | 2.787869 | 2.044906 | 2.251569 | 1.78448 |
| PCTUC_413 | 4.823759 | 4.816738 | 2.444866 | 2.557981 | 2.332555 |
| PCTUC_414 | 4.140165 | 4.372881 | 2.41847 | 2.4379 | 2.252348 |
| PCTUC_415 | 2.581056 | 2.333065 | 1.748048 | 2.177715 | 1.781241 |
| PCTUC_416 | 3.979205 | 4.425482 | 3.179835 | 2.49232 | 2.257952 |
| PCTUC_417 | 3.114341 | 3.119072 | 2.283371 | 2.453368 | 2.115911 |
| PCTUC_418 | 3.380672 | 2.566587 | 2.098064 | 1.985276 | 1.816855 |
| PCTUC_419 | 2.318419 | 2.760706 | 2.229328 | 2.329481 | 1.766098 |
| PCTUC_420 | 2.963531 | 3.143115 | 2.18586 | 2.355827 | 2.258371 |
| PCTUC_421 | 3.523689 | 2.163615 | 1.651499 | 2.514414 | 1.430685 |
| PCTUC_422 | 2.236862 | 2.181313 | 2.204773 | 1.380721 | 1.466239 |
| PCTUC_423 | 3.100164 | 2.768556 | 2.230654 | 2.389357 | 2.029327 |
| PCTUC_424 | 4.047059 | 3.834085 | 2.527061 | 2.484142 | 2.170724 |
| PCTUC_425 | 1.785719 | 1.839733 | 1.472349 | 2.018828 | 1.833956 |
| PCTUC_426 | 6.098773 | 5.734037 | 3.16041 | 2.664088 | 2.467161 |
| PCTUC_427 | 2.271291 | 2.464738 | 1.668215 | 1.816736 | 1.593545 |
| PCTUC_428 | 5.065524 | 5.161148 | 3.054286 | 2.644954 | 2.300476 |
| PCTUC_429 | 4.544531 | 5.866208 | 2.376281 | 2.569743 | 2.496617 |
| PCTUC_430 | 6.353233 | 5.8785 | 2.560364 | 2.614165 | 2.184317 |
| PCTUC_431 | 3.771823 | 4.187628 | 2.359161 | 2.35775 | 2.100648 |
| PCTUC_432 | 3.056736 | 2.814484 | 2.227544 | 2.181698 | 2.094198 |
| PCTUC_433 | 4.054384 | 4.009579 | 2.622651 | 2.591521 | 2.812049 |
| PCTUC_434 | 2.326007 | 2.291512 | 2.000558 | 2.329474 | 2.327149 |
| PCTUC_435 | 3.45679 | 3.138741 | 2.380019 | 2.03344 | 1.681855 |
| PCTUC_436 | 2.63824 | 2.092904 | 1.829563 | 1.843457 | 1.680504 |
| PCTUC_437 | 6.731214 | 8.586503 | 3.54067 | 2.651634 | 3.064444 |
| PCTUC_438 | 4.981229 | 5.375686 | 2.421715 | 2.319799 | 2.014582 |
| PCTUC_439 | 2.697179 | 2.422815 | 2.011376 | 1.926572 | 2.499886 |
| PCTUC_440 | 2.149141 | 2.022499 | 1.430869 | 2.1492 | 1.660266 |
| PCTUC_441 | 4.051781 | 3.311647 | 2.314 | 2.437911 | 2.113835 |
| PCTUC_442 | 3.187954 | 2.164001 | 2.019149 | 2.336673 | 1.726152 |
| PCTUC_443 | 2.877242 | 2.275949 | 2.151833 | 1.90094 | 1.556428 |
| PCTUC_444 | 2.654798 | 2.379096 | 1.644744 | 2.113579 | 1.462735 |
| PCTUC_445 | 2.696902 | 2.734087 | 2.162968 | 2.128721 | 1.531066 |
| PCTUC_446 | 3.552812 | 3.808604 | 2.448 | 2.377787 | 2.223854 |
| PCTUC_447 | 2.457076 | 2.397561 | 1.660214 | 1.962635 | 1.665535 |
| PCTUC_448 | 2.260049 | 1.830025 | 2.049749 | 2.111274 | 1.336249 |
| PCTUC_449 | 5.494178 | 6.597764 | 2.759527 | 2.631596 | 2.232384 |
| PCTUC_450 | 5.396477 | 5.850169 | 2.957937 | 2.551245 | 2.323228 |
| PCTUC_451 | 3.234099 | 3.824688 | 1.805116 | 1.960044 | 1.567665 |
| PCTUC_452 | 1.988279 | 1.911949 | 1.746898 | 1.9211 | 1.410218 |
| PCTUC_453 | 4.156703 | 3.429154 | 2.668119 | 2.437835 | 1.985651 |
| PCTUC_454 | 1.863835 | 1.840781 | 1.565095 | 2.077737 | 1.72461 |
| PCTUC_455 | 3.180538 | 3.255673 | 2.373674 | 2.493548 | 2.303203 |
| PCTUC_456 | 2.603995 | 2.660861 | 1.828479 | 2.013692 | 2.134072 |
| PCTUC_457 | 1.951543 | 1.738451 | 1.314122 | 1.450489 | 1.474622 |
| PCTUC_458 | 3.082505 | 3.1755 | 1.641161 | 1.860139 | 2.340101 |
| PCTUC_459 | 2.093832 | 2.06715 | 1.780446 | 1.808486 | 1.52498 |
| PCTUC_460 | 4.152094 | 3.292449 | 2.278043 | 2.468725 | 2.022536 |
| PCTUC_461 | 2.949867 | 3.067491 | 2.152803 | 1.982133 | 1.933115 |
| PCTUC_462 | 5.260846 | 3.310389 | 3.009029 | 2.364296 | 2.113845 |
| PCTUC_464 | 3.028954 | 3.059962 | 2.519372 | 1.971144 | 1.852329 |
| PCTUC_467 | 1.851681 | 1.727232 | 1.185358 | 1.913809 | 1.664958 |
| PCTUC_468 | 5.289123 | 4.978931 | 2.630347 | 3.07653 | 2.621873 |
| PCTUC_469 | 3.783153 | 4.114516 | 2.305927 | 2.928126 | 2.397941 |
| PCTUC_470 | 7.936601 | 6.850856 | 3.652995 | 2.918136 | 2.82882 |
| PCTUC_471 | 2.180746 | 2.761191 | 2.199378 | 2.584434 | 2.013092 |
| PCTUC_472 | 5.085942 | 5.354563 | 3.110618 | 2.8712 | 2.575828 |
| PCTUC_473 | 4.312363 | 3.72427 | 2.239403 | 2.415173 | 1.983299 |
| PCTUC_474 | 3.696658 | 3.291336 | 2.905436 | 2.244899 | 2.026702 |
| PCTUC_475 | 5.070798 | 5.750005 | 3.2816 | 2.710166 | 2.482052 |
| PCTUC_476 | 4.073975 | 4.069614 | 2.38597 | 2.549321 | 2.176326 |
| PCTUC_477 | 2.846882 | 2.862958 | 2.415767 | 2.441686 | 2.023011 |
| PCTUC_478 | 1.198274 | 6.471813 | 2.447198 | 2.589143 | 2.060662 |
| PCTUC_479 | 6.380529 | 6.312082 | 2.940724 | 2.148456 | 2.107159 |
| PCTUC_480 | 3.605897 | 2.601701 | 2.728055 | 2.094268 | 1.77564 |
| PCTUC_481 | 4.150877 | 2.25544 | 2.163176 | 2.125869 | 1.578758 |
| PCTUC_482 | 4.905541 | 6.253034 | 2.660336 | 2.411183 | 1.90211 |
| PCTUC_483 | 3.804823 | 4.135355 | 1.910409 | 2.431383 | 1.665717 |
| PCTUC_484 | 2.982269 | 2.854497 | 1.857548 | 1.770715 | 1.687256 |
| PCTUC_485 | 2.400091 | 2.499066 | 2.048137 | 2.125371 | 1.763658 |
| PCTUC_486 | 5.368877 | 6.171281 | 2.094893 | 2.564498 | 1.828952 |
| PCTUC_487 | 4.33764 | 3.811306 | 2.244857 | 2.606614 | 1.838906 |
| PCTUC_488 | 5.781398 | 4.944817 | 2.550974 | 2.317226 | 1.946061 |
| PCTUC_489 | 4.129999 | 4.597306 | 2.073404 | 2.376219 | 1.772075 |
| PCTUC_490 | 2.533209 | 2.249715 | 1.902172 | 2.280487 | 1.291751 |
| PCTUC_491 | 4.349143 | 3.271342 | 1.968882 | 2.122043 | 1.913929 |
| PCTUC_492 | 3.442778 | 3.200904 | 2.026083 | 2.070875 | 1.888175 |
| PCTUC_493 | 2.000497 | 3.239336 | 1.994512 | 1.847443 | 1.764806 |
| PCTUC_494 | 2.670316 | 2.293422 | 1.684396 | 1.868083 | 1.564883 |
| PCTUC_495 | 1.597283 | 1.984764 | 1.610974 | 1.99927 | 1.399811 |
| PCTUC_496 | 1.775757 | 1.56946 | 1.436648 | 2.088016 | 1.193644 |
| PCTUC_497 | 2.093349 | 1.9671 | 2.180265 | 2.309402 | 1.536103 |
| PCTUC_498 | 2.722987 | 2.821209 | 2.018141 | 2.142493 | 1.737708 |
| PCTUC_499 | 2.779592 | 2.158303 | 1.893526 | 2.309402 | 1.44936 |
| PCTUC_500 | 3.574841 | 3.073884 | 2.401034 | 2.240357 | 2.004712 |
| PCTUC_501 | 2.54213 | 2.105277 | 2.337602 | 2.628652 | 2.246022 |
| PCTUC_502 | 2.178911 | 2.285843 | 1.789459 | 2.164132 | 1.590264 |
| PCTUC_503 | 2.414157 | 1.944002 | 1.772574 | 2.32658 | 1.672217 |
| PCTUC_504 | 2.463425 | 2.447765 | 2.163759 | 2.089343 | 1.930668 |
| PCTUC_505 | 2.12314 | 1.912607 | 1.443313 | 1.437833 | 1.400606 |
| PCTUC_506 | 2.791865 | 2.085928 | 1.808019 | 2.124345 | 2.001914 |
| PCTUC_507 | 1.894859 | 1.972295 | 1.660342 | 1.781779 | 1.749316 |
| PCTUC_508 | 1.639329 | 2.087199 | 1.918195 | 1.927565 | 1.960287 |
| PCTUC_509 | 1.92661 | 2.409046 | 1.17896 | 2.502893 | 1.697634 |
| PCTUC_510 | 2.74997 | 2.181224 | 1.981297 | 2.163146 | 1.72972 |
| PCTUC_511 | 2.141737 | 2.395646 | 2.027365 | 2.038215 | 1.75701 |
| PCTUC_512 | 2.174905 | 1.714814 | 2.488783 | 2.689739 | 1.448017 |
| PCTUC_513 | 3.039541 | 2.820119 | 2.028585 | 2.125969 | 2.057135 |
| PCTUC_514 | 3.052588 | 2.991949 | 2.247591 | 2.51345 | 2.333359 |
| PCTUC_515 | 2.034073 | 2.038699 | 1.665666 | 1.59906 | 1.481016 |
| PCTUC_516 | 2.264423 | 2.08314 | 2.138243 | 2.297952 | 1.845813 |
| PCTUC_517 | 4.065478 | 3.998617 | 2.456918 | 2.576724 | 2.081958 |
| PCTUC_518 | 5.685041 | 1.377179 | 2.230373 | 2.437649 | 1.905197 |
| PCTUC_519 | 4.014813 | 3.038095 | 2.627206 | 2.849769 | 2.049812 |
| PCTUC_520 | 2.454347 | 2.33136 | 2.101032 | 2.413781 | 1.580209 |
| PCTUC_521 | 4.696158 | 3.993624 | 1.967548 | 2.5262 | 1.740719 |
| PCTUC_522 | 2.955145 | 3.214377 | 2.28823 | 2.614905 | 2.264642 |
| PCTUC_523 | 2.031883 | 2.267176 | 1.740335 | 2.357091 | 1.943224 |
| PCTUC_524 | 3.117527 | 3.173575 | 1.999416 | 2.450931 | 1.852275 |
| PCTUC_525 | 2.676281 | 2.769179 | 1.85005 | 1.882107 | 1.618436 |
| PCTUC_526 | 1.666611 | 2.198318 | 1.581107 | 2.010444 | 1.593466 |
| PCTUC_527 | 6.939546 | 6.183732 | 3.480864 | 2.834689 | 2.319295 |
| PCTUC_528 | 4.949556 | 4.691981 | 3.093936 | 2.604392 | 2.059476 |
| PCTUC_529 | 2.075038 | 1.719615 | 1.560014 | 1.896456 | 1.631452 |
| PCTUC_530 | 4.147139 | 3.497023 | 2.339375 | 2.565444 | 2.050107 |
| PCTUC_531 | 2.343305 | 2.01423 | 2.036322 | 2.090427 | 1.881663 |
| PCTUC_532 | 4.510367 | 4.525547 | 2.959393 | 2.010432 | 1.662161 |
| PCTUC_533 | 3.849342 | 3.696642 | 2.538885 | 2.515058 | 2.033303 |
| PCTUC_534 | 1.767598 | 2.102775 | 1.631302 | 2.020012 | 1.818509 |
| PCTUC_535 | 4.195316 | 4.086377 | 2.345238 | 2.621754 | 1.959155 |
| PCTUC_536 | 6.243456 | 2.785664 | 2.43465 | 2.10029 | 2.731689 |
| PCTUC_537 | 5.38413 | 4.90169 | 2.114623 | 2.51 | 2.008325 |
| PCTUC_538 | 2.602264 | 2.538037 | 2.19584 | 2.023753 | 1.577312 |
| PCTUC_539 | 3.54853 | 2.237731 | 2.088234 | 2.511449 | 1.925883 |
| PCTUC_540 | 2.888831 | 2.882751 | 2.434141 | 2.23018 | 1.710604 |
| PCTUC_541 | 3.269265 | 2.895229 | 2.268465 | 2.370985 | 1.904343 |
| PCTUC_542 | 2.44896 | 2.29352 | 2.003177 | 2.059909 | 2.051723 |
| PCTUC_543 | 2.512787 | 1.711784 | 2.086737 | 2.002021 | 1.785412 |
| PCTUC_544 | 2.681708 | 2.466954 | 2.712077 | 2.575649 | 2.445763 |
| PCTUC_545 | 2.533259 | 2.391037 | 2.119718 | 2.084042 | 1.633101 |
| PCTUC_546 | 2.556877 | 2.404527 | 2.076303 | 2.310916 | 2.3094 |
| PCTUC_547 | 2.186541 | 2.087202 | 1.802749 | 2.354466 | 1.737011 |
| PCTUC_548 | 2.414651 | 2.029257 | 2.061789 | 2.085276 | 1.74288 |
| PCTUC_549 | 1.670813 | 1.868073 | 1.944966 | 1.7995 | 3.077506 |
| PCTUC_550 | 2.035058 | 2.595207 | 2.075765 | 2.609532 | 1.910092 |
| PCTUC_551 | 2.274966 | 2.047911 | 1.7206 | 2.051094 | 1.47953 |
| PCTUC_552 | 2.638577 | 2.758001 | 2.067063 | 2.31942 | 1.843889 |
| PCTUC_553 | 2.981036 | 2.50471 | 1.883204 | 2.115905 | 1.82132 |
| PCTUC_554 | 2.436244 | 2.485835 | 1.824445 | 2.12429 | 1.804349 |
| PCTUC_555 | 2.944834 | 2.212094 | 1.637195 | 2.050716 | 1.812436 |
| PCTUC_556 | 2.714275 | 2.715855 | 1.841325 | 2.15853 | 2.076278 |
| PCTUC_557 | 3.131188 | 2.954755 | 2.162656 | 2.40551 | 1.870438 |
| PCTUC_558 | 2.132947 | 2.228063 | 1.419358 | 1.864631 | 1.463866 |
| PCTUC_559 | 2.332394 | 2.15486 | 1.495926 | 1.887638 | 1.915631 |
| PCTUC_560 | 3.196013 | 2.914045 | 2.145384 | 2.59745 | 1.823627 |
| PCTUC_561 | 2.1513 | 1.96924 | 1.761033 | 1.414338 | −1.168462 |
| PCTUC_562 | 1.659784 | 1.154773 | 1.048913 | 1.024868 | −1.336392 |
| PCTUC_563 | 2.585276 | 2.190079 | 1.313003 | 1.250177 | −1.309359 |
| PCTUC_564 | 2.817678 | 3.787903 | 1.135624 | 1.872162 | 1.369615 |
| PCTUC_565 | 1.641978 | 1.358107 | −1.036329 | 1.016858 | −1.118048 |
| PCTUC_566 | 1.548184 | 1.57239 | 1.177027 | 1.234061 | −1.024877 |
| PCTUC_568 | 1.654475 | 1.291164 | 1.043306 | 1.184206 | −1.104657 |
| PCTUC_569 | 1.405539 | 2.485698 | 1.313684 | −1.19257 | 1.149279 |
| PCTUC_570 | 1.676607 | 1.560754 | 1.085764 | 1.030557 | −1.101693 |
| PCTUC_571 | 1.513431 | 2.937735 | 2.57267 | 2.722686 | 1.00085 |
| PCTUC_572 | 1.663791 | −1.06477 | 1.593715 | 2.486905 | 1.926183 |
| PCTUC_573 | 1.767246 | 1.481849 | −1.021755 | 1.165484 | −1.078355 |
| PCTUC_574 | 1.291006 | 1.252516 | 1.008709 | 1.066555 | −1.042773 |
| PCTUC_575 | 1.69959 | 1.726639 | 1.383058 | 1.334899 | −1.044352 |
| PCTUC_576 | 1.858294 | 1.387836 | 1.157076 | 1.364982 | 1.10785 |
| PCTUC_577 | 1.900154 | 1.671832 | 1.240975 | 1.305402 | −1.030198 |
| PCTUC_578 | 2.089452 | 1.667765 | 1.670322 | 1.539216 | −1.080703 |
| PCTUC_579 | 3.319847 | 3.063403 | 2.058318 | 1.82173 | −1.477657 |
| PCTUC_580 | 2.527649 | 1.235273 | 1.142485 | 1.536292 | −1.102233 |
| PCTUC_581 | 4.171175 | 1.930353 | 1.289021 | 1.280861 | 1.790144 |
| PCTUC_582 | 2.060718 | 2.143747 | 2.221659 | −1.132255 | 1.311752 |
| PCTUC_583 | 1.955715 | 2.752954 | 2.15881 | 1.1520894 | −1.178174 |
| PCTUC_584 | 1.368932 | 1.918412 | 2.436657 | 2.091652 | 1.266287 |
| PCTUC_585 | 3.066615 | 1.758414 | 3.530922 | 2.242071 | 1.113677 |
| PCTUC_586 | 2.625219 | 2.259675 | 1.576642 | 1.534752 | −1.178215 |
| PCTUC_587 | 2.562503 | 1.885402 | 2.097867 | 2.085679 | −1.083323 |
| PCTUC_588 | 1.908116 | 1.08511 | 1.324322 | 1.262651 | −1.337089 |
| PCTUC_589 | 1.973591 | 1.75518 | 1.465789 | 1.763982 | 1.125532 |
| PCTUC_590 | 2.289264 | 1.587785 | 1.488175 | 1.429079 | 1.038875 |
| PCTUC_591 | 2.166435 | 1.574134 | 2.066997 | 1.512965 | 1.347126 |
| PCTUC_592 | 2.391229 | 1.668447 | 2.206781 | 1.895666 | 1.297054 |
| PCTUC_593 | 2.993244 | 2.143421 | 1.786839 | 1.82173 | 1.211386 |
| PCTUC_594 | 2.266927 | 1.81015 | 1.432413 | 1.377111 | 1.069318 |
| PCTUC_595 | 1.529021 | 1.283073 | −1.078631 | 1.000447 | −1.157923 |
| PCTUC_596 | 1.972231 | 1.668146 | 1.377539 | 1.459993 | −1.060956 |
| PCTUC_597 | 2.14585 | 4.201519 | 2.722095 | 1.036502 | 1.011944 |
| PCTUC_598 | 2.354877 | 1.619132 | 1.433926 | 1.77209 | −1.100912 |
| PCTUC_599 | 2.414222 | 2.455986 | 1.397936 | 1.834194 | 1.076061 |
| PCTUC_600 | 1.898288 | 2.353238 | 2.2936 | 2.959539 | 1.338679 |
| PCTUC_601 | 2.799655 | 4.394316 | 1.374927 | 1.371902 | −1.181136 |
| PCTUC_602 | 1.966814 | 2.086259 | 1.620849 | 1.557261 | 1.013303 |
| PCTUC_603 | 5.415817 | −1.825225 | 4.445374 | 7.686296 | −1.623484 |
| PCTUC_604 | 4.548471 | 3.68559 | 1.787653 | 2.5105 | −1.029682 |
| PCTUC_605 | 2.45415 | 2.507529 | 1.857248 | 3.294002 | 1.253527 |
| PCTUC_606 | 1.457075 | 2.362059 | 2.69411 | 1.668789 | 1.28103 |
| PCTUC_607 | 2.371922 | 1.77947 | 1.71337 | 1.366738 | −1.082459 |
| PCTUC_608 | 2.356047 | 2.154961 | 2.013607 | 1.76495 | 1.503782 |
| PCTUC_609 | 3.341621 | 2.040385 | 1.907361 | 1.673439 | 1.192257 |
| PCTUC_610 | 6.863899 | 3.046129 | 3.930916 | 2.956858 | 1.672432 |
| PCTUC_611 | 1.772479 | 3.18666 | 1.441249 | 1.51261 | 1.350461 |
| PCTUC_612 | 2.453625 | 2.656935 | 1.42877 | 1.68846 | 1.177155 |
| PCTUC_613 | 2.322756 | 1.295258 | 1.398793 | 1.632847 | 1.081717 |
| PCTUC_614 | 2.105337 | 2.060017 | 1.605743 | 1.710592 | 1.31056 |
| PCTUC_615 | 2.061213 | 2.15023 | 1.301226 | 1.267069 | 1.225575 |
| PCTUC_616 | 2.308388 | 1.574555 | 1.407101 | 1.429502 | 1.100921 |
| PCTUC_617 | 1.576303 | 1.238666 | 1.102804 | 1.104352 | 1.037544 |
| PCTUC_618 | 5.2704 | 3.445125 | 2.291641 | 2.158062 | 1.304951 |
| PCTUC_619 | 3.6311 | 1.682945 | 2.344108 | 2.325649 | 1.262526 |
| PCTUC_620 | 2.279748 | 1.49322 | 1.640359 | 1.400027 | 1.118628 |
| PCTUC_621 | 2.725315 | 2.27424 | 1.951459 | 1.822087 | 1.321089 |
| PCTUC_622 | 3.367776 | 2.442924 | 2.535629 | 2.154737 | 1.430283 |
| PCTUC_623 | 3.551299 | 2.625053 | 1.846458 | 2.015363 | 1.363958 |
| PCTUC_624 | 2.990381 | 1.391522 | 1.569815 | 2.308152 | 1.489686 |
| PCTUC_625 | 3.241178 | 2.398982 | 1.696991 | 2.166973 | 1.479425 |
| PCTUC_626 | 4.263879 | 3.245743 | 2.479307 | 2.482792 | 1.639614 |
| PCTUC_627 | 2.120695 | 1.950867 | 1.15705 | 1.526368 | 1.375283 |
| PCTUC_628 | 2.234193 | 1.721639 | 1.572728 | 1.672325 | 1.329832 |
| PCTUC_629 | 2.645262 | 2.009622 | 1.87541 | 2.068085 | 1.504461 |
| PCTUC_630 | 2.896469 | 2.463484 | 1.901627 | 1.923349 | 1.147038 |
| PCTUC_631 | 2.064842 | 1.621446 | 1.339284 | 1.385534 | 1.301967 |
| PCTUC_632 | 1.955281 | 1.482409 | 1.149627 | 1.092045 | 1.111003 |
| PCTUC_633 | 2.089976 | 1.619433 | 1.290514 | 1.629664 | 1.165089 |
| PCTUC_634 | 2.999586 | 1.967814 | 1.3107 | 1.652533 | 1.417344 |
| PCTUC_635 | 2.765555 | 1.540217 | 1.356881 | 1.681852 | 1.160647 |
| PCTUC_636 | 1.97347 | 1.371112 | 1.547874 | 1.554828 | 1.186887 |
| PCTUC_637 | 1.900493 | 1.412755 | 1.229323 | 1.277215 | −1.006073 |
| PCTUC_638 | 2.561628 | 1.908089 | 1.464314 | 1.384407 | 1.220221 |
| PCTUC_639 | 2.060063 | 1.545589 | 1.45735 | 1.561649 | 1.582865 |
| PCTUC_640 | 3.636007 | 2.997755 | 2.108412 | 2.242866 | 1.648748 |
| PCTUC_641 | 4.679021 | 3.565046 | 2.195632 | 2.465471 | 1.670103 |
| PCTUC_642 | 3.39597 | 2.37976 | 1.849144 | 2.319398 | 1.659982 |
| PCTUC_643 | 2.769052 | 2.057714 | 1.999222 | 2.00004 | 1.418435 |
| PCTUC_644 | 3.130126 | 1.843459 | 1.786604 | 1.997668 | 1.446098 |
| PCTUC_645 | 2.248375 | 2.071626 | 1.630426 | 1.708583 | 1.466932 |
| PCTUC_646 | 2.482285 | 2.26653 | 1.393158 | 1.702992 | 1.361391 |
| PCTUC_647 | 2.157234 | 1.842401 | 1.396858 | 1.534424 | 1.129591 |
| PCTUC_648 | 1.696494 | 1.639976 | 1.340072 | 1.360999 | 1.36889 |
| PCTUC_649 | 3.796501 | 3.261933 | 2.28008 | 2.205014 | 1.615482 |
| PCTUC_650 | 3.289895 | 3.550014 | 1.972488 | 1.88842 | 1.351876 |
| PCTUC_651 | 1.996156 | 1.805128 | 1.404317 | 1.432118 | 1.394295 |
| PCTUC_652 | 4.039923 | 3.381759 | 2.282342 | 2.25035 | 1.624183 |
| PCTUC_653 | 4.071556 | 3.266201 | 2.354722 | 2.154996 | 1.544724 |
| PCTUC_654 | 1.984298 | 1.555235 | 1.53037 | 1.545385 | 1.389834 |
| PCTUC_655 | 2.574506 | 1.984158 | 1.59359 | 1.721093 | 1.11024 |
| PCTUC_656 | 2.892828 | 2.117047 | 1.873724 | 1.873271 | 1.416158 |
| PCTUC_657 | 2.411529 | 1.795964 | 1.78192 | 2.371935 | 1.692294 |
| PCTUC_658 | 2.09202 | 1.744102 | 1.745396 | 1.762151 | 1.482022 |
| PCTUC_659 | 2.575266 | 2.124049 | 1.560475 | 1.727308 | 1.270648 |
| PCTUC_660 | 2.520167 | 1.745141 | 1.594287 | 1.69775 | 1.350521 |
| PCTUC_661 | 2.134085 | 1.940671 | 1.53473 | 1.50667 | 1.288514 |
| PCTUC_662 | 4.724458 | 3.946936 | 2.465677 | 2.121572 | 1.348008 |
| PCTUC_663 | 3.248355 | 3.44881 | 1.944542 | 1.979683 | 1.275257 |
| PCTUC_664 | 2.197084 | 1.883653 | 1.327784 | 1.438995 | 1.167273 |
| PCTUC_665 | 3.614111 | 1.87647 | 2.062585 | 2.114008 | 1.184832 |
| PCTUC_666 | 5.643526 | 4.451371 | 2.833776 | 2.63493 | 1.439823 |
| PCTUC_667 | 2.185947 | 1.815849 | 1.536835 | 1.63991 | 1.30789 |
| PCTUC_668 | 1.913589 | 1.460395 | 1.120916 | 1.262651 | 1.194898 |
| PCTUC_669 | 2.221644 | 2.111672 | 1.170494 | 1.612767 | 1.245904 |
| PCTUC_670 | 1.803643 | 1.555918 | 1.270898 | 1.483201 | 1.452556 |
| PCTUC_671 | 1.667533 | 1.414151 | 1.012784 | 1.153869 | 1.035625 |
| PCTUC_672 | 3.765201 | 4.324469 | 2.213268 | 1.789587 | 1.390585 |
| PCTUC_673 | 2.413774 | 1.597572 | 1.470126 | 1.148103 | 1.251977 |
| PCTUC_674 | 4.901073 | 3.630356 | 2.421033 | 2.328926 | 1.511821 |
| PCTUC_675 | 2.02757 | 1.687118 | 1.462588 | 1.634331 | 1.275855 |
| PCTUC_676 | 1.919538 | 1.742238 | 1.581826 | 1.585945 | 1.31395 |
| PCTUC_677 | 2.011766 | 1.351125 | 1.310936 | 1.382537 | 1.077973 |
| PCTUC_678 | 2.721872 | 1.77266 | 1.812868 | 1.940869 | 1.428293 |
| PCTUC_679 | 2.092978 | 1.779302 | 1.515586 | 1.668664 | 1.36376 |
| PCTUC_680 | 2.760168 | 1.714808 | 1.395004 | 1.473272 | 1.462381 |
| PCTUC_681 | 3.005869 | 1.905482 | 1.623226 | 1.913392 | 1.464779 |
| PCTUC_682 | 2.932731 | 2.279735 | 1.696851 | 1.844617 | 1.441928 |
| PCTUC_683 | 2.931927 | 2.512429 | 1.638134 | 1.535175 | 1.35907 |
| PCTUC_684 | 3.377182 | 2.464607 | 2.122759 | 2.173514 | 1.549637 |
| PCTUC_685 | 2.92051 | 1.826244 | 2.23447 | 2.417694 | 1.675354 |
| PCTUC_686 | 3.751326 | 2.864389 | 1.669852 | 1.850708 | 1.330981 |
| PCTUC_687 | 2.542475 | 1.384002 | 2.253859 | 2.250025 | 1.424351 |
| PCTUC_688 | 2.500217 | 1.454033 | 1.51167 | 1.553329 | 1.572351 |
| PCTUC_689 | 2.798229 | 1.898112 | 1.7912 | 1.784557 | 1.556316 |
| PCTUC_690 | 3.62354 | 2.586582 | 1.978384 | 2.035194 | 1.708843 |
| PCTUC_691 | 1.980327 | 1.529974 | 1.627668 | 1.647607 | 1.52965 |
| PCTUC_692 | 2.207429 | 2.023349 | 1.621345 | 1.557046 | 1.375703 |
| PCTUC_693 | 2.573091 | 1.854801 | 1.521731 | 1.559489 | 1.622517 |
| PCTUC_694 | 2.661108 | 1.611918 | 1.733396 | 1.820798 | 1.583537 |
| PCTUC_695 | 3.084925 | 1.906792 | 1.716335 | 1.835684 | 1.446206 |
| PCTUC_696 | 3.436488 | 2.050998 | 1.556213 | 1.780796 | 1.26221 |
| PCTUC_697 | 2.846267 | 2.169852 | 1.739728 | 1.886953 | 1.394527 |
| PCTUC_698 | 2.902011 | 2.257164 | 1.820701 | 1.706544 | 1.408133 |
| PCTUC_699 | 4.827313 | 4.018908 | 2.272471 | 2.292536 | 1.480471 |
| PCTUC_700 | 2.390521 | 1.464474 | 1.576155 | 1.860255 | 1.427529 |
| PCTUC_701 | 3.311536 | 2.376002 | 1.731801 | 1.61275 | 1.301387 |
| PCTUC_702 | 2.624016 | 2.448785 | 1.653695 | 1.713574 | 1.383952 |
| PCTUC_703 | 5.139153 | 4.081909 | 2.450374 | 2.374609 | 1.702067 |
| PCTUC_704 | 2.309258 | 2.014421 | 1.651519 | 1.676327 | 1.409805 |
| PCTUC_705 | 2.665751 | 1.414355 | 1.617702 | 1.691659 | 1.355501 |
| PCTUC_706 | 2.287762 | 1.813085 | 1.379834 | 1.514168 | 1.28103 |
| PCTUC_707 | 4.413056 | 2.800452 | 1.930365 | 2.110764 | 1.410492 |
| PCTUC_708 | 2.598162 | 1.997955 | 1.603416 | 1.690229 | 1.241806 |
| PCTUC_709 | 4.610568 | 3.121099 | 1.945206 | 2.056421 | 1.340026 |
| PCTUC_710 | 3.801278 | 3.21265 | 1.997177 | 1.977971 | 1.311692 |
| PCTUC_711 | 2.359209 | 1.549437 | 1.459169 | 1.554681 | 1.345212 |
| PCTUC_712 | 2.518226 | 1.681685 | 1.821278 | 1.801401 | 1.557828 |
| PCTUC_713 | 3.620613 | 2.880119 | 1.938276 | 2.031514 | 1.295043 |
| PCTUC_714 | 2.705708 | 2.028827 | 1.58479 | 1.802496 | 1.267622 |
| PCTUC_715 | 2.566382 | 1.848065 | 1.289421 | 1.405011 | 1.082506 |
| PCTUC_716 | 5.121842 | 4.239795 | 2.482671 | 2.399158 | 1.467867 |
| PCTUC_717 | 2.995538 | 2.487843 | 1.807189 | 2.102638 | 1.598794 |
| PCTUC_718 | 3.134496 | 2.21508 | 2.111608 | 2.288491 | 1.542992 |
| PCTUC_719 | 2.800922 | 2.135409 | 1.739122 | 1.755105 | −1.152661 |
| PCTUC_720 | 3.266792 | 1.098088 | 1.647018 | 2.574326 | 1.945548 |
| PCTUC_721 | 2.279879 | 1.470384 | 1.464171 | 1.332593 | 2.18729 |
| PCTUC_722 | 2.864947 | 2.957419 | 1.898244 | 2.449121 | 2.823838 |
| PCTUC_723 | 2.936209 | 1.665902 | 1.822441 | 2.500832 | 2.126406 |
| PCTUC_724 | 6.322812 | 4.141862 | 3.652369 | 2.620724 | 1.336264 |
| PCTUC_725 | 3.180355 | 5.149987 | 5.441726 | 3.571592 | 4.900834 |
| PCTUC_726 | 2.312671 | 3.094301 | 1.870246 | 1.929492 | 4.47831 |
| PCTUC_727 | 3.400119 | 5.56307 | 2.538855 | 4.798176 | 1.16597 |
| PCTUC_728 | 2.245501 | 2.481267 | 1.936934 | 2.452436 | 2.517091 |
| PCTUC_729 | 3.68157 | 2.719935 | 4.756963 | 4.780087 | 2.451955 |
| PCTUC_730 | 2.54578 | 1.70516 | 1.376145 | 1.063746 | 1.484946 |
| PCTUC_731 | 1.650166 | 2.149801 | 2.230358 | −1.192853 | 3.374881 |
| PCTUC_732 | 1.173051 | 2.470797 | 1.96913 | 2.981646 | 4.636403 |
| PCTUC_733 | 1.545922 | 2.714017 | 2.174145 | 1.535983 | 1.445219 |
| PCTUC_734 | 6.250961 | 2.613591 | 2.367622 | 1.395285 | 3.012707 |
| PCTUC_735 | 1.941943 | −1.154395 | 1.336703 | 2.721427 | 2.473974 |
| PCTUC_736 | 2.193176 | 1.875473 | 2.309137 | 2.229304 | 1.749662 |
| PCTUC_737 | 2.341751 | 1.418063 | 2.449272 | 2.472956 | 1.947165 |
| PCTUC_738 | 2.088114 | 1.828001 | 5.574064 | 6.420503 | 2.571054 |
| PCTUC_739 | 1.909534 | 2.02831 | 2.202837 | 1.52346 | 2.025769 |
| PCTUC_740 | 1.957884 | 2.764184 | 3.193176 | 2.302767 | 1.341441 |
| PCTUC_741 | 1.431072 | 2.457295 | 2.102171 | 1.919661 | 3.254123 |
| PCTUC_742 | 2.752174 | 2.719285 | 2.902712 | 1.786477 | 3.561341 |
| PCTUC_743 | 2.374025 | 2.252629 | 1.611526 | 1.306457 | 2.242702 |
| PCTUC_744 | 2.208758 | 1.106154 | 2.55469 | 1.622825 | 2.047873 |
| PCTUC_745 | 2.619822 | 2.234076 | 2.125212 | 1.175748 | 1.801113 |
| PCTUC_746 | −2.604011 | −4.336377 | −1.289437 | −2.013778 | 4.400435 |
| PCTUC_747 | 1.996631 | −1.253024 | −1.283601 | 1.034031 | 3.964703 |
| PCTUC_748 | 1.378674 | 2.536747 | 3.586713 | 1.966716 | 1.091169 |
| PCTUC_749 | 2.69839 | 5.340216 | 3.960504 | 1.565272 | 1.828033 |
| PCTUC_750 | 3.70196 | 4.375464 | 1.684335 | 1.790254 | 2.591611 |
| PCTUC_751 | 1.434497 | 2.616543 | 2.421894 | 1.107728 | 1.966931 |
| PCTUC_752 | 2.611563 | 2.241413 | 2.252592 | 1.833555 | 9.644817 |
| PCTUC_753 | 1.172065 | 1.9564 | 2.087169 | 2.44259 | 1.617989 |
| PCTUC_754 | 3.315586 | 5.507189 | 2.818951 | 1.428701 | 2.854979 |
| PCTUC_755 | 1.385515 | 2.299191 | 2.11237 | 1.901943 | 1.481955 |
| PCTUC_756 | 1.513908 | 1.812244 | 2.275906 | 1.890749 | 2.026129 |
| PCTUC_757 | 4.822169 | 3.194647 | 2.069062 | 6.764375 | 1.062056 |
| PCTUC_758 | 1.931267 | 2.721672 | 3.427641 | 2.313735 | 2.068174 |
| PCTUC_759 | 2.034523 | 1.959083 | 2.928546 | 3.031246 | 1.546934 |
| PCTUC_760 | 1.498347 | 2.639704 | 2.019559 | −1.061474 | 1.700927 |
| PCTUC_761 | 1.584407 | 1.682904 | 3.221693 | 2.118881 | 3.939364 |
| PCTUC_762 | 2.547231 | −1.006767 | 1.712516 | 3.774214 | 3.514404 |
| PCTUC_763 | 2.682344 | 2.138069 | 2.196179 | 1.553065 | 2.311897 |
| PCTUC_764 | 1.903514 | 1.984084 | 1.90328 | 1.87638 | 2.203765 |
| PCTUC_765 | 2.765059 | 2.873031 | 2.372725 | 1.795411 | 2.257401 |
| PCTUC_766 | 2.970002 | 1.421156 | 2.223881 | 2.091841 | 2.138762 |
| PCTUC_767 | 3.131984 | 1.92316 | 2.102419 | 1.974468 | 2.576102 |
| PCTUC_768 | 2.021438 | 1.613102 | 2.162979 | 2.390166 | 1.978501 |
| PCTUC_769 | 2.666843 | 2.697247 | 3.103855 | 1.741283 | 2.923714 |
| PCTUC_770 | 2.126302 | 1.367927 | 1.821058 | 2.06508 | 1.92668 |
| PCTUC_771 | 9.066208 | 2.301069 | 32.466282 | 9.306285 | 2.641976 |
| PCTUC_772 | 2.542206 | 2.138214 | 2.170891 | 2.752532 | 1.818219 |
| PCTUC_773 | 2.140708 | 1.910514 | 1.868318 | 1.171023 | 1.791513 |
| PCTUC_774 | 2.170943 | 1.04411 | 1.569475 | 1.657634 | 2.391124 |
| PCTUC_775 | 1.67631 | 1.744011 | 1.153107 | 1.063258 | 2.223874 |
| PCTUC_776 | 1.180588 | 2.774556 | 1.708677 | −1.116192 | 2.445475 |
| PCTUC_777 | 2.2542 | 2.193221 | 1.918728 | 1.949192 | 1.985048 |
| PCTUC_778 | 2.304341 | 1.986533 | 2.36534 | 2.001854 | 2.164915 |
| PCTUC_779 | 3.319706 | 7.10986 | 4.295543 | 1.431373 | 3.739748 |
| PCTUC_780 | 2.182786 | 2.654104 | 2.052293 | 1.226816 | 1.797102 |
| PCTUC_781 | 2.002184 | 2.124835 | 2.563484 | 2.123987 | 1.910713 |
| PCTUC_782 | 1.982726 | 2.339215 | 2.723196 | 1.605118 | 2.213815 |
| PCTUC_783 | 5.412443 | 3.235496 | 5.063339 | 2.247368 | 4.10765 |
| PCTUC_784 | 1.523491 | 1.954146 | 2.511079 | 1.829886 | 2.085814 |
| PCTUC_785 | 3.088835 | 3.454606 | 4.478419 | 4.437982 | 2.777349 |
| PCTUC_786 | 3.020984 | 2.177902 | 1.285365 | 1.111271 | 2.810368 |
| PCTUC_787 | 1.579647 | 2.126352 | 2.621198 | 1.891533 | 3.694917 |
| PCTUC_788 | 1.989318 | 1.677531 | 1.929438 | 3.418436 | 2.037659 |
| PCTUC_789 | 2.948468 | 3.183348 | 2.201415 | 2.286198 | 2.866979 |
| PCTUC_790 | 2.147948 | 1.806976 | 2.417625 | 2.035112 | 1.626123 |
| PCTUC_791 | 1.986498 | 2.576382 | 1.954842 | 1.568862 | 1.966789 |
| PCTUC_792 | 3.196796 | 3.486831 | 3.260377 | 2.264079 | 2.886792 |
| PCTUC_793 | 3.769532 | 4.108984 | 3.562748 | 2.284717 | 3.681486 |
| PCTUC_794 | 2.002344 | 1.894897 | 1.788498 | 1.56391 | 2.133116 |
| PCTUC_795 | 3.044567 | 3.152989 | 3.166747 | 1.968495 | 2.934963 |
| PCTUC_796 | 3.184995 | 3.117129 | 3.047694 | 2.60312 | 8.595053 |
| PCTUC_797 | 3.851431 | 4.428571 | 4.33976 | 2.782248 | 3.783499 |
| PCTUC_798 | 3.482885 | 4.161508 | 3.216578 | 2.304008 | 3.654118 |
| PCTUC_799 | 5.14745 | 5.773758 | 4.428958 | 2.696134 | 4.204111 |
| PCTUC_800 | 4.496764 | 4.59942 | 4.781262 | 2.728859 | 4.622788 |
| PCTUC_801 | 4.790975 | 6.2401 | 5.334881 | 2.849744 | 5.284999 |
| PCTUC_802 | 4.483533 | 5.933342 | 4.402905 | 2.849242 | 4.738077 |
| PCTUC_803 | 3.927758 | 5.220841 | 5.310245 | 2.649704 | 4.138473 |
| PCTUC_804 | 2.382821 | 2.906737 | 2.819774 | 2.152793 | 2.339993 |
| PCTUC_805 | 2.018582 | 2.140931 | 2.448375 | 2.322326 | 2.203802 |
| PCTUC_806 | 3.712907 | 4.013764 | 4.981676 | 2.92389 | 3.971395 |
| PCTUC_807 | 2.668381 | 3.176708 | 3.365095 | 3.510781 | 2.829598 |
| PCTUC_808 | 5.563641 | 6.477875 | 5.692748 | 2.900728 | 5.47545 |
| PCTUC_809 | 3.700055 | 3.973909 | 4.959146 | 2.291433 | 2.488166 |
| PCTUC_810 | 2.242853 | 2.325493 | 2.37969 | 1.993197 | 2.071771 |
| PCTUC_811 | 3.831634 | 4.074454 | 3.860348 | 2.405638 | 3.12156 |
| PCTUC_812 | 3.288 | 3.867888 | 3.940192 | 2.396031 | 3.229351 |
| PCTUC_813 | 3.754223 | 3.77283 | 3.865026 | 2.398146 | 3.256464 |
| PCTUC_814 | 4.75541 | 5.228207 | 4.819787 | 2.861704 | 5.149302 |
| PCTUC_815 | 3.892281 | 4.406302 | 4.380984 | 2.8373 | 4.870093 |
| PCTUC_816 | 4.379927 | 4.996663 | 4.362504 | 2.736067 | 5.081002 |
| PCTUC_817 | 3.106359 | 3.238691 | 3.38937 | 2.47535 | 3.133699 |
| PCTUC_818 | 4.434761 | 4.46969 | 4.046491 | 3.016147 | 4.799383 |
| PCTUC_819 | 4.909876 | 5.234096 | 5.850842 | 3.322591 | 5.497626 |
| PCTUC_820 | 2.304303 | 2.138123 | 2.074894 | 2.295133 | 2.166532 |
| PCTUC_821 | 2.834139 | 2.745442 | 2.990574 | 1.933787 | 2.992724 |
| PCTUC_822 | 5.200898 | 6.422604 | 5.118377 | 2.91676 | 5.344371 |
| PCTUC_823 | 2.53588 | 2.995658 | 4.46937 | 2.562591 | 1.622748 |
| PCTUC_824 | 2.933777 | 3.42145 | 3.242542 | 2.405094 | 3.023423 |
| PCTUC_825 | 3.186176 | 2.917574 | 2.62689 | 2.378563 | 3.827872 |
| PCTUC_826 | 1.919053 | 2.475648 | 1.850094 | 1.928537 | 2.086126 |
| PCTUC_827 | 1.904114 | 1.956388 | 1.952929 | 1.326964 | 1.775423 |
| PCTUC_828 | 2.92745 | 3.599613 | 3.223438 | 2.173024 | 3.13715 |
| PCTUC_829 | 1.974123 | 2.175198 | 2.062895 | 1.914626 | 2.036808 |
| PCTUC_830 | 3.077779 | 4.19744 | 2.897306 | 2.005722 | 3.079901 |
| PCTUC_831 | 1.840324 | 2.44461 | 1.915517 | 2.425321 | 1.885715 |
| PCTUC_832 | 2.225947 | 2.367063 | 2.522229 | 1.897432 | 2.548975 |
| PCTUC_833 | 2.858343 | 2.674582 | 2.619268 | 2.118242 | 2.951528 |
| PCTUC_834 | 2.9596 | 2.374317 | 2.542884 | 2.410777 | 3.259532 |
| PCTUC_835 | 3.010594 | 3.311469 | 3.328452 | 2.455323 | 3.43195 |
| PCTUC_836 | 2.007784 | 2.085062 | 2.016944 | 1.841439 | 2.027111 |
| PCTUC_837 | 2.796955 | 2.807929 | 3.865616 | 2.395671 | 3.712218 |
| PCTUC_838 | 1.494369 | 1.963341 | 5.039379 | 2.152365 | 1.19218 |
| PCTUC_839 | 4.239632 | 3.829796 | 4.241501 | 2.527571 | 5.13077 |
| PCTUC_840 | 2.061444 | 1.848708 | 2.171947 | 1.677039 | 2.955164 |
| PCTUC_841 | 2.054037 | 1.62282 | 1.568904 | 1.818563 | 2.585692 |
| PCTUC_842 | 3.325278 | 2.201624 | 2.83394 | 2.779279 | 2.899318 |
| PCTUC_843 | 1.788604 | 2.545392 | 2.462816 | 2.152621 | 2.521353 |
| PCTUC_844 | 2.402184 | 1.850049 | 1.960408 | 2.116551 | 2.600048 |
| PCTUC_845 | 2.50498 | 2.196354 | 2.359953 | 2.038789 | 2.122147 |
| PCTUC_846 | 1.928036 | 1.792006 | 2.050615 | 2.294138 | 2.265651 |
| PCTUC_847 | 2.329573 | 2.669091 | 2.7908 | 2.09974 | 2.517365 |
| PCTUC_848 | 2.48907 | 1.635645 | 3.754775 | 3.079755 | 1.910938 |
| PCTUC_849 | 2.128594 | 2.421387 | 2.235757 | 1.88999 | 2.596102 |
| PCTUC_850 | 2.250095 | 2.870839 | 2.845568 | 1.836877 | 3.054814 |
| PCTUC_851 | 2.480321 | 2.003515 | 2.084979 | 2.261478 | 2.775162 |
| PCTUC_852 | 3.791353 | 2.885709 | 4.59536 | 4.799927 | 1.82336 |
| PCTUC_853 | 2.130593 | 2.395275 | 2.332972 | 3.390371 | 1.420116 |
| PCTUC_854 | 4.050172 | 3.927672 | 3.382661 | 2.926788 | 3.400673 |
| PCTUC_855 | 2.517597 | 4.010342 | 2.627365 | 2.16504 | 2.749827 |
| PCTUC_856 | 4.521049 | 4.759143 | 5.94944 | 3.528028 | 4.510493 |
| PCTUC_857 | 2.118167 | 1.667272 | 1.584278 | 2.004081 | 2.715339 |
| PCTUC_858 | 3.618611 | 3.923041 | 3.748677 | 2.279956 | 4.36315 |
| PCTUC_859 | 2.535355 | 2.672349 | 3.022804 | 2.061067 | 3.411787 |
| PCTUC_860 | 2.018386 | 2.602673 | 2.390009 | 1.920446 | 1.678945 |
| PCTUC_861 | 2.705428 | 2.904839 | 2.577471 | 2.28519 | 2.138755 |
| PCTUC_862 | 2.109637 | 2.062488 | 2.299455 | 1.913848 | 2.063145 |
| PCTUC_863 | 1.402514 | 1.913175 | 2.036407 | 2.504151 | 3.015167 |
| PCTUC_864 | 2.99618 | 3.233393 | 2.876639 | 2.230565 | 3.5081 |
| PCTUC_865 | 2.690218 | 2.717674 | 2.690491 | 1.992791 | 2.795335 |
| PCTUC_866 | 1.996733 | 1.609049 | 2.308326 | 2.546587 | 2.202009 |
| PCTUC_867 | 2.818447 | 3.778686 | 3.51761 | 2.102258 | 3.744934 |
| PCTUC_868 | 1.991561 | 2.991961 | 2.213984 | 1.770526 | 2.169716 |
| PCTUC_869 | 2.25384 | 1.99884 | 2.021969 | 3.166457 | 2.211639 |
| PCTUC_870 | 2.254735 | 1.975595 | 2.232454 | 2.071242 | 2.097088 |
| PCTUC_871 | 2.01151 | 2.790665 | 2.513632 | 1.841401 | 2.546699 |
| PCTUC_872 | 2.010319 | 2.272945 | 2.635181 | 2.545468 | 2.5213 |
| PCTUC_873 | 3.252205 | 2.109674 | 3.160142 | 2.223053 | 2.871609 |
| PCTUC_874 | 1.741674 | 1.985395 | 1.40981 | −1.0323 | 1.717773 |
| PCTUC_875 | 2.694372 | 2.621971 | 2.075549 | 1.654308 | 3.340908 |
| PCTUC_876 | 2.534711 | 2.628107 | 2.776704 | 2.619621 | 2.670912 |
| PCTUC_877 | 2.125987 | 1.825273 | 3.556471 | 2.425424 | 2.202742 |
| PCTUC_878 | 2.143905 | 2.173444 | 2.548723 | 1.926992 | 1.945828 |
| PCTUC_879 | 3.017557 | 3.313776 | 3.23282 | 2.519569 | 4.085382 |
| PCTUC_880 | 2.332958 | 2.74189 | 2.615978 | 1.927811 | 2.540155 |
| PCTUC_881 | 3.719461 | 3.623826 | 3.19083 | 2.295216 | 3.808763 |
| PCTUC_882 | 3.375582 | 3.323253 | 3.632514 | 2.259738 | 3.998963 |
| PCTUC_883 | 3.460237 | 3.49788 | 4.037887 | 2.49328 | 4.323845 |
| PCTUC_884 | 2.009108 | 2.153635 | 2.529886 | 2.029703 | 2.670727 |
| PCTUC_885 | 2.66158 | 2.072809 | 2.160885 | 1.721565 | −1.103153 |
| PCTUC_886 | 2.972494 | 2.924612 | 3.118183 | 2.282334 | 3.49861 |
| PCTUC_887 | 2.027975 | 2.38398 | 2.296002 | 1.924612 | 2.42382 |
| PCTUC_888 | 2.436039 | 3.457342 | 2.737972 | 2.126336 | 1.551599 |
| PCTUC_889 | 2.248439 | 2.221073 | 2.669714 | 2.05175 | 2.822394 |
| PCTUC_890 | 2.203458 | 2.155574 | 2.361843 | 1.701684 | 2.477744 |
| PCTUC_891 | 3.155087 | 2.883648 | 2.752733 | 2.458148 | 3.186105 |
| PCTUC_892 | 2.343731 | 1.338486 | 2.692445 | 2.506622 | 1.907094 |
| PCTUC_893 | 2.915632 | 2.590539 | 3.622392 | 3.905265 | 4.47831 |
| PCTUC_894 | 4.506998 | 3.59058 | 3.962988 | 2.844126 | 4.481072 |
| PCTUC_895 | 2.269523 | 1.780666 | 2.160403 | 2.08125 | 2.08296 |
| PCTUC_896 | 2.865829 | 2.840475 | 2.428004 | 2.385588 | 2.747852 |
| PCTUC_897 | 2.345222 | 1.942502 | 1.96854 | 2.697916 | 1.00369 |
| PCTUC_898 | 2.265094 | 2.938895 | 2.243422 | 2.234162 | 2.681554 |
| PCTUC_899 | 3.240594 | 2.89024 | 3.19629 | 1.643464 | 4.062936 |
| PCTUC_900 | 2.158255 | 2.225039 | 1.958986 | 1.337243 | 2.284375 |
| PCTUC_901 | 1.704198 | 2.000071 | 2.057588 | 2.11317 | 1.742318 |
| PCTUC_902 | 1.700514 | 1.979201 | 2.911566 | 2.402822 | 2.639393 |
| PCTUC_903 | 1.971537 | 1.805452 | 1.941796 | 1.408055 | 2.242712 |
| PCTUC_904 | 2.582627 | 2.451558 | 2.073478 | 1.945297 | 2.805823 |
| PCTUC_905 | 2.486936 | 2.48598 | 2.785518 | 2.234389 | 2.725869 |
| PCTUC_906 | 2.348454 | 3.648733 | 3.524524 | 2.959175 | 3.327241 |
| PCTUC_907 | 4.418301 | 5.429524 | 5.189786 | 2.601276 | 5.046936 |
| PCTUC_908 | 2.923208 | 3.753517 | 3.50064 | 2.171186 | 3.947329 |
| PCTUC_909 | 2.172991 | 2.597694 | 2.453735 | 1.788767 | 2.241053 |
| PCTUC_910 | 1.632832 | 2.259243 | 2.020136 | 1.770561 | 2.368836 |
| PCTUC_911 | 3.628025 | 3.382685 | 3.266242 | 1.433443 | 2.065969 |
| PCTUC_912 | 1.708289 | 4.094818 | 3.834928 | 1.347942 | 2.497629 |
| PCTUC_913 | −1.046016 | 2.240158 | 2.899449 | 3.032311 | 1.341131 |
| PCTUC_914 | −1.027773 | 1.19017 | −1.465852 | −1.152883 | −1.047242 |
| PCTUC_915 | 1.725658 | 1.366501 | 1.683899 | 2.436776 | 2.106251 |
| PCTUC_916 | 1.327684 | 2.027969 | 2.144469 | 2.198778 | 1.659379 |
| PCTUC_917 | 1.483645 | 1.60136 | 2.103386 | 2.447269 | 1.95535 |
| PCTUC_918 | 1.771259 | 1.22116 | 2.070256 | 1.986665 | 1.580713 |
| PCTUC_919 | 2.918718 | 2.124875 | 1.756297 | 1.53907 | 1.104604 |
| PCTUC_920 | 1.897873 | 2.063434 | 1.176537 | 1.403987 | 1.222612 |
| PCTUC_921 | 3.146559 | 1.290703 | 1.689708 | 2.295315 | 2.378711 |
| PCTUC_922 | 1.300489 | 1.381081 | 1.467731 | 2.008957 | 1.973597 |
| PCTUC_923 | 1.191862 | 1.585463 | 1.143061 | 2.031052 | 2.405917 |
| PCTUC_924 | 1.518692 | 3.641808 | 3.494821 | 1.361807 | 1.99471 |
| PCTUC_925 | 2.424894 | 2.040161 | 2.190496 | 2.154025 | 1.278334 |
| PCTUC_926 | 1.072146 | 1.919559 | 2.084503 | 1.99245 | 1.011251 |
| PCTUC_927 | −1.101332 | 1.821126 | 1.98096 | 2.582406 | 1.806138 |
| PCTUC_928 | 1.238585 | 2.209098 | 2.432987 | 2.179409 | 1.149451 |
| PCTUC_929 | 2.16326 | 1.219258 | 1.393823 | 3.200348 | 2.760397 |
| PCTUC_930 | 1.507631 | 2.192262 | 2.35394 | 2.122252 | 1.663228 |
| PCTUC_931 | −1.02405 | 1.359214 | 1.94306 | 2.289215 | 3.077495 |
| PCTUC_932 | 1.975673 | −1.065396 | 1.775844 | 2.857529 | 2.110501 |
| PCTUC_933 | 1.989775 | 2.301899 | 2.211407 | 2.059944 | 1.641539 |
| PCTUC_934 | 2.596033 | 1.930659 | 2.12377 | 1.602472 | 1.913087 |
| PCTUC_935 | 2.121555 | 2.019863 | 3.910625 | 2.859906 | 1.295485 |
| PCTUC_936 | 2.90906 | −1.051498 | 2.174735 | 5.179016 | 4.19084 |
| PCTUC_937 | 2.907178 | 9.230203 | 2.312996 | 1.276643 | 1.504857 |
| PCTUC_938 | 1.899924 | 3.569331 | 2.190631 | 1.89375 | 2.05869 |
| PCTUC_939 | 3.068882 | 2.580992 | 1.638408 | 2.981934 | 11.334661 |
| PCTUC_940 | 4.369292 | 2.150146 | 2.492596 | 2.16282 | 1.811484 |
| PCTUC_941 | 6.789319 | 2.83798 | 2.739652 | −1.141878 | 1.460348 |
| PCTUC_942 | 8.814911 | −2.69703 | 6.393185 | 12.043472 | −1.465271 |
| PCTUC_943 | 2.181615 | 3.609258 | 2.070167 | 1.696407 | 1.338924 |
| PCTUC_944 | 2.302399 | 2.098599 | 1.513715 | −1.04266 | 2.621862 |
| PCTUC_945 | 1.999231 | 2.014684 | 3.082334 | 2.063655 | 1.362715 |
| PCTUC_946 | 2.545056 | 2.827518 | 2.61495 | 1.211746 | 2.104344 |
| PCTUC_947 | 2.561492 | 3.223751 | 2.778957 | 1.930789 | 1.553573 |
| PCTUC_948 | 1.706577 | −1.308394 | −1.237869 | 1.039971 | 3.865446 |
| PCTUC_949 | 1.43762 | 3.289446 | 1.772405 | 1.788377 | 2.09428 |
| PCTUC_950 | 13.208265 | 9.352516 | 4.609438 | 8.549457 | 1.956832 |
| PCTUC_951 | 2.494127 | 1.998511 | 1.590633 | −1.031617 | 2.656538 |
| PCTUC_952 | 2.82671 | 2.233259 | 3.037292 | 2.039842 | 2.084847 |
| PCTUC_953 | 2.853971 | 3.279671 | 4.436422 | 1.663468 | 3.558529 |
| PCTUC_954 | 3.898259 | 3.25054 | 3.100874 | 2.376224 | 2.463332 |
| PCTUC_955 | 2.017269 | 1.446693 | 1.930404 | 1.320767 | 2.193144 |
| PCTUC_956 | 2.800478 | 5.191406 | 2.169827 | 2.550036 | 1.613759 |
| PCTUC_957 | 2.047268 | 2.226898 | 1.30777 | 2.2121 | 2.037518 |
| PCTUC_958 | 2.344756 | 2.871189 | 3.006427 | 2.284117 | 4.198602 |
| PCTUC_959 | 1.986256 | 2.648532 | 2.317293 | 2.659039 | 1.70162 |
| PCTUC_960 | 2.235276 | 1.97325 | 1.955242 | 1.238362 | 2.183643 |
| PCTUC_961 | 1.63631 | 1.274917 | 2.890156 | −1.242321 | 3.175415 |
| PCTUC_962 | 2.321866 | 1.862551 | 1.916661 | 1.87765 | 2.2245 |
| PCTUC_963 | 1.999053 | 1.822242 | 1.93074 | 1.022611 | 2.052344 |
| PCTUC_964 | 2.92635 | 2.622254 | 2.912582 | 3.961787 | 1.85012 |
| PCTUC_965 | 2.240051 | 1.577813 | 2.167076 | 1.196908 | 2.714298 |
| PCTUC_966 | 8.739191 | 1.618101 | 17.573606 | 7.109097 | 1.762113 |
| PCTUC_967 | 3.445451 | 2.098926 | 3.399137 | 1.719636 | 2.686562 |
| PCTUC_968 | 2.356605 | 2.231912 | 1.267656 | 1.811389 | 3.422028 |
| PCTUC_969 | 2.303658 | 2.29328 | 1.62459 | 1.166594 | 1.631738 |
| PCTUC_970 | 3.712332 | 3.554731 | 3.460251 | 1.426326 | 2.987715 |
| PCTUC_971 | 2.520978 | 2.121438 | 1.94307 | 1.326363 | 1.768682 |
| PCTUC_972 | 2.542714 | 2.971309 | 2.511884 | 1.574862 | 2.593775 |
| PCTUC_973 | 2.079656 | 1.617495 | 1.915495 | 1.297526 | 2.815989 |
| PCTUC_974 | 1.637691 | 3.281691 | 4.147404 | 3.280086 | 1.723133 |
| PCTUC_975 | 1.908338 | 1.326548 | 1.354787 | 1.804381 | 3.117997 |
| PCTUC_976 | 2.122945 | 2.807116 | 2.32995 | 2.880146 | 1.539895 |
| PCTUC_977 | 2.142424 | 4.477849 | 3.614886 | 1.303536 | 1.630268 |
| PCTUC_978 | 1.844867 | 4.566318 | 3.164039 | 1.116617 | 1.187718 |
| PCTUC_979 | 3.689453 | 1.997519 | 1.491258 | 1.672252 | 2.057643 |
| PCTUC_980 | 1.886542 | 2.248851 | 2.074873 | 1.414794 | 2.233084 |
| PCTUC_981 | 2.08836 | 2.049679 | 2.05979 | 1.236637 | 2.006662 |
| PCTUC_982 | 9.422415 | 5.445676 | 4.121441 | 3.337268 | −1.077134 |
| PCTUC_983 | 2.169163 | 1.979837 | 3.120249 | 3.177388 | 1.344734 |
| PCTUC_984 | 2.545414 | 3.161671 | −1.048021 | −1.020995 | 1.377628 |
| PCTUC_985 | 3.494484 | 5.996723 | 4.444107 | 3.225024 | 2.333731 |
| PCTUC_986 | 6.475299 | 5.153673 | 5.127562 | 1.588725 | 3.348588 |
| PCTUC_987 | 2.460184 | 2.194276 | 1.690811 | 1.674193 | 2.135545 |
| PCTUC_988 | 2.052341 | 2.031957 | 3.182646 | 2.178561 | 1.434476 |
| PCTUC_989 | 2.641716 | 2.272612 | 1.178549 | 2.740248 | 2.787188 |
| PCTUC_990 | 2.228094 | 2.029221 | 1.743631 | 2.108763 | 1.605475 |
| PCTUC_991 | −1.120914 | 2.574555 | 1.406036 | −2.033588 | 2.358146 |
| PCTUC_992 | 2.587451 | 2.230571 | 2.801737 | 2.085155 | 1.888326 |
| PCTUC_993 | 2.389752 | 1.481784 | 1.738086 | 1.470533 | 3.590099 |
| PCTUC_994 | 3.214524 | 3.677742 | 4.622314 | 3.995251 | 2.352907 |
| PCTUC_995 | 1.299307 | 2.503965 | 1.912283 | −1.000929 | 1.964259 |
| PCTUC_996 | 3.145576 | 2.075071 | 1.719235 | 1.5831 | −1.106453 |
| PCTUC_997 | 3.614506 | 2.11737 | 2.371607 | 2.323291 | 1.895755 |
| PCTUC_998 | 1.503244 | 2.900391 | 2.956001 | 2.59196 | 1.536935 |
| PCTUC_999 | 1.54409 | 2.00904 | 2.189009 | 1.112385 | 6.224452 |
| PCTUC_1000 | 2.257364 | 1.945727 | 3.821304 | 2.174996 | 1.875551 |
| PCTUC_1001 | 1.935329 | 2.216833 | 1.58268 | 3.462853 | 2.968328 |
| PCTUC_1002 | 2.510861 | 1.434915 | 2.16613 | 1.911476 | 1.278224 |
| PCTUC_1003 | 2.83421 | 1.655505 | 1.6249 | 2.138236 | 6.283207 |
| PCTUC_1004 | 2.979737 | 2.171031 | 2.224337 | 1.850705 | 1.290176 |
| PCTUC_1005 | 1.557493 | 2.305755 | 2.095499 | 2.149415 | 1.390719 |
| PCTUC_1006 | 5.535388 | 7.73051 | 3.142701 | −1.084378 | 3.258003 |
| PCTUC_1007 | 2.615719 | 1.901241 | 1.692383 | 1.787937 | 2.608618 |
| PCTUC_1008 | 1.663619 | 1.794802 | 6.374212 | 1.840365 | 4.460235 |
| PCTUC_1009 | 2.892305 | 2.170208 | 1.440984 | 1.408635 | 2.93398 |
| PCTUC_1010 | 1.880008 | 1.988196 | 2.131428 | 2.353244 | 1.907687 |
| PCTUC_1011 | 2.377302 | 2.395559 | 2.353736 | 1.510876 | 2.469237 |
| PCTUC_1012 | 7.20619 | 3.67785 | 3.583454 | 2.960135 | 3.33169 |
| PCTUC_1013 | 2.295973 | 1.924709 | 2.110733 | 2.214748 | 1.67961 |
| PCTUC_1014 | 1.945588 | 6.071303 | 6.141302 | 4.545424 | 1.493324 |
| PCTUC_1015 | 2.437442 | 2.363077 | 1.907915 | 1.24577 | 2.015562 |
| PCTUC_1016 | 2.648614 | 2.691739 | 2.125413 | 1.510101 | 1.551306 |
| PCTUC_1017 | 2.08836 | 2.478742 | 2.411507 | 1.85197 | 2.521672 |
| PCTUC_1018 | 2.211551 | 2.414348 | 2.150821 | 1.263532 | 2.090107 |
| PCTUC_1019 | 2.243053 | 2.254891 | 2.0381 | 1.362609 | 2.175798 |
| PCTUC_1020 | 2.178661 | 2.363133 | 1.977522 | 1.234102 | 2.011053 |
| PCTUC_1021 | 3.163341 | 2.58837 | 2.621589 | 1.408393 | 3.120556 |
| PCTUC_1022 | 2.174315 | 2.401368 | 2.06502 | 1.434312 | 1.691736 |
| PCTUC_1023 | 2.03722 | 1.811963 | 2.094636 | 1.388881 | 2.020649 |
| PCTUC_1024 | 2.445513 | 2.453608 | 1.804029 | 3.237316 | 1.742311 |
| PCTUC_1025 | 2.117916 | 2.15395 | 2.049663 | 1.506818 | 1.973658 |
| PCTUC_1026 | 2.10133 | 1.767012 | 2.766461 | 1.130868 | 2.016076 |
| PCTUC_1027 | 2.237257 | 2.367525 | 2.2373 | 1.669054 | 1.671289 |
| PCTUC_1028 | 1.694805 | 3.393244 | 2.780445 | 1.023844 | 1.428 |
| PCTUC_1029 | 3.43763 | 2.470928 | 2.477139 | 1.661261 | 1.722777 |
| PCTUC_1030 | 1.676745 | 3.116692 | 1.956181 | 2.168289 | 1.894927 |
| PCTUC_1031 | 2.563475 | 2.375423 | 2.750607 | 1.337144 | 2.270017 |
| PCTUC_1032 | 1.701297 | 7.355005 | 8.588897 | 6.225047 | 1.699016 |
| PCTUC_1033 | 2.254854 | 2.130038 | 1.321677 | 1.750454 | 2.423863 |
| PCTUC_1034 | 2.101815 | 2.041247 | 1.677848 | 1.32662 | 2.254946 |
| PCTUC_1035 | 2.007621 | 2.009369 | 2.074745 | 1.271912 | 2.072435 |
| PCTUC_1036 | 2.671005 | 2.959068 | 3.898445 | 2.528466 | 2.557439 |
| PCTUC_1037 | 2.553691 | 1.57382 | 1.76822 | 2.366851 | 1.942184 |
| PCTUC_1038 | 2.632271 | 2.32554 | 1.337809 | 2.459163 | 3.139472 |
| PCTUC_1039 | 2.077623 | 1.529966 | 1.836295 | 1.394481 | 1.958322 |
| PCTUC_1040 | 2.194537 | 2.399347 | 2.303395 | 1.89259 | 2.729439 |
| PCTUC_1041 | 2.330321 | 2.353049 | 2.368518 | 1.439722 | 1.806904 |
| PCTUC_1042 | 1.512719 | 1.910023 | 2.468123 | 1.859708 | 2.13613 |
| PCTUC_1043 | 2.258492 | 3.003565 | 2.174273 | 1.297437 | 2.415284 |
| PCTUC_1044 | 3.830532 | 2.841184 | 3.037506 | 1.615478 | 3.303318 |
| PCTUC_1045 | 2.521084 | 2.768955 | 2.359327 | 1.532786 | 2.363462 |
| PCTUC_1046 | 2.691287 | 2.029598 | 2.164129 | 1.569587 | 2.17121 |
| PCTUC_1047 | 4.148441 | 3.724395 | 2.311642 | 2.541887 | 2.194607 |
| PCTUC_1048 | 3.402202 | 3.200241 | 2.513871 | 2.593382 | 1.939163 |
| PCTUC_1049 | 2.740732 | 2.697594 | 2.084831 | 2.384646 | 2.168709 |
| PCTUC_1050 | 1.640787 | 2.482057 | 1.476933 | 1.563183 | 2.135159 |
| PCTUC_1051 | 1.358934 | 2.087856 | 1.620875 | 1.902402 | 1.913099 |
| PCTUC_1052 | 2.165557 | 1.650278 | 1.46462 | 1.314437 | 1.562692 |
| PCTUC_1053 | 2.334652 | 3.100399 | 1.809538 | 1.788479 | 3.325128 |
| PCTUC_1054 | 2.429376 | 1.579998 | 1.683261 | 1.886491 | 1.238793 |
| PCTUC_1055 | 1.001255 | 4.122614 | 1.903675 | 1.909667 | 2.93935 |
| PCTUC_1056 | 5.635899 | 4.017169 | 2.059601 | 2.393082 | 1.742979 |
| PCTUC_1057 | 3.696463 | 3.913398 | 2.197404 | 2.421372 | 1.805608 |
| PCTUC_1058 | 2.074971 | 2.023162 | 1.930327 | 1.872757 | 1.92556 |
| PCTUC_1059 | 3.01785 | 2.41626 | 2.067343 | 1.854001 | 1.630117 |
| PCTUC_1060 | 3.432813 | 3.74384 | 2.526108 | 2.254666 | 1.940598 |
| PCTUC_1061 | 3.597329 | 3.734005 | 2.229716 | 2.217949 | 1.926729 |
| PCTUC_1062 | 2.533764 | 1.934094 | 2.167086 | 2.696558 | 1.715214 |
| PCTUC_1063 | 2.157444 | 2.482796 | 1.867876 | 1.765957 | 1.589517 |
| PCTUC_1064 | 4.09897 | 3.738977 | 2.442533 | 2.412839 | 2.164216 |
| PCTUC_1065 | 2.054724 | 2.228052 | 2.512652 | 1.919982 | 2.701186 |
| PCTUC_1066 | 2.084735 | 2.24061 | 1.429457 | 1.82487 | 2.75413 |
| PCTUC_1067 | 2.933006 | 4.749996 | 2.385475 | 1.909325 | 4.58545 |
| PCTUC_1068 | 2.701608 | 1.184968 | 1.307782 | 2.119545 | 3.026683 |
| PCTUC_1069 | 2.625726 | 4.286357 | 2.167391 | 2.290825 | 4.321199 |
| PCTUC_1070 | 1.584965 | 1.882021 | 1.744875 | 2.283847 | 1.837694 |
| PCTUC_1071 | 1.470987 | 2.153697 | 1.495836 | 1.419932 | 5.155255 |
| PCTUC_1072 | 2.802821 | 2.533034 | 1.67031 | 2.088873 | 1.771321 |
| PCTUC_1073 | 2.478014 | 2.234136 | 1.961086 | 1.38199 | 5.264887 |
| PCTUC_1074 | 2.892866 | 2.33099 | 1.988019 | 2.946748 | 1.108592 |
| PCTUC_1075 | 4.04627 | 4.70859 | 2.551507 | 1.56122 | 1.826874 |
| PCTUC_1076 | 3.606797 | 4.151217 | 1.930236 | 2.696807 | 5.593647 |
| PCTUC_1077 | 1.616496 | 3.197529 | 3.108619 | 1.130574 | 1.346149 |
| PCTUC_1078 | 1.422069 | 4.019051 | 1.909229 | 1.279646 | 1.15715 |
| PCTUC_1079 | 2.122901 | 1.275714 | 1.809766 | 3.093876 | 3.302016 |
| PCTUC_1080 | 1.828537 | 1.957328 | 1.229454 | 1.199278 | 1.232905 |
| PCTUC_1081 | 6.836148 | 5.102329 | 2.717062 | 2.208524 | 2.300102 |
| PCTUC_1082 | 6.903161 | 1.482253 | 2.091234 | 6.780239 | 1.029036 |
| PCTUC_1083 | 2.452948 | 1.82755 | 2.15254 | 2.260958 | 1.523636 |
| PCTUC_1084 | 2.247821 | 2.066517 | 2.982403 | 1.608464 | 2.04356 |
| PCTUC_1085 | 1.874127 | 1.955559 | 1.498886 | 1.842026 | 1.538427 |
| PCTUC_1086 | 2.847633 | 6.075811 | 4.381285 | 1.511456 | 1.868587 |
| PCTUC_1087 | 1.3664 | 2.123451 | 2.9151 | 2.105061 | 2.410319 |
| PCTUC_1088 | 1.936453 | 1.911005 | 2.479018 | 1.812969 | 2.036546 |
| PCTUC_1089 | 1.24914 | 1.909665 | 1.100831 | −1.024006 | 1.882236 |
| PCTUC_1090 | 2.427823 | 3.291917 | 2.632262 | −1.157801 | 2.557583 |
| PCTUC_1091 | −1.135505 | −1.247456 | −1.045071 | −1.085618 | 2.864153 |
| PCTUC_1092 | 3.410679 | 6.492293 | 1.777087 | 1.695987 | 2.327668 |
| PCTUC_1093 | 3.755767 | 4.141311 | 4.201713 | 2.539634 | 3.961076 |
| PCTUC_1094 | 2.617909 | 2.304399 | 2.340273 | 2.152541 | 2.739291 |
| PCTUC_1095 | 3.191518 | 2.927253 | 2.048578 | 1.963189 | 1.810486 |
| PCTUC_1096 | 2.476109 | 2.670372 | 1.640251 | 1.433016 | 1.183916 |
| PCTUC_1097 | 2.792058 | 1.011758 | 2.032809 | 4.583232 | 2.712476 |
| PCTUC_1098 | 3.151127 | 3.072883 | 3.069124 | 2.292287 | 3.349233 |
| PCTUC_1099 | 1.913735 | 1.955109 | 2.239843 | 2.06643 | 1.812519 |
| PCTUC_1100 | 2.463933 | 2.147176 | 3.594009 | 3.065532 | 4.90844 |
| PCTUC_1101 | 2.644637 | 2.395418 | 2.492429 | 1.721552 | 2.093062 |
| PCTUC_1102 | 1.794169 | 4.295766 | 1.950269 | 1.521305 | 3.031758 |
| PCTUC_1103 | 3.468701 | 3.695937 | 2.574288 | 3.952629 | 14.716324 |
| PCTUC_1104 | 1.125964 | 2.803037 | 2.456732 | 2.102422 | −1.074769 |
| PCTUC_1105 | 2.568763 | 2.527151 | 2.689841 | 2.754335 | 1.771922 |
| PCTUC_1106 | 2.643478 | 2.604252 | 3.498866 | 3.627512 | 1.705972 |
| PCTUC_1107 | 1.526503 | 3.075097 | 1.460604 | 1.517412 | 2.6556 |
| PCTUC_1108 | 2.463843 | 2.560478 | 3.215582 | 1.354654 | 1.810031 |
| PCTUC_1109 | 3.588415 | 2.108878 | 2.986275 | 1.881867 | 2.134955 |
| PCTUC_1110 | 2.204521 | 2.448262 | 2.352178 | 2.286165 | 1.417835 |
| PCTUC_1111 | 2.069705 | 2.8492 | 1.984487 | 2.394027 | 3.019645 |
| PCTUC_1112 | 5.447097 | 2.774286 | 1.580806 | 1.491514 | 1.893947 |
| PCTUC_1113 | 7.077715 | 9.720737 | 2.134717 | 4.605519 | 9.415028 |
| PCTUC_1114 | 1.609405 | 3.273106 | 1.546363 | 1.695939 | 2.102199 |
| PCTUC_1115 | 2.537809 | 2.632147 | 1.810424 | 3.062999 | 2.56556 |
| PCTUC_1116 | 1.964747 | 2.737338 | 1.587556 | 1.63718 | 2.788908 |
| PCTUC_1117 | 1.654424 | 3.967134 | −1.022176 | 1.599265 | 1.611094 |
| PCTUC_1118 | 1.423818 | 1.82765 | 1.933905 | 1.546609 | 2.088507 |
| PCTUC_1119 | 1.956617 | 1.911459 | 1.960848 | 1.916424 | 1.27242 |
| PCTUC_1120 | 2.248806 | 2.269157 | 1.875802 | 1.964867 | 1.680379 |

| | Colon | Colon | Colon | Colon | Colon |
|---|---|---|---|---|---|
| Internal Id | Tumor 6 | Tumor 7 | Tumor 8 | Tumor 9 | Tumor 10 |
| PCTUC_1 | 2.489082 | 1.179339 | 1.210582 | −1.500949 | 1.525775 |
| PCTUC_2 | 4.119026 | 2.455277 | 2.637006 | 1.209973 | −1.000567 |
| PCTUC_3 | 1.005496 | 5.875144 | 6.30145 | −1.159219 | 2.552166 |
| PCTUC_4 | 1.199631 | 3.034428 | 2.040424 | 2.191431 | 2.560127 |
| PCTUC_5 | 1.860806 | 2.206577 | 5.10363 | 6.760265 | 4.802891 |
| PCTUC_6 | 1.212183 | 1.667211 | 3.338447 | 1.982268 | 1.572041 |
| PCTUC_7 | 2.602364 | 1.40409 | 3.219049 | 1.47228 | 1.677557 |
| PCTUC_8 | 4.990086 | 1.091259 | 5.882385 | 3.828326 | 1.331393 |
| PCTUC_9 | −1.36474 | 1.922685 | 2.5084 | 1.303766 | 2.178063 |
| PCTUC_10 | 1.969926 | 2.081224 | 2.411553 | 1.657971 | −1.743247 |
| PCTUC_11 | 2.568045 | 1.596274 | 4.858005 | 2.774633 | 1.975318 |
| PCTUC_12 | 3.78894 | 2.013017 | 2.542768 | 2.626356 | 1.422498 |
| PCTUC_13 | 1.86401 | 2.481613 | 2.293715 | 1.852841 | 1.855033 |
| PCTUC_14 | 1.693567 | 2.454003 | 1.683912 | 1.03871 | 2.257919 |
| PCTUC_15 | 1.914858 | 2.013299 | 5.353806 | 4.08829 | 2.007689 |
| PCTUC_16 | 1.732865 | 2.3366 | 4.066249 | 3.479868 | 3.223681 |
| PCTUC_17 | 1.254072 | −1.096253 | −1.756643 | 1.935014 | −3.121983 |
| PCTUC_18 | 1.683823 | 2.186993 | 2.96542 | 2.487542 | 3.251436 |
| PCTUC_19 | 1.508808 | 1.818975 | 2.720885 | 3.012143 | 3.537117 |
| PCTUC_20 | 1.709264 | 2.094551 | 2.094095 | 1.963154 | 2.195627 |
| PCTUC_21 | 1.437381 | 2.023834 | 2.282534 | 1.277144 | 1.666151 |
| PCTUC_22 | 1.707798 | 2.027604 | 2.128659 | 2.171994 | 2.981197 |
| PCTUC_23 | 1.597093 | 2.13367 | 2.338216 | 2.116655 | 2.618075 |
| PCTUC_24 | −1.119074 | 1.240807 | 1.92722 | 2.278076 | 2.961248 |
| PCTUC_25 | 1.648136 | 2.679808 | 2.685308 | 2.532363 | 2.311755 |
| PCTUC_27 | 2.039945 | 2.314785 | 2.445415 | 1.99636 | 2.038934 |
| PCTUC_28 | 1.678706 | 2.822846 | 1.88328 | 1.953373 | 2.189538 |
| PCTUC_29 | 1.368914 | 2.383155 | 1.000889 | 2.234657 | 1.527855 |
| PCTUC_30 | 1.760434 | 2.268146 | 2.543274 | 1.988293 | 2.669211 |
| PCTUC_31 | 1.334197 | 2.871417 | 2.122144 | 1.3468 | 1.960979 |
| PCTUC_32 | 1.218089 | 1.774198 | 2.142013 | 2.850726 | 2.695594 |
| PCTUC_33 | 1.389436 | 2.702812 | 2.145138 | 1.920181 | 2.031137 |
| PCTUC_34 | 2.308969 | 1.788974 | 1.978217 | 2.045808 | 1.735026 |
| PCTUC_35 | 1.765146 | 2.861895 | 3.731195 | 2.581271 | 3.074587 |
| PCTUC_36 | 1.833468 | 2.432657 | 2.726498 | 2.961284 | 2.017774 |
| PCTUC_37 | 1.597116 | 1.618725 | 2.926616 | 2.415369 | 2.998529 |
| PCTUC_38 | 1.568038 | 1.956689 | 1.717832 | 1.848987 | 3.147673 |
| PCTUC_39 | 1.901959 | 3.708238 | 1.892787 | 3.094937 | 2.192067 |
| PCTUC_40 | 1.399132 | 2.258712 | 2.419852 | 1.798392 | 1.924235 |
| PCTUC_41 | 1.144015 | 1.825707 | 2.340699 | 1.925134 | 1.971642 |
| PCTUC_42 | 1.426665 | 2.701805 | 2.495222 | 3.045982 | 3.19735 |
| PCTUC_43 | 4.018248 | −1.717588 | 3.282683 | 1.355019 | −2.227438 |
| PCTUC_44 | 1.901663 | 2.288847 | 3.732934 | 1.771478 | 2.074904 |
| PCTUC_45 | 1.920817 | 2.960815 | 3.469161 | 2.769985 | 3.980089 |
| PCTUC_46 | 1.389416 | 4.811975 | 6.741828 | 1.411192 | 4.160511 |
| PCTUC_47 | 2.28666 | −1.026749 | 1.882731 | −1.020302 | −1.161207 |
| PCTUC_48 | 2.37839 | 2.51831 | 4.070007 | 2.886431 | 2.084877 |
| PCTUC_49 | 1.263168 | 3.046843 | 3.630741 | 3.958366 | 4.64426 |
| PCTUC_50 | 1.410489 | 2.236377 | 1.987109 | 2.029714 | 2.282976 |
| PCTUC_51 | 1.60263 | 2.121905 | 1.692895 | 1.738349 | 2.351656 |
| PCTUC_52 | 2.385026 | 1.081419 | 2.46315 | 1.161641 | −1.600941 |
| PCTUC_53 | 1.587455 | 2.536895 | 3.264168 | 3.314468 | 3.182655 |
| PCTUC_54 | 1.107599 | 2.914414 | 2.088041 | 2.297055 | 2.521916 |
| PCTUC_55 | 1.428883 | 2.366578 | 2.212243 | 2.574092 | 3.033405 |
| PCTUC_56 | 1.669315 | 2.29392 | 2.890783 | 3.26451 | 3.500622 |
| PCTUC_57 | 1.410287 | 2.059224 | 2.085696 | 1.64198 | 2.190387 |
| PCTUC_58 | 1.394989 | 2.557589 | 2.430643 | 2.140668 | 2.649637 |
| PCTUC_59 | 1.799848 | 1.85432 | 2.296163 | 1.586154 | 1.753843 |
| PCTUC_60 | 1.712394 | 2.212893 | 2.592989 | 1.856218 | 2.779451 |
| PCTUC_61 | 1.727563 | 1.623219 | 2.784914 | 2.158502 | 2.995003 |
| PCTUC_62 | 1.412706 | 2.11299 | 1.986623 | 1.438089 | 2.129747 |
| PCTUC_63 | 1.6105 | 4.15876 | 2.426561 | 2.206344 | 3.127447 |
| PCTUC_64 | 1.236849 | 2.340298 | 2.036072 | 2.087404 | 2.694909 |
| PCTUC_65 | 1.235732 | 2.317672 | 2.306382 | 2.003266 | 2.126323 |
| PCTUC_66 | 1.619317 | 2.178478 | 2.276473 | 2.05338 | 2.074758 |
| PCTUC_67 | 1.753909 | 1.97785 | 1.79724 | 1.433013 | 2.074351 |
| PCTUC_68 | 1.358542 | 2.104213 | 2.034427 | 1.717962 | 1.406569 |
| PCTUC_69 | 1.639055 | 1.641071 | 2.025247 | 1.196433 | 1.558159 |
| PCTUC_70 | 1.921972 | 2.268832 | 2.628053 | 1.66275 | 2.378746 |
| PCTUC_71 | 2.267135 | 1.564448 | 1.615601 | 2.039741 | 1.579345 |
| PCTUC_72 | 1.760416 | 3.557688 | 2.643089 | 2.44463 | 3.665364 |
| PCTUC_73 | 1.683902 | 1.809353 | 2.241871 | 2.040006 | 2.312258 |
| PCTUC_74 | 1.421482 | 1.944983 | 2.06476 | 1.296222 | 1.954839 |
| PCTUC_75 | 1.437059 | 2.601595 | 2.122126 | 2.107793 | 1.98714 |
| PCTUC_76 | 1.942441 | 1.555217 | 1.902045 | −1.09762 | −1.010611 |
| PCTUC_77 | 1.195854 | 2.903624 | 2.60441 | 2.066324 | 2.714115 |
| PCTUC_78 | 1.181195 | 1.810331 | 2.232351 | 1.67842 | 2.220953 |
| PCTUC_79 | 1.621712 | 2.469737 | 2.889681 | 2.831694 | 3.861511 |
| PCTUC_80 | 1.369115 | 1.454227 | 1.95301 | 1.843292 | 2.085274 |
| PCTUC_81 | 1.760196 | 2.054171 | 1.836205 | 1.987947 | 3.053376 |
| PCTUC_82 | 2.259899 | 4.030933 | 5.108284 | 1.813017 | 1.846668 |
| PCTUC_83 | 3.053387 | 2.580104 | 7.283586 | 3.504228 | 3.625079 |
| PCTUC_84 | 2.01469 | 3.056976 | 8.148343 | 3.72328 | 6.142403 |
| PCTUC_85 | 1.307712 | 1.589812 | 1.99927 | 1.847969 | 2.260472 |
| PCTUC_86 | 2.220748 | 3.581237 | 5.052755 | 5.616655 | 5.332936 |
| PCTUC_87 | 1.699534 | 2.314521 | 1.886742 | 1.394717 | 1.442726 |
| PCTUC_88 | 1.929106 | 2.036797 | 3.083732 | 3.142027 | 3.882488 |
| PCTUC_89 | 1.316076 | 1.530518 | 2.001615 | 1.930774 | 1.798015 |
| PCTUC_90 | 1.857735 | 2.417712 | 2.532709 | 1.813916 | 2.823546 |
| PCTUC_91 | 1.63355 | 3.832869 | 2.333097 | 1.372882 | 2.447965 |
| PCTUC_92 | 1.666357 | 2.701302 | 1.772067 | 1.774099 | 2.143625 |
| PCTUC_93 | 2.607343 | 4.430416 | 5.798988 | 5.255655 | 7.660719 |
| PCTUC_94 | 1.715865 | 2.232659 | 2.694269 | 2.328191 | 3.154522 |
| PCTUC_95 | 1.843929 | 1.533536 | 2.207211 | 1.42266 | 1.867618 |
| PCTUC_96 | 1.826181 | 1.444591 | 1.594736 | 1.656573 | 2.302421 |
| PCTUC_97 | 1.151094 | 1.998184 | 2.523026 | 2.472415 | 3.031885 |
| PCTUC_98 | 1.489856 | 3.310011 | 3.274649 | 2.506988 | 3.68643 |
| PCTUC_99 | 2.06415 | 2.539989 | 3.015937 | 2.478161 | 3.691117 |
| PCTUC_100 | 1.589476 | 2.987672 | 2.551001 | 2.194593 | 2.946532 |
| PCTUC_101 | 1.367315 | 1.511417 | 1.960072 | 1.568574 | 1.827056 |
| PCTUC_102 | 2.120865 | 2.018083 | 1.663197 | −1.251624 | 1.116722 |
| PCTUC_103 | 1.657802 | 1.825109 | 2.626874 | 2.153862 | 2.633694 |
| PCTUC_104 | 1.727754 | 1.908005 | 2.199311 | 1.763109 | 2.300518 |
| PCTUC_105 | 1.774548 | 2.498841 | 2.219475 | 2.53344 | 3.003811 |
| PCTUC_106 | 1.986182 | 2.1625 | 1.853343 | 1.876295 | 2.219061 |
| PCTUC_107 | 1.428675 | 3.43222 | 3.999756 | 2.34375 | 1.125152 |
| PCTUC_108 | 2.390612 | 2.187789 | 2.57185 | −2.25182 | −1.321648 |
| PCTUC_109 | 2.270507 | 4.797082 | 4.657402 | 4.699218 | 5.555741 |
| PCTUC_110 | 1.475036 | 1.720918 | 1.949022 | 1.091809 | 2.113063 |
| PCTUC_111 | 1.356621 | 2.66051 | 2.240773 | 1.417537 | 2.266325 |
| PCTUC_112 | 1.436257 | 2.905006 | 3.577459 | 3.043684 | 3.659114 |
| PCTUC_113 | 1.979509 | 2.612262 | 4.243793 | 3.004574 | 5.308458 |
| PCTUC_114 | 2.578598 | 4.425914 | 5.114237 | 5.027148 | 7.201783 |
| PCTUC_115 | 2.113645 | 3.79364 | 4.499948 | 3.998406 | 5.093233 |
| PCTUC_116 | 2.177961 | 2.663367 | 3.985358 | 3.658729 | 4.065497 |
| PCTUC_117 | 2.324831 | 3.71894 | 5.194387 | 4.871262 | 8.52777 |
| PCTUC_118 | 1.073007 | 2.146897 | 2.531411 | 2.827695 | 3.864724 |
| PCTUC_119 | 1.417364 | 3.733404 | 2.569529 | 1.923712 | 2.616817 |
| PCTUC_120 | 2.043426 | 3.579566 | 3.415488 | 3.988481 | 5.692827 |
| PCTUC_121 | 2.193191 | 3.784323 | 3.251114 | 2.983277 | 4.42307 |
| PCTUC_122 | 1.833187 | 2.382438 | 2.799391 | 2.247619 | 3.287899 |
| PCTUC_123 | 1.773008 | 2.15826 | 2.126261 | 1.823674 | 2.55418 |
| PCTUC_124 | 2.436061 | 3.979022 | 4.108718 | 3.802742 | 5.029344 |
| PCTUC_125 | 2.573088 | 3.251187 | 3.020403 | 2.911322 | 4.168266 |
| PCTUC_126 | 2.581049 | 3.680543 | 4.021809 | 3.870054 | 4.986007 |
| PCTUC_127 | 2.522426 | 4.309034 | 4.886045 | 4.733597 | 5.523011 |
| PCTUC_128 | 2.152378 | 4.22779 | 4.702429 | 4.055894 | 6.302035 |
| PCTUC_129 | 1.707048 | 2.728328 | 4.065711 | 2.544175 | 4.100242 |
| PCTUC_130 | 1.266083 | 2.077873 | 1.683113 | 1.734082 | 2.06349 |
| PCTUC_131 | 1.270988 | 2.04825 | 2.313275 | 2.000083 | 2.74953 |
| PCTUC_132 | 1.579236 | 1.830217 | 2.785681 | 2.457472 | 3.307127 |
| RCTUC_133 | 1.731929 | 3.150585 | 4.064429 | 2.606395 | 3.244058 |
| PCTUC_134 | 1.612822 | 4.448137 | 4.003712 | 3.285781 | 3.945924 |
| PCTUC_135 | 1.846059 | 1.863919 | 2.309483 | 1.794108 | 2.744771 |
| PCTUC_136 | 1.355217 | 1.798358 | 2.22741 | 1.796116 | 2.565216 |
| PCTUC_137 | 1.35991 | 2.570772 | 2.806133 | 2.604188 | 3.888691 |
| PCTUC_138 | 1.524161 | 1.745744 | 2.406214 | 2.042087 | 2.619121 |
| PCTUC_139 | 1.143266 | 2.066499 | 1.963953 | 1.841994 | 2.064502 |
| PCTUC_140 | 2.414967 | 4.98026 | 4.444968 | 4.510729 | 4.851974 |
| PCTUC_141 | 1.889636 | 2.541389 | 3.602278 | 2.748523 | 4.301198 |
| PCTUC_142 | 2.397714 | 4.855232 | 6.581445 | 5.864189 | 5.030571 |
| PCTUC_143 | 1.403801 | 2.190016 | 2.591157 | 2.568045 | 3.211911 |
| PCTUC_144 | 1.647328 | 2.414353 | 2.496427 | 2.541264 | 3.560737 |
| PCTUC_145 | 1.660398 | 2.395132 | 2.796336 | 2.654901 | 3.40539 |
| PCTUC_146 | 1.584713 | 2.94323 | 2.317092 | 2.006493 | 2.575637 |
| PCTUC_147 | 2.001094 | 2.210781 | 2.228616 | 2.339426 | |
| PCTUC_148 | 1.509576 | 2.475563 | 3.592372 | 3.070454 | 4.243995 |
| PCTUC_149 | 1.758313 | 2.260378 | 2.22741 | 1.93171 | 2.465638 |
| PCTUC_150 | 2.146984 | 3.071743 | 2.980247 | 2.838894 | 3.951463 |
| PCTUC_151 | 1.118945 | 1.690904 | 1.656675 | 2.072012 | 2.469634 |
| PCTUC_152 | 1.834308 | 2.092884 | 1.902702 | 1.615941 | 2.529564 |
| PCTUC_153 | 1.342434 | 2.327278 | 1.829888 | 1.51327 | 2.055782 |
| PCTUC_154 | 2.087219 | 5.444258 | 4.698549 | 3.542736 | 4.80305 |
| PCTUC_155 | 1.900503 | 3.86909 | 2.834414 | 2.736624 | 3.900374 |
| PCTUC_156 | 1.355351 | 3.034428 | 2.511339 | 2.120448 | 3.005841 |
| PCTUC_157 | 1.448459 | 2.420711 | 2.545386 | 1.995685 | 2.728806 |
| PCTUC_158 | 1.941758 | 2.007342 | 2.254047 | 1.631933 | 2.410079 |
| PCTUC_159 | 1.578095 | 3.848518 | 3.110776 | 2.391282 | 3.468199 |
| PCTUC_160 | 1.71481 | 3.247859 | 2.328171 | 2.225173 | 2.633327 |
| PCTUC_161 | 1.64334 | 1.970826 | 1.135816 | 1.408153 | 1.169709 |
| PCTUC_162 | 1.516161 | 3.428723 | 2.54344 | 1.760029 | 2.468676 |
| PCTUC_163 | 1.488808 | 3.324039 | 3.002135 | 2.720615 | 4.085257 |
| PCTUC_164 | 1.778077 | 3.709771 | 3.123746 | 3.284263 | 4.40286 |
| PCTUC_165 | 1.981667 | 3.475828 | 3.158709 | 3.283108 | 4.339454 |
| PCTUC_166 | 1.569606 | 2.37525 | 2.220013 | 1.636322 | 2.418371 |
| PCTUC_167 | 1.912417 | 3.642718 | 3.581518 | 4.185241 | 3.87418 |
| PCTUC_168 | 1.634055 | 2.771515 | 2.105632 | 1.719537 | 1.637775 |
| PCTUC_169 | 1.930187 | 3.72207 | 3.529785 | 2.909162 | 3.166899 |
| PCTUC_170 | 1.781 | 3.341992 | 3.305236 | 2.792567 | 3.879703 |
| PCTUC_171 | 2.919611 | 4.642657 | 4.902596 | 4.809088 | 5.596507 |
| PCTUC_172 | 1.550726 | 2.163172 | 2.490952 | 2.218841 | 3.090771 |
| PCTUC_173 | 2.903394 | 4.465715 | 5.46153 | 5.120931 | 6.050416 |
| PCTUC_174 | 1.861225 | 4.508595 | 3.761544 | 3.31325 | 4.360519 |
| PCTUC_175 | 1.755859 | 4.167829 | 2.880966 | 2.685777 | 3.379845 |
| PCTUC_176 | 2.262967 | 3.221856 | 3.569772 | 3.500499 | 4.628857 |
| PCTUC_177 | 1.524353 | 1.523746 | 2.458048 | 1.864571 | 2.685582 |
| PCTUC_178 | 1.847285 | 4.919324 | 4.203074 | 3.863744 | 4.174652 |
| PCTUC_179 | 1.747305 | 2.065585 | 2.24778 | 1.608568 | 2.147894 |
| PCTUC_180 | 1.946993 | 4.605177 | 4.671301 | 4.640214 | 5.030571 |
| PCTUC_181 | 1.425805 | 2.630357 | 1.739557 | 1.460964 | 2.22836 |
| PCTUC_182 | 1.996502 | 3.521755 | 2.171139 | 1.867099 | 2.465678 |
| PCTUC_183 | 2.458078 | 4.124454 | 2.703356 | 2.967947 | 3.801535 |
| PCTUC_184 | 2.076441 | 4.521826 | 5.001291 | 4.169762 | 4.55684 |
| PCTUC_185 | 2.14845 | 3.046543 | 3.044092 | 3.03148 | 3.943398 |
| PCTUC_186 | 1.769513 | 2.771634 | 2.852953 | 2.28604 | 2.764569 |
| PCTUC_187 | 1.613911 | 2.586738 | 2.64335 | 2.376528 | 3.03701 |
| PCTUC_188 | 2.045036 | 1.946169 | 2.901283 | 2.062389 | 1.39598 |
| PCTUC_189 | 1.169676 | 3.114371 | 2.026224 | 2.357244 | 2.775983 |
| PCTUC_190 | 1.856854 | 2.230163 | 3.423967 | 2.430517 | 2.049917 |
| PCTUC_191 | 1.381624 | 2.178181 | 1.989367 | 1.190545 | 2.491852 |
| PCTUC_192 | 1.482075 | 5.03285 | 2.084232 | 3.26451 | 4.011952 |
| PCTUC_193 | 1.29112 | 2.100079 | 2.480937 | 18.116383 | 2.875305 |
| PCTUC_194 | 2.001654 | 1.817391 | 2.171206 | 1.196635 | 2.300121 |
| PCTUC_195 | 1.900214 | 1.651488 | 1.142722 | 1.179752 | 1.38506 |
| PCTUC_196 | 1.73353 | 2.588496 | 3.12993 | 2.885138 | 3.500921 |
| PCTUC_197 | 2.348229 | 4.14646 | 4.193355 | 4.024918 | 5.287393 |
| PCTUC_198 | 1.261323 | 2.727162 | 2.404391 | 1.558354 | 2.043277 |
| PCTUC_199 | 1.487119 | 3.480046 | 2.848711 | 2.610347 | 3.40059 |
| PCTUC_200 | 1.388631 | 3.663344 | 3.536279 | 2.864961 | 3.865406 |
| PCTUC_201 | 1.994174 | 3.598878 | 3.87792 | 2.983387 | 4.888649 |
| PCTUC_202 | 2.062144 | 2.676319 | 2.53176 | 2.045086 | 3.460951 |
| PCTUC_203 | 1.880652 | 2.334102 | 2.838404 | 2.443658 | 3.758187 |
| PCTUC_204 | 1.656442 | 3.938874 | 5.426718 | 2.76839 | 4.144833 |
| PCTUC_205 | 1.670652 | 4.146744 | 4.298879 | 3.8508 | 5.717604 |
| PCTUC_206 | 1.62402 | 1.605383 | 1.612099 | 1.599941 | 2.471186 |
| PCTUC_207 | 1.797917 | 2.291486 | 3.536181 | 2.614953 | 4.12153 |
| PCTUC_208 | 1.490423 | 3.274915 | 3.02393 | 3.642722 | 3.909081 |
| PCTUC_209 | 1.66403 | 2.464782 | 2.728186 | 2.398898 | 3.647652 |
| PCTUC_210 | 1.723869 | 2.429967 | 2.551201 | 1.83207 | 2.613226 |
| PCTUC_211 | 3.216175 | 3.178193 | 3.426056 | 3.472931 | |
| PCTUC_212 | 2.177626 | 2.152791 | 2.324348 | 1.772685 | 2.773665 |
| PCTUC_213 | 1.631742 | 2.49734 | 2.631736 | 2.803728 | 3.287899 |
| PCTUC_214 | 2.201351 | 1.695774 | 1.932895 | 1.341692 | 2.163093 |
| PCTUC_215 | 1.464003 | 1.928169 | 2.393773 | 2.346943 | 2.913078 |
| PCTUC_216 | 1.803161 | 3.202755 | 3.098863 | 2.928031 | 4.003825 |
| PCTUC_217 | 1.096922 | 3.429597 | 1.901818 | 2.632384 | 2.647131 |
| PCTUC_218 | 1.901643 | 3.28671 | 2.752961 | 1.685245 | 2.549436 |
| PCTUC_219 | 1.47931 | 1.774027 | 2.026216 | 1.411155 | 1.786487 |
| PCTUC_220 | 2.250299 | 3.696237 | 3.884892 | 4.250008 | 5.088533 |
| PCTUC_221 | 1.8683 | 2.429364 | 2.863629 | 2.223653 | 3.416208 |
| PCTUC_222 | 2.575146 | 1.528597 | 1.305798 | 1.858591 | 1.029718 |
| PCTUC_223 | 2.1189 | 1.757274 | 2.368792 | 1.870888 | 2.201073 |
| PCTUC_224 | 1.143885 | 2.746074 | 1.721361 | 1.142136 | 3.933807 |
| PCTUC_225 | 3.947801 | 9.441274 | 14.251806 | 4.210367 | 6.532535 |
| PCTUC_226 | 2.55933 | 4.571705 | 3.583985 | 1.245965 | 2.777815 |
| PCTUC_227 | 2.978041 | 1.250008 | 2.425125 | 1.503769 | 1.170118 |
| PCTUC_228 | 1.813294 | 4.047213 | 2.051134 | 1.956453 | 1.732637 |
| PCTUC_229 | 7.329924 | 1.756996 | 6.414836 | 1.554824 | 2.703673 |
| PCTUC_230 | 2.291451 | 2.00425 | 3.101055 | 2.16244 | 2.591086 |
| PCTUC_231 | 1.650768 | 2.27758 | 2.859699 | 2.394677 | 3.114586 |
| PCTUC_232 | 1.851298 | 1.967491 | 2.211849 | 1.470639 | 1.909648 |
| PCTUC_233 | 3.503407 | 1.214912 | 2.087436 | 1.201315 | 1.306118 |
| PCTUC_234 | 1.744023 | 2.810245 | 2.624959 | 1.974127 | 2.182724 |
| PCTUC_235 | 1.967334 | 1.391752 | 2.217333 | 1.605545 | −1.062253 |
| PCTUC_236 | 1.125475 | −1.08361 | 1.131917 | 1.769547 | 2.291027 |
| PCTUC_237 | 1.95317 | 1.941142 | 2.974464 | 1.987368 | 2.460801 |
| PCTUC_238 | 1.761459 | 1.938436 | 2.437879 | 1.578815 | 1.985001 |
| PCTUC_239 | 2.327703 | 2.005099 | 2.31158 | −1.602604 | −1.592889 |
| PCTUC_240 | 1.797664 | 2.260158 | 1.986103 | 2.107779 | 1.786581 |
| PCTUC_241 | 1.93955 | 1.398541 | 2.159003 | 1.30178 | −1.097629 |
| PCTUC_242 | 2.19824 | 1.927631 | 2.652801 | 1.922133 | 2.28963 |
| PCTUC_243 | 3.253193 | 1.019366 | 1.921647 | −1.062837 | −1.102913 |
| PCTUC_244 | 1.588954 | 1.097882 | 2.41193 | 2.428367 | 2.164614 |
| PCTUC_245 | 1.736485 | 1.296248 | 2.383676 | −1.035178 | −1.085292 |
| PCTUC_246 | 1.596717 | 2.920444 | 2.129318 | 1.721134 | 1.554376 |
| PCTUC_247 | 1.619696 | 3.745029 | 5.322661 | 2.411938 | 3.973129 |
| PCTUC_248 | 1.355851 | 1.913746 | 2.992553 | 2.44081 | 4.331967 |
| PCTUC_249 | 1.296018 | 1.905911 | 2.062311 | 1.5673 | 2.105443 |
| PCTUC_250 | 2.773936 | 1.646017 | 2.262158 | 1.381524 | 1.037847 |
| PCTUC_251 | 1.488182 | 2.978148 | 2.1869 | 1.264823 | 2.8339 |
| PCTUC_252 | 1.3715228 | 1.023197 | 1.937373 | 2.682085 | 1.127316 |
| PCTUC_253 | 2.167365 | 1.996771 | 3.35515 | 1.913116 | 2.732446 |
| PCTUC_254 | 1.82616 | 2.351354 | 2.65802 | 1.413939 | 2.305251 |
| PCTUC_255 | 2.049325 | 2.728652 | 3.495365 | 2.180295 | 3.135996 |
| PCTUC_256 | 2.121907 | 2.668546 | 3.13753 | 2.356709 | 3.027172 |
| PCTUC_257 | 1.593813 | 2.171247 | 2.688064 | 1.854916 | 2.297249 |
| PCTUC_258 | 1.953386 | 3.046884 | 3.450625 | 2.276681 | 3.445494 |
| PCTUC_259 | 2.657611 | 1.752061 | 1.874493 | 1.991144 | 2.031666 |
| PCTUC_260 | 2.835871 | 4.359889 | 5.454328 | 2.956482 | 5.089366 |
| PCTUC_261 | 1.299203 | 1.699995 | 2.17389 | 2.328848 | 3.134634 |
| PCTUC_262 | 2.161551 | 2.553585 | 2.656883 | 1.799782 | 3.511413 |
| PCTUC_263 | 2.628037 | 2.22115 | 3.208899 | 1.673071 | 2.896297 |
| PCTUC_264 | 1.718884 | 2.355733 | 3.086609 | 2.092861 | 2.543259 |
| PCTUC_265 | 1.84443 | 1.990054 | 2.112158 | 1.604318 | 2.454317 |
| PCTUC_266 | 2.752462 | 5.271327 | 7.15142 | 6.201518 | 6.192584 |
| PCTUC_267 | 2.407998 | 2.606209 | 3.376878 | 2.233774 | 3.645929 |
| PCTUC_268 | 2.274251 | 2.286787 | 2.648614 | 2.672675 | 2.851749 |
| PCTUC_269 | 2.225441 | 2.327417 | 2.774195 | 1.849681 | 2.537223 |
| PCTUC_270 | 2.661012 | 3.828418 | 4.091862 | 3.042243 | 5.057569 |
| PCTUC_271 | 1.796683 | 2.179518 | 2.032843 | 2.390726 | 1.577298 |
| PCTUC_272 | 2.978599 | 1.343153 | 2.262219 | 1.054891 | 2.047256 |
| PCTUC_273 | 2.013162 | 2.119559 | 2.517091 | 1.56865 | 2.645934 |
| PCTUC_274 | 2.355896 | 2.007947 | 2.673893 | 1.531729 | 2.325394 |
| PCTUC_275 | 1.900366 | 2.590191 | 3.224328 | 1.998324 | 3.024211 |
| PCTUC_276 | 2.943753 | 6.126108 | 4.642348 | 5.059815 | 5.372001 |
| PCTUC_277 | 1.723907 | 3.269939 | 2.696898 | 2.057062 | 3.182218 |
| PCTUC_278 | 2.094828 | 2.38265 | 2.796767 | 1.854717 | 3.154387 |
| PCTUC_279 | 2.005874 | 2.978029 | 3.26697 | 2.05299 | 2.785948 |
| PCTUC_280 | 1.86396 | 1.957028 | 2.051702 | 1.616621 | 2.17304 |
| PCTUC_281 | 2.376895 | 3.270395 | 3.587495 | 2.29148 | 3.428254 |
| PCTUC_282 | 2.054744 | 2.199522 | 2.702134 | 2.041137 | 2.736642 |
| PCTUC_283 | 1.939767 | 2.589601 | 1.594674 | 1.63263 | 1.671657 |
| PCTUC_284 | 1.987288 | 2.528478 | 2.862927 | 1.735835 | 2.854227 |
| PCTUC_285 | 3.079297 | 6.083429 | 10.085707 | 6.538405 | 6.643817 |
| PCTUC_286 | 2.753213 | 3.59893 | 6.45603 | 5.407847 | 6.728555 |
| PCTUC_287 | 2.047107 | 2.906386 | 3.33462 | 3.347987 | 3.105007 |
| PCTUC_288 | 2.029243 | 1.85862 | 2.220732 | 1.430543 | 1.504025 |
| PCTUC_289 | 2.304952 | 2.399266 | 3.131665 | 1.835088 | 2.654428 |
| PCTUC_290 | 3.251477 | 4.356605 | 5.606828 | 3.09261 | 4.484571 |
| PCTUC_291 | 2.076605 | 2.595305 | 3.074471 | 1.621296 | 3.430257 |
| PCTUC_292 | 1.975275 | 2.718234 | 2.889359 | 3.352547 | 3.627154 |
| PCTUC_293 | 1.694107 | 2.019536 | 2.452825 | 1.697461 | 1.909641 |
| PCTUC_294 | 3.0436 | 2.595452 | 3.706271 | 2.084171 | 3.342356 |
| PCTUC_295 | 2.251134 | 2.904371 | 4.252948 | 2.152901 | 2.987439 |
| PCTUC_296 | 2.087629 | 3.49325 | 4.494589 | 1.443877 | 2.62743 |
| PCTUC_297 | 3.604622 | 7.745868 | 8.334743 | 4.608661 | 6.148468 |
| PCTUC_298 | 1.888779 | 2.302903 | 2.906211 | 1.626086 | 2.653658 |
| PCTUC_299 | 2.391559 | 5.756627 | 5.500046 | 3.411536 | 4.336478 |
| PCTUC_300 | 2.233243 | 2.326694 | 2.733778 | 2.099969 | 2.461344 |
| PCTUC_301 | 1.923591 | 1.734049 | 1.743211 | 1.667224 | 1.550048 |
| PCTUC_302 | 3.607384 | 3.005309 | 4.17177 | 3.054622 | 4.566369 |
| PCTUC_303 | 2.456815 | 1.899414 | 2.440792 | 2.188243 | 1.609168 |
| PCTUC_304 | 2.04325 | 2.31896 | 2.464632 | 1.583972 | 2.481459 |
| PCTUC_305 | 1.96945 | 1.37899 | 2.044353 | 1.11567 | 1.291094 |
| PCTUC_306 | 2.810436 | 3.373838 | 3.438033 | 2.450403 | 3.788159 |
| PCTUC_307 | 1.561316 | 2.133034 | 2.136498 | 1.667441 | 2.387891 |
| PCTUC_308 | 2.46688 | 5.101476 | 5.459764 | 3.302731 | 5.212498 |
| PCTUC_309 | 2.06269 | 3.498287 | 6.679554 | 3.305283 | 5.179197 |
| PCTUC_310 | 1.857961 | 2.588842 | 2.414808 | 2.136176 | 2.981805 |
| PCTUC_311 | 1.936857 | 2.728184 | 2.549392 | 2.345132 | 2.817231 |
| PCTUC_312 | 1.03898 | 1.911259 | 1.606964 | 1.840382 | 3.202574 |
| PCTUC_313 | 2.205216 | 2.254331 | 2.391725 | 1.496214 | 2.017714 |
| PCTUC_314 | 2.204293 | 2.621585 | 2.727868 | 2.266493 | 3.507617 |
| PCTUC_315 | 1.02131 | 1.873907 | 1.595523 | 2.2302 | 3.104499 |
| PCTUC_316 | 2.977269 | 5.084843 | 5.690078 | 4.364601 | 5.182178 |
| PCTUC_317 | 2.332324 | 3.953731 | 4.390101 | 2.765639 | 4.208732 |
| PCTUC_318 | 2.783622 | 5.77487 | 7.181798 | 3.896205 | 5.578665 |
| PCTUC_319 | 3.675624 | 5.221754 | 9.011139 | 1.676277 | 5.093641 |
| PCTUC_320 | 2.882559 | 2.693735 | 3.040426 | 2.081827 | 3.438727 |
| PCTUC_321 | 2.140977 | 1.388092 | 2.195915 | 1.118666 | 1.742483 |
| PCTUC_322 | 2.509368 | 4.285131 | 4.96098 | 3.772761 | 4.72897 |
| PCTUC_323 | 1.807721 | 2.053201 | 2.764767 | 1.650641 | 2.163632 |
| PCTUC_324 | 2.291451 | 2.072965 | 3.084313 | 1.686068 | 2.212211 |
| PCTUC_325 | 1.43054 | 2.912849 | 2.96758 | 3.002518 | 4.049307 |
| PCTUC_326 | 1.678896 | 2.176286 | 2.333714 | 1.653947 | 1.985383 |
| PCTUC_327 | 2.860305 | 4.50753 | 5.71555 | 3.221009 | 5.424201 |
| PCTUC_328 | 3.117953 | 3.532043 | 4.398926 | 2.240857 | 4.636419 |
| PCTUC_329 | 1.938489 | 2.246803 | 2.420075 | 1.885388 | 2.232656 |
| PCTUC_330 | 2.184758 | 1.576406 | 2.325558 | 1.450989 | 2.131205 |
| PCTUC_331 | 2.015503 | 2.105836 | 1.982838 | 1.824007 | 2.105958 |
| PCTUC_332 | 2.157558 | 2.35697 | 2.949601 | 1.824357 | 2.690155 |
| PCTUC_333 | 1.747717 | 2.934819 | 3.938158 | 1.939798 | 2.05878 |
| PCTUC_334 | 1.541244 | 2.08288 | 1.975025 | 1.536793 | 1.95171 |
| PCTUC_335 | 2.159401 | 2.32575 | 2.904458 | 1.605049 | 2.342069 |
| PCTUC_336 | 2.004049 | 2.530914 | 2.241112 | 1.911488 | 2.97038 |
| PCTUC_337 | 1.765664 | 2.402218 | 2.383659 | 1.561228 | 2.706952 |
| PCTUC_338 | 1.932377 | 2.605035 | 2.630431 | 1.97032 | 2.888035 |
| PCTUC_339 | 2.267443 | 2.164635 | 2.673887 | 1.663999 | 2.394497 |
| PCTUC_340 | 2.799647 | 2.489953 | 8.540805 | 2.280929 | 2.772973 |
| PCTUC_341 | 1.771648 | 1.997451 | 2.114003 | 1.511254 | 2.001948 |
| PCTUC_342 | 1.799832 | 2.274296 | 2.631532 | 2.505168 | 3.127952 |
| PCTUC_343 | 1.54815 | 1.723506 | 5.615517 | 2.155862 | 2.497843 |
| PCTUC_344 | 2.388751 | 3.892942 | 4.121072 | 2.506671 | 3.81235 |
| PCTUC_345 | 1.680449 | 1.65429 | 2.882919 | 2.379074 | 2.50676 |
| PCTUC_346 | 2.176567 | 2.489427 | 2.665195 | 2.374072 | 2.848118 |
| PCTUC_347 | 2.884108 | 4.174658 | 5.172139 | 3.595544 | 4.704914 |
| PCTUC_348 | 2.101132 | 2.69651 | 2.75628 | 2.03811 | 2.714414 |
| PCTUC_349 | 2.689832 | 3.726952 | 3.759751 | 2.906675 | 4.511586 |
| PCTUC_350 | 2.086607 | 2.639501 | 3.024416 | 2.016236 | 3.336448 |
| PCTUC_351 | 2.091848 | 2.217803 | 2.631467 | 1.692517 | 2.682757 |
| PCTUC_352 | 2.032929 | 3.296501 | 3.034418 | 2.017581 | 2.898892 |
| PCTUC_353 | 1.889645 | 2.48882 | 2.557603 | 2.039747 | 2.342551 |
| PCTUC_354 | 3.953862 | 6.095821 | 8.179487 | 4.740977 | 6.752993 |
| PCTUC_355 | 2.880915 | 6.314769 | 7.584483 | 4.459912 | 6.731706 |
| PCTUC_356 | 2.344102 | 3.970764 | 3.44365 | 2.745494 | 4.373598 |
| PCTUC_357 | 2.308369 | 2.443851 | 3.30145 | 1.698394 | 2.902148 |
| PCTUC_358 | 1.941134 | 2.458909 | 2.1209 | 1.535128 | 2.417566 |
| PCTUC_359 | 1.974657 | 2.151726 | 2.475451 | 1.630787 | 2.947569 |
| PCTUC_360 | 3.011921 | 3.695467 | 3.223783 | 2.485509 | 3.957333 |
| PCTUC_361 | 1.989847 | 2.630999 | 2.800455 | 1.736644 | 2.927565 |
| PCTUC_362 | 1.756287 | 2.323909 | 2.335853 | 1.741052 | 2.414361 |
| PCTUC_363 | 2.37175 | 2.724983 | 2.471031 | 1.969971 | 2.817292 |
| PCTUC_364 | 2.99462 | 7.012845 | 6.196832 | 4.300729 | 5.887486 |
| PCTUC_365 | 2.21789 | 2.560786 | 2.625995 | 1.882286 | 2.305182 |
| PCTUC_366 | 2.990927 | 3.261907 | 4.190991 | 3.234589 | 4.369407 |
| PCTUC_367 | 2.475569 | 2.606431 | 3.400553 | 2.48203 | 4.506122 |
| PCTUC_368 | 2.480668 | 3.439268 | 3.868262 | 3.597644 | 4.642876 |
| PCTUC_369 | −3.459225 | 3.245049 | 4.811554 | 5.0354 | |
| PCTUC_370 | 2.586246 | 3.630464 | 3.817188 | 2.615044 | 3.206606 |
| PCTUC_371 | 2.042603 | 1.004502 | 1.733275 | −1.294235 | −1.174134 |
| PCTUC_372 | 1.782903 | 2.419052 | 1.935022 | 1.625869 | 2.765687 |
| PCTUC_373 | 3.25888 | 2.063588 | 2.796654 | 1.830389 | 3.526159 |
| PCTUC_374 | 1.902272 | 2.083588 | 2.688988 | 1.345831 | 2.268873 |
| PCTUC_375 | 1.955749 | 1.43164 | 2.129483 | 1.254105 | 1.893949 |
| PCTUC_376 | 1.323888 | 2.214069 | 3.194154 | 2.050849 | 3.22952 |
| PCTUC_377 | 1.422594 | 2.470438 | 2.485046 | 2.02962 | 2.999551 |
| PCTUC_378 | 1.764847 | 2.037436 | 1.920452 | 1.353655 | 2.452314 |
| PCTUC_379 | 1.901509 | 2.479268 | 2.474901 | 1.896021 | 2.365896 |
| PCTUC_380 | 1.992917 | 1.760941 | 1.825539 | 1.185322 | 1.118641 |
| PCTUC_381 | 2.835555 | 4.980717 | 5.563924 | 4.130403 | 4.843069 |
| PCTUC_382 | 2.440718 | 3.481834 | 3.938086 | 2.907352 | 3.69767 |
| PCTUC_383 | 2.346514 | 2.061869 | 2.414692 | 1.614143 | 2.345685 |
| PCTUC_384 | 2.00554 | 2.358657 | 2.839284 | 2.372556 | 2.932624 |
| PCTUC_385 | 3.006696 | 2.695292 | 3.407489 | 2.419821 | 2.835872 |
| PCTUC_386 | 2.763716 | 4.548329 | 4.304034 | 2.650025 | 4.261168 |
| PCTUC_387 | 2.20499 | 3.004974 | 4.803649 | 3.60179 | 5.225955 |
| PCTUC_388 | 2.022286 | 2.602177 | 2.56764 | 1.487003 | 2.454711 |
| PCTUC_389 | 2.252397 | 1.349243 | 1.221948 | 1.085022 | 1.005355 |
| PCTUC_390 | 1.984737 | 1.886508 | 2.053055 | 1.276402 | 2.213593 |
| PCTUC_391 | 3.243428 | 4.212927 | 5.989446 | 3.823582 | 4.467264 |
| PCTUC_392 | 2.458363 | 2.53442 | 2.895445 | 2.231894 | 2.968892 |
| PCTUC_393 | 3.080195 | 2.562047 | 3.199905 | 1.644047 | 3.09701 |
| PCTUC_394 | 2.052922 | 1.485163 | 1.791144 | 1.482122 | 1.784639 |
| PCTUC_395 | 1.253237 | 1.754702 | 1.58624 | 1.737612 | 2.136938 |
| PCTUC_396 | 1.881169 | 2.456162 | 2.70796 | 2.091553 | 3.12313 |
| PCTUC_397 | 2.737342 | 2.910623 | 3.234478 | 2.156349 | 3.741645 |
| PCTUC_398 | 2.22809 | 1.460816 | 1.831952 | 1.041979 | 1.742374 |
| PCTUC_399 | 2.078429 | 2.442215 | 2.528577 | 1.249963 | 2.603135 |
| PCTUC_400 | 2.335688 | 1.93176 | 2.166172 | 1.336475 | 2.095809 |
| PCTUC_402 | 1.977784 | 1.942955 | 2.510741 | 1.574066 | 2.099593 |
| PCTUC_403 | 2.107548 | 2.115424 | 2.946819 | 1.726075 | 2.634579 |
| PCTUC_404 | 2.710852 | 2.382079 | 2.82944 | 2.009431 | 3.281803 |
| PCTUC_406 | 2.587248 | 2.348621 | 2.849712 | 1.686074 | 3.148104 |
| PCTUC_407 | 2.549039 | 3.362665 | 3.465319 | 2.534939 | 3.891845 |
| PCTUC_408 | 2.040769 | 2.560151 | 2.755626 | 1.725474 | 2.550859 |
| PCTUC_409 | 1.32847 | 1.948556 | 2.156865 | 1.743222 | 2.468351 |
| PCTUC_410 | 3.361394 | 4.898874 | 2.030213 | 2.812655 | |
| PCTUC_411 | 2.79521 | 1.793024 | 2.306354 | 1.328428 | 2.059772 |
| PCTUC_412 | 2.34141 | 2.07978 | 2.673887 | 1.493052 | 2.363667 |
| PCTUC_413 | 2.890792 | 4.322053 | 6.235146 | 2.594052 | 4.245276 |
| PCTUC_414 | 2.853143 | 3.271439 | 4.594022 | 2.097916 | 3.826796 |
| PCTUC_415 | 2.271866 | 2.004488 | 2.366559 | 1.588667 | 2.669858 |
| PCTUC_416 | 2.875755 | 3.032918 | 3.50868 | 2.250973 | 3.846255 |
| PCTUC_417 | 2.123813 | 2.482459 | 3.225545 | 1.897321 | 3.052733 |
| PCTUC_418 | 2.402113 | 2.174567 | 2.789737 | 2.576604 | 3.115066 |
| PCTUC_419 | 2.443327 | 2.057769 | 2.229288 | 1.63313 | 2.138522 |
| PCTUC_420 | 2.391559 | 2.380276 | 3.909662 | 1.890675 | 3.059867 |
| PCTUC_421 | 1.077536 | 2.594693 | 2.030645 | 2.401612 | 3.816474 |
| PCTUC_422 | 2.4507 | 1.751342 | 1.914153 | −1.994129 | −1.623521 |
| PCTUC_423 | 2.489001 | 2.474573 | 3.171004 | 1.814375 | 2.506032 |
| PCTUC_424 | 2.635959 | 6.207118 | 3.261327 | 2.182814 | 3.921246 |
| PCTUC_425 | 1.936564 | 2.257473 | 2.348564 | 1.621156 | 2.343169 |
| PCTUC_426 | 2.787 | 6.023342 | 6.340659 | 3.623966 | 5.023 |
| PCTUC_427 | 1.963182 | 2.59146 | 3.218375 | 1.981801 | 3.276546 |
| PCTUC_428 | 2.811955 | 4.697448 | 6.932314 | 3.533367 | 6.558954 |
| PCTUC_429 | 2.936883 | 4.995127 | 1.960985 | 2.883196 | 5.37323 |
| PCTUC_430 | 2.638209 | 4.390141 | 5.727512 | 2.911126 | 4.250409 |
| PCTUC_431 | 2.476889 | 3.380067 | 4.27829 | 2.543056 | 3.997764 |
| PCTUC_432 | 2.911814 | 1.989612 | 2.598813 | 1.571292 | 2.986079 |
| PCTUC_433 | 2.806021 | 3.194487 | 5.832902 | 3.000723 | 4.362042 |
| PCTUC_434 | 2.40136 | 3.17905 | 4.093861 | 1.955503 | 3.334149 |
| PCTUC_435 | 2.182042 | 2.924546 | 2.469926 | 2.081802 | 2.473711 |
| PCTUC_436 | 2.012068 | 1.624249 | 1.893484 | 1.304093 | 2.132982 |
| PCTUC_437 | 2.809489 | 8.348773 | 7.651482 | 5.333182 | 8.055189 |
| PCTUC_438 | 2.717542 | 4.266325 | 3.728613 | 3.076244 | 4.921318 |
| PCTUC_439 | 2.298005 | 2.509704 | 3.263963 | 1.49895 | 2.93489 |
| PCTUC_440 | 1.964941 | 2.645994 | 2.86284 | 1.821599 | 2.942023 |
| PCTUC_441 | 2.465924 | 2.763208 | 3.583939 | 2.550073 | 3.728398 |
| PCTUC_442 | 2.008205 | 2.701962 | 5.927336 | 1.823643 | 2.574186 |
| PCTUC_443 | 1.985571 | 1.791218 | 1.924718 | 1.65177 | 2.492881 |
| PCTUC_444 | 1.684871 | 2.639715 | 3.409781 | 1.514741 | 1.895138 |
| PCTUC_445 | 1.630364 | 3.215146 | 4.502505 | 3.277213 | 4.260717 |
| PCTUC_446 | 2.321074 | 3.346376 | 3.889021 | 2.635538 | 3.747683 |
| PCTUC_447 | 1.961918 | 2.018709 | 2.225815 | 1.469813 | 2.509177 |
| PCTUC_448 | 1.269913 | 2.187302 | 2.122471 | 1.998752 | 3.300487 |
| PCTUC_449 | 2.727314 | 6.261316 | 6.267126 | 4.693824 | 5.379424 |
| PCTUC_450 | 3.197335 | 4.956213 | 5.617073 | 3.907706 | 5.66407 |
| PCTUC_451 | 2.0707 | 3.137932 | 2.992602 | 1.916229 | 2.724454 |
| PCTUC_452 | 1.984737 | 1.36334 | 1.379513 | 1.280376 | 1.428708 |
| PCTUC_453 | 1.876505 | 3.497016 | 3.878442 | 2.477252 | 3.548843 |
| PCTUC_454 | 1.910458 | 1.869281 | 2.309147 | 1.478462 | 2.101685 |
| PCTUC_455 | 2.793179 | 3.666803 | 4.352312 | 2.484562 | 4.184809 |
| PCTUC_456 | −1.00204 | 1.287843 | 1.276185 | 1.068495 | 1.467703 |
| PCTUC_457 | 1.918738 | 2.538246 | 2.341118 | 1.655009 | 2.175128 |
| PCTUC_458 | 1.962578 | 3.124843 | 2.788344 | 1.466358 | 2.728292 |
| PCTUC_459 | 2.111142 | 1.830646 | 2.40658 | 1.115848 | 2.081882 |
| PCTUC_460 | 2.965396 | 3.735419 | 3.885926 | 2.441958 | 4.66639 |
| PCTUC_461 | 1.967691 | 2.630139 | 3.61916 | 2.220422 | 4.227802 |
| PCTUC_462 | 2.688921 | 1.809321 | 2.077108 | −1.119729 | −1.12931 |
| PCTUC_464 | 1.986388 | 3.7569 | 4.555263 | 1.884179 | 3.803697 |
| PCTUC_467 | 1.740369 | 2.339638 | 2.211905 | 1.92208 | 2.029057 |
| PCTUC_468 | 2.748851 | 3.820414 | 5.352636 | 3.791035 | 4.472126 |
| PCTUC_469 | 2.591887 | 3.058516 | 4.370934 | 2.663233 | 3.267893 |
| PCTUC_470 | 3.424826 | 8.002387 | 11.349638 | 7.409304 | 7.896651 |
| PCTUC_471 | 2.095686 | 2.172105 | 3.294361 | 1.948187 | 3.010162 |
| PCTUC_472 | 3.56209 | 4.849838 | 5.889228 | 4.071405 | 6.342055 |
| PCTUC_473 | 2.348436 | 3.795649 | 4.221846 | 2.31603 | 3.951533 |
| PCTUC_474 | 2.14509 | 3.216147 | 4.660154 | 2.7524 | 5.420599 |
| PCTUC_475 | 3.35751 | 6.479264 | 8.310672 | 5.376397 | 6.879448 |
| PCTUC_476 | 2.671123 | 4.650983 | 5.04074 | 3.253613 | 4.819563 |
| PCTUC_477 | 2.387151 | 2.945137 | 3.255189 | 2.492054 | 2.513826 |
| PCTUC_478 | 2.645504 | 4.693357 | 7.934476 | 4.227252 | 5.576314 |
| PCTUC_479 | 2.699603 | 5.376858 | 7.113347 | 4.423897 | 5.450957 |
| PCTUC_480 | 2.322522 | 2.093338 | 2.796594 | 1.619356 | 2.265154 |
| PCTUC_481 | 1.346006 | 3.905318 | 2.776636 | 3.143842 | 3.796467 |
| PCTUC_482 | 2.862653 | 6.053042 | 5.972333 | 4.49482 | 5.577618 |
| PCTUC_483 | 2.420212 | 3.421625 | 3.582682 | 2.395104 | 3.517098 |
| PCTUC_484 | 2.371871 | 3.803153 | 3.727755 | 2.296599 | 3.348165 |
| PCTUC_485 | 2.112958 | 1.907894 | 2.063742 | 1.316474 | 1.965625 |
| PCTUC_486 | 2.540716 | 5.23061 | 5.541661 | 4.039124 | 4.86787 |
| PCTUC_487 | 2.620514 | 3.613905 | 4.692163 | 2.593385 | 4.190191 |
| PCTUC_488 | 2.670145 | 4.679341 | 5.579667 | 3.789751 | 4.612708 |
| PCTUC_489 | 2.434191 | 3.85106 | 4.174643 | 2.855676 | 4.725146 |
| PCTUC_490 | 1.00053 | 2.110267 | 1.543375 | 2.442942 | 3.5069 |
| PCTUC_491 | 2.92511 | 2.435802 | 2.331618 | 1.966097 | 2.968132 |
| PCTUC_492 | 2.268654 | 2.685976 | 2.430065 | 1.627878 | 2.18276 |
| PCTUC_493 | 2.431231 | 1.919509 | 2.458445 | 1.699867 | 1.642623 |
| PCTUC_494 | 1.785818 | 1.975354 | 1.989132 | 1.672897 | 2.13476 |
| PCTUC_495 | 1.660831 | 2.083885 | 2.297657 | 2.914436 | 3.204953 |
| PCTUC_496 | −1.007082 | 2.12366 | 1.591845 | 2.178094 | 2.857278 |
| PCTUC_497 | 1.323915 | 1.730687 | 2.357525 | 1.763247 | 3.182555 |
| PCTUC_498 | 1.819995 | 2.924548 | 2.83141 | 1.952073 | 2.535718 |
| PCTUC_499 | 1.103923 | 1.935671 | 1.822613 | 2.506015 | 2.594218 |
| PCTUC_500 | 2.108564 | 2.599634 | 3.275906 | 2.786753 | 4.39258 |
| PCTUC_501 | 2.202682 | 1.428914 | 1.944631 | 1.495945 | 2.239097 |
| PCTUC_502 | 1.511049 | 2.666108 | 2.601538 | 2.911196 | 3.460927 |
| PCTUC_503 | 2.016269 | 1.952843 | 1.82467 | 1.463055 | 2.126049 |
| PCTUC_504 | 2.329668 | 2.567232 | 2.692213 | 1.521157 | 2.197148 |
| PCTUC_505 | 1.591243 | 2.751235 | 2.449544 | 1.66492 | 2.427388 |
| PCTUC_506 | 2.183582 | 1.918865 | 1.706821 | 1.224784 | 1.548311 |
| PCTUC_507 | 2.121192 | 2.149194 | 2.676733 | 1.717022 | 2.754955 |
| PCTUC_508 | 1.958871 | 1.943062 | 1.667803 | 1.494308 | 2.107172 |
| PCTUC_509 | 1.933163 | 1.725295 | 1.748657 | −1.138867 | 1.133237 |
| PCTUC_510 | 1.468092 | 2.099357 | 1.940111 | 1.872053 | 2.135083 |
| PCTUC_511 | 2.154159 | 2.097517 | 2.410887 | 1.261737 | 2.364517 |
| PCTUC_512 | 1.147774 | 1.783252 | 2.252219 | 2.044234 | 3.814058 |
| PCTUC_513 | 2.47837 | 2.887622 | 3.551895 | 1.792477 | 2.975644 |
| PCTUC_514 | 2.867124 | 2.561581 | 2.992617 | 1.677437 | 2.835756 |
| PCTUC_515 | 1.627145 | 2.312069 | 2.810126 | 1.996169 | 2.617225 |
| PCTUC_516 | 1.726031 | 2.281983 | 3.149352 | 2.109466 | 3.559903 |
| PCTUC_517 | 2.849573 | 3.00185 | 3.49281 | 2.722194 | 4.174789 |
| PCTUC_518 | 2.27343 | 4.425375 | 6.756968 | 4.773911 | 6.573621 |
| PCTUC_519 | 2.797114 | 3.013171 | 2.941275 | 2.861877 | 3.896014 |
| PCTUC_520 | 2.145546 | 1.51115 | 2.011956 | 1.136702 | 1.394944 |
| PCTUC_521 | 2.110756 | 3.817168 | 3.857475 | 2.626673 | 4.603748 |
| PCTUC_522 | 2.805435 | 2.143939 | 2.405287 | 1.943805 | 3.157292 |
| PCTUC_523 | 2.232193 | 2.524796 | 2.664089 | 1.784314 | 2.867758 |
| PCTUC_524 | 2.433354 | 2.742706 | 3.018785 | 2.42759 | 3.763151 |
| PCTUC_525 | 2.042445 | 3.087261 | 3.427543 | 2.131444 | 3.138867 |
| PCTUC_526 | 1.819044 | 1.85444 | 2.008788 | 1.4249 | 2.145726 |
| PCTUC_527 | 3.182961 | 6.456305 | 7.971256 | 7.174463 | 6.581938 |
| PCTUC_528 | 2.512796 | 5.234399 | 6.328454 | 4.522633 | 6.481423 |
| PCTUC_529 | 1.915459 | 1.857537 | 1.934087 | 1.285698 | 1.914062 |
| PCTUC_530 | 2.551965 | 3.05574 | 3.508546 | 2.27785 | 4.110225 |
| PCTUC_531 | 2.315542 | 2.948552 | 3.496778 | 1.73247 | 2.323464 |
| PCTUC_532 | 2.570683 | 2.284815 | 2.635896 | 2.962807 | 2.741162 |
| PCTUC_533 | 3.050366 | 2.7762 | 3.603807 | 3.006189 | 3.681454 |
| PCTUC_534 | 1.973636 | 2.828246 | 3.083603 | 1.815389 | 3.059071 |
| PCTUC_535 | 2.341728 | 3.394196 | 4.137745 | 3.503355 | 3.483258 |
| PCTUC_536 | 5.155655 | 5.988683 | 5.516017 | 6.628252 | |
| PCTUC_537 | 2.447649 | 5.432794 | 4.880079 | 3.579062 | 5.154306 |
| PCTUC_538 | 2.296285 | 2.399659 | 2.631008 | 1.729843 | 2.877178 |
| PCTUC_539 | 1.995681 | 1.760945 | 1.987853 | 1.698475 | 2.458658 |
| PCTUC_540 | 2.941353 | 1.844049 | 2.175175 | 1.444954 | 1.928482 |
| PCTUC_541 | 2.275076 | 2.124308 | 2.580506 | 1.574504 | 2.246783 |
| PCTUC_542 | 2.051276 | 1.402258 | 2.145409 | 1.243739 | 2.369804 |
| PCTUC_543 | 1.959295 | 1.987292 | 2.511984 | 1.558444 | 2.087383 |
| PCTUC_544 | 2.557583 | 3.232352 | 3.451574 | 2.111259 | 3.616122 |
| PCTUC_545 | 2.308393 | 2.793809 | 3.41116 | 1.722354 | 3.360532 |
| PCTUC_546 | 2.525068 | 2.366782 | 3.208857 | 1.82044 | 3.108346 |
| PCTUC_547 | 2.245233 | 1.469712 | 1.604138 | −1.492083 | −1.072894 |
| PCTUC_548 | 2.260957 | 2.562115 | 3.803141 | 1.176394 | 1.92117 |
| PCTUC_549 | 1.578194 | 2.249098 | 2.658492 | −1.194468 | 1.463275 |
| PCTUC_550 | 2.306434 | 2.517305 | 2.607248 | 1.930816 | 2.87325 |
| PCTUC_551 | 1.988129 | 1.546736 | 1.716089 | 1.394948 | 1.991959 |
| PCTUC_552 | 2.573594 | 2.512912 | 2.64596 | 1.937291 | 2.563375 |
| PCTUC_553 | 2.185575 | 2.258192 | 2.0861 | 1.424623 | 2.222347 |
| PCTUC_554 | 2.341026 | 4.148862 | 4.517709 | 2.382762 | 4.91679 |
| PCTUC_555 | 2.190103 | 2.354151 | 2.306093 | 1.419395 | 2.184773 |
| PCTUC_556 | 2.630554 | 2.111118 | 2.346193 | 1.319616 | 2.781438 |
| PCTUC_557 | 2.332639 | 2.480744 | 2.815126 | 1.940635 | 2.798939 |
| PCTUC_558 | 1.665789 | 2.06459 | 2.231466 | 1.619472 | 2.350911 |
| PCTUC_559 | 2.205838 | 2.108767 | 2.029673 | 1.304332 | 2.039695 |
| PCTUC_560 | 2.452425 | 4.978587 | 4.384961 | 3.288052 | 3.699426 |
| PCTUC_561 | 3.724798 | 2.758653 | 1.581049 | −1.079886 | 1.651879 |
| PCTUC_562 | 1.458079 | 2.06474 | 2.293924 | 2.117243 | 2.045964 |
| PCTUC_563 | 1.330289 | 2.358853 | 2.966393 | 3.292261 | 2.070739 |
| PCTUC_564 | 1.398376 | 1.335047 | 2.796356 | 3.870261 | 3.021988 |
| PCTUC_565 | 1.642279 | 2.27982 | 2.227257 | 2.123359 | 2.019439 |
| PCTUC_566 | 1.794402 | 2.498483 | 2.64332 | 2.308097 | 2.197079 |
| PCTUC_568 | 1.744485 | 2.015622 | 2.518641 | 2.257252 | 2.248926 |
| PCTUC_569 | 2.901726 | −1.50788 | 3.395245 | 2.128505 | −1.653142 |
| PCTUC_570 | 1.150738 | 4.060815 | 3.067239 | 3.349717 | 2.06671 |
| PCTUC_571 | 2.047153 | −1.008306 | 1.45744 | −1.386172 | −2.112743 |
| PCTUC_572 | −1.194861 | 3.665618 | −1.085841 | 3.47144 | 3.62577 |
| PCTUC_573 | 1.879792 | 2.448401 | 2.685976 | 2.137358 | 2.185858 |
| PCTUC_574 | 1.815429 | 2.230381 | 2.324055 | 1.982066 | 1.956972 |
| PCTUC_575 | 2.050044 | 2.155466 | 2.329984 | 2.099358 | 2.332833 |
| PCTUC_576 | 1.846621 | 2.27799 | 3.027295 | 1.976763 | 2.776882 |
| PCTUC_577 | 1.747903 | 2.632668 | 2.756806 | 2.36739 | 2.146093 |
| PCTUC_578 | 2.186524 | 2.089006 | 2.601211 | 1.727324 | 1.500667 |
| PCTUC_579 | 2.210933 | 1.89179 | 4.36574 | 2.389901 | 2.087243 |
| PCTUC_580 | 2.106504 | 1.186451 | 2.692309 | 2.473882 | 1.343175 |
| PCTUC_581 | 1.456263 | 3.17164 | 11.32385 | 2.588184 | 5.754287 |
| PCTUC_582 | 2.614078 | 1.645149 | 1.820179 | −2.838872 | −2.738889 |
| PCTUC_583 | 3.318897 | 1.427448 | 1.565843 | 1.878233 | 1.184871 |
| PCTUC_584 | 1.954525 | 1.144457 | −1.009171 | 1.257005 | −1.396045 |
| PCTUC_585 | 2.765282 | 2.180095 | 2.33827 | 2.243864 | 2.836234 |
| PCTUC_586 | 2.133562 | 2.115911 | 3.00543 | 1.91642 | 1.857386 |
| PCTUC_587 | 2.549655 | 1.498298 | −1.012961 | −1.919216 | −2.147842 |
| PCTUC_588 | 1.542745 | 3.807242 | 2.343264 | 2.309335 | 2.6663 |
| PCTUC_589 | 2.052527 | 2.493824 | 2.967375 | 2.424135 | 2.66706 |
| PCTUC_590 | 1.623075 | 3.704542 | 3.459654 | 2.542809 | 2.654304 |
| PCTUC_591 | 1.983467 | 2.169764 | 1.540234 | 1.987544 | −1.006504 |
| PCTUC_592 | 2.239261 | 2.481292 | 2.64519 | 1.770056 | 1.99023 |
| PCTUC_593 | 2.071805 | 2.562777 | 4.070767 | 3.268451 | 2.346879 |
| PCTUC_594 | 1.74096 | 3.361343 | 3.544527 | 3.864242 | 2.743535 |
| PCTUC_595 | 1.559802 | 1.904926 | 2.265164 | 2.075392 | 1.901483 |
| PCTUC_596 | 1.784124 | 2.275661 | 3.585576 | 2.084074 | 2.095021 |
| PCTUC_597 | 3.159435 | −1.697335 | 4.054702 | 1.388331 | −8.53404 |
| PCTUC_598 | 1.75033 | 2.718441 | 2.196595 | 2.09338 | 1.579785 |
| PCTUC_599 | 2.099691 | 2.677208 | 2.061399 | 2.272578 | 1.872414 |
| PCTUC_600 | 2.130302 | 1.908707 | 2.488858 | 1.399387 | −1.326734 |
| PCTUC_601 | 1.462605 | 3.064583 | 3.174861 | 1.935508 | 9.527042 |
| PCTUC_602 | 1.700839 | 1.833059 | 2.171972 | 2.079844 | 1.662449 |
| PCTUC_603 | −1.584713 | −1.579554 | 1.63215 | −1.17958 | 1.944797 |
| PCTUC_604 | 2.68338 | 2.241231 | 4.40873 | 2.714834 | 2.437561 |
| PCTUC_605 | 1.898492 | 1.350918 | 2.222734 | 1.083346 | 1.33569 |
| PCTUC_606 | 1.581753 | 2.66108 | 2.529084 | 2.030086 | 1.452565 |
| PCTUC_607 | 1.654599 | 2.324419 | 2.59917 | 2.451451 | 2.103469 |
| PCTUC_608 | 2.086731 | 3.106404 | 4.167883 | 2.874574 | 1.804172 |
| PCTUC_609 | 1.772983 | 3.051194 | 4.351245 | 2.107334 | 2.227571 |
| PCTUC_610 | 3.519794 | 5.237543 | 3.504589 | 2.221707 | 2.152098 |
| PCTUC_611 | 2.276961 | 3.20788 | 3.081503 | 2.240111 | 1.793533 |
| PCTUC_612 | 1.659303 | 2.216207 | 2.995906 | 2.361989 | 1.927937 |
| PCTUC_613 | 1.55503 | 2.021138 | 2.085583 | 2.246955 | 2.521659 |
| PCTUC_614 | 2.65174 | 3.898009 | 4.171721 | 3.421649 | 3.565886 |
| PCTUC_615 | 1.978197 | 4.354056 | 1.955533 | −1.100134 | −1.1857 |
| PCTUC_616 | 1.862664 | 2.006837 | 2.45286 | 2.204572 | 2.003495 |
| PCTUC_617 | 1.696718 | 2.119472 | 2.426069 | 2.377271 | 2.046716 |
| PCTUC_618 | 2.89914 | 4.475184 | 7.241369 | 5.205436 | 4.131087 |
| PCTUC_619 | 2.321978 | 3.009422 | 3.721688 | 3.073065 | 2.042396 |
| PCTUC_620 | 2.167707 | 2.134956 | 2.298337 | 1.714914 | 2.049021 |
| PCTUC_621 | 2.116471 | 6.734607 | 4.110401 | 3.675307 | 3.180559 |
| PCTUC_622 | 2.514078 | 2.875581 | 3.276011 | 2.336778 | 3.035044 |
| PCTUC_623 | 2.605199 | 3.487387 | 3.974484 | 4.055721 | 4.236562 |
| PCTUC_624 | 2.118815 | 3.457612 | 3.882709 | 2.828475 | 2.821918 |
| PCTUC_625 | 2.081784 | 3.337394 | −1.713592 | 3.08825 | 2.179591 |
| PCTUC_626 | 3.007421 | 5.284403 | 6.718605 | 5.273727 | 5.698007 |
| PCTUC_627 | 1.7371 | 2.475792 | 2.327755 | 1.602649 | 2.6975 |
| PCTUC_628 | 2.56408 | 2.984585 | 3.741057 | 3.25906 | 2.476948 |
| PCTUC_629 | 2.044725 | 1.800382 | 3.497649 | 1.133335 | 1.157579 |
| PCTUC_630 | 2.048723 | 2.555925 | 3.197447 | 2.384872 | 2.957937 |
| PCTUC_631 | 1.987264 | 2.027627 | 2.22932 | 1.486664 | 1.569272 |
| PCTUC_632 | 1.508266 | 2.289024 | 2.49168 | 1.969036 | 1.942292 |
| PCTUC_633 | 1.711891 | 2.335012 | 2.855269 | 2.77205 | 2.872453 |
| PCTUC_634 | 1.85976 | 2.048713 | 2.137747 | 1.426612 | 1.078268 |
| PCTUC_635 | 1.950333 | 1.993799 | 2.140466 | 2.074382 | 2.244986 |
| PCTUC_636 | 1.790172 | 2.568077 | 2.913422 | 2.262483 | 2.405525 |
| PCTUC_637 | 1.079308 | 2.512235 | 1.88564 | 2.880826 | 2.823866 |
| PCTUC_638 | 2.067919 | 1.923353 | 2.435373 | 2.128093 | 2.20338 |
| PCTUC_639 | 1.966816 | 3.490128 | 3.441771 | 2.172453 | 1.944276 |
| PCTUC_640 | 2.489141 | 8.613799 | 5.361654 | 4.848388 | 3.89614 |
| PCTUC_641 | 2.574851 | 5.807697 | 5.468801 | 4.90076 | 4.871609 |
| PCTUC_642 | 2.463501 | 4.48316 | 4.04062 | 3.272967 | 3.171938 |
| PCTUC_643 | 2.352794 | 3.681963 | 4.545543 | 4.167448 | 3.945667 |
| PCTUC_644 | 2.748792 | 4.362951 | 6.57235 | 4.583384 | 4.447282 |
| PCTUC_645 | 2.083058 | 3.503422 | 3.768635 | 3.508003 | 2.443652 |
| PCTUC_646 | 2.187366 | 2.567158 | 3.634599 | 3.011877 | 2.432719 |
| PCTUC_647 | 1.962954 | 3.286385 | 3.175565 | 2.436413 | 3.026659 |
| PCTUC_648 | 1.610516 | 2.408909 | 2.309618 | 2.284194 | 1.903609 |
| PCTUC_649 | 2.738749 | 5.635113 | 8.772574 | 6.176256 | 5.03567 |
| PCTUC_650 | 2.598258 | 4.604433 | 4.868678 | 5.932552 | 3.396743 |
| PCTUC_651 | 1.925645 | 3.539554 | 2.666421 | 2.136865 | 2.279523 |
| PCTUC_652 | 2.913148 | 4.555728 | 1.156568 | 4.910597 | 4.307831 |
| PCTUC_653 | 2.767277 | 5.956202 | 1.555341 | 6.13508 | 5.008499 |
| PCTUC_654 | 2.331898 | 2.444826 | 2.8718 | 2.382006 | 2.741199 |
| PCTUC_655 | 1.224894 | 2.950068 | 2.167558 | 3.359763 | 3.099351 |
| PCTUC_656 | 2.362089 | 3.633137 | 4.458858 | 3.959656 | 3.985284 |
| PCTUC_657 | 2.150204 | 2.150625 | 3.353007 | 2.31164 | 2.392572 |
| PCTUC_658 | 1.644417 | 2.892346 | 2.837056 | 2.655083 | 2.161197 |
| PCTUC_659 | 1.973494 | 1.78177 | 2.413365 | 2.113657 | 1.750194 |
| PCTUC_660 | 2.166205 | 2.403092 | 3.011296 | 1.741263 | 1.859095 |
| PCTUC_661 | 1.715786 | 2.752077 | 2.625633 | 2.526097 | 2.552588 |
| PCTUC_662 | 2.663858 | 5.457253 | 6.366603 | 5.702743 | 5.768478 |
| PCTUC_663 | 2.356379 | 4.800281 | 5.678669 | 5.463997 | 4.923267 |
| PCTUC_664 | 1.986381 | 1.856272 | 2.469981 | 2.215134 | 2.151767 |
| PCTUC_665 | 2.495689 | 3.434863 | 3.68608 | 3.742092 | 3.742618 |
| PCTUC_666 | 3.220485 | 6.93086 | 9.153201 | 5.997135 | 5.199389 |
| PCTUC_667 | 1.611768 | 2.271733 | 2.909057 | 3.039583 | 2.493955 |
| PCTUC_668 | 1.648326 | 1.919215 | 2.367546 | 1.940866 | 2.018715 |
| PCTUC_669 | 1.908394 | 2.337281 | 2.577075 | 1.47188 | 1.641456 |
| PCTUC_670 | 2.070219 | 2.981058 | 3.041791 | 2.489966 | 2.225878 |
| PCTUC_671 | 1.709244 | 2.416751 | 2.559589 | 2.054646 | 2.06568 |
| PCTUC_672 | 2.727472 | 4.420219 | 4.758027 | 4.863633 | 4.530597 |
| PCTUC_673 | 1.934935 | 2.240915 | 3.633712 | 2.146384 | 2.153216 |
| PCTUC_674 | 2.743376 | 5.589533 | 5.753892 | 4.882175 | 5.347692 |
| PCTUC_675 | 2.173746 | 2.78693 | 2.843419 | 2.028366 | 1.98453 |
| PCTUC_676 | 2.187662 | 2.288546 | 2.932474 | 2.404961 | 2.084628 |
| PCTUC_677 | 1.438577 | 2.052207 | 1.749069 | 2.615633 | 2.229117 |
| PCTUC_678 | 2.203922 | 2.936104 | 3.330144 | 3.415096 | 3.076645 |
| PCTUC_679 | 1.973946 | 3.2316 | 2.814185 | 4.022079 | 2.479098 |
| PCTUC_680 | 2.046929 | 2.532627 | 2.774914 | 1.819864 | 1.925472 |
| PCTUC_681 | 2.276395 | 3.817427 | 4.516187 | 2.855823 | 3.798191 |
| PCTUC_682 | 2.315317 | 3.596598 | 4.557338 | 3.690554 | 3.464538 |
| PCTUC_683 | 2.056906 | 4.222321 | 4.727066 | 3.607633 | 3.268795 |
| PCTUC_684 | 2.503668 | 4.190849 | 5.953754 | 4.558081 | 4.440939 |
| PCTUC_685 | 2.345897 | 2.715857 | 3.245863 | 2.231445 | 2.50942 |
| PCTUC_686 | 2.165409 | 4.294438 | 4.208843 | 3.325176 | 2.755725 |
| PCTUC_687 | 2.074088 | 1.869166 | 2.815679 | 1.490269 | 1.342491 |
| PCTUC_688 | 1.99125 | 1.958521 | 2.333184 | 1.27325 | −1.066773 |
| PCTUC_689 | 2.131139 | 2.383163 | 2.505348 | 2.325377 | 2.199408 |
| PCTUC_690 | 2.50947 | 5.384008 | 6.459625 | 5.017322 | 4.32398 |
| PCTUC_691 | 2.205507 | 2.210307 | 2.322275 | 2.126769 | 2.07638 |
| PCTUC_692 | 2.224838 | 2.85757 | 3.214349 | 3.428101 | 2.408178 |
| PCTUC_693 | 1.548608 | 2.368227 | 2.242102 | 2.130813 | 1.939746 |
| PCTUC_694 | 2.199028 | 2.382027 | 2.465793 | 1.909728 | 2.059906 |
| PCTUC_695 | 2.287057 | 3.326586 | 3.396893 | 2.944132 | 2.388609 |
| PCTUC_696 | 1.970905 | 2.222844 | 2.797527 | 3.212934 | 2.826079 |
| PCTUC_697 | 2.347556 | 3.192369 | 4.477496 | 3.643991 | 3.435004 |
| PCTUC_698 | 2.125621 | 2.448727 | 3.056476 | 2.241038 | 2.410933 |
| PCTUC_699 | 2.635933 | 4.609246 | 6.675708 | 4.95645 | 4.966762 |
| PCTUC_700 | 1.983465 | 2.705402 | 2.346061 | 1.430664 | −1.167211 |
| PCTUC_701 | 2.144452 | 3.534742 | 4.30228 | 3.916895 | 3.484727 |
| PCTUC_702 | 2.109074 | 3.338197 | 4.553086 | 3.53563 | 3.263711 |
| PCTUC_703 | 2.852373 | 5.956202 | 6.243945 | 4.714651 | 5.417841 |
| PCTUC_704 | 2.132345 | 2.505468 | 3.092073 | 2.572824 | 2.661616 |
| PCTUC_705 | 1.778648 | 2.980165 | 2.849719 | 2.00298 | 1.713915 |
| PCTUC_706 | 1.814586 | 2.2015 | 2.705321 | 1.909649 | 2.313449 |
| PCTUC_707 | 2.458768 | 5.149868 | 4.922929 | 4.477264 | 5.023233 |
| PCTUC_708 | 2.184737 | 2.946659 | 3.779886 | 2.632446 | 2.432719 |
| PCTUC_709 | 2.622874 | 4.778035 | 5.593929 | 4.240022 | 4.561161 |
| PCTUC_710 | 2.287708 | 4.084493 | 4.953694 | 3.663384 | 4.028213 |
| PCTUC_711 | 2.115186 | 2.804591 | 2.285582 | 2.297939 | 2.056486 |
| PCTUC_712 | 2.684022 | 2.781386 | 3.591925 | 2.617531 | 2.670167 |
| PCTUC_713 | 2.00528 | 3.891295 | 3.109384 | 2.850291 | 3.159817 |
| PCTUC_714 | 2.099078 | 2.875953 | 3.59224 | 2.31435 | 2.403818 |
| PCTUC_715 | 1.59096 | 4.816008 | 3.40176 | 4.762467 | 3.724533 |
| PCTUC_716 | 2.474753 | 5.810473 | 6.93972 | 6.273273 | 5.95307 |
| PCTUC_717 | 2.849058 | 4.082054 | 5.516239 | 4.204436 | 3.742789 |
| PCTUC_718 | 2.47499 | 3.832772 | 4.199984 | 4.278993 | 3.647059 |
| PCTUC_719 | 2.358 | 3.678965 | 4.428831 | 3.953455 | 3.784203 |
| PCTUC_720 | −1.205677 | −1.063135 | 1.223399 | 2.529553 | −1.518103 |
| PCTUC_721 | 1.219077 | 2.001222 | 2.165595 | −1.026659 | 1.008794 |
| PCTUC_722 | 1.96705 | 2.055324 | 1.437751 | −1.41294 | 1.360393 |
| PCTUC_723 | 2.562115 | 1.727666 | 2.153008 | 1.840006 | 1.938897 |
| PCTUC_724 | 1.682504 | 3.997535 | 6.661172 | 3.369624 | 7.058978 |
| PCTUC_725 | 3.101324 | 2.563077 | 3.681123 | 1.793499 | 1.51067 |
| PCTUC_726 | 1.67782 | 2.292115 | 1.848348 | −1.219312 | −1.166466 |
| PCTUC_727 | 3.559063 | 1.578317 | 1.594204 | 1.814655 | 1.531729 |
| PCTUC_728 | 2.806376 | 2.952748 | 4.708348 | 1.765438 | 1.495801 |
| PCTUC_729 | 2.330432 | 1.917511 | 3.005132 | 4.83178 | −1.258102 |
| PCTUC_730 | 1.080247 | 2.14124 | 2.324177 | 2.076872 | 3.588565 |
| PCTUC_731 | 1.015105 | 5.222567 | 2.830412 | 1.246106 | 2.175415 |
| PCTUC_732 | 1.851799 | 2.169836 | 1.784843 | 1.559113 | −1.25609 |
| PCTUC_733 | 1.904821 | 1.443 | 1.663835 | 2.115776 | 1.236191 |
| PCTUC_734 | 1.171522 | 2.305167 | 5.020079 | 3.221029 | 1.091173 |
| PCTUC_735 | 1.017259 | 1.672028 | 2.921675 | 1.218728 | 1.246331 |
| PCTUC_736 | 2.629588 | 1.524143 | 1.566741 | −1.201418 | −1.951975 |
| PCTUC_737 | 1.388693 | 1.779508 | 2.146775 | 1.104491 | −1.046858 |
| PCTUC_738 | 1.098163 | 2.774363 | 1.621336 | 1.516934 | 7.845816 |
| PCTUC_739 | 1.247938 | −1.26988 | −1.005211 | 1.587222 | −1.134634 |
| PCTUC_740 | 2.857754 | 1.181018 | 1.024162 | 1.563654 | −1.183276 |
| PCTUC_741 | 1.579953 | 1.597549 | 1.411438 | 1.444184 | 1.019456 |
| PCTUC_742 | 1.485681 | 1.734902 | 3.267958 | 1.198231 | −1.066429 |
| PCTUC_743 | 2.018359 | −1.174056 | 1.525324 | −1.349442 | −1.594422 |
| PCTUC_744 | 1.545292 | 2.761388 | 2.804563 | −1.050354 | −1.752185 |
| PCTUC_745 | 2.807861 | 1.011148 | 2.10842 | 1.66104 | 1.228528 |
| PCTUC_746 | −2.675976 | 1.970285 | 2.396611 | −2.847489 | 2.130628 |
| PCTUC_747 | −1.25556 | 2.387692 | 4.348555 | −1.255805 | 2.368414 |
| PCTUC_748 | 2.050708 | −2.072285 | −1.318467 | 1.513265 | 1.065084 |
| PCTUC_749 | 4.927153 | −1.143048 | 5.439857 | 3.837492 | −3.545734 |
| PCTUC_750 | 2.446485 | 1.367795 | 1.933932 | 1.386537 | 1.142848 |
| PCTUC_751 | 1.737975 | −1.241683 | 2.058157 | 1.067161 | −2.030771 |
| PCTUC_752 | 1.593348 | 4.761776 | 3.719502 | 1.503111 | 2.153079 |
| PCTUC_753 | 2.066443 | 1.033129 | 1.516422 | −1.335804 | −2.518059 |
| PCTUC_754 | 2.397538 | −1.066542 | 3.14355 | 2.478858 | −1.195199 |
| PCTUC_755 | 1.970024 | 1.185407 | 1.18702 | 1.219573 | −1.653884 |
| PCTUC_756 | 2.882849 | 2.101217 | 2.946557 | 1.692976 | −1.316299 |
| PCTUC_757 | 1.187444 | 1.511025 | 4.881807 | 5.212888 | 2.193957 |
| PCTUC_758 | 1.505924 | 1.555706 | 1.666933 | 2.959011 | 1.118693 |
| PCTUC_759 | 1.735673 | 1.381833 | 1.541585 | 1.737289 | −2.07133 |
| PCTUC_760 | 2.398042 | −1.407117 | 1.76619 | 2.031062 | −2.262535 |
| PCTUC_761 | 1.33241 | 1.004258 | 2.610148 | 1.57589 | −1.525089 |
| PCTUC_762 | 1.036089 | 3.905444 | 1.182394 | 3.137581 | 3.109285 |
| PCTUC_763 | 1.623725 | 1.003853 | 1.479892 | 1.332373 | −1.336425 |
| PCTUC_764 | 1.443503 | 1.122172 | 1.359252 | 1.011896 | −1.182106 |
| PCTUC_765 | 2.852201 | 3.815464 | 3.295881 | 3.430123 | 1.612201 |
| PCTUC_766 | 1.751701 | 1.370024 | 1.566602 | 1.051677 | −1.430663 |
| PCTUC_767 | 1.688487 | −1.026691 | 1.496795 | 1.156978 | −1.046688 |
| PCTUC_768 | 1.804999 | 1.775698 | 1.30813 | 1.117862 | 1.079834 |
| PCTUC_769 | 2.228969 | 2.045903 | 1.798744 | 1.413123 | −1.011207 |
| PCTUC_770 | 1.780768 | 2.462475 | 1.889457 | 1.767707 | −1.141445 |
| PCTUC_771 | 2.005732 | 1.116398 | 34.002872 | 1.488342 | 1.233248 |
| PCTUC_772 | 1.754995 | −1.437288 | 1.556602 | −1.705196 | −1.570951 |
| PCTUC_773 | 1.537386 | 2.238718 | 2.355336 | 1.640442 | 1.361949 |
| PCTUC_774 | 1.32997 | 3.654829 | 2.754861 | 2.30835 | 1.262997 |
| PCTUC_775 | 1.630996 | 2.98714 | 2.822254 | 2.15646 | 2.017799 |
| PCTUC_776 | 3.07617 | −2.487576 | 3.56929 | 2.108594 | −2.786735 |
| PCTUC_777 | 1.751589 | 1.397667 | 1.306645 | 1.239567 | −1.283668 |
| PCTUC_778 | 1.510138 | 1.526597 | 1.664457 | 1.440165 | −1.032099 |
| PCTUC_779 | 3.748428 | 1.452387 | 7.42728 | 4.553708 | −1.07404 |
| PCTUC_780 | 2.157646 | −1.205891 | 1.907031 | 1.53636 | −2.304723 |
| PCTUC_781 | 1.722384 | 1.527703 | 1.481272 | 1.215339 | −1.406521 |
| PCTUC_782 | 1.617926 | 1.552256 | 2.451002 | 1.04707 | 1.072533 |
| PCTUC_783 | 2.737198 | 2.23809 | 2.347927 | 1.955506 | −1.07352 |
| PCTUC_784 | 2.944778 | 1.815663 | 3.105924 | 1.291081 | −1.739954 |
| PCTUC_785 | 3.654921 | 2.573428 | 2.848108 | 1.267785 | 1.625115 |
| PCTUC_786 | 2.435968 | 4.543569 | 1.591059 | 3.410156 | 1.128185 |
| PCTUC_787 | 1.637091 | 3.55331 | 3.154013 | 1.750237 | 1.272859 |
| PCTUC_788 | 1.606908 | −1.181581 | 1.337417 | −2.098886 | −2.480899 |
| PCTUC_789 | 2.483153 | 1.444229 | 1.86178 | −1.248986 | −1.504003 |
| PCTUC_790 | 1.447673 | 1.045415 | 1.208749 | 2.23128 | 1.276644 |
| PCTUC_791 | 1.592721 | 1.349893 | 3.158833 | 1.447821 | 1.399047 |
| PCTUC_792 | 2.085161 | 2.20713 | 2.344924 | 2.470099 | 1.817712 |
| PCTUC_793 | 2.10013 | 1.513724 | 2.635218 | 2.137043 | 1.708771 |
| PCTUC_794 | 1.863609 | 2.198983 | 2.666669 | 1.811442 | 1.939686 |
| PCTUC_795 | 2.127643 | 2.492941 | 2.642769 | 2.282253 | 2.362006 |
| PCTUC_796 | 2.647675 | 2.326116 | 2.575455 | 1.61441 | 1.680482 |
| PCTUC_797 | 2.603868 | 2.269858 | 2.853786 | 2.478076 | 1.905879 |
| PCTUC_798 | 2.457634 | 2.696164 | 2.928414 | 2.720635 | 2.714809 |
| PCTUC_799 | 2.351586 | 2.502443 | 4.14609 | 3.89303 | 2.335182 |
| PCTUC_800 | 2.514232 | 3.562574 | 3.872903 | 3.111842 | 2.51209 |
| PCTUC_801 | 2.584845 | 2.455776 | 5.196043 | 3.88422 | 2.451636 |
| PCTUC_802 | 2.568384 | 2.687358 | 4.062475 | 4.073182 | 2.542692 |
| PCTUC_803 | 2.467542 | 2.698226 | 3.995141 | 3.278665 | 2.342741 |
| PCTUC_804 | 2.423423 | 1.384956 | 2.123258 | 1.957864 | 1.386133 |
| PCTUC_805 | 2.672317 | 1.401187 | 1.540846 | 1.152146 | −1.175308 |
| PCTUC_806 | 3.325083 | 2.04527 | 2.802911 | 2.473777 | 1.819835 |
| PCTUC_807 | 2.345594 | 1.253775 | 1.38732 | 3.626277 | 1.921228 |
| PCTUC_808 | 2.74287 | 3.69523 | 5.083086 | 4.782139 | 2.761177 |
| PCTUC_809 | 1.961826 | 2.048956 | 1.790611 | 3.117042 | 2.17878 |
| PCTUC_810 | 1.738072 | 1.312312 | 1.851987 | 1.688317 | 1.826226 |
| PCTUC_811 | 2.479734 | 2.148855 | 2.703484 | 2.424147 | 1.854368 |
| PCTUC_812 | 2.216486 | 1.603243 | 2.525568 | 2.223361 | 1.836276 |
| PCTUC_813 | 2.244768 | 1.638971 | 2.436859 | 2.326672 | 1.8383 |
| PCTUC_814 | 2.599626 | 3.107804 | 4.748261 | 3.481901 | 2.720379 |
| PCTUC_815 | 2.731606 | 2.866852 | 3.780127 | 2.582704 | 2.17795 |
| PCTUC_816 | 2.782779 | 2.519143 | 3.337878 | 3.713792 | 2.460799 |
| PCTUC_817 | 2.539845 | 1.612676 | 2.570179 | 1.924375 | 1.690343 |
| PCTUC_818 | 2.94142 | 2.551051 | 3.184934 | 2.801994 | 2.33619 |
| PCTUC_819 | 2.593287 | 3.521731 | 4.601146 | 3.217127 | 2.405624 |
| PCTUC_820 | 1.869347 | 1.156877 | 1.535996 | 1.159733 | 1.049716 |
| PCTUC_821 | 2.347548 | 1.943487 | 2.602382 | 1.883901 | 1.635784 |
| PCTUC_822 | 2.762829 | 3.264891 | 5.359295 | 4.502306 | 3.029265 |
| PCTUC_823 | 1.709181 | 1.903973 | 1.224162 | 2.378757 | 1.939935 |
| PCTUC_824 | 2.227166 | 1.707171 | 2.192069 | 1.810446 | 1.541394 |
| PCTUC_825 | 2.623839 | 1.395692 | 2.159526 | 1.437781 | 1.424438 |
| PCTUC_826 | 1.645178 | −1.212428 | 1.368683 | −1.04751 | −1.335886 |
| PCTUC_827 | 1.546281 | 1.382322 | 2.004729 | 1.195291 | 1.218382 |
| PCTUC_828 | 2.18352 | 1.813284 | 2.32015 | 1.509559 | 1.480408 |
| PCTUC_829 | 1.874654 | 1.125268 | 1.435859 | −1.304285 | −1.312731 |
| PCTUC_830 | 2.283218 | 1.781524 | 2.266755 | 1.943294 | 1.646191 |
| PCTUC_831 | 2.391593 | −1.009171 | 1.332706 | −1.261596 | −1.61064 |
| PCTUC_832 | 1.880277 | 1.458405 | 1.539495 | 1.278124 | 1.443973 |
| PCTUC_833 | 1.751907 | 1.79457 | 1.72199 | 1.301647 | 1.022377 |
| PCTUC_834 | 1.976311 | 1.460713 | 2.062928 | 1.372882 | 1.3302 |
| PCTUC_835 | 2.281342 | 2.204623 | 2.371787 | 2.323638 | 1.765945 |
| PCTUC_836 | 1.45514 | −1.0925 | 1.160709 | 1.031877 | −1.113493 |
| PCTUC_837 | 2.231949 | 1.66407 | 2.161517 | 1.429815 | 1.354722 |
| PCTUC_838 | 2.005671 | −2.169267 | −1.164172 | −1.842256 | −2.396542 |
| PCTUC_839 | 2.297453 | 2.766795 | 4.081153 | 2.158186 | 1.795967 |
| PCTUC_840 | 1.678983 | 1.204796 | 2.335048 | 1.335965 | 2.376273 |
| PCTUC_841 | 2.266033 | 2.716181 | 2.149279 | 1.463845 | 1.134204 |
| PCTUC_842 | 2.482376 | 1.619383 | 2.270337 | −1.229555 | −1.389874 |
| PCTUC_843 | 1.871004 | 1.006036 | 1.009794 | 1.274239 | −1.385184 |
| PCTUC_844 | 1.738249 | 1.103569 | 1.422457 | −1.057936 | −1.378921 |
| PCTUC_845 | 1.679917 | 1.342282 | 1.664368 | 2.089621 | 1.064222 |
| PCTUC_846 | 1.85247 | 1.315385 | 1.280974 | 1.452513 | −1.129171 |
| PCTUC_847 | 1.9447 | 1.183061 | 1.32463 | 1.112224 | −1.025501 |
| PCTUC_848 | 2.019166 | 2.091423 | 1.847378 | 3.154582 | 1.44493 |
| PCTUC_849 | 1.743695 | 1.90351 | 1.688092 | 1.963297 | 1.678683 |
| PCTUC_850 | 2.045105 | 1.648624 | 2.308564 | 1.954925 | 1.453773 |
| PCTUC_851 | 1.562211 | 1.136459 | 1.845817 | −1.086125 | −1.357593 |
| PCTUC_852 | 1.889697 | 1.312998 | 2.271817 | 1.761846 | −1.842876 |
| PCTUC_853 | 1.676356 | 1.374857 | 1.613049 | 2.606546 | 1.058314 |
| PCTUC_854 | 2.649884 | 2.259034 | 2.604694 | 2.801454 | 2.114191 |
| PCTUC_855 | 2.364338 | 1.62134 | 2.291844 | 1.883181 | 1.499188 |
| PCTUC_856 | 2.907776 | 2.257864 | 4.008818 | 3.392172 | 2.427414 |
| PCTUC_857 | 1.504321 | 1.376339 | 2.058645 | −1.784745 | 1.963081 |
| PCTUC_858 | 2.044061 | 2.004635 | 3.352611 | 2.537826 | 1.762131 |
| PCTUC_859 | 2.013783 | 1.536712 | 2.006352 | 1.351533 | 1.26096 |
| PCTUC_860 | 2.13201 | −1.115348 | 1.203492 | −1.029419 | −1.134879 |
| PCTUC_861 | 1.813424 | 1.637124 | 2.287569 | 2.369354 | 1.098582 |
| PCTUC_862 | 1.783912 | 1.357527 | 1.117161 | 1.014917 | −1.249444 |
| PCTUC_863 | 1.639549 | 1.782294 | 1.222373 | 1.319296 | −1.975291 |
| PCTUC_864 | 1.90779 | 1.773835 | 2.173387 | 1.771235 | 1.285498 |
| PCTUC_865 | 1.871908 | 1.736728 | 1.634273 | 2.278668 | 1.260973 |
| PCTUC_866 | 1.52375 | −1.07945 | 1.211952 | 1.089258 | −1.249161 |
| PCTUC_867 | 1.877495 | 1.917157 | 2.066668 | 2.591593 | 1.722827 |
| PCTUC_868 | 2.112046 | 2.219779 | 1.913224 | 1.858551 | 1.03877 |
| PCTUC_869 | 1.625932 | −1.098634 | 1.378695 | −1.768543 | −1.969913 |
| PCTUC_870 | 1.96413 | 1.421129 | 2.013952 | 1.004407 | −1.038988 |
| PCTUC_871 | 1.890775 | 1.185761 | 1.517475 | 1.007294 | −1.046279 |
| PCTUC_872 | 1.859905 | 1.622411 | 1.984478 | −1.119956 | −1.143552 |
| PCTUC_873 | 1.969205 | 2.159228 | 2.561842 | 1.304826 | −1.011093 |
| PCTUC_874 | 1.809296 | 3.093593 | 2.048956 | 2.165634 | 1.434358 |
| PCTUC_875 | 1.976093 | 1.667331 | 1.503174 | 1.410736 | 1.373617 |
| PCTUC_876 | 1.698732 | 1.905596 | 2.541518 | 1.739503 | −1.078499 |
| PCTUC_877 | 2.082926 | 1.377183 | 2.487029 | −1.016485 | −1.140493 |
| PCTUC_878 | 1.541651 | 1.458844 | 1.476125 | 1.730811 | 1.382746 |
| PCTUC_879 | 2.209875 | 1.955096 | 2.333605 | 2.3839 | 1.646064 |
| PCTUC_880 | 1.951771 | 1.6827 | 2.031071 | 1.627983 | 1.584582 |
| PCTUC_881 | 1.954496 | 2.04587 | 2.55217 | 2.220093 | 1.588366 |
| PCTUC_882 | 2.301353 | 1.994945 | 2.153584 | 2.236753 | 1.649811 |
| PCTUC_883 | 2.325608 | 2.589049 | 2.499343 | 2.33114 | 1.720812 |
| PCTUC_884 | 1.708056 | 1.789686 | 1.79987 | 1.421518 | −1.111179 |
| PCTUC_885 | 1.94465 | −1.539719 | −2.02752 | 1.242959 | −1.255616 |
| PCTUC_886 | 2.163645 | 2.68261 | 2.434486 | 1.358898 | 1.236363 |
| PCTUC_887 | 2.023344 | 1.535118 | 1.598191 | 1.46662 | 1.114935 |
| PCTUC_888 | 3.805361 | −1.069556 | 1.609747 | −1.07591 | −1.619559 |
| PCTUC_889 | 2.091429 | 1.468055 | 1.245638 | 1.022078 | −1.027285 |
| PCTUC_890 | 1.921732 | 1.48676 | 2.127379 | 1.295536 | 1.247707 |
| PCTUC_891 | 2.363246 | 1.666287 | 1.836035 | 2.044549 | 1.476581 |
| PCTUC_892 | 1.910954 | 1.459881 | 1.555845 | −1.049927 | −1.618603 |
| PCTUC_893 | 2.822666 | 2.622807 | 3.53729 | 1.375128 | 1.160408 |
| PCTUC_894 | 2.52524 | 2.373235 | 3.208908 | 2.510066 | 2.15306 |
| PCTUC_895 | 1.962498 | 1.407028 | 1.535472 | 1.424376 | 1.051004 |
| PCTUC_896 | 2.271611 | 1.191141 | 2.048356 | 1.421338 | 1.191054 |
| PCTUC_897 | 1.553053 | −1.974929 | −1.903705 | 1.486964 | 1.508478 |
| PCTUC_898 | 2.549773 | 3.184214 | 4.649253 | 2.011571 | 1.58673 |
| PCTUC_899 | 2.158757 | 2.411463 | 2.41099 | 1.354378 | 1.042022 |
| PCTUC_900 | 1.788992 | 2.553147 | 2.451661 | 1.704656 | 1.721045 |
| PCTUC_901 | 1.938191 | 1.050664 | 1.215151 | −1.046125 | 1.061172 |
| PCTUC_902 | 2.177013 | 1.063489 | 1.191732 | −1.36473 | −1.708799 |
| PCTUC_903 | 1.873974 | 1.978962 | 2.267684 | 1.318745 | 1.435156 |
| PCTUC_904 | 1.778571 | 1.686699 | 1.426102 | −1.205492 | −1.405787 |
| PCTUC_905 | 2.266052 | 1.302131 | 1.820607 | 1.647485 | 1.116222 |
| PCTUC_906 | 2.751729 | 2.016082 | 2.143599 | 1.745673 | 1.411276 |
| PCTUC_907 | 2.856837 | 4.284631 | 3.878442 | 3.326426 | 2.825075 |
| PCTUC_908 | 1.95905 | 1.64949 | 3.431271 | 2.31849 | 1.451118 |
| PCTUC_909 | 1.691228 | 1.255651 | 2.059265 | 1.018969 | −1.236387 |
| PCTUC_910 | 2.426663 | 1.193135 | 1.550878 | 1.26375 | −2.27989 |
| PCTUC_911 | 4.532208 | 2.238964 | 2.900606 | −2.700652 | −2.005532 |
| PCTUC_912 | 1.54193 | 1.019645 | −1.82718 | 2.099053 | −3.007764 |
| PCTUC_913 | 2.648759 | 1.394977 | 1.270218 | 1.774617 | 1.057235 |
| PCTUC_914 | 1.128493 | 2.531108 | 2.016811 | 3.008403 | 3.898096 |
| PCTUC_915 | 1.801934 | 2.191015 | 3.068439 | 1.352042 | 1.298424 |
| PCTUC_916 | 2.121188 | 1.570392 | 1.336953 | 1.167939 | −1.554178 |
| PCTUC_917 | 1.965218 | 1.922846 | 1.327187 | 1.025806 | −1.7507 |
| PCTUC_918 | 1.995978 | 1.821286 | 2.205084 | −1.13903 | −1.191393 |
| PCTUC_919 | 1.322658 | 2.104843 | 2.861712 | 1.681921 | 2.872681 |
| PCTUC_920 | 2.698585 | 1.970061 | 1.211588 | 3.268852 | 2.365577 |
| PCTUC_921 | 1.727702 | 1.722659 | 1.76901 | 1.971489 | 1.591412 |
| PCTUC_922 | 2.134946 | 2.808802 | 3.163258 | 1.398622 | 1.656875 |
| PCTUC_923 | 3.874743 | −1.967023 | 3.358653 | 1.087691 | 1.532369 |
| PCTUC_924 | 1.343629 | −1.03239 | −1.781882 | 2.09737 | −2.384392 |
| PCTUC_925 | 1.416624 | 1.590514 | 2.341637 | 1.477663 | 2.725187 |
| PCTUC_926 | 2.278423 | −1.304067 | 1.084692 | −1.079548 | −1.80759 |
| PCTUC_927 | −1.212027 | 1.171116 | 1.829475 | 1.941464 | 2.180511 |
| PCTUC_928 | 2.340422 | 1.355329 | 1.415694 | 1.08397 | −1.223843 |
| PCTUC_929 | 2.15377 | 1.618364 | 2.005306 | 1.092598 | −1.651047 |
| PCTUC_930 | 2.479362 | −1.213437 | 1.090769 | −1.149222 | −1.471385 |
| PCTUC_931 | 1.645102 | 2.155922 | 1.039858 | 1.343799 | 1.362534 |
| PCTUC_932 | 1.937046 | 1.845351 | 2.05657 | −1.258158 | −1.216066 |
| PCTUC_933 | 2.929791 | 1.407341 | 1.54831 | 1.010886 | −1.266786 |
| PCTUC_934 | 1.616478 | 1.237305 | 1.177842 | 1.013334 | −1.807534 |
| PCTUC_935 | 2.428755 | −1.44736 | 1.060927 | 1.645897 | −1.674838 |
| PCTUC_936 | 1.094927 | 5.459811 | 1.070757 | 3.521735 | 3.525177 |
| PCTUC_937 | 3.064947 | 1.020142 | 1.660377 | 1.513395 | −1.338045 |
| PCTUC_938 | 2.338089 | 1.109879 | 1.828194 | −1.334607 | −1.661552 |
| PCTUC_939 | 1.983513 | 2.87102 | 3.14574 | 1.457221 | 1.217682 |
| PCTUC_940 | 2.83538 | 1.388755 | 1.554512 | −1.103216 | −1.020415 |
| PCTUC_941 | 3.385786 | 3.089068 | 2.907079 | 1.402766 | 2.203065 |
| PCTUC_942 | −1.412977 | −1.811306 | 1.675567 | 1.103626 | 3.157614 |
| PCTUC_943 | 3.666498 | 1.075061 | 1.531565 | 1.111984 | −1.632703 |
| PCTUC_944 | 1.843041 | 2.498397 | 1.6392 | 1.850137 | 1.347077 |
| PCTUC_945 | 1.522345 | 1.550846 | 1.137903 | 1.755582 | 1.180404 |
| PCTUC_946 | 3.172343 | −1.24995 | 1.939802 | 1.777056 | −1.791084 |
| PCTUC_947 | 2.288532 | 1.312973 | 1.379707 | 1.309444 | −1.475509 |
| PCTUC_948 | −1.124776 | 2.213994 | 4.330243 | −1.445415 | 2.07831 |
| PCTUC_949 | 3.346842 | −1.188788 | 1.263286 | 1.919915 | −1.14363 |
| PCTUC_950 | 5.91396 | 2.479738 | 8.393818 | 6.084914 | 4.093964 |
| PCTUC_951 | 1.871348 | 2.61188 | 1.729258 | 1.833634 | 1.36509 |
| PCTUC_952 | 2.036369 | 1.827873 | 1.442905 | 2.043129 | 1.025536 |
| PCTUC_953 | 3.07094 | 2.967804 | 2.867549 | 3.798386 | 2.524811 |
| PCTUC_954 | 3.129348 | 1.128209 | 1.538464 | 1.22886 | −1.176202 |
| PCTUC_955 | 2.363069 | 2.394411 | 2.180458 | −1.101003 | −1.863439 |
| PCTUC_956 | 4.62408 | 1.552741 | 2.699866 | 1.611055 | 1.67697 |
| PCTUC_957 | 1.703468 | 1.262165 | 1.267325 | −1.487167 | |
| PCTUC_958 | 2.874749 | 9.091537 | 2.579849 | 2.25185 | −1.038772 |
| PCTUC_959 | 2.090051 | 1.305155 | 1.579253 | 1.503822 | −1.386404 |
| PCTUC_960 | 2.845635 | 1.507949 | 1.179313 | 1.183302 | −1.464932 |
| PCTUC_961 | 1.530236 | 3.29414 | 2.872372 | 1.096841 | 1.829785 |
| PCTUC_962 | 2.019459 | 1.862188 | 1.227619 | 1.190924 | −1.438797 |
| PCTUC_963 | 2.151981 | 1.134676 | 1.309617 | 1.415813 | −1.234398 |
| PCTUC_964 | 2.46002 | 1.865439 | 2.370393 | −1.09332 | −3.343158 |
| PCTUC_965 | 2.357135 | 1.837095 | 1.809527 | 1.743313 | −1.162524 |
| PCTUC_966 | 2.646736 | −1.230297 | 16.329386 | −1.143001 | −1.221426 |
| PCTUC_967 | 2.171907 | 1.72484 | 1.534128 | 1.603964 | 1.367996 |
| PCTUC_968 | 4.739057 | 1.944878 | 2.618026 | −1.846025 | −2.014114 |
| PCTUC_969 | 1.907598 | 1.905547 | 1.442495 | 1.500899 | 1.457679 |
| PCTUC_970 | 3.316134 | 2.077369 | 1.906654 | 2.141966 | −1.005555 |
| PCTUC_971 | 2.386012 | 1.093453 | 1.315324 | 1.454897 | −1.284141 |
| PCTUC_972 | 3.227783 | −1.009619 | 1.343919 | −1.032079 | −1.399772 |
| PCTUC_973 | 1.251395 | 1.961825 | 2.074857 | −1.041606 | −1.257309 |
| PCTUC_974 | 2.587815 | 1.378796 | 1.798498 | 1.758659 | 1.092618 |
| PCTUC_975 | 1.943511 | 2.282181 | 2.703775 | 1.038239 | −1.779081 |
| PCTUC_976 | 1.623674 | 1.062539 | 1.261934 | 1.465839 | 1.609397 |
| PCTUC_977 | 4.600121 | −1.597071 | 3.386886 | 1.982753 | −5.036413 |
| PCTUC_978 | 5.154942 | −1.644976 | 3.034494 | 1.65744 | −1.747205 |
| PCTUC_979 | 1.716536 | −1.087847 | 1.360934 | 2.241092 | 1.105519 |
| PCTUC_980 | 2.386343 | 1.795594 | 1.492045 | 1.670228 | −1.506544 |
| PCTUC_981 | 1.674169 | 1.624496 | 1.309471 | 2.548435 | −1.017204 |
| PCTUC_982 | 1.818088 | 2.996705 | 5.079009 | 3.42635 | 8.203172 |
| PCTUC_983 | 2.53358 | 1.54159 | 1.62574 | 1.1435 | −2.666574 |
| PCTUC_984 | 2.02023 | 1.97024 | −1.190315 | 1.653198 | −1.202102 |
| PCTUC_985 | 4.662121 | 1.90137 | 2.882533 | 1.816393 | 3.0195 |
| PCTUC_986 | 8.491511 | 4.16046 | 8.290752 | 9.11205 | 1.558066 |
| PCTUC_987 | 1.509953 | 1.227295 | 1.284975 | 2.07757 | 1.044685 |
| PCTUC_988 | 2.254812 | 1.719378 | 1.654111 | 1.818505 | −1.854655 |
| PCTUC_989 | 2.697165 | 1.935913 | −1.211672 | −1.055024 | −1.235915 |
| PCTUC_990 | 1.922588 | −1.141932 | 1.454403 | −1.655129 | −1.738252 |
| PCTUC_991 | 4.337499 | −2.529357 | 3.53756 | 1.902414 | −5.891994 |
| PCTUC_992 | 5.608949 | 2.492292 | 1.717627 | 1.557444 | 1.716952 |
| PCTUC_993 | 1.986168 | 2.308209 | 5.121046 | −1.67648 | −2.187591 |
| PCTUC_994 | 5.024633 | 2.315602 | 2.597577 | −1.086497 | 1.236312 |
| PCTUC_995 | 2.535233 | 1.611994 | 1.390142 | 1.288071 | −1.528931 |
| PCTUC_996 | 1.28686 | 2.158292 | 2.788701 | 2.345606 | 5.569685 |
| PCTUC_997 | 2.178464 | −1.029413 | 1.827458 | 1.145476 | −1.355086 |
| PCTUC_998 | 1.976933 | 1.262073 | 1.608358 | 1.431333 | −1.122224 |
| PCTUC_999 | 1.493959 | 1.989163 | 1.052016 | 1.194044 | −1.147475 |
| PCTUC_1000 | 2.20571 | 1.6934 | 1.670557 | 1.617663 | −1.023672 |
| PCTUC_1001 | 1.130081 | 2.155692 | 3.082375 | 2.100547 | −1.204434 |
| PCTUC_1002 | 2.096987 | −1.294777 | 1.078077 | −1.129435 | −1.530266 |
| PCTUC_1003 | 1.4735 | 2.15687 | 1.821478 | 1.463017 | 1.283592 |
| PCTUC_1004 | 3.601119 | −1.07069 | 1.343604 | −1.143005 | −2.140174 |
| PCTUC_1005 | 2.16352 | 1.304812 | 1.170797 | −1.375762 | −2.103141 |
| PCTUC_1006 | 9.251185 | 1.590991 | 5.711594 | 4.310499 | −1.261379 |
| PCTUC_1007 | 2.718569 | 1.268528 | 2.513555 | −1.982436 | −1.091678 |
| PCTUC_1008 | 1.276398 | 1.204101 | 1.638292 | 4.717824 | 3.206786 |
| PCTUC_1009 | 1.652535 | 1.975113 | 2.175845 | −1.12018 | −1.256014 |
| PCTUC_1010 | 2.162597 | 1.310228 | 1.180317 | 1.430186 | 1.023308 |
| PCTUC_1011 | 2.407776 | 1.636206 | 1.314562 | 1.337553 | −1.441501 |
| PCTUC_1012 | 4.84285 | 2.186353 | 2.722289 | −1.262478 | −1.152552 |
| PCTUC_1013 | 2.04709 | 1.407198 | 1.560425 | 1.47126 | 1.312585 |
| PCTUC_1014 | 6.928616 | 1.055858 | 1.73243 | 2.155555 | 1.436217 |
| PCTUC_1015 | 1.976716 | 1.164299 | 1.108067 | 1.452288 | −1.039557 |
| PCTUC_1016 | 1.382746 | 1.236182 | 2.766599 | 3.571078 | 2.098697 |
| PCTUC_1017 | 2.340813 | 1.321283 | 1.651027 | −1.085383 | −1.248735 |
| PCTUC_1018 | 1.948146 | 1.027787 | 1.058867 | 1.177399 | −1.100196 |
| PCTUC_1019 | 2.038241 | 1.053319 | 1.24096 | 1.37697 | 1.005457 |
| PCTUC_1020 | 2.229994 | 1.313133 | 1.350871 | 1.360874 | −1.033352 |
| PCTUC_1021 | 3.197343 | 2.049208 | 1.526544 | 1.221605 | −1.309624 |
| PCTUC_1022 | 1.934023 | 1.118641 | 1.445595 | 1.440211 | −1.104546 |
| PCTUC_1023 | 2.286244 | 1.199824 | 1.239642 | 1.410419 | −1.164296 |
| PCTUC_1024 | 2.001559 | 1.172226 | 1.705722 | −1.206812 | −1.054981 |
| PCTUC_1025 | 2.404986 | 1.106891 | 1.008177 | −1.131864 | −1.427144 |
| PCTUC_1026 | 2.11835 | −1.035554 | 1.054889 | 1.15996 | −1.218929 |
| PCTUC_1027 | 2.190092 | 1.41212 | 1.34154 | 1.687925 | −1.292349 |
| PCTUC_1028 | 3.870844 | −1.713348 | 3.093784 | 1.692219 | −7.325786 |
| PCTUC_1029 | 2.591095 | 1.170344 | 1.749711 | 1.783676 | −1.138249 |
| PCTUC_1030 | 2.480039 | 1.690515 | 1.771112 | 1.444623 | −1.221338 |
| PCTUC_1031 | 2.688212 | 1.639698 | 1.950274 | 1.999945 | 1.067321 |
| PCTUC_1032 | 8.662786 | 1.111851 | 1.969947 | 1.904457 | 1.343916 |
| PCTUC_1033 | 2.168242 | 1.83769 | −1.03914 | 1.615783 | 1.27235 |
| PCTUC_1034 | 2.150622 | 1.288981 | 1.17577 | 1.315346 | −1.072515 |
| PCTUC_1035 | 1.867998 | 1.425122 | 1.339841 | 1.516976 | −1.286181 |
| PCTUC_1036 | 3.322184 | 1.418265 | 1.509088 | 2.627051 | 1.135941 |
| PCTUC_1037 | 1.914963 | 1.260587 | 1.324501 | 1.198238 | −1.551652 |
| PCTUC_1038 | 2.619273 | 2.006016 | −1.109492 | 1.349718 | 1.086261 |
| PCTUC_1039 | 2.990175 | 1.283478 | 1.915295 | 1.754567 | −1.138346 |
| PCTUC_1040 | 2.314548 | 1.527413 | 1.683415 | 1.129444 | −1.559905 |
| PCTUC_1041 | 2.059229 | 1.561613 | 1.930949 | 1.902644 | −1.386571 |
| PCTUC_1042 | 3.089431 | 1.932695 | 3.129811 | 1.719934 | −1.320877 |
| PCTUC_1043 | 2.218593 | 2.379896 | 2.101869 | 1.637499 | 1.196839 |
| PCTUC_1044 | 3.22484 | 1.934236 | 2.257307 | 2.405261 | 1.09738 |
| PCTUC_1045 | 2.879969 | 1.782096 | 1.488046 | 1.917997 | 1.11014 |
| PCTUC_1046 | 2.405005 | 1.380251 | 1.708364 | 2.180275 | 1.085321 |
| PCTUC_1047 | 2.321114 | 3.516298 | 4.373227 | 3.01992 | 4.139297 |
| PCTUC_1048 | 2.195277 | 3.486061 | 5.021372 | 2.803599 | 4.429878 |
| PCTUC_1049 | 2.188752 | 2.981335 | 3.233166 | 1.950319 | 3.128412 |
| PCTUC_1050 | 1.613317 | 1.971067 | 2.743932 | 1.618722 | 2.855495 |
| PCTUC_1051 | 2.123477 | 1.513347 | 1.615171 | −1.039459 | 1.834816 |
| PCTUC_1052 | 1.744405 | 2.047574 | 2.076573 | 1.551619 | 1.922986 |
| PCTUC_1053 | 2.097268 | 3.603894 | 3.217664 | 2.832975 | 3.028847 |
| PCTUC_1054 | 2.507241 | 2.729321 | 3.044449 | 3.602959 | 2.788986 |
| PCTUC_1055 | 2.402552 | 1.661436 | 2.673273 | 1.955399 | 2.420513 |
| PCTUC_1056 | 2.051499 | 3.139189 | 3.432348 | 2.196797 | 2.255609 |
| PCTUC_1057 | 2.215758 | 2.988161 | 3.701127 | 2.786364 | 3.401309 |
| PCTUC_1058 | 2.13731 | 1.384542 | 2.027797 | 1.059753 | 2.095294 |
| PCTUC_1059 | 1.343765 | 2.219798 | 2.697312 | 2.333196 | 3.102681 |
| PCTUC_1060 | 2.088285 | 2.860373 | 3.085011 | 2.016002 | 3.103222 |
| PCTUC_1061 | 2.242374 | 2.994492 | 2.948725 | 2.4391886 | 3.150199 |
| PCTUC_1062 | 1.60416 | 1.591494 | 2.034929 | 1.556863 | 2.421872 |
| PCTUC_1063 | 1.987271 | 1.375089 | 1.998077 | 2.016782 | 1.889572 |
| PCTUC_1064 | 2.755367 | 4.411705 | 5.011181 | 3.46172 | 5.131229 |
| PCTUC_1065 | 1.490694 | 2.590107 | 2.38876 | 2.363459 | 4.526927 |
| PCTUC_1066 | 1.794536 | 1.984237 | 2.119993 | 1.470474 | 2.544308 |
| PCTUC_1067 | 2.013362 | 4.187653 | 3.594945 | 3.306826 | 4.448132 |
| PCTUC_1068 | 1.308447 | 2.572794 | 2.035954 | 1.168742 | −1.62542 |
| PCTUC_1069 | 2.068573 | 2.871417 | 4.054071 | 2.894181 | 4.689563 |
| PCTUC_1070 | 2.119418 | 2.070433 | 2.04862 | 1.577364 | 2.154318 |
| PCTUC_1071 | 1.388731 | 3.577526 | 3.684772 | 1.714653 | 3.6624 |
| PCTUC_1072 | 2.028962 | 2.494959 | 2.520422 | 1.52116 | 2.786445 |
| PCTUC_1073 | 1.354737 | 5.133123 | 5.65145 | −1.088542 | 2.657814 |
| PCTUC_1074 | 1.538652 | −2.114757 | −1.878364 | 1.578808 | 1.499023 |
| PCTUC_1075 | 5.673028 | 1.487911 | 3.36078 | 1.996255 | −1.226855 |
| PCTUC_1076 | 1.079724 | −1.086661 | 1.298582 | −2.48976 | −1.297809 |
| PCTUC_1077 | 3.755108 | −1.717643 | 3.507391 | 2.243764 | −6.207491 |
| PCTUC_1078 | 2.035899 | −1.0433 | 3.818222 | 1.377906 | −1.549799 |
| PCTUC_1079 | 1.837077 | 1.661645 | 3.01799 | −1.788427 | −1.698747 |
| PCTUC_1080 | 1.44747 | 1.984165 | 2.345122 | 1.636858 | 1.950357 |
| PCTUC_1081 | 2.589691 | 5.375653 | 6.566166 | 5.535592 | 6.460448 |
| PCTUC_1082 | 5.148818 | 1.46638 | 4.037243 | 3.383032 | 2.662901 |
| PCTUC_1083 | 1.742403 | 2.213345 | 3.029419 | 1.749504 | 1.541367 |
| PCTUC_1084 | 5.43197 | 2.509498 | 1.651137 | 2.294021 | 2.377209 |
| PCTUC_1085 | 1.892009 | 2.399117 | 2.119305 | 1.629489 | 2.086697 |
| PCTUC_1086 | 5.73202 | −1.514249 | 5.886267 | 2.988024 | −5.816788 |
| PCTUC_1087 | 2.234142 | −1.571367 | 1.514389 | 1.084099 | −1.197264 |
| PCTUC_1088 | 1.200861 | −1.078458 | 1.518761 | 1.225581 | −1.545213 |
| PCTUC_1089 | 1.151515 | 1.412061 | 2.449138 | 2.136675 | 2.094553 |
| PCTUC_1090 | 4.012289 | −1.088193 | 3.326313 | 1.816009 | −1.895476 |
| PCTUC_1091 | −1.468186 | 2.400961 | 3.376961 | −1.036417 | 2.125376 |
| PCTUC_1092 | 1.86228 | 1.19823 | 2.141057 | 2.600541 | −1.064478 |
| PCTUC_1093 | 2.556667 | 2.182766 | 2.833895 | 2.716894 | 1.994166 |
| PCTUC_1094 | 1.917973 | 1.256288 | 1.742476 | 1.373519 | −1.040928 |
| PCTUC_1095 | 2.621555 | 2.247114 | 3.160852 | 1.67503 | 3.055504 |
| PCTUC_1096 | 2.855342 | 3.209525 | 3.223669 | 2.431496 | 1.144113 |
| PCTUC_1097 | 1.188027 | 7.007524 | 2.897435 | 4.072117 | 3.561213 |
| PCTUC_1098 | 2.235905 | 3.822684 | 3.685224 | 2.116626 | 1.788512 |
| PCTUC_1099 | 1.330936 | 1.352804 | 1.032184 | 1.913205 | 1.142619 |
| PCTUC_1100 | 3.01892 | 3.443123 | 4.852659 | 3.883996 | −1.016162 |
| PCTUC_1101 | 1.497275 | −1.124809 | 1.461785 | −1.018584 | −1.077507 |
| PCTUC_1102 | 1.789655 | 3.265229 | 4.170906 | 3.68111 | 3.87418 |
| PCTUC_1103 | 3.608782 | 5.252712 | 8.523944 | 3.121104 | 4.45085 |
| PCTUC_1104 | 2.588284 | 1.178718 | 1.538689 | −1.197036 | −1.466973 |
| PCTUC_1105 | 1.527503 | 1.550702 | 1.517095 | 3.307125 | −1.187629 |
| PCTUC_1106 | 1.770228 | 1.134965 | 1.313179 | 2.075559 | −1.295864 |
| PCTUC_1107 | 1.523458 | 2.687189 | 2.26406 | 2.135197 | 2.66312 |
| PCTUC_1108 | 2.183624 | 2.147157 | 2.902527 | −1.371394 | 1.466713 |
| PCTUC_1109 | 2.225928 | 2.196385 | 2.044623 | 2.626373 | 2.64584 |
| PCTUC_1110 | 1.354471 | 1.193941 | 1.553985 | 1.463523 | −1.03174 |
| PCTUC_1111 | 1.685433 | 2.241103 | 1.567154 | −1.032259 | −1.041822 |
| PCTUC_1112 | 2.082848 | 1.4844 | 2.695112 | 1.403345 | −1.788018 |
| PCTUC_1113 | 2.861085 | 1.342187 | 4.543931 | 4.665126 | 3.338884 |
| PCTUC_1114 | 1.582655 | 2.503864 | 4.677818 | 2.280037 | 7.309894 |
| PCTUC_1115 | 1.654223 | 1.319917 | 1.579363 | 1.971646 | −1.049361 |
| PCTUC_1116 | 1.374163 | 2.717865 | 2.334999 | 1.905592 | 2.724711 |
| PCTUC_1117 | 2.810961 | 2.866595 | 9.361756 | −1.230064 | 2.835042 |
| PCTUC_1118 | 2.992826 | 2.338832 | 2.094304 | 1.685008 | 2.157194 |
| PCTUC_1119 | 1.330389 | 1.214922 | 1.499869 | 1.134647 | 1.376153 |
| PCTUC_1120 | 1.943912 | 1.350693 | 1.483964 | −1.036967 | 1.453104 |

TABLE 4

| | Colon | Colon | Colon | Colon | Colon |
|---|---|---|---|---|---|
| Internal Id | Tumor 1 | Tumor 2 | Tumor 3 | Tumor 4 | Tumor 5 |
| PCTDC_1 | −3.686332 | −3.764107 | −2.186606 | −3.680799 | −3.84198 |
| PCTDC_2 | −1.77226 | −1.420686 | −1.947258 | −2.290254 | −1.328009 |
| PCTDC_3 | −2.830777 | −2.188274 | −2.38472 | −2.302896 | −2.112362 |
| PCTDC_4 | −1.323522 | −2.135768 | −1.45575 | −1.914081 | 1.438666 |
| PCTDC_5 | −2.235407 | −1.80509 | −2.016836 | −2.171651 | −1.418959 |
| PCTDC_6 | −2.667534 | −1.160172 | −2.10837 | −1.32617 | −2.153253 |
| PCTDC_7 | 1.021256 | −25.105358 | 1.439239 | −7.869633 | −1.171115 |
| PCTDC_8 | −2.234816 | −1.242266 | −1.243916 | −1.636976 | −1.064982 |
| PCTDC_9 | −16.672455 | −36.952839 | −14.68694 | −31.487497 | −32.52441 |
| PCTDC_10 | −1.434666 | −1.613476 | −2.514822 | −2.946794 | −2.143325 |
| PCTDC_11 | −9.385355 | −4.410996 | −4.533406 | −13.051781 | −9.788757 |
| PCTDC_12 | −3.030862 | 1.265934 | −2.241221 | 1.405932 | −2.084695 |
| PCTDC_13 | −5.984219 | −3.497875 | −3.012388 | −5.099751 | −4.836649 |
| PCTDC_14 | −1.413376 | −1.002366 | 1.268685 | 1.365882 | −1.425659 |
| PCTDC_15 | −2.996104 | −1.091542 | −1.403562 | −1.430543 | −2.256678 |
| PCTDC_16 | −2.943326 | −2.249868 | −2.035691 | −1.535672 | −1.933099 |
| PCTDC_17 | −1.54203 | −2.192574 | −2.076071 | −1.693325 | −1.265747 |
| PCTDC_18 | −2.54153 | −1.691014 | −1.825847 | −2.509501 | −2.54944 |
| PCTDC_19 | −2.096784 | −2.959664 | −1.619618 | −1.55181 | −1.288303 |
| PCTDC_20 | −4.713184 | −2.644333 | −2.191245 | −3.963356 | −7.454573 |
| PCTDC_21 | −2.334953 | 1.145789 | −1.030932 | −2.206662 | −2.146682 |
| PCTDC_22 | 1.237133 | 2.211828 | −1.071583 | 1.450114 | −2.12071 |
| PCTDC_23 | −7.005491 | −9.202102 | −4.653239 | −6.05403 | −13.13538 |
| PCTDC_24 | −5.951272 | −3.290831 | −3.232419 | −4.298961 | −4.875925 |
| PCTDC_25 | −2.081615 | −6.085269 | −1.765094 | −2.220059 | 1.845746 |
| PCTDC_26 | −2.293379 | −1.050853 | −1.285113 | −1.328853 | −1.934643 |
| PCTDC_27 | −2.839561 | 1.611821 | −2.185083 | −5.724341 | −1.866924 |
| PCTDC_28 | −1.563559 | −1.277798 | −1.635946 | −1.273865 | −1.36657 |
| PCTDC_29 | 1.078378 | 1.039777 | −1.32094 | 1.030855 | −1.184824 |
| PCTDC_30 | −1.29269 | −1.946759 | −2.059295 | −2.439818 | −1.469364 |
| PCTDC_31 | −1.012831 | −1.271201 | −1.522388 | −1.413658 | −1.035629 |
| PCTDC_32 | −1.400573 | −2.489946 | −2.067323 | −2.041809 | −2.21355 |
| PCTDC_33 | −1.792293 | −1.204384 | −1.955245 | −2.213695 | −1.2927 |
| PCTDC_34 | −9.176607 | −4.869056 | −4.837561 | −9.30142 | −6.392067 |
| PCTDC_35 | −1.325917 | −1.476112 | −1.903347 | −1.844246 | −1.166259 |
| PCTDC_36 | −2.577685 | −2.057103 | −2.070463 | −2.040741 | −2.200454 |
| PCTDC_37 | −6.387117 | −3.830329 | −3.541164 | −5.297482 | −5.487053 |
| PCTDC_38 | −1.808332 | −1.838845 | −1.399662 | −1.668483 | −2.744579 |
| PCTDC_39 | −2.020502 | −1.953096 | −2.389464 | −2.185357 | −1.589978 |
| PCTDC_40 | −2.345016 | −1.103264 | −1.3714 | −2.098808 | −1.36887 |
| PCTDC_41 | −1.30085 | −1.293364 | −2.020555 | −1.683774 | −1.218266 |
| PCTDC_42 | −6.69437 | −3.298533 | −3.746257 | −6.221455 | −5.266223 |
| PCTDC_43 | −2.282477 | −2.061087 | −2.344448 | −2.35339 | −1.983304 |
| PCTDC_44 | −2.485822 | −1.833041 | −2.228038 | −2.318379 | −1.774187 |
| PCTDC_45 | −2.598734 | −1.185102 | −2.771417 | −3.295656 | −2.129327 |
| PCTDC_46 | −6.373006 | −9.560293 | −2.788558 | −3.045106 | −6.789342 |
| PCTDC_47 | −4.87862 | −2.657794 | −2.552702 | −4.304638 | −4.328265 |
| PCTDC_48 | −1.898269 | −1.156685 | −1.726415 | −1.402836 | −1.626888 |
| PCTDC_49 | −1.255479 | 1.182284 | −1.860565 | −1.56444 | −2.199043 |
| PCTDC_50 | −1.2934993 | −5.623239 | −5.310431 | −13.596727 | −12.429982 |
| PCTDC_51 | −5.198398 | −4.477626 | −4.28704 | −4.892914 | −3.817191 |
| PCTDC_52 | −2.349524 | −1.820608 | −2.454295 | −2.495358 | −2.524858 |
| PCTDC_53 | −1.195486 | −1.319256 | −1.530677 | −1.392 | −1.095178 |
| PCTDC_54 | −1.265201 | −1.569405 | −2.220051 | −2.484167 | −1.76688 |
| PCTDC_55 | −1.88841 | −5.731989 | −2.436103 | −3.851297 | −3.033818 |
| PCTDC_56 | −6.924843 | −4.498032 | −4.446744 | −6.123724 | −5.885492 |
| PCTDC_57 | −1.721724 | −5.243209 | −2.373629 | −3.036423 | −3.06899 |
| PCTDC_58 | −1.942217 | −2.25106 | −2.107988 | −1.765252 | −1.959601 |
| PCTDC_59 | −2.031986 | 2.064397 | −1.413948 | −1.241656 | −1.031326 |
| PCTDC_60 | −7.579762 | −4.952477 | −5.026139 | −8.268243 | −6.976716 |
| PCTDC_61 | −1.848968 | −1.066639 | −1.398534 | −1.855107 | −1.062309 |
| PCTDC_62 | −2.240544 | −1.273063 | −2.48321 | −1.429924 | −1.609147 |
| PCTDC_63 | −2.854465 | −2.842449 | −3.035436 | −2.914622 | 1.10521 |
| PCTDC_64 | −3.893095 | −2.440034 | −1.984535 | −3.534865 | −4.673214 |
| PCTDC_65 | −1.732418 | −1.829374 | −1.810178 | −2.663752 | −1.023093 |
| PCTDC_66 | −2.16481 | −1.621258 | −1.986666 | −1.997742 | −1.548889 |
| PCTDC_67 | −1.901971 | −1.24001 | −1.747679 | −1.402124 | −1.510826 |
| PCTDC_68 | −2.217162 | −1.195414 | −1.493331 | −1.936626 | −1.071265 |
| PCTDC_69 | −1.245448 | −1.448996 | −1.999391 | −1.5595 | −1.106344 |
| PCTDC_70 | −1.998234 | −2.018207 | −2.033922 | −1.801316 | −1.697051 |
| PCTDC_71 | −1.511911 | −1.458437 | −1.272488 | −1.458376 | −2.154458 |
| PCTDC_72 | −1.203158 | −2.797158 | −1.235114 | 1.005051 | 1.023688 |
| PCTDC_73 | −1.541916 | −1.903445 | −1.544858 | −2.257688 | −1.289162 |
| PCTDC_74 | −4.853553 | −3.635271 | −5.199815 | −6.211979 | −5.240062 |
| PCTDC_75 | −3.273766 | −14.657293 | −2.862239 | −1.764461 | −1.760385 |
| PCTDC_76 | −1.217171 | −1.296736 | −1.144786 | 1.079204 | −1.260905 |
| PCTDC_77 | −1.797696 | 1.103401 | −1.335014 | −2.339793 | −1.511612 |
| PCTDC_78 | −2.456534 | −1.875372 | −2.855611 | −2.716163 | −2.373691 |
| PCTDC_79 | −2.525538 | −2.232876 | −3.110879 | −3.264385 | −3.456531 |
| PCTDC_80 | −1.630138 | −1.35257 | −1.631509 | −1.196468 | −4.494679 |
| PCTDC_81 | −1.929872 | −1.113005 | 1.031929 | 1.007586 | −2.347475 |
| PCTDC_82 | −1.70836 | −1.282488 | −1.783215 | −1.979933 | −1.949185 |
| PCTDC_83 | −1.243828 | −1.987254 | −1.764672 | −2.130092 | −1.901367 |
| PCTDC_84 | 1.232643 | −2.07259 | −1.662058 | −1.68123 | −1.850402 |
| PCTDC_85 | −3.765696 | −3.829437 | −3.300366 | −1.734307 | −2.509329 |
| PCTDC_86 | −6.189479 | −3.353586 | −3.170256 | −5.04737 | −5.663467 |
| PCTDC_87 | −2.475857 | −3.51374 | −2.164196 | −1.447658 | −2.618937 |
| PCTDC_88 | 1.944102 | −1.259746 | −3.338257 | 1.38276 | −1.214961 |
| PCTDC_89 | −5.932095 | −3.275715 | −2.963247 | −5.379752 | −6.126326 |
| PCTDC_90 | −4.716655 | −5.929635 | −3.482047 | −3.96486 | −6.801842 |
| PCTDC_91 | −4.519485 | −5.358361 | −3.661997 | −4.073708 | −6.157125 |
| PCTDC_92 | −1.685087 | −1.691207 | −1.860737 | −1.845194 | −1.751631 |
| PCTDC_93 | −1.492022 | −1.006958 | −1.228877 | −1.906211 | −1.943167 |
| PCTDC_94 | 1.237337 | −1.164018 | −1.261906 | −1.155859 | 1.205331 |
| PCTDC_95 | −2.12835 | −2.041079 | −1.984469 | −2.045971 | −1.898901 |
| PCTDC_96 | −2.078055 | −2.330517 | −1.327108 | −1.441385 | −3.743099 |
| PCTDC_97 | −1.334297 | −2.087266 | −2.462863 | −1.61449 | −1.632635 |
| PCTDC_98 | −2.000543 | −1.611817 | −1.535503 | −1.859716 | −2.520927 |
| PCTDC_99 | −3.419719 | −1.580899 | −2.110636 | −3.050031 | −3.421836 |
| PCTDC_100 | −2.379466 | −1.420795 | −1.926739 | −2.998748 | −2.869548 |
| PCTDC_101 | −2.000934 | −1.77734 | −1.552652 | −2.221759 | −2.488121 |
| PCTDC_102 | −3.985031 | −5.559905 | −3.089806 | −5.037643 | −9.124702 |
| PCTDC_103 | −1.916361 | −1.481127 | −1.837724 | −2.897688 | −1.658977 |
| PCTDC_104 | −2.531153 | −2.705933 | −2.725935 | −3.236739 | −2.795705 |
| PCTDC_105 | 1.216257 | −1.641811 | −1.067287 | 1.311882 | 1.061105 |
| PCTDC_106 | −2.073981 | −1.348887 | −1.531421 | −1.910613 | −2.255851 |
| PCTDC_107 | −2.031476 | −1.595985 | −1.77897 | −1.50754 | −2.122092 |
| PCTDC_108 | −2.335722 | −2.427527 | −2.178308 | −2.322503 | −3.071886 |
| PCTDC_109 | −1.91579 | 1.12028 | −1.746202 | −3.056107 | −3.911528 |
| PCTDC_110 | −11.250849 | −5.493386 | −5.459946 | −6.066711 | −7.821973 |
| PCTDC_111 | −4.779661 | −2.762719 | −2.382575 | −4.451578 | −4.830759 |
| PCTDC_112 | −4.049462 | −5.77829 | −3.279663 | −4.752324 | −7.3581 |
| PCTDC_113 | −4.345043 | −3.027864 | −2.915782 | −2.857641 | −3.202537 |
| PCTDC_114 | −1.556411 | −1.673897 | −1.841856 | −2.564856 | −1.955312 |
| PCTDC_115 | −1.410966 | −1.522985 | −1.206489 | −1.477594 | −2.159136 |
| PCTDC_116 | −1.671688 | −1.465093 | −1.653697 | −1.982813 | −2.208729 |
| PCTDC_117 | −4.229105 | −5.642348 | −3.183808 | −4.386364 | −6.983963 |
| PCTDC_118 | −1.964442 | 1.805349 | −1.85113 | −4.038047 | −1.632577 |
| PCTDC_119 | −2.186803 | −1.022619 | −2.035141 | −2.479424 | −2.150818 |
| PCTDC_120 | −1.430576 | −1.102015 | −1.132152 | −1.812828 | −3.149442 |
| PCTDC_121 | −4.904629 | −2.872402 | −2.430766 | −5.292966 | −5.347709 |
| PCTDC_122 | −3.6904 | −2.680579 | −1.969049 | −4.20231 | −7.732623 |
| PCTDC_123 | −4.49448 | −6.431859 | 3.257086 | −5.054696 | −8.789499 |
| PCTDC_124 | −1.744154 | −1.817764 | −1.186199 | −1.778908 | −1.913888 |
| PCTDC_125 | −4.713228 | −6.320646 | −3.339272 | −5.216887 | −8.649884 |
| PCTDC_126 | −4.696732 | −6.627284 | −3.523528 | −5.722506 | −10.116261 |
| PCTDC_127 | −1.108407 | −1.057277 | −1.179754 | −1.385472 | −1.268484 |
| PCTDC_128 | −4.545651 | −2.638318 | −2.41323 | −5.386978 | −4.731944 |
| PCTDC_129 | −5.034372 | −2.967628 | −2.568805 | −4.976381 | −5.099236 |
| PCTDC_130 | −2.051642 | −2.098477 | −1.837924 | −1.707663 | −1.703864 |
| PCTDC_131 | 2.627375 | −2.67068 | 1.834017 | −1.765682 | −2.783329 |
| PCTDC_132 | −4.665012 | −3.317816 | −3.19454 | −6.633753 | −6.66045 |
| PCTDC_133 | −2.946086 | −2.116852 | −1.72241 | −3.087247 | −3.948884 |
| PCTDC_134 | −3.058105 | −2.498846 | −2.002397 | −2.88534 | −3.806821 |
| PCTDC_135 | −1.687653 | −1.845589 | −1.873229 | −2.343037 | −1.830697 |
| PCTDC_136 | −2.200974 | −2.152797 | −1.148252 | −1.83632 | −1.976489 |
| PCTDC_137 | −10.828956 | −5.270857 | −4.860933 | −5.900774 | −9.482695 |
| PCTDC_138 | −2.895159 | −2.965245 | 2.169616 | −1.927329 | −3.188821 |
| PCTDC_139 | −3.934355 | −2.826199 | −2.375097 | −3.812811 | −3.661432 |
| PCTDC_140 | −1.904256 | −2.79029 | −1.253306 | −1.295051 | −2.622534 |
| PCTDC_141 | −3.08367 | −3.345164 | −2.524613 | −2.622477 | −3.134956 |
| PCTDC_142 | −2.957271 | −1.985764 | −2.006083 | −2.92826 | −3.725083 |
| PCTDC_143 | −1.066036 | −1.67104 | −1.015772 | −1.546854 | −1.530618 |
| PCTDC_144 | −1.445624 | −1.807576 | −1.111409 | −1.48605 | −3.227776 |
| PCTDC_145 | −3.07717 | −2.146355 | −1.129324 | −2.287934 | −2.854114 |
| PCTDC_146 | −7.009215 | −3.635918 | −3.136031 | −5.086275 | −5.066755 |
| PCTDC_147 | −4.207016 | −5.540858 | −3.094626 | −4.090336 | −6.743286 |
| PCTDC_148 | −1.351986 | −1.336251 | −1.004627 | −1.225727 | −1.128605 |
| PCTDC_149 | −4.108362 | −2.621138 | −2.309178 | −4.294799 | −4.056478 |
| PCTDC_150 | −5.98528 | −2.98137 | −3.386578 | −2.302485 | −4.899428 |
| PCTDC_151 | −3.855859 | −2.506654 | −2.1212 | −3.633645 | −6.417445 |
| PCTDC_152 | 5.204085 | −5.826153 | −3.315112 | −4.699694 | −9.195724 |
| PCTDC_153 | −2.591242 | −3.623291 | −1.582283 | −2.397384 | −4.042944 |
| PCTDC_154 | −1.988355 | −1.956847 | −1.400108 | −1.495448 | −1.414439 |
| PCTDC_155 | −3.045794 | −2.256173 | −1.79689 | −2.80938 | −3.849687 |
| PCTDC_156 | −1.985825 | −1.668599 | −1.772614 | −1.848234 | −1.905182 |
| PCTDC_157 | −24.091743 | −6.946621 | −7.996484 | −26.935823 | −23.703266 |
| PCTDC_158 | −1.650762 | −2.664782 | −1.50441 | −1.354878 | −2.137988 |
| PCTDC_159 | −1.149911 | −1.36099 | −1.640492 | −1.368059 | −1.395896 |
| PCTDC_160 | −3.750376 | −2.5433 | −2.277271 | −3.585937 | −4.150171 |
| PCTDC_161 | −1.889016 | −1.961895 | −1.7557 | −1.8793 | −1.652045 |
| PCTDC_162 | −3.621216 | −2.569228 | −2.185721 | −3.014324 | −3.992921 |
| PCTDC_163 | −1.837535 | −1.75843 | −1.906056 | −2.043089 | −2.040604 |
| PCTDC_164 | −1.248544 | −2.1374 | −1.981841 | −1.865184 | −1.622861 |
| PCTDC_165 | −1.095199 | −1.295839 | −1.833439 | −1.770437 | −1.344037 |
| PCTDC_166 | −2.718937 | −2.558548 | −3.240606 | −3.848436 | −3.650884 |
| PCTDC_167 | −4.016697 | −2.559193 | −2.349691 | −3.895859 | −4.55859 |
| PCTDC_168 | −2.680113 | −2.068863 | −1.738906 | −2.575072 | −3.449777 |
| PCTDC_169 | −1.661317 | −1.56804 | −1.022683 | −1.889948 | −2.182787 |
| PCTDC_170 | −2.058542 | −2.438631 | −2.294661 | −2.185661 | −2.612388 |
| PCTDC_171 | −1.668753 | −1.852991 | −1.614027 | −1.467932 | −2.244333 |
| PCTDC_172 | −1.337232 | −1.28112 | −1.140765 | −1.241679 | −1.518697 |
| PCTDC_173 | −1.23684 | −1.628725 | −1.725804 | −1.686747 | −2.797905 |
| PCTDC_174 | −1.578831 | −1.530159 | −1.323276 | −1.448158 | −3.484801 |
| PCTDC_175 | −6.860311 | −8.596538 | −6.015227 | −7.395287 | −6.868768 |
| PCTDC_176 | 1.416791 | −1.700415 | −1.08375 | −1.233969 | −1.22021 |
| PCTDC_177 | −1.947781 | −1.898147 | −1.996725 | −1.230632 | −2.043674 |
| PCTDC_178 | −1.914441 | −1.735873 | −1.912667 | −2.333658 | −2.213283 |
| PCTDC_179 | −1.584291 | −4.533706 | −1.981395 | −1.249368 | −1.442302 |
| PCTDC_180 | −1.035091 | −1.290039 | −1.996445 | −2.50551 | −1.144317 |
| PCTDC_181 | −1.88717 | −2.132685 | −2.245895 | −1.921092 | −1.720108 |
| PCTDC_182 | −7.096983 | −11.581309 | −3.847387 | −6.296889 | −7.922583 |
| PCTDC_183 | −1.641561 | −2.313724 | −1.780369 | −2.408584 | −1.815283 |
| PCTDC_184 | −1.761954 | −3.35066 | −1.288622 | −1.667949 | −2.856786 |
| PCTDC_185 | −1.25742 | −1.907512 | −1.662129 | −1.913995 | −1.710638 |
| PCTDC_186 | −1.340019 | −1.549101 | −1.453753 | −1.605042 | −1.663931 |
| PCTDC_187 | −1.803373 | −1.169177 | −1.637415 | −1.929255 | −1.797708 |
| PCTDC_188 | −5.764035 | −8.496846 | −4.912166 | −7.168073 | −5.870468 |
| PCTDC_189 | −3.328565 | −3.022969 | −3.115447 | −4.255837 | −4.161739 |
| PCTDC_190 | −1.254828 | −1.472193 | −1.478995 | −1.744422 | −1.826931 |
| PCTDC_191 | −2.159151 | −3.128419 | −1.950118 | −2.926632 | −1.847003 |
| PCTDC_192 | 1.236833 | −1.962486 | 1.002009 | −1.568607 | −1.256739 |
| PCTDC_193 | −3.631537 | −2.738209 | −2.246517 | −3.775388 | −6.564242 |
| PCTDC_194 | −4.487526 | −3.650918 | −3.31222 | −5.680427 | −6.258502 |
| PCTDC_195 | −1.8363 | −1.64694 | −1.935888 | −2.529458 | −2.259551 |
| PCTDC_196 | −2.474186 | −3.365656 | −1.343502 | −3.501091 | −4.264999 |
| PCTDC_197 | −1.807832 | −2.29473 | −1.370198 | −3.162165 | −5.352941 |
| PCTDC_198 | −2.344617 | −2.959151 | −2.17081 | −2.163698 | −2.65943 |
| PCTDC_199 | −4.386623 | −2.638841 | −2.480235 | −4.108781 | −4.452411 |
| PCTDC_200 | −2.09309 | −1.775309 | −1.933533 | −2.33589 | −2.088803 |
| PCTDC_201 | −1.450764 | −1.617985 | −1.051829 | −1.23726 | −3.690548 |
| PCTDC_202 | −3.046799 | −1.879969 | −2.648411 | −4.555435 | −3.545973 |
| PCTDC_203 | 1.108333 | −1.612469 | −1.357317 | 1.574016 | −1.019122 |
| PCTDC_204 | −8.862192 | −8.515929 | −3.496506 | −7.670859 | −13.681435 |
| PCTDC_205 | −1.075394 | −5.071301 | −1.509261 | −1.556702 | −1.422097 |
| PCTDC_206 | −1.737375 | −1.89541 | −1.9666 | −1.855576 | −1.827961 |
| PCTDC_207 | −2.82381 | −6.325802 | −2.511963 | −3.987634 | −2.676592 |
| PCTDC_208 | −1.656419 | −2.065864 | −1.284579 | −3.513086 | −1.361242 |
| PCTDC_209 | −5.483701 | −11.85375 | −3.020115 | −4.377883 | −5.433315 |
| PCTDC_210 | −1.15939 | −1.060312 | −1.423204 | −1.415902 | −1.342489 |
| PCTDC_211 | −4.027411 | −2.378188 | −2.177797 | −3.458907 | −3.85693 |
| PCTDC_212 | −1.658192 | −1.55431 | −1.589337 | −1.643705 | −1.989426 |
| PCTDC_213 | −1.72558 | −1.483346 | −1.542145 | −1.676729 | −2.45592 |
| PCTDC_214 | −2.161695 | −2.26164 | −2.339913 | −2.524176 | −2.457808 |
| PCTDC_215 | −2.529369 | −3.011394 | −2.542804 | −2.727487 | −3.088225 |
| PCTDC_216 | −1.656439 | −1.592109 | −1.819538 | −2.245645 | −1.654958 |
| PCTDC_217 | −1.847873 | −2.74288 | −1.248616 | −1.452109 | −2.042847 |
| PCTDC_218 | −1.166439 | −1.756858 | −1.308203 | −1.469586 | 1.014225 |
| PCTDC_219 | −1.252568 | −1.684199 | −1.155907 | 1.40619 | −1.54444 |
| PCTDC_220 | −4.17908 | −2.748761 | −2.267626 | −3.527103 | −3.513831 |
| PCTDC_221 | −4.836594 | −7.768823 | −3.52599 | −4.42607 | −10.136181 |
| PCTDC_222 | −3.906126 | −2.907072 | −2.068267 | −3.461873 | −6.774076 |
| PCTDC_223 | −1.100584 | −2.114033 | −1.601058 | −1.638044 | −1.699998 |
| PCTDC_224 | −4.242598 | −2.839127 | −2.379757 | −3.524704 | −3.613482 |
| PCTDC_225 | −4.777097 | −7.94543 | −3.501098 | −4.389704 | −10.008644 |
| PCTDC_226 | −4.540014 | −7.1293 | −3.391137 | −4.181539 | −9.32625 |
| PCTDC_227 | −1.422441 | −1.1647 | −1.218198 | −1.332996 | −1.117286 |
| PCTDC_228 | −3.922249 | −5.863266 | −3.065356 | −3.744807 | −6.576053 |
| PCTDC_229 | −5.263347 | −3.294776 | −2.501016 | −4.652264 | −4.555173 |
| PCTDC_230 | −3.925831 | −2.796558 | −2.098749 | −3.53759 | −7.084292 |
| PCTDC_231 | −5.242747 | −8.259526 | −3.789936 | −4.696026 | −11.373317 |
| PCTDC_232 | −2.294399 | −1.745048 | −1.850713 | −2.168706 | −2.485562 |
| PCTDC_233 | −1.238592 | −1.419183 | 1.100986 | −1.086468 | −1.028074 |
| PCTDC_234 | −2.238329 | −1.225333 | −1.22022 | −1.634064 | −1.887437 |
| PCTDC_235 | −26.014566 | −26.899576 | −22.260601 | −14.416063 | −30.63615 |
| PCTDC_236 | −1.525063 | −1.403936 | −1.457327 | 1.779269 | −2.068593 |
| PCTDC_237 | −1.342953 | −1.138019 | −1.133785 | −1.373282 | −1.051208 |
| PCTDC_238 | −1.501538 | −1.644368 | −1.863785 | −1.657531 | −1.664238 |
| PCTDC_239 | −1.904965 | −1.884623 | −1.672115 | −1.567592 | −1.527552 |
| PCTDC_240 | −2.111756 | −1.555367 | −1.523962 | −1.949346 | −1.554618 |
| PCTDC_241 | −2.691587 | −1.19034 | −1.934832 | −2.253114 | −2.510615 |
| PCTDC_242 | −2.298609 | −1.381359 | −1.308637 | −1.46631 | −1.679047 |
| PCTDC_243 | −1.402621 | 1.085415 | 1.062659 | −1.091258 | −1.358562 |
| PCTDC_244 | −3.540937 | −1.836178 | −2.367527 | −3.391082 | −2.486673 |
| PCTDC_245 | −2.214928 | −1.84093 | −1.84495 | −2.022239 | −1.820758 |
| PCTDC_246 | −1.76635 | −1.63073 | −1.682651 | −1.966334 | −1.6663 |
| PCTDC_247 | −1.778498 | 1.015742 | −1.128503 | −1.557932 | −1.658755 |
| PCTDC_248 | −4.171661 | −4.078993 | −3.07633 | −1.408247 | −2.28533 |
| PCTDC_249 | −5.12327 | −7.903827 | −3.478949 | −4.345335 | −9.133308 |
| PCTDC_250 | −4.108335 | −2.688739 | −2.270979 | −3.676643 | −3.862636 |
| PCTDC_251 | −4.484256 | −2.09902 | −2.316149 | −4.576964 | −2.427416 |
| PCTDC_252 | 1.110065 | −3.469609 | −1.558385 | −1.43239 | −1.210027 |
| PCTDC_253 | −1.699842 | −1.454623 | −1.422735 | −1.761389 | −1.237906 |
| PCTDC_254 | −1.831613 | −1.491833 | −1.792443 | 2.372589 | −5.5415 |
| PCTDC_255 | −2.389394 | −1.260788 | −1.077165 | −2.063594 | −1.515193 |
| PCTDC_256 | −2.79098 | −3.093994 | −1.257646 | −2.697717 | 1.756046 |
| PCTDC_257 | −2.007114 | −2.008637 | −1.469927 | −1.515188 | −1.6455 |
| PCTDC_258 | −1.608199 | −2.027372 | −1.10816 | −1.153414 | −2.64577 |
| PCTDC_259 | −2.354438 | −1.372294 | 1.226844 | −2.133314 | −1.354503 |
| PCTDC_260 | −2.604011 | −4.336377 | −1.289437 | −2.013778 | 4.400435 |
| PCTDC_261 | −1.537797 | −1.141336 | −1.495266 | −1.746315 | −1.160505 |
| PCTDC_262 | −2.753373 | −2.140275 | −2.532053 | −2.554981 | −1.703069 |
| PCTDC_263 | −1.785774 | −1.551533 | −1.656989 | −2.030993 | −1.766316 |
| PCTDC_264 | −3.580837 | −2.326493 | −2.58249 | −2.59469 | −2.352746 |
| PCTDC_265 | −2.374061 | −3.093873 | −1.422594 | −3.02511 | −1.326881 |
| PCTDC_266 | −3.302186 | −2.145091 | −2.473056 | −3.285471 | −2.608865 |
| PCTDC_267 | −2.346302 | −1.468917 | −1.721191 | −1.507031 | −1.709475 |
| PCTDC_268 | −2.107902 | −1.580947 | −2.055259 | −2.15739 | −1.497125 |
| PCTDC_269 | −2.541823 | −2.595453 | −3.66834 | −3.796033 | −3.32306 |
| PCTDC_270 | −2.54968 | −1.897837 | −1.5039 | −4.030859 | −2.933698 |
| PCTDC_271 | −4.312228 | −2.626342 | −3.335603 | −4.055226 | −3.386051 |
| PCTDC_272 | −2.036159 | −1.363548 | −1.613196 | −2.056096 | −1.644075 |
| PCTDC_273 | −1.466514 | −1.47301 | −1.619885 | −1.322114 | −2.217748 |
| PCTDC_274 | −2.826242 | −2.691233 | −2.291895 | −4.644922 | −2.121244 |
| PCTDC_275 | −1.862883 | −5.515562 | −2.056687 | −4.536572 | −1.252029 |
| PCTDC_276 | −4.188195 | −2.804357 | −2.14192 | −3.4069 | −7.087134 |
| PCTDC_277 | −3.316963 | −2.355866 | −2.018188 | −3.210177 | −2.994663 |
| PCTDC_278 | −1.59519 | −1.208626 | −1.073164 | −1.516793 | −1.36901 |
| PCTDC_279 | −3.328695 | −2.402078 | −2.674182 | −3.365015 | −2.838426 |
| PCTDC_280 | −2.00892 | −1.959798 | −1.418687 | −1.551801 | −1.356092 |
| PCTDC_281 | −2.085441 | −1.306356 | −1.934703 | −2.34709 | −1.849548 |
| PCTDC_282 | −2.031199 | −4.178926 | −1.501072 | −2.093809 | −1.027688 |
| PCTDC_283 | −2.256392 | −1.16297 | −1.590518 | −2.591235 | −2.012158 |
| PCTDC_284 | −2.433178 | −2.244144 | −1.806869 | −2.403887 | −3.270381 |
| PCTDC_285 | −2.107364 | −1.348654 | −1.292773 | −2.284883 | −1.276845 |
| PCTDC_286 | −1.011685 | −1.400408 | −1.559272 | −1.384357 | −1.122806 |
| PCTDC_287 | −1.979708 | −1.298204 | −1.255026 | −2.021377 | −1.188739 |
| PCTDC_288 | −2.305089 | −1.880831 | −2.239136 | −2.554946 | −2.08069 |
| PCTDC_289 | −1.486956 | −1.98167 | −1.036458 | −1.09955 | −2.816411 |
| PCTDC_290 | −3.562744 | −3.871484 | −1.923246 | −2.427721 | −3.265605 |
| PCTDC_291 | −3.097702 | −2.600365 | −2.303037 | −2.929789 | −3.116413 |
| PCTDC_292 | −1.56978 | −1.183312 | −1.520002 | −1.641381 | −1.129012 |
| PCTDC_293 | −10.18624 | −3.780659 | −5.276081 | −11.313716 | −10.131436 |
| PCTDC_294 | −3.900229 | −4.336713 | −1.672553 | −2.304041 | −6.733215 |
| PCTDC_295 | −1.209046 | −1.122768 | −1.587951 | −1.498479 | 1.044804 |
| PCTDC_296 | −3.698761 | −2.350756 | −2.777413 | −3.755806 | −2.51192 |
| PCTDC_297 | −3.093647 | −3.761977 | −2.186037 | −1.308696 | −2.641925 |
| PCTDC_298 | −2.297807 | −1.410159 | −1.9196 | −1.950024 | −1.650355 |
| PCTDC_299 | −1.89521 | −1.354424 | −1.123448 | −1.873263 | −1.36766 |
| PCTDC_300 | −1.42162 | −1.602237 | −1.268964 | −1.253886 | −1.127024 |
| PCTDC_301 | −2.943285 | −2.148288 | −1.191947 | −2.763554 | 2.763937 |
| PCTDC_302 | −6.406439 | −2.028337 | −2.167836 | −2.019916 | −1.109315 |
| PCTDC_303 | −2.817563 | −2.154164 | −1.78874 | −2.779721 | −4.163968 |
| PCTDC_304 | −7.464938 | −3.133507 | −4.03298 | −7.604817 | −5.315706 |
| PCTDC_305 | −2.014723 | −2.095896 | −1.131347 | −2.008184 | −2.41007 |
| PCTDC_306 | −3.969199 | −2.738114 | −2.292288 | −3.43873 | −3.428852 |
| PCTDC_307 | −1.417446 | −1.376043 | −1.435508 | −1.638834 | −1.476408 |
| PCTDC_308 | −8.653403 | −20.58325 | −4.370862 | −5.277433 | −15.810896 |
| PCTDC_309 | −3.137505 | −3.304923 | −3.245285 | −3.165118 | −2.716445 |
| PCTDC_310 | −12.061647 | −5.272011 | −5.720038 | −12.138116 | −11.271891 |
| PCTDC_311 | −2.115049 | −1.485084 | −1.570765 | −2.19385 | −1.463544 |
| PCTDC_312 | 1.481472 | −1.618008 | −1.014678 | −1.243736 | −1.10675 |
| PCTDC_313 | −1.273086 | 1.043674 | −1.00678 | −1.091739 | 1.003629 |
| PCTDC_314 | −2.49528 | −1.981606 | −2.53176 | −2.166479 | −1.675072 |
| PCTDC_315 | −4.246729 | −5.756291 | −3.252714 | −3.78408 | −6.582098 |
| PCTDC_316 | −1.270286 | 1.212425 | −1.341502 | 1.115258 | −1.351564 |
| PCTDC_317 | 1.354967 | −1.090403 | 1.094382 | 1.319759 | 1.075027 |
| PCTDC_318 | −1.418532 | −1.182988 | −1.410398 | −1.462745 | −1.37438 |
| PCTDC_319 | −2.847453 | −1.610835 | −2.137122 | −4.008525 | −2.580153 |
| PCTDC_320 | −1.570447 | −1.646244 | −1.15019 | −1.643979 | −1.055569 |
| PCTDC_321 | −1.523534 | 1.03465 | −1.379848 | −1.80666 | −1.725373 |
| PCTDC_322 | −1.959616 | −1.836321 | −1.791546 | −1.885724 | −2.337018 |
| PCTDC_323 | −1.759654 | −1.876256 | −1.381283 | −2.053537 | −1.730219 |
| PCTDC_324 | −4.241111 | −3.697052 | −3.347769 | −3.735688 | −3.168041 |
| PCTDC_325 | −4.293188 | −3.750995 | −2.755883 | −2.867415 | −5.334981 |
| PCTDC_326 | −2.483399 | −1.89337 | −1.533312 | −2.253657 | −2.249147 |
| PCTDC_327 | −1.918311 | −1.479681 | −2.0809 | −2.437624 | −2.628933 |
| PCTDC_328 | −4.101023 | −2.454861 | −1.939533 | −2.924037 | −3.540413 |
| PCTDC_329 | −3.325883 | −3.202878 | −2.660003 | −2.620374 | −2.884403 |
| PCTDC_330 | −4.325046 | −5.603637 | −2.757072 | −3.08421 | −6.878116 |
| PCTDC_331 | −2.936653 | −2.221632 | −1.802164 | −2.695677 | −4.706359 |
| PCTDC_332 | −1.649843 | −1.262365 | −1.228164 | −1.48323 | −1.408435 |
| PCTDC_333 | −2.141754 | −2.134733 | −1.757711 | −2.809459 | −1.735782 |
| PCTDC_334 | −3.501326 | −4.491755 | −2.473015 | −2.947388 | −6.778422 |
| PCTDC_335 | −1.227933 | −1.317701 | −1.295 | −1.258839 | −1.293943 |
| PCTDC_336 | −3.148972 | −2.179465 | −1.555655 | −2.493189 | −5.507409 |
| PCTDC_337 | −3.966367 | −2.473189 | −1.953672 | −3.182715 | −4.397205 |
| PCTDC_338 | −1.899115 | −1.978631 | −1.556313 | −1.552081 | −1.990791 |
| PCTDC_339 | −2.671378 | −1.864997 | −1.524507 | −2.402448 | −4.48918 |
| PCTDC_340 | −2.728256 | −2.135133 | −1.584932 | −2.20714 | −3.585228 |
| PCTDC_341 | −3.673474 | −4.037199 | −2.388833 | −3.204217 | −6.281793 |
| PCTDC_342 | −3.58528 | −2.442878 | −1.646449 | −2.521667 | −6.100334 |
| PCTDC_343 | −1.679677 | −1.991002 | −1.348143 | −1.318828 | −1.511339 |
| PCTDC_344 | −3.23739 | −3.437972 | −1.895346 | −3.190621 | −1.744413 |
| PCTDC_345 | −1.644668 | −2.779138 | −1.517523 | 1.364956 | −1.674406 |
| PCTDC_346 | −2.173687 | −1.90845 | −2.221743 | −2.314945 | −1.510086 |
| PCTDC_347 | −3.215951 | −2.527133 | −2.775464 | −3.561818 | −3.815771 |
| PCTDC_348 | −1.723149 | −1.577233 | 1.023316 | 1.173152 | −2.244136 |
| PCTDC_349 | −4.606752 | −5.350285 | 1.07467 | −3.393382 | 3.494553 |
| PCTDC_350 | −1.673569 | −2.053359 | 1.517985 | −1.509558 | −1.985934 |
| PCTDC_351 | −2.15345 | −2.781823 | 1.5802 | −1.603137 | −2.436613 |
| PCTDC_352 | −1.918688 | −4.757151 | 1.269333 | −2.993961 | −3.911849 |
| PCTDC_353 | −1.068852 | −2.075098 | −1.336365 | −1.345524 | −2.028439 |
| PCTDC_354 | −1.008482 | −1.545598 | −1.110947 | −1.199497 | 1.067307 |
| PCTDC_355 | −1.020998 | −1.740442 | −1.828657 | −1.44221 | −1.683714 |
| PCTDC_356 | −4.541613 | −6.258914 | −1.221775 | −2.06907 | 1.022882 |
| PCTDC_357 | −1.893087 | −2.572712 | −1.617558 | −1.725631 | −2.670305 |
| PCTDC_358 | −3.481641 | −2.246628 | −1.780498 | −2.936959 | −3.004195 |
| PCTDC_359 | −3.538222 | −3.849429 | −2.165769 | −3.021411 | −5.941804 |
| PCTDC_360 | −1.793573 | −2.921968 | −1.638617 | −1.843308 | −2.364318 |
| PCTDC_361 | −2.291991 | −1.61443 | −1.443631 | −1.733552 | −1.618359 |
| PCTDC_362 | −1.670359 | −1.499924 | −1.285046 | −1.541224 | −1.269314 |
| PCTDC_363 | −1.802833 | −1.689108 | −1.911253 | −2.12268 | −2.399896 |
| PCTDC_364 | −1.898284 | −1.981644 | −1.924549 | −1.549852 | −2.081147 |
| PCTDC_365 | 4.22351 | −9.226021 | 3.084101 | −1.705691 | −11.104012 |
| PCTDC_366 | −1.888896 | −1.85332 | −2.192266 | −2.289319 | −2.119952 |
| PCTDC_367 | −1.97838 | −1.367645 | −1.522653 | −2.071325 | −2.921427 |
| PCTDC_368 | −1.759534 | −1.378262 | −1.961315 | −1.663318 | −1.758865 |
| PCTDC_369 | −1.840346 | −2.035742 | −2.124624 | −1.275016 | −1.85635 |
| PCTDC_370 | −2.007089 | 1.096472 | −1.096968 | −1.650896 | −1.568698 |
| PCTDC_371 | −3.043957 | −2.778382 | −3.056535 | −4.018412 | −6.229258 |
| PCTDC_372 | −1.488775 | −1.266047 | −1.503389 | −1.584015 | −1.764421 |
| PCTDC_373 | −2.474675 | −1.855865 | −2.397671 | −3.08373 | −4.650255 |
| PCTDC_374 | −1.477882 | −2.920208 | −1.197682 | 1.409172 | −1.21861 |
| PCTDC_375 | −1.345356 | −1.468122 | −1.632299 | −1.213916 | −1.686642 |
| PCTDC_376 | −2.18548 | −3.157719 | −1.975049 | −2.149141 | −2.804915 |
| PCTDC_377 | −3.444881 | −3.297239 | −2.220726 | −4.062848 | −2.433287 |
| PCTDC_378 | −4.045188 | −1.286256 | −1.490966 | −2.940182 | −1.901902 |
| PCTDC_379 | −4.225448 | −2.540783 | −1.936715 | −3.018784 | −3.767159 |
| PCTDC_380 | −2.938715 | −3.974298 | −1.779847 | −2.547801 | −1.31499 |
| PCTDC_381 | −4.600126 | −2.575117 | −2.007174 | −3.468507 | −4.303986 |
| PCTDC_382 | −3.937433 | −5.777416 | −2.985915 | −3.02475 | −6.739953 |
| PCTDC_383 | −3.820225 | −5.31635 | −2.647508 | −3.152502 | −6.585879 |
| PCTDC_384 | −2.898999 | −2.564559 | −2.41546 | −2.562159 | −2.392565 |
| PCTDC_385 | −1.971279 | −1.510526 | −1.926565 | −1.778569 | −1.995005 |
| PCTDC_386 | −3.693599 | −5.350247 | −2.611449 | −2.99702 | −6.727289 |
| PCTDC_387 | −3.671165 | −2.35536 | −1.846964 | −2.844131 | −3.411843 |
| PCTDC_388 | −6.418478 | −2.875051 | −2.671779 | −6.267906 | −8.002217 |
| PCTDC_389 | −3.510195 | −2.252676 | −1.83295 | −2.903546 | −3.303936 |
| PCTDC_390 | −2.303 | −1.856063 | −1.523551 | −2.242671 | −3.465624 |
| PCTDC_391 | −1.787495 | −2.155359 | −1.689284 | −1.394923 | −1.520547 |
| PCTDC_392 | −2.296905 | −1.875615 | −1.871569 | −1.92093 | −1.96803 |
| PCTDC_393 | −1.118759 | −1.34588 | −1.05869 | −1.307118 | 1.076997 |
| PCTDC_394 | −14.147042 | −27.16382 | −10.344748 | −10.396039 | −17.129059 |
| PCTDC_395 | −1.990524 | 1.161036 | −1.893522 | −2.769256 | −2.91797 |
| PCTDC_396 | −1.563106 | −2.419688 | −1.19848 | −2.27685 | 1.625479 |
| PCTDC_397 | −1.65502 | −3.812934 | −1.760821 | −1.792365 | 1.010416 |
| PCTDC_398 | −1.810502 | −1.8462 | −1.610123 | −1.943737 | −1.689724 |
| PCTDC_399 | −1.897382 | −2.459177 | −1.758665 | −2.345544 | −1.599726 |
| PCTDC_400 | −1.745117 | 1.215976 | 1.159521 | −2.366141 | −1.683216 |
| PCTDC_401 | −2.316692 | −2.547475 | −1.865808 | −2.672911 | −2.25369 |
| PCTDC_402 | −1.895008 | −2.318722 | −1.982123 | −1.897704 | −1.610391 |
| PCTDC_403 | −2.206829 | −1.992888 | −1.958218 | −2.322778 | −1.765563 |
| PCTDC_404 | −3.24259 | 1.105085 | 1.600145 | −1.331533 | −1.211876 |
| PCTDC_405 | −1.461492 | −3.163721 | −1.309089 | 1.201983 | −1.057724 |
| PCTDC_406 | −3.758136 | −5.656469 | −1.197139 | −4.133626 | −2.615841 |
| PCTDC_407 | 1.043213 | −5.557227 | −1.607802 | −2.326464 | −21.611631 |
| PCTDC_408 | −1.865685 | −2.494514 | −1.59753 | −1.834113 | −1.55844 |
| PCTDC_409 | −5.336955 | −2.904762 | −2.434043 | −4.500535 | −4.37821 |
| PCTDC_410 | −2.80336 | −2.559432 | −1.839973 | −1.461133 | −3.095735 |
| PCTDC_411 | −1.90841 | −1.111871 | −1.727242 | −2.36153 | −1.560955 |
| PCTDC_412 | −6.033443 | −5.572865 | −2.395153 | −5.529053 | −7.323551 |
| PCTDC_413 | −2.243366 | −2.065153 | −1.848375 | −2.587145 | −1.635368 |
| PCTDC_414 | 2.023767 | 1.331859 | 2.169444 | −1.916078 | −1.403027 |
| PCTDC_415 | −3.920775 | −2.252436 | −3.213345 | −5.994901 | −4.956508 |
| PCTDC_416 | −7.442655 | −2.28381 | −2.242605 | −3.477119 | −2.146398 |
| PCTDC_417 | −2.607719 | −2.390978 | −2.229146 | −2.052244 | −2.063334 |
| PCTDC_418 | −4.090001 | −2.633664 | −2.038903 | −3.328784 | −6.567248 |
| PCTDC_419 | −2.023584 | 1.352602 | −1.670136 | −2.5473 | −2.245735 |
| PCTDC_420 | −2.251421 | 1.800712 | −1.947923 | −4.515185 | −1.667579 |
| PCTDC_421 | −2.21586 | −2.398456 | −2.138798 | −2.086025 | 1.06367 |
| PCTDC_422 | −5.115602 | −9.702243 | −1.006393 | −6.4676 | −3.524682 |
| PCTDC_423 | −1.831691 | 1.008045 | −1.119537 | −1.713724 | 1.0263 |
| PCTDC_424 | −1.11881 | −2.558552 | −1.29532 | −1.944752 | −1.721406 |
| PCTDC_425 | −3.606344 | −1.584107 | −1.723869 | −5.365775 | −3.688865 |
| PCTDC_426 | −2.116754 | 1.178401 | −1.201768 | −2.278917 | −1.92316 |
| PCTDC_427 | −3.06407 | −3.861369 | −3.700923 | −3.038453 | −2.201797 |
| PCTDC_428 | −1.974874 | 1.030132 | 1.112083 | −2.272229 | −1.298947 |
| PCTDC_429 | −2.138915 | −10.298188 | −2.214486 | −1.305676 | −1.288579 |
| PCTDC_430 | −2.040858 | 1.937925 | −1.854622 | −3.497624 | −1.320876 |
| PCTDC_431 | −4.125475 | −2.652308 | −2.345984 | −3.330373 | −3.384879 |
| PCTDC_432 | −1.75307 | −1.49367 | −1.544939 | −1.801559 | −1.458804 |
| PCTDC_433 | −2.272839 | −2.53438 | −1.61612 | −2.163939 | −2.00098 |
| PCTDC_434 | −1.28826 | −1.575161 | −1.58858 | −1.686772 | −1.054567 |
| PCTDC_435 | −5.220012 | −7.5921 | −3.568733 | −4.668806 | −8.816271 |
| PCTDC_436 | −1.194001 | −1.267171 | −1.202947 | −1.160666 | −1.904211 |
| PCTDC_437 | −4.118565 | −2.392381 | −1.881881 | −3.254918 | −3.592949 |
| PCTDC_438 | −2.799394 | −2.12613 | −1.812833 | −2.694802 | −3.01254 |
| PCTDC_439 | −2.606339 | −2.348985 | −2.160693 | −2.992317 | −2.952945 |
| PCTDC_440 | −1.644827 | −1.259022 | −1.456172 | −1.209592 | −1.548498 |
| PCTDC_441 | −2.326669 | −1.780098 | −2.208816 | −2.820748 | −2.577038 |
| PCTDC_442 | −2.462439 | −2.061104 | −1.573771 | −2.394099 | −2.343858 |
| PCTDC_443 | −9.429247 | −4.145243 | −4.631259 | −9.646797 | −7.486014 |
| PCTDC_444 | −3.822721 | −2.7215 | −1.607755 | −3.124867 | −3.169854 |
| PCTDC_445 | −4.733167 | −5.118199 | −2.359844 | −4.211793 | −4.978528 |
| PCTDC_446 | −2.242095 | −2.142577 | −1.814265 | −2.243981 | −1.820491 |
| PCTDC_447 | −1.873643 | −2.16026 | −1.803395 | −2.071719 | −1.670508 |
| PCTDC_448 | −1.228048 | −1.156409 | −1.297073 | −1.128794 | −1.177185 |
| PCTDC_449 | −1.243277 | −1.308936 | −1.231462 | −1.932546 | −1.105554 |
| PCTDC_450 | −1.825526 | −1.926383 | −1.841183 | −2.168026 | −1.812707 |
| PCTDC_451 | −1.960918 | −2.016697 | −1.027948 | −1.270407 | −1.836382 |
| PCTDC_452 | −1.013147 | −1.118609 | −1.454832 | −1.847407 | −1.027153 |
| PCTDC_453 | −1.859579 | −1.580305 | −1.656572 | −2.144008 | −1.537008 |
| PCTDC_454 | −2.86018 | −2.866956 | −1.411291 | −2.079742 | −2.578795 |
| PCTDC_455 | −1.18778 | −1.118609 | −1.298338 | −1.736544 | 1.111227 |
| PCTDC_456 | −1.202898 | −1.348395 | −1.104627 | −1.09717 | 1.032196 |
| PCTDC_457 | −3.921933 | −2.491198 | −2.09695 | −3.465019 | −3.414396 |
| PCTDC_458 | −4.198047 | −2.623072 | −2.234297 | −3.627238 | −3.373092 |
| PCTDC_459 | −4.147692 | −2.674235 | −2.27989 | −3.407578 | −3.197421 |
| PCTDC_460 | −4.249775 | −2.763192 | −2.221259 | −3.763872 | −3.620351 |
| PCTDC_461 | −5.280007 | −7.671776 | −3.699308 | −4.451082 | −8.672559 |
| PCTDC_462 | −3.57887 | −2.278907 | −1.935006 | −3.113895 | −3.381089 |
| PCTDC_463 | −2.981488 | −2.488562 | −2.14119 | −3.487246 | −3.115881 |
| PCTDC_464 | −2.379064 | −2.893599 | −1.953627 | −2.104885 | −2.612707 |
| PCTDC_465 | −2.479421 | −3.298689 | −2.407963 | −2.989532 | −3.314621 |
| PCTDC_466 | −1.410138 | −1.44473 | −1.258094 | −1.596811 | −1.593217 |
| PCTDC_467 | −2.390594 | −2.345222 | −1.961068 | −2.361172 | −2.357436 |
| PCTDC_468 | −1.680336 | −3.259171 | −1.838008 | −2.652272 | −1.206303 |
| PCTDC_469 | −2.045475 | −1.709187 | −1.800676 | −2.270922 | −1.779019 |
| PCTDC_470 | −1.983869 | −3.163117 | −1.795463 | −2.738596 | −1.964095 |
| PCTDC_471 | −2.451482 | −1.574182 | −1.89738 | −2.73348 | −1.87724 |
| PCTDC_472 | −1.037877 | −1.923896 | −2.101907 | −3.020603 | −1.84166 |
| PCTDC_473 | −1.751684 | −1.556972 | −1.634768 | −2.287308 | −1.514002 |
| PCTDC_474 | −4.04888 | −3.69383 | −2.858062 | −3.35046 | −4.122208 |
| PCTDC_475 | −2.757834 | −2.932736 | −1.846393 | −2.068793 | −2.324373 |
| PCTDC_476 | −1.312905 | −1.293242 | −1.655551 | −1.990178 | −1.110877 |
| PCTDC_477 | −2.038354 | −1.61711 | −1.890354 | −4.704314 | −1.76905 |
| PCTDC_478 | −2.165554 | −1.980344 | −1.793583 | −1.943708 | −1.758711 |
| PCTDC_479 | −4.170393 | −2.573389 | −2.156788 | −3.468242 | −3.309002 |
| PCTDC_480 | −1.707033 | −1.169422 | −1.193386 | −1.478945 | −1.621326 |
| PCTDC_481 | −3.940004 | −2.535416 | −2.119418 | −3.3859 | −3.558542 |
| PCTDC_482 | −2.601739 | −1.264894 | −1.697132 | −3.261934 | −2.244717 |
| PCTDC_483 | −2.718768 | −2.185249 | −1.651059 | −1.869576 | −2.812357 |
| PCTDC_484 | −2.39852 | −1.631899 | −1.848976 | −2.475552 | −1.284731 |
| PCTDC_485 | −2.057471 | −2.066126 | −2.464797 | −2.260879 | −1.797202 |
| PCTDC_486 | −1.164735 | −1.380944 | −1.368693 | −1.605741 | −1.046789 |
| PCTDC_487 | 1.351533 | −1.199158 | −1.211928 | −1.358015 | 1.112984 |
| PCTDC_488 | −2.173894 | −1.688027 | −1.826145 | −2.155279 | −1.247267 |
| PCTDC_489 | −6.81174 | −4.885068 | −3.691052 | −6.952291 | −5.914726 |
| PCTDC_490 | −2.693876 | −2.168933 | −1.795236 | −2.225158 | −1.464391 |
| PCTDC_491 | 1.022384 | −1.962803 | −1.111503 | −1.53311 | −1.457028 |
| PCTDC_492 | −4.435233 | −4.72611 | −2.31092 | −4.114769 | −5.46749 |
| PCTDC_493 | −1.726497 | 1.237203 | −1.684367 | −2.214422 | −2.250615 |
| PCTDC_494 | −3.986704 | −10.214438 | −2.235956 | −1.289291 | −3.902556 |
| PCTDC_495 | −9.662001 | −4.017264 | −4.912549 | −7.437357 | −8.433582 |
| PCTDC_496 | −1.931297 | −2.426281 | −1.483111 | −1.651267 | −1.59488 |
| PCTDC_497 | −4.386757 | −2.803833 | −2.258645 | −3.774412 | −3.796883 |
| PCTDC_498 | −1.910983 | −1.800007 | −1.728982 | −1.602626 | −2.152866 |
| PCTDC_499 | −3.801859 | −2.794723 | −1.966794 | −3.332434 | −6.132774 |
| PCTDC_500 | −3.766929 | −2.549677 | −1.934078 | −3.285396 | −5.195549 |
| PCTDC_501 | −2.497117 | −1.798926 | −1.79396 | −1.974637 | −3.059483 |
| PCTDC_502 | −4.022521 | −1.818765 | −1.942933 | −2.244681 | −1.375072 |
| PCTDC_503 | −1.384349 | −1.163179 | −1.623914 | −1.462417 | −1.09429 |
| PCTDC_504 | −3.270537 | −2.029771 | −1.740615 | −2.643592 | −2.993415 |
| PCTDC_505 | −5.003045 | −3.663749 | −4.378886 | −3.824932 | −5.549801 |
| PCTDC_506 | −1.345389 | −1.281099 | −1.152219 | −1.296648 | −1.080703 |
| PCTDC_507 | −4.757814 | −7.498409 | −1.651077 | −5.082231 | −10.403072 |
| PCTDC_508 | −2.800021 | −2.040236 | −1.515285 | −2.37896 | −2.617315 |
| PCTDC_509 | −2.934403 | −4.523464 | −1.688992 | −3.833131 | −7.293571 |
| PCTDC_510 | −1.476941 | −2.454367 | −1.741225 | −1.378503 | −1.920962 |
| PCTDC_511 | −1.973351 | −1.731608 | −1.729295 | −1.56888 | −1.714635 |
| PCTDC_512 | −2.655767 | −19.708242 | −1.225767 | −3.286163 | −18.513933 |
| PCTDC_513 | −3.147304 | −47.15366 | −1.472986 | −4.223797 | −53.499794 |
| PCTDC_514 | −2.896986 | −42.338341 | −1.397007 | −4.191507 | −44.630997 |
| PCTDC_515 | −2.789012 | −2.157998 | −1.996718 | −1.580221 | −2.797045 |
| PCTDC_516 | −1.372227 | −2.083936 | −1.553563 | −1.535993 | −1.558006 |
| PCTDC_517 | −2.612071 | −2.37371 | −2.24176 | −2.073574 | −2.146933 |
| PCTDC_518 | −3.503985 | −1.595497 | −1.11622 | −2.607165 | −1.074117 |
| PCTDC_519 | −5.198984 | −9.360762 | −7.042399 | −10.017647 | −5.152067 |
| PCTDC_520 | −1.335818 | 1.267761 | −2.617651 | 1.228106 | −1.36805 |
| PCTDC_521 | −2.380674 | −2.470443 | −2.565333 | −2.267503 | −2.319755 |
| PCTDC_522 | −1.350376 | −1.445063 | −1.346426 | −1.360597 | −1.340386 |
| PCTDC_523 | −2.445678 | −2.15636 | −2.008549 | −1.974647 | −2.243389 |
| PCTDC_524 | −3.786515 | −2.643449 | −2.021266 | −2.914837 | −3.078571 |
| PCTDC_525 | −5.028649 | −1.391998 | −1.738997 | −3.433631 | −2.018528 |
| PCTDC_526 | 1.012703 | −1.66488 | −1.121852 | −1.066909 | −1.140691 |
| PCTDC_527 | −4.322382 | −5.28279 | −2.794171 | −3.210779 | −6.969234 |
| PCTDC_528 | −1.037666 | −1.481409 | −1.933469 | −1.923975 | −1.000878 |
| PCTDC_529 | −4.203119 | −2.413176 | −1.883237 | −3.05964 | −3.565671 |
| PCTDC_530 | −3.489052 | −1.022411 | −1.351068 | −3.08581 | −2.888702 |
| PCTDC_531 | −2.788715 | 1.008337 | −1.266413 | −2.898742 | −2.418625 |
| PCTDC_532 | −1.599621 | −1.732217 | −1.172744 | −1.01763 | −1.412733 |
| PCTDC_533 | −3.127232 | −3.188901 | −3.005321 | −2.579616 | −2.570059 |
| PCTDC_534 | −1.528137 | −1.314179 | −2.057945 | −2.069454 | −1.574109 |
| PCTDC_535 | −1.786282 | −2.101565 | −2.098175 | −2.198452 | −1.403034 |
| PCTDC_536 | −1.177096 | −1.031142 | −1.319487 | −1.140883 | 1.343581 |
| PCTDC_537 | −3.799112 | −2.701041 | −1.966207 | −3.6021 | −6.962352 |
| PCTDC_538 | −1.977907 | −1.31051 | −1.444315 | −1.750597 | −1.304521 |
| PCTDC_539 | −3.053032 | −2.333641 | −1.411762 | −2.712081 | −3.116833 |
| PCTDC_540 | 2.15045 | −2.562178 | 1.666789 | −1.648637 | −2.499994 |
| PCTDC_541 | −2.255368 | −2.092699 | −2.72356 | −2.457666 | −2.074179 |
| PCTDC_542 | −3.057606 | −2.365003 | −1.936607 | −2.646039 | −3.576478 |
| PCTDC_543 | −2.02795 | −1.1764 | −1.487945 | −1.991581 | −1.52056 |
| PCTDC_544 | −2.35955 | −3.624348 | 1.159183 | −3.163264 | −2.038474 |
| PCTDC_545 | −3.578978 | −2.29385 | −2.024204 | −2.684515 | −2.701118 |
| PCTDC_546 | −4.24607 | −3.474969 | −4.85251 | −6.135162 | −5.773375 |
| PCTDC_547 | −1.790011 | −2.155357 | −2.026831 | −2.01 | −1.520742 |
| PCTDC_548 | −2.02119 | −2.574125 | 1.103573 | −2.249537 | −2.783807 |
| PCTDC_549 | −1.087209 | −1.240978 | −1.428093 | −1.7115 | 1.06794 |
| PCTDC_550 | −3.323033 | −2.491836 | −1.86641 | −3.041298 | −5.106065 |
| PCTDC_551 | −2.214859 | −1.530053 | −1.05263 | −1.959219 | −1.045903 |
| PCTDC_552 | −1.231717 | −2.050832 | −2.720342 | −2.200618 | −1.941845 |
| PCTDC_553 | −1.025631 | −2.275231 | −1.452125 | −2.132788 | −1.666659 |
| PCTDC_554 | −1.135718 | −1.081424 | −1.407248 | −1.361144 | 1.153963 |
| PCTDC_555 | −3.005455 | −4.273493 | −2.095281 | −1.541273 | −4.177554 |
| PCTDC_556 | −2.298774 | −2.56509 | −1.304017 | −1.308648 | −3.126355 |
| PCTDC_557 | −2.642847 | −3.578011 | −1.344106 | −3.135075 | −6.856468 |
| PCTDC_558 | −2.091945 | −2.207287 | −2.653325 | −2.720855 | −2.248579 |
| PCTDC_559 | −2.178046 | −1.506931 | −1.37275 | −2.056458 | −1.884107 |
| PCTDC_560 | −3.988228 | −2.979001 | −3.621165 | −5.204254 | −3.823349 |
| PCTDC_561 | −26.0333 | −8.337242 | −9.079122 | −24.277685 | −22.783827 |
| PCTDC_562 | −1.108282 | 1.164169 | 1.039573 | −1.053774 | 1.113347 |
| PCTDC_563 | −1.573374 | −1.054869 | 1.167521 | 1.08473 | −1.576548 |
| PCTDC_564 | −3.087662 | −2.850971 | −3.033665 | −3.457917 | −3.067259 |
| PCTDC_565 | −2.004796 | −1.672195 | −1.761897 | −1.906638 | −1.521024 |
| PCTDC_566 | −1.573435 | −1.588811 | −1.115372 | −1.014231 | 1.284361 |
| PCTDC_567 | −1.904652 | −1.555524 | −1.938543 | −1.984841 | −1.28467 |
| PCTDC_568 | −1.068629 | 1.213866 | −1.78508 | −1.641857 | −2.221032 |
| PCTDC_569 | −2.375146 | −2.102661 | −2.029639 | −1.663989 | −1.420596 |
| PCTDC_570 | −9.608533 | −8.61835 | −3.753283 | −9.15265 | −17.243759 |
| PCTDC_571 | −3.498796 | 1.100427 | 1.52337 | −1.171291 | −1.235893 |
| PCTDC_572 | −1.714235 | −1.05276 | −1.427239 | −2.068653 | −1.311309 |

| | Colon | Colon | Colon | Colon | Colon |
|---|---|---|---|---|---|
| Internal Id | Tumor 6 | Tumor 7 | Tumor 8 | Tumor 9 | Tumor 10 |
| PCTDC_1 | −2.083407 | −8.688679 | −9.78441 | −14.029575 | −6.670203 |
| PCTDC_2 | −2.114774 | −4.047634 | −2.835961 | −2.071182 | −3.214962 |
| PCTDC_3 | −1.927832 | −3.16564 | −2.886491 | −2.446894 | −1.904502 |
| PCTDC_4 | −1.855833 | −1.014881 | −6.896226 | −1.656962 | −2.489081 |
| PCTDC_5 | −1.842869 | −3.031669 | −2.735307 | −3.107583 | −2.954397 |
| PCTDC_6 | −1.232452 | −1.392996 | −1.036028 | −1.758322 | −3.107631 |
| PCTDC_7 | −8.855359 | −1.216792 | −24.185755 | 1.414277 | −18.743429 |
| PCTDC_8 | −1.019038 | −1.904193 | −1.840326 | −2.781147 | −3.551286 |
| PCTDC_9 | −16.077021 | −59.434616 | −38.668518 | −51.549026 | −43.448406 |
| PCTDC_10 | −2.379478 | −3.532775 | −1.889631 | −2.401799 | −4.232121 |
| PCTDC_11 | −5.102507 | −20.116104 | −7.378749 | −9.099745 | −8.085587 |
| PCTDC_12 | 1.072907 | −1.407256 | 1.180162 | −1.905116 | −3.182707 |
| PCTDC_13 | −4.354877 | −4.62117 | −11.826143 | −3.442251 | −5.913578 |
| PCTDC_14 | −1.56822 | −2.118925 | −1.938972 | −1.961342 | −1.970369 |
| PCTDC_15 | −2.433607 | −5.873315 | −2.510856 | −1.735214 | −5.508246 |
| PCTDC_16 | −3.29295 | −6.804746 | −2.026004 | −2.810378 | −1.862623 |
| PCTDC_17 | −2.036594 | −2.364163 | −4.195938 | −3.319173 | −3.066366 |
| PCTDC_18 | −1.872667 | −4.038313 | −2.298284 | −2.932148 | −2.821428 |
| PCTDC_19 | −2.898973 | −3.003338 | −2.332875 | −1.90442 | −2.035478 |
| PCTDC_20 | −4.287554 | −3.738031 | −19.061356 | −3.511957 | −10.332695 |
| PCTDC_21 | −1.115742 | −5.497366 | 1.296405 | −3.967105 | −4.493529 |
| PCTDC_22 | 1.448982 | −2.034846 | −2.080018 | −1.381942 | −6.800498 |
| PCTDC_23 | −5.260255 | −5.300622 | −19.321873 | −11.009766 | −11.427928 |
| PCTDC_24 | −3.6086 | −7.682777 | −4.40144 | −4.022962 | −2.925095 |
| PCTDC_25 | −3.207089 | −2.655337 | −3.553803 | −6.520929 | −3.446985 |
| PCTDC_26 | −1.940149 | −3.591095 | −1.951122 | −1.37034 | −3.077018 |
| PCTDC_27 | 3.660588 | −10.868382 | 1.926474 | −1.630994 | −8.4668 |
| PCTDC_28 | −1.321581 | −3.660369 | −1.933117 | −2.098044 | −4.096041 |
| PCTDC_29 | −1.005047 | −2.532266 | −2.431369 | −2.85346 | −3.993455 |
| PCTDC_30 | −1.960798 | −2.788447 | −3.257213 | −2.917638 | −2.279913 |
| PCTDC_31 | −1.46709 | −2.706539 | −3.315894 | −3.28479 | −1.964129 |
| PCTDC_32 | −1.97579 | −2.158711 | −2.641496 | −2.999132 | −2.193696 |
| PCTDC_33 | −1.526485 | −1.673905 | −1.723348 | −2.351685 | −2.155787 |
| PCTDC_34 | −4.70703 | −12.4248 | −6.442802 | −6.281425 | −5.773592 |
| PCTDC_35 | −1.891547 | −2.539454 | −2.715621 | −3.328795 | −1.898967 |
| PCTDC_36 | −2.28446 | −2.584173 | −2.668974 | −1.717271 | −1.928648 |
| PCTDC_37 | −4.428577 | −9.50749 | −5.010199 | −4.99507 | −3.553512 |
| PCTDC_38 | −1.792163 | −3.028126 | −2.257229 | −2.170509 | −4.585708 |
| PCTDC_39 | −2.005652 | −4.330509 | −2.296262 | −4.873521 | −2.819757 |
| PCTDC_40 | −1.772813 | −4.667068 | −2.386149 | −2.537639 | −3.865486 |
| PCTDC_41 | 1.564349 | −2.61227 | −3.514743 | −3.92785 | −2.121385 |
| PCTDC_42 | −3.7603 | −9.58972 | −4.996074 | −4.661958 | −4.888981 |
| PCTDC_43 | −2.077527 | −3.233304 | −2.467427 | −2.282286 | −2.178664 |
| PCTDC_44 | −2.102059 | −3.104153 | −4.135684 | −3.281705 | −3.038427 |
| PCTDC_45 | −2.089204 | −2.438284 | −5.021548 | −2.835689 | −5.339724 |
| PCTDC_46 | −2.472825 | 1.256632 | −5.916982 | −13.388905 | −2.907615 |
| PCTDC_47 | −3.70784 | −4.090519 | −10.020152 | −4.129423 | −6.91205 |
| PCTDC_48 | −1.450668 | −2.800931 | −2.528788 | −2.254879 | −2.278187 |
| PCTDC_49 | −1.29667 | −2.640137 | −1.01344 | −2.202153 | −3.045769 |
| PCTDC_50 | −6.235202 | −18.762634 | −8.1091263 | −8.401872 | −8.138474 |
| PCTDC_51 | −4.412484 | −9.920094 | −5.504246 | −5.859859 | −4.390945 |
| PCTDC_52 | −2.228088 | −4.798067 | −4.327576 | −5.975545 | −4.351716 |
| PCTDC_53 | −1.588446 | −3.653877 | −2.09207 | −2.502064 | −3.282234 |
| PCTDC_54 | −1.68808 | −2.522207 | −2.462492 | −2.462886 | −2.378968 |
| PCTDC_55 | −2.748683 | −9.275631 | −7.243145 | −6.042737 | −1.017954 |
| PCTDC_56 | −4.223862 | −9.964831 | −5.768355 | −6.104057 | −5.487343 |
| PCTDC_57 | −2.605145 | −8.748384 | −6.431192 | −5.936039 | 1.013381 |
| PCTDC_58 | −1.912733 | −2.253616 | −2.626545 | −1.998992 | −1.811559 |
| PCTDC_59 | −1.583744 | −2.902067 | −2.160966 | −2.559188 | −2.171652 |
| PCTDC_60 | −4.594235 | −9.801021 | −6.549976 | −8.366997 | −6.324865 |
| PCTDC_61 | −1.721365 | −2.107157 | −2.72991 | −2.706889 | −2.094767 |
| PCTDC_62 | −1.656653 | −2.405285 | −2.427855 | −1.90805 | −1.702626 |
| PCTDC_63 | −2.059103 | −1.267867 | −2.900948 | −5.406435 | −2.089989 |
| PCTDC_64 | −3.331235 | −3.260733 | −8.222388 | −2.997291 | −5.712786 |
| PCTDC_65 | −1.749293 | −1.990184 | −2.138188 | −2.393138 | −4.281384 |
| PCTDC_66 | −1.418086 | −2.319613 | −2.252599 | −1.721568 | −1.527662 |
| PCTDC_67 | −1.44361 | −2.3941 | −2.584812 | −2.320981 | −2.128469 |
| PCTDC_68 | −1.453052 | −3.169803 | −2.645681 | −3.19537 | −2.847229 |
| PCTDC_69 | −1.607833 | −2.533608 | −3.170008 | −3.630129 | −2.208691 |
| PCTDC_70 | −1.883712 | −4.296963 | −4.564299 | −5.013136 | −3.408126 |
| PCTDC_71 | −1.567635 | −2.335572 | −1.933825 | −2.3208 | −4.172569 |
| PCTDC_72 | −1.275921 | −1.417767 | −3.189466 | −2.712248 | −2.466872 |
| PCTDC_73 | −2.065163 | −1.510612 | −3.397659 | −2.772774 | −2.795055 |
| PCTDC_74 | −4.120387 | −8.364182 | −5.820191 | −8.033478 | −5.058536 |
| PCTDC_75 | −5.651796 | −2.522639 | −11.103881 | −8.209849 | −2.939723 |
| PCTDC_76 | −1.300726 | −2.012938 | −2.021577 | −2.267975 | −2.526409 |
| PCTDC_77 | 1.156325 | −2.42797 | −1.702881 | −2.322926 | −2.977733 |
| PCTDC_78 | −1.994662 | −4.118546 | −3.399733 | −4.519724 | −3.063789 |
| PCTDC_79 | −3.005263 | −3.964085 | −3.305238 | −6.023715 | −3.777092 |
| PCTDC_80 | −2.22871 | −2.531804 | −2.986517 | −2.009376 | 1.426795 |
| PCTDC_81 | −1.029887 | −2.659047 | −1.291147 | −1.465626 | −2.568156 |
| PCTDC_82 | −1.456901 | −2.558453 | −1.890956 | −1.654315 | −2.291716 |
| PCTDC_83 | −1.483869 | −1.774219 | −1.906184 | −2.040671 | −1.55863 |
| PCTDC_84 | −1.488962 | −1.738312 | −2.997032 | −3.06989 | −2.522394 |
| PCTDC_85 | −3.397085 | −1.800004 | −4.670069 | −5.907787 | −4.243998 |
| PCTDC_86 | −4.629498 | −5.268767 | −16.325615 | −5.504815 | −10.1651917 |
| PCTDC_87 | −2.965783 | −4.651131 | −4.426116 | −5.074196 | −3.048017 |
| PCTDC_88 | 1.176969 | −1.816881 | −2.221296 | −2.040161 | −3.435726 |
| PCTDC_89 | −4.648353 | −5.023491 | −16.478586 | −5.259194 | −10.594707 |
| PCTDC_90 | −3.987328 | −3.317574 | −10.907522 | −7.503156 | −7.047785 |
| PCTDC_91 | −4.017776 | −3.727969 | −10.129467 | −7.625894 | −6.47775 |
| PCTDC_92 | −1.55329 | −2.539011 | −2.670223 | −2.961054 | −2.324611 |
| PCTDC_93 | 1.239372 | −2.519451 | −1.614062 | −2.468781 | −2.956497 |
| PCTDC_94 | −1.568057 | −2.680018 | −2.582005 | −3.017979 | −2.11765 |
| PCTDC_95 | −1.838165 | −2.816427 | −3.051432 | −3.385318 | −2.692921 |
| PCTDC_96 | −2.54813 | −2.836381 | −3.19527 | −1.011483 | −1.329053 |
| PCTDC_97 | −1.734123 | −2.982916 | −2.245812 | −4.641543 | −2.431019 |
| PCTDC_98 | −1.863264 | −4.766246 | −2.206263 | −2.463233 | −2.087883 |
| PCTDC_99 | −2.330333 | −8.410342 | −5.218665 | −4.778626 | −4.32383 |
| PCTDC_100 | −1.487802 | −6.952064 | −3.395472 | −3.807495 | −5.184178 |
| PCTDC_101 | −2.103176 | −2.342718 | −3.634921 | −1.789127 | −3.305705 |
| PCTDC_102 | −3.95919 | −3.523541 | −15.429191 | −9.570858 | −11.63169 |
| PCTDC_103 | −1.597318 | −3.302787 | −1.97656 | −1.831488 | −2.571605 |
| PCTDC_104 | −2.179013 | −8.550433 | −4.822043 | −9.241587 | −8.620716 |
| PCTDC_105 | −1.186902 | −2.28356 | −1.916777 | −2.520292 | −4.656952 |
| PCTDC_106 | −1.574975 | −4.112132 | −2.460182 | −2.674212 | −2.620046 |
| PCTDC_107 | −1.285797 | −4.282806 | −2.194912 | −2.682937 | −2.991414 |
| PCTDC_108 | −1.536358 | −5.271469 | −2.617043 | −3.87438 | −5.142908 |
| PCTDC_109 | −1.098137 | −6.498635 | −2.601364 | −4.266908 | −7.241029 |
| PCTDC_110 | −5.741544 | −20.645773 | −8.901743 | −13.201766 | −9.474846 |
| PCTDC_111 | −3.784938 | −4.775443 | −13.710231 | −4.736529 | −9.753855 |
| PCTDC_112 | −3.770256 | −3.896379 | −13.006416 | −8.495649 | −9.174363 |
| PCTDC_113 | −3.182243 | −5.411848 | −3.122021 | −6.497146 | −5.564974 |
| PCTDC_114 | −1.430802 | −1.9482 | −1.666666 | −2.218147 | −1.823165 |
| PCTDC_115 | −1.466078 | −2.758297 | −2.215887 | −2.567012 | −4.594712 |
| PCTDC_116 | −1.510808 | −4.142076 | −2.876519 | −3.256964 | −2.992515 |
| PCTDC_117 | −3.690495 | −4.032526 | −12.182355 | −8.187602 | −9.412992 |
| PCTDC_118 | 3.702723 | −6.816492 | 1.465504 | −3.411267 | −5.558865 |
| PCTDC_119 | −1.699052 | −2.68473 | −3.509726 | −3.446977 | −3.184815 |
| PCTDC_120 | −2.18336 | 1.201436 | −5.29367 | −5.691936 | −5.728442 |
| PCTDC_121 | −4.335685 | −4.980919 | −17.322721 | −5.555618 | −13.936533 |
| PCTDC_122 | −3.943798 | −4.159578 | −20.717194 | −3.961968 | −13.265989 |
| PCTDC_123 | −4.220544 | −4.458473 | −16.951117 | −10.633861 | −11.06091 |
| PCTDC_124 | −1.876516 | −2.220029 | −2.524925 | −1.514067 | −1.913248 |
| PCTDC_125 | −4.156865 | −3.94276 | −15.586668 | −9.926511 | −11.538962 |
| PCTDC_126 | −4.275777 | −4.537924 | −15.922481 | −9.872524 | −11.471142 |
| PCTDC_127 | −1.158254 | −2.990276 | −2.605285 | −3.485199 | −3.114484 |
| PCTDC_128 | −4.151817 | −4.414096 | −17.50596 | −5.337328 | −13.212199 |
| PCTDC_129 | −4.137128 | −5.355405 | −15.638588 | −5.499931 | −11.5353 |
| PCTDC_130 | −2.287815 | −2.981069 | −2.060238 | −1.37465 | −1.877548 |
| PCTDC_131 | −1.387256 | −4.366215 | −2.788288 | −2.56777 | −2.021314 |
| PCTDC_132 | −3.80588 | −7.436905 | −4.451353 | −5.046357 | −3.825623 |
| PCTDC_133 | −2.73899 | −3.233242 | −6.990095 | −2.77934 | −5.504802 |
| PCTDC_134 | −2.891005 | −3.158316 | −5.96714 | −3.160015 | −4.867879 |
| PCTDC_135 | −1.486067 | −4.24235 | −2.986602 | 1.492916 | −3.130219 |
| PCTDC_136 | −2.07305 | −2.560523 | −2.921531 | −1.495705 | −2.227009 |
| PCTDC_137 | −6.709738 | −16.989042 | −8.53929 | −10.65459 | −8.351618 |
| PCTDC_138 | −2.49371 | −5.257616 | −2.912396 | −3.793311 | −2.631904 |
| PCTDC_139 | −3.092424 | −3.94329 | −7.177196 | −3.531577 | −5.426573 |
| PCTDC_140 | −2.155357 | −2.246449 | −2.258769 | −2.737894 | −2.926592 |
| PCTDC_141 | −2.443242 | −3.105939 | −4.254194 | −3.02592 | −3.054046 |
| PCTDC_142 | −2.753375 | −2.748035 | −5.325381 | −3.462896 | −4.431551 |
| PCTDC_143 | −1.801544 | −2.933015 | −2.477804 | −2.959098 | −2.615129 |
| PCTDC_144 | −2.065048 | −2.531502 | −3.04369 | −1.003939 | −1.357381 |
| PCTDC_145 | −2.326507 | −2.595412 | −4.664679 | −2.699193 | −3.707924 |
| PCTDC_146 | −2.906322 | −11.119669 | −4.22236 | −7.007662 | −4.997516 |
| PCTDC_147 | −3.452254 | −3.596967 | −10.395126 | −6.16955 | −7.261432 |
| PCTDC_148 | −1.380893 | −3.824272 | −2.648127 | −2.450598 | −2.995833 |
| PCTDC_149 | −3.255341 | −4.28165 | −9.910971 | −4.116663 | −7.631859 |
| PCTDC_150 | −3.171614 | −9.085684 | −4.92853 | −4.902088 | −5.884509 |
| PCTDC_151 | −3.698747 | −4.310469 | −15.277804 | −4.509701 | −10.009133 |
| PCTDC_152 | −4.23119 | −4.110284 | −20.236582 | −11.094976 | −12.877276 |
| PCTDC_153 | −2.712866 | −6.123426 | −3.677176 | −8.167227 | −3.230167 |
| PCTDC_154 | −1.471138 | −2.662261 | −2.621493 | −3.384066 | −3.308468 |
| PCTDC_155 | −2.993447 | −2.75769 | −5.764012 | −3.377901 | −5.71154 |
| PCTDC_156 | −1.974599 | −2.316575 | −2.065084 | −1.892309 | −1.669646 |
| PCTDC_157 | −13.417576 | −52.650387 | −14.037018 | −28.02939 | −13.408166 |
| PCTDC_158 | −1.553338 | −1.968661 | −2.381855 | −2.390924 | −2.822145 |
| PCTDC_159 | −1.276863 | −2.318608 | −2.180403 | −2.889938 | −2.466328 |
| PCTDC_160 | −3.548278 | −3.781662 | −7.474132 | −3.371794 | −6.091012 |
| PCTDC_161 | −1.527334 | −2.943324 | −3.102524 | −2.419431 | −2.648917 |
| PCTDC_162 | −3.127769 | −3.394859 | −6.636523 | −2.867695 | −4.432214 |
| PCTDC_163 | −2.006225 | −2.901773 | −2.346002 | −2.248675 | −2.00109 |
| PCTDC_164 | −1.405297 | −2.948033 | −2.934754 | −4.30079 | −3.859725 |
| PCTDC_165 | −1.588076 | −2.47727 | −2.414314 | −3.631488 | −3.532092 |
| PCTDC_166 | −2.931838 | −4.336748 | −3.24646 | −4.411035 | −2.980717 |
| PCTDC_167 | −3.570338 | −4.178797 | −7.782403 | −3.813373 | −5.671333 |
| PCTDC_168 | −2.688166 | −2.663015 | −4.319777 | −2.573629 | −3.171301 |
| PCTDC_169 | −2.058129 | −2.473203 | −2.952778 | −4.40996 | −3.264037 |
| PCTDC_170 | −2.169586 | −4.30589 | −3.911129 | −4.591723 | −2.672218 |
| PCTDC_171 | −2.225409 | −2.251348 | −2.092346 | −1.972791 | −2.16491 |
| PCTDC_172 | −1.669519 | −2.704463 | −2.297935 | −1.919122 | −1.941853 |
| PCTDC_173 | −2.048641 | −3.10866 | −2.673926 | −2.901284 | −3.034959 |
| PCTDC_174 | −2.081222 | −2.096023 | −2.054026 | −1.053168 | −1.085007 |
| PCTDC_175 | −4.774598 | −12.734313 | −8.883727 | −9.912612 | −8.323947 |
| PCTDC_176 | −1.258965 | −2.632138 | −1.913653 | −2.185067 | −2.483676 |
| PCTDC_177 | −1.939379 | −4.530967 | −2.359891 | −4.078347 | −2.764213 |
| PCTDC_178 | −2.055301 | −5.208098 | −2.614315 | −3.639303 | −3.584429 |
| PCTDC_179 | −2.900685 | −1.154608 | −2.03717 | −6.044252 | −2.34479 |
| PCTDC_180 | −1.415126 | −1.386274 | −1.791795 | −2.670586 | −2.395692 |
| PCTDC_181 | −1.714957 | −2.479498 | −2.910652 | −2.405157 | −3.129045 |
| PCTDC_182 | −6.934667 | −19.723858 | −12.640163 | −17.473091 | −12.771142 |
| PCTDC_183 | −2.414614 | −1.648399 | −1.437117 | −3.31257 | −2.936945 |
| PCTDC_184 | −2.78603 | −2.557558 | −4.300115 | −2.994413 | −3.691492 |
| PCTDC_185 | −1.525948 | −2.479607 | −2.37295 | −3.136964 | −3.129661 |
| PCTDC_186 | −1.246323 | −2.614822 | −2.054178 | −2.435119 | −1.90128 |
| PCTDC_187 | −1.816847 | −4.147898 | −2.062153 | −3.158015 | −2.938852 |
| PCTDC_188 | −4.396978 | −15.750997 | −7.304055 | −15.64049 | −11.103608 |
| PCTDC_189 | −2.94891 | −8.228148 | −5.483788 | −5.084009 | −4.167959 |
| PCTDC_190 | −1.768481 | −3.537523 | −2.037857 | −2.598359 | −2.147279 |
| PCTDC_191 | −2.4262 | −2.804561 | −1.730608 | −5.975543 | −4.760255 |
| PCTDC_192 | −1.741019 | −2.644385 | −3.044426 | −3.826642 | −2.357721 |
| PCTDC_193 | −4.156188 | −4.158795 | −18.791737 | −4.027184 | −9.387738 |
| PCTDC_194 | −5.13086 | −9.800389 | −4.697654 | −6.405068 | −6.736834 |
| PCTDC_195 | −1.81423 | −2.198194 | −1.737582 | −2.735552 | −2.158796 |
| PCTDC_196 | −5.080579 | −4.471194 | −7.231489 | −18.427435 | −9.806072 |
| PCTDC_197 | −2.324542 | −5.903867 | −3.229978 | −17.273748 | −6.400387 |
| PCTDC_198 | −1.932416 | −2.653923 | −3.757628 | −2.735156 | −1.817282 |
| PCTDC_199 | −3.623217 | −4.536964 | −8.831078 | −4.463152 | −6.401208 |
| PCTDC_200 | −2.100615 | −5.586963 | −1.843135 | −5.846531 | −3.071575 |
| PCTDC_201 | −2.379844 | −2.473764 | −2.870435 | −1.025683 | −1.222568 |
| PCTDC_202 | −1.911155 | −8.100895 | −3.645935 | −5.755246 | −7.075781 |
| PCTDC_203 | −2.278131 | −2.146923 | −2.403642 | −3.497723 | −2.42467 |
| PCTDC_204 | −5.798268 | −34.06509 | −12.273384 | −17.905043 | −34.228233 |
| PCTDC_205 | −1.469821 | −2.113142 | −2.577702 | −3.65754 | −2.8461 |
| PCTDC_206 | −1.890783 | −3.518015 | −2.87097 | −3.271979 | −3.320841 |
| PCTDC_207 | −2.473858 | −4.771787 | −4.502133 | −6.395139 | −6.571494 |
| PCTDC_208 | −1.2463 | −2.302852 | −1.63261 | −4.727072 | −3.248115 |
| PCTDC_209 | −5.330461 | −15.063501 | −11.671608 | −15.723096 | −11.474429 |
| PCTDC_210 | −1.569534 | −2.567336 | −2.081995 | −2.780438 | −2.044163 |
| PCTDC_211 | −3.454089 | −3.890421 | −7.289502 | −4.10517 | −6.346364 |
| PCTDC_212 | −1.814972 | −2.492545 | −2.12127 | −2.077773 | −1.769448 |
| PCTDC_213 | −1.928891 | −2.556381 | −2.162709 | −1.529102 | −1.17182 |
| PCTDC_214 | −2.837507 | −5.943567 | −1.049267 | −1.058182 | −4.723122 |
| PCTDC_215 | −3.338695 | −6.675632 | −3.509163 | −4.088458 | −3.590021 |
| PCTDC_216 | −1.727204 | −2.826245 | −1.483557 | −3.021882 | −2.146228 |
| PCTDC_217 | −1.819851 | −2.80383 | −2.80035 | −1.412471 | −1.811291 |
| PCTDC_218 | −1.678976 | −2.935515 | −2.474926 | −3.653771 | −2.784799 |
| PCTDC_219 | −2.652697 | −3.281955 | −1.350641 | −3.027711 | −3.31431 |
| PCTDC_220 | −3.300081 | −3.680073 | −8.556771 | −3.592223 | −5.737456 |
| PCTDC_221 | −4.794336 | −3.827177 | −21.278063 | −11.837326 | −11.247367 |
| PCTDC_222 | −4.288795 | −3.640255 | −17.291485 | −3.768052 | −9.4419 |
| PCTDC_223 | −2.298889 | −2.346835 | −2.776059 | −2.945078 | −2.02338 |
| PCTDC_224 | −3.46923 | −4.02571 | −9.015471 | −3.785506 | −6.03347 |
| PCTDC_225 | −4.385356 | −3.423778 | −20.784225 | −11.31271 | −10.954226 |
| PCTDC_226 | −4.607494 | −3.244585 | −20.744709 | −11.417688 | −10.372003 |
| PCTDC_227 | −1.352514 | −2.258559 | −2.182549 | −2.125192 | −2.144093 |
| PCTDC_228 | −3.884015 | −3.288769 | −12.3219 | −8.174881 | −7.634091 |
| PCTDC_229 | −4.333633 | −4.448777 | −16.964617 | −5.066412 | −9.85986 |
| PCTDC_230 | −4.194637 | −3.671257 | −18.332588 | −3.799327 | −10.502856 |
| PCTDC_231 | −4.70225 | −3.937051 | −19.540285 | −10.537539 | −10.05743 |
| PCTDC_232 | −2.128672 | −5.706282 | −4.30805 | −5.31519 | −5.17726 |
| PCTDC_233 | −1.519963 | −3.768478 | −2.549499 | −2.301235 | −2.82662 |
| PCTDC_234 | −1.264152 | −4.115586 | −4.426901 | −5.132766 | −2.441659 |
| PCTDC_235 | −10.398974 | −35.84404 | −37.890846 | −28.436022 | −40.04533 |
| PCTDC_236 | −1.935501 | −3.037173 | −1.855584 | −1.150821 | −2.050454 |
| PCTDC_237 | −1.274877 | −2.369792 | −2.281013 | −2.097712 | −2.207632 |
| PCTDC_238 | −1.913187 | −4.210544 | −2.658317 | −3.280815 | −3.646399 |
| PCTDC_239 | −1.841173 | −2.362227 | −2.348137 | −1.84079 | −2.010021 |
| PCTDC_240 | −1.687986 | −3.038711 | −2.072264 | −2.204134 | −1.849882 |
| PCTDC_241 | −2.218499 | −3.646227 | −2.971531 | −1.572821 | −1.789804 |
| PCTDC_242 | −2.039107 | −2.208811 | −2.28638 | −1.971873 | −2.074498 |
| PCTDC_243 | −1.318499 | −3.639507 | −2.84055 | −3.161946 | −3.535114 |
| PCTDC_244 | −1.748289 | −7.997188 | −3.286743 | −4.133771 | −4.60195 |
| PCTDC_245 | −1.832195 | −4.165719 | −2.28764 | −1.761182 | −2.417527 |
| PCTDC_246 | −1.595434 | −2.634676 | −2.23967 | −2.666174 | −2.305202 |
| PCTDC_247 | −1.349474 | −2.380194 | −1.999325 | −2.180002 | −2.680629 |
| PCTDC_248 | −3.88447 | −1.569351 | −4.786776 | −5.813709 | −4.900392 |
| PCTDC_249 | −4.527576 | −3.343985 | −19.883654 | −11.058222 | −10.261432 |
| PCTDC_250 | −3.840485 | −3.851627 | −10.762053 | −4.526831 | −8.764568 |
| PCTDC_251 | −2.679081 | −6.497992 | −3.447102 | −3.482586 | −5.451421 |
| PCTDC_252 | −1.821194 | −6.009986 | −1.983636 | −4.484305 | −4.349729 |
| PCTDC_253 | −1.516983 | −3.239355 | −2.960478 | −2.969269 | −2.921623 |
| PCTDC_254 | −3.804338 | −3.96088 | −2.255185 | −2.441548 | −4.56314 |
| PCTDC_255 | −1.348214 | −4.322912 | −2.479361 | −3.79677 | −10.963726 |
| PCTDC_256 | −1.42124 | −1.874196 | −1.572451 | −4.164904 | −2.942757 |
| PCTDC_257 | −1.935026 | −1.793478 | −2.261944 | −1.706943 | −1.84242 |
| PCTDC_258 | −2.164887 | −2.138947 | −2.635155 | 1.101481 | 1.001589 |
| PCTDC_259 | −1.462832 | −1.777587 | −1.151733 | −3.669216 | −5.133826 |
| PCTDC_260 | −2.675976 | 1.970285 | 2.396611 | −2.847489 | 2.130628 |
| PCTDC_261 | −1.368188 | −2.891441 | −1.965796 | −2.099375 | −2.274744 |
| PCTDC_262 | −2.032704 | −4.865805 | −3.031115 | −4.299381 | −3.680209 |
| PCTDC_263 | −1.670561 | −2.093797 | −1.586664 | −2.302789 | −2.545252 |
| PCTDC_264 | −2.172579 | −7.356969 | −4.616506 | −5.189599 | −4.719282 |
| PCTDC_265 | −2.993881 | −10.082272 | −5.890966 | −6.007321 | −7.178654 |
| PCTDC_266 | −2.065178 | −5.472369 | −2.766854 | −3.566468 | −4.449538 |
| PCTDC_267 | −1.508112 | −3.803641 | −2.603237 | −2.931435 | −4.010838 |
| PCTDC_268 | −1.700791 | −2.035907 | −1.889752 | −1.469181 | −1.708773 |
| PCTDC_269 | −2.379198 | −4.543904 | −2.730455 | −4.025674 | −4.433538 |
| PCTDC_270 | 1.069363 | −6.909536 | −1.596846 | −2.536486 | −5.071217 |
| PCTDC_271 | −3.260807 | −6.212852 | −4.560222 | −5.647288 | −3.819784 |
| PCTDC_272 | −1.492992 | −2.927971 | −1.627886 | −1.546699 | −2.915572 |
| PCTDC_273 | −1.438038 | −2.148547 | −2.178608 | −2.320278 | −2.776088 |
| PCTDC_274 | −2.247778 | −3.46386 | −2.572843 | −3.629104 | −3.713943 |
| PCTDC_275 | −1.003338 | −11.410631 | −1.151153 | −11.397514 | −5.726798 |
| PCTDC_276 | −4.217476 | −3.132212 | −19.35323 | −3.765451 | −10.062749 |
| PCTDC_277 | −2.995564 | −2.991639 | −5.738002 | −3.121268 | −4.617397 |
| PCTDC_278 | −1.36545 | −3.049989 | −2.161933 | −2.534092 | −3.634981 |
| PCTDC_279 | −2.094618 | −4.847474 | −3.21493 | −3.531053 | −3.276093 |
| PCTDC_280 | −1.693354 | −2.413334 | −2.310149 | −2.435938 | −2.811727 |
| PCTDC_281 | −1.588091 | −2.687367 | −2.350503 | −2.975265 | −2.875559 |
| PCTDC_282 | −2.549859 | −2.611744 | −2.844443 | −6.270815 | −5.109812 |
| PCTDC_283 | −1.131231 | −4.185311 | −1.866927 | −3.823021 | −5.471189 |
| PCTDC_284 | −2.617707 | −2.880339 | −4.826176 | −2.403895 | −3.537261 |
| PCTDC_285 | −1.262789 | −4.501634 | −2.47389 | −3.228784 | −2.666664 |
| PCTDC_286 | −2.165796 | −3.470174 | −2.341007 | −3.268555 | −2.995115 |
| PCTDC_287 | −1.278287 | −3.824776 | −2.377869 | −3.071996 | −2.574481 |
| PCTDC_288 | −1.875589 | −6.210633 | −3.432205 | −4.671096 | −3.117131 |
| PCTDC_289 | −2.192947 | −2.139038 | −2.965462 | 1.117521 | −1.113814 |
| PCTDC_290 | −2.611839 | −5.507403 | −5.024969 | −4.783923 | −4.289661 |
| PCTDC_291 | −2.479932 | −8.477123 | −5.651296 | −4.749075 | −4.153006 |
| PCTDC_292 | −1.532378 | −2.67058 | −2.009878 | −2.491136 | −2.341999 |
| PCTDC_293 | −6.433547 | −16.108952 | −9.149508 | −9.620059 | −6.977403 |
| PCTDC_294 | −4.070741 | −3.169832 | −5.821867 | −4.436994 | −7.281688 |
| PCTDC_295 | −1.457103 | −2.545094 | −1.963598 | −2.240566 | −2.224381 |
| PCTDC_296 | −2.703047 | −7.823654 | −4.022834 | −5.51972 | −4.912844 |
| PCTDC_297 | −3.034877 | −5.773628 | −4.317933 | −4.100777 | −3.488915 |
| PCTDC_298 | −1.795715 | −1.93728 | −2.387764 | −2.474603 | −2.129225 |
| PCTDC_299 | −1.333848 | −6.838379 | −2.556827 | −2.964774 | −3.187869 |
| PCTDC_300 | −1.224164 | −1.913381 | −2.007162 | −2.272181 | −3.411546 |
| PCTDC_301 | −1.612864 | 1.421686 | 1.108202 | −5.495829 | −2.529128 |
| PCTDC_302 | −3.375747 | 1.517615 | −1.047322 | 1.03283 | −1.205198 |
| PCTDC_303 | −2.98236 | −2.123022 | −4.972325 | −2.771994 | −4.887907 |
| PCTDC_304 | −3.748991 | −8.648803 | −4.297132 | −4.222451 | −4.112308 |
| PCTDC_305 | −2.067976 | −3.095366 | −3.241502 | −4.974222 | −4.456541 |
| PCTDC_306 | −3.433239 | −3.852857 | −8.218064 | −3.541728 | −5.608873 |
| PCTDC_307 | −1.510144 | −2.174652 | −1.960471 | −2.305311 | −2.085746 |
| PCTDC_308 | −5.709903 | −7.506292 | −21.021177 | −18.16979 | −15.282486 |
| PCTDC_309 | −2.538695 | −5.427696 | −3.96977 | −5.34938 | −3.434406 |
| PCTDC_310 | −8.351592 | −15.544457 | −6.618097 | −9.989495 | −7.883212 |
| PCTDC_311 | −1.568636 | −2.643091 | −1.923311 | −2.491152 | −2.016061 |
| PCTDC_312 | 1.07741 | −2.589733 | −2.607608 | −2.462168 | −2.47417 |
| PCTDC_313 | −1.149116 | −2.445372 | −1.996363 | −2.240741 | −2.395751 |
| PCTDC_314 | −1.83386 | −4.455273 | −2.660103 | −3.904015 | −3.88134 |
| PCTDC_315 | −4.020957 | −3.663386 | −11.000549 | −7.362528 | −6.767858 |
| PCTDC_316 | −1.129105 | −3.416999 | −2.177473 | −2.653117 | −2.919225 |
| PCTDC_317 | −1.322192 | −2.006308 | −2.169702 | −2.077435 | −2.53624 |
| PCTDC_318 | −1.068702 | −3.100096 | −2.509806 | −3.332818 | −2.666414 |
| PCTDC_319 | −1.447103 | −7.838775 | −3.137043 | −3.309639 | −6.645592 |
| PCTDC_320 | −1.517501 | −2.395785 | −2.585518 | −2.073397 | −4.045468 |
| PCTDC_321 | −1.211345 | −2.963809 | −2.20423 | −2.320638 | −3.414114 |
| PCTDC_322 | −1.753529 | −4.687747 | −2.546606 | −2.777194 | −2.816231 |
| PCTDC_323 | −1.74737 | −4.210407 | −2.178987 | −3.938169 | −4.408377 |
| PCTDC_324 | −3.110309 | −4.810374 | −3.284321 | −4.389349 | −4.177819 |
| PCTDC_325 | −3.492937 | −7.161442 | −4.917929 | −5.236836 | −5.740018 |
| PCTDC_326 | −2.239092 | −2.973208 | −2.988244 | −1.376134 | −1.895988 |
| PCTDC_327 | −1.870174 | −2.171522 | −1.891829 | −1.711816 | −2.050615 |
| PCTDC_328 | −3.433633 | −3.484589 | −9.24229 | −3.640446 | −5.59146 |
| PCTDC_329 | −3.235591 | −4.306441 | −2.874235 | −3.416734 | −3.269137 |
| PCTDC_330 | −3.690394 | −3.485615 | −11.029022 | −6.335642 | −6.479433 |
| PCTDC_331 | −3.417982 | −2.846125 | −7.170537 | −2.894992 | −5.80286 |
| PCTDC_332 | −1.123622 | −3.094638 | −1.961519 | −3.331235 | −2.068638 |
| PCTDC_333 | −1.905308 | −2.71019 | −2.492548 | −2.638774 | −2.736861 |
| PCTDC_334 | −3.391635 | −2.57456 | −11.102236 | −5.849842 | −6.251497 |
| PCTDC_335 | −1.319804 | −2.484284 | −1.942393 | −2.102638 | −2.640838 |
| PCTDC_336 | −3.612695 | −2.795166 | −11.60204 | −3.320234 | −7.553721 |
| PCTDC_337 | −3.64587 | −3.302513 | −11.300043 | −3.332641 | −6.622171 |
| PCTDC_338 | −2.036446 | −2.547056 | −2.315574 | −1.672926 | −1.673005 |
| PCTDC_339 | −3.058439 | −2.272604 | −7.023274 | −2.677456 | −4.512324 |
| PCTDC_340 | −2.827927 | −2.610447 | −5.930333 | −2.285218 | −3.446473 |
| PCTDC_341 | −3.741079 | −2.618799 | −11.512197 | −6.588821 | −6.15581 |
| PCTDC_342 | −3.831348 | −2.693085 | −15.116555 | −3.094456 | −8.320878 |
| PCTDC_343 | −1.699405 | −2.993683 | −3.541289 | −2.123977 | −2.629834 |
| PCTDC_344 | −3.803385 | −2.496838 | −1.944487 | −1.251222 | −1.08661 |
| PCTDC_345 | −4.892186 | −2.066659 | −1.488773 | −2.048109 | 1.271424 |
| PCTDC_346 | −2.01679 | −2.0381 | −1.794273 | −1.987858 | −1.776335 |
| PCTDC_347 | −3.61717 | −4.590827 | −3.510527 | −3.444241 | −2.840451 |
| PCTDC_348 | −1.163732 | −1.765558 | −2.098776 | −2.404756 | −3.516867 |
| PCTDC_349 | −3.776194 | −8.061971 | −3.085753 | 2.783463 | −4.163679 |
| PCTDC_350 | −1.74484 | −1.054856 | −2.273646 | −1.22114 | −2.518731 |
| PCTDC_351 | −2.126303 | −1.074242 | −2.7906 | −1.168993 | −2.61287 |
| PCTDC_352 | −3.276581 | −8.238791 | −5.228536 | −5.113611 | −4.634111 |
| PCTDC_353 | −1.569322 | −2.203796 | −2.300594 | −2.143878 | −2.061727 |
| PCTDC_354 | −1.148932 | −2.006905 | −2.270097 | −2.420032 | −2.935613 |
| PCTDC_355 | −1.382682 | −2.570274 | −2.082243 | −2.0819 | −2.80178 |
| PCTDC_356 | −2.5360111 | −1.625364 | −2.540703 | −6.342539 | −2.618487 |
| PCTDC_357 | −2.200665 | −1.738183 | −3.051713 | −2.957093 | −3.627696 |
| PCTDC_358 | −2.949249 | −2.735868 | −6.206738 | −3.133926 | −4.475304 |
| PCTDC_359 | −3.461999 | −2.455908 | −11.913185 | −7.039542 | −6.29104 |
| PCTDC_360 | −2.771786 | −4.532234 | −3.035789 | −3.96584 | −4.225999 |
| PCTDC_361 | −2.112897 | −2.089313 | −2.453935 | −1.761781 | −1.766528 |
| PCTDC_362 | −1.370505 | −2.758603 | −2.459529 | −3.364921 | −3.779385 |
| PCTDC_363 | −1.640921 | −3.121555 | −2.376389 | −2.138602 | −2.144983 |
| PCTDC_364 | −1.83666 | −2.059931 | −3.211289 | −2.022543 | −4.047491 |
| PCTDC_365 | −1.263447 | −16.474352 | −3.833677 | −3.515598 | −1.455502 |
| PCTDC_366 | −1.962621 | −2.683585 | −1.982845 | −2.539793 | −2.131283 |
| PCTDC_367 | −1.754204 | −3.520776 | −1.917178 | −2.957508 | −3.464419 |
| PCTDC_368 | −1.539361 | −2.633818 | −2.408538 | −2.715942 | −2.475481 |
| PCTDC_369 | −1.684391 | −3.190459 | −2.843073 | −2.785024 | −2.324296 |
| PCTDC_370 | −1.065779 | −3.231136 | −2.119581 | −2.370553 | −3.640322 |
| PCTDC_371 | −4.206266 | −4.134809 | −2.572417 | −4.510403 | −3.98005 |
| PCTDC_372 | −1.497509 | −3.433379 | −2.431221 | −2.62591 | −2.364552 |
| PCTDC_373 | −2.701431 | −5.541125 | −3.740372 | −4.711283 | −5.363891 |
| PCTDC_374 | −2.062943 | −2.468417 | −2.218792 | −3.703695 | −4.524557 |
| PCTDC_375 | −1.677184 | −3.231339 | −2.047146 | −2.547986 | −2.687473 |
| PCTDC_376 | −2.772744 | −4.423054 | −2.28992 | −4.693399 | −4.732909 |
| PCTDC_377 | −2.563527 | −4.492982 | −3.069068 | −4.294678 | −4.562173 |
| PCTDC_378 | −2.282956 | −2.071853 | −1.218522 | −3.795834 | −2.348081 |
| PCTDC_379 | −3.556599 | −3.71274 | −9.53263 | −3.796503 | −6.069323 |
| PCTDC_380 | −2.849799 | 2.296765 | 1.223609 | −4.261389 | −2.095827 |
| PCTDC_381 | −4.106955 | −3.716886 | −13.196062 | −4.619672 | −8.85029 |
| PCTDC_382 | −3.647153 | −2.899484 | −12.142699 | −6.762471 | −6.210923 |
| PCTDC_383 | −3.781007 | −2.757492 | −12.215237 | −7.54418 | −6.675462 |
| PCTDC_384 | −2.579501 | −2.90819 | −2.191753 | −2.543473 | −2.438313 |
| PCTDC_385 | −1.83999 | −3.014101 | −2.088584 | −1.839071 | −2.999907 |
| PCTDC_386 | −3.633339 | −2.769401 | −10.876658 | −6.694985 | −6.252499 |
| PCTDC_387 | −3.199733 | −3.12726 | −8.164774 | −2.955156 | −4.983577 |
| PCTDC_388 | −3.747109 | −9.926229 | −4.492403 | −4.01534 | −4.989985 |
| PCTDC_389 | −3.158527 | −2.956561 | −7.561758 | −2.747388 | −4.7964 |
| PCTDC_390 | −2.548517 | −2.250637 | −5.084722 | −2.436353 | −4.205762 |
| PCTDC_391 | −1.793494 | −3.306055 | −2.495369 | −2.562027 | −1.9559 |
| PCTDC_392 | −1.988252 | −3.171811 | −2.27029 | −2.387262 | −1.971462 |
| PCTDC_393 | −1.131981 | −2.47366 | −1.950791 | −2.979702 | −5.808935 |
| PCTDC_394 | −7.902956 | −16.22966 | −24.564844 | −23.351175 | −24.049793 |
| PCTDC_395 | −1.128561 | −5.602054 | −2.269211 | −4.024747 | −7.990259 |
| PCTDC_396 | −1.128652 | −2.310545 | −1.895955 | −2.949045 | −3.943671 |
| PCTDC_397 | −1.957875 | −1.435545 | −2.289376 | −2.088355 | −1.497819 |
| PCTDC_398 | −1.463656 | −2.536437 | −2.327757 | −3.223704 | −2.32261 |
| PCTDC_399 | −1.57208 | −2.217453 | −2.250325 | −2.891168 | −3.103871 |
| PCTDC_400 | 1.350873 | −3.66434 | −1.566388 | −2.472184 | −7.061352 |
| PCTDC_401 | −1.778172 | −3.41019 | −2.286762 | −2.406163 | −3.021344 |
| PCTDC_402 | −1.779176 | −2.560131 | −2.858688 | −4.338165 | −7.435108 |
| PCTDC_403 | −1.618405 | −2.691217 | −2.074394 | −2.555042 | −2.342018 |
| PCTDC_404 | −1.801164 | −2.295932 | −2.429118 | −3.811723 | −6.599621 |
| PCTDC_405 | −1.866313 | −2.623849 | −2.376353 | −5.042603 | −5.362969 |
| PCTDC_406 | −2.968811 | −5.255373 | −4.357278 | −5.601061 | −7.791493 |
| PCTDC_407 | −2.518974 | −20.609203 | −4.392604 | −6.316997 | −2.722303 |
| PCTDC_408 | −1.581567 | −3.825832 | −5.100918 | −3.272011 | −4.100996 |
| PCTDC_409 | −4.205354 | −4.244617 | −17.269669 | −5.153924 | −9.591729 |
| PCTDC_410 | −2.744054 | −2.665254 | −5.23113 | −2.69788 | −3.688508 |
| PCTDC_411 | −1.378731 | −3.97056 | −2.251723 | −4.60925 | −5.852931 |
| PCTDC_412 | −2.345133 | −15.503913 | −12.429821 | −19.225355 | −16.954866 |
| PCTDC_413 | −1.65605 | −3.256246 | −3.128621 | −2.342679 | −3.900469 |
| PCTDC_414 | 1.740423 | −3.666911 | −2.733058 | −2.509844 | −2.838648 |
| PCTDC_415 | −3.304775 | −9.66715 | −5.797694 | −7.218999 | −9.178728 |
| PCTDC_416 | −1.816274 | −4.544723 | −3.432478 | −13.144384 | −9.472692 |
| PCTDC_417 | −2.147236 | −3.477573 | −2.670616 | −2.360998 | −2.176139 |
| PCTDC_418 | −4.023874 | −3.216345 | −17.795725 | −3.685657 | −9.244202 |
| PCTDC_419 | −1.132564 | −3.282859 | −1.452853 | −2.953099 | −4.224542 |
| PCTDC_420 | 3.835113 | −7.88019 | 1.879768 | −1.602296 | −7.187496 |
| PCTDC_421 | −1.577877 | −1.032754 | −1.617107 | −2.510909 | −1.548298 |
| PCTDC_422 | −6.743957 | −10.768804 | −10.759794 | −14.728729 | −15.24783 |
| PCTDC_423 | −1.16685 | −3.696149 | −2.139006 | −4.231594 | −4.755056 |
| PCTDC_424 | −1.983778 | −3.899375 | −2.577879 | −2.61241 | −4.033377 |
| PCTDC_425 | −1.723426 | −11.907057 | −2.609665 | −6.127047 | −12.738211 |
| PCTDC_426 | −1.578315 | −3.836931 | −2.091437 | −3.109696 | −3.228948 |
| PCTDC_427 | −2.820146 | −5.154231 | −4.331721 | −5.67382 | −4.049896 |
| PCTDC_428 | −1.095983 | −3.818708 | −1.517295 | −5.526316 | −3.639863 |
| PCTDC_429 | −4.286278 | −2.442652 | −6.461126 | −6.962633 | −2.643682 |
| PCTDC_430 | 4.064054 | −6.751593 | 1.581492 | −2.944198 | −7.217455 |
| PCTDC_431 | −3.300669 | −3.426686 | −8.326731 | −3.519214 | −4.977574 |
| PCTDC_432 | −1.873488 | −3.154468 | −2.206955 | −1.948518 | −3.039134 |
| PCTDC_433 | −2.01857 | −3.910663 | −2.551511 | −3.928213 | −4.674957 |
| PCTDC_434 | −1.390829 | −2.92367 | −2.333632 | −2.249653 | −2.199577 |
| PCTDC_435 | −4.404173 | −4.128571 | −17.027718 | −9.717108 | −9.731349 |
| PCTDC_436 | −1.261343 | −1.759856 | −1.927743 | −2.225657 | −3.104023 |
| PCTDC_437 | −3.464413 | −3.505827 | −9.926498 | −4.018203 | −6.815197 |
| PCTDC_438 | −2.508386 | −5.663525 | −1.898467 | −3.320766 | −2.685038 |
| PCTDC_439 | −2.404006 | −2.682554 | −2.355178 | −2.451406 | −2.047588 |
| PCTDC_440 | −1.364202 | −2.640613 | −2.060328 | −2.039139 | −2.62871 |
| PCTDC_441 | −1.457507 | −2.585803 | −1.890205 | −2.121005 | −2.109571 |
| PCTDC_442 | −2.25791 | −3.318656 | −3.370878 | −1.485723 | −2.232206 |
| PCTDC_443 | −4.522355 | −14.677288 | −5.488232 | −9.310837 | −7.388904 |
| PCTDC_444 | −2.673374 | −6.160846 | −4.838342 | −2.959502 | −6.198854 |
| PCTDC_445 | −3.292587 | −10.502336 | −10.050386 | −3.505228 | −10.975809 |
| PCTDC_446 | −1.72304 | −2.456413 | −1.992708 | −2.33442 | −2.443974 |
| PCTDC_447 | −1.525621 | −2.269687 | −1.663389 | −2.176325 | −1.870524 |
| PCTDC_448 | −1.138005 | −1.9805 | −1.965061 | −2.759055 | −2.718367 |
| PCTDC_449 | 1.247501 | −2.14472 | −1.708293 | −2.419002 | −2.378231 |
| PCTDC_450 | −1.238025 | −3.797982 | −2.002429 | −2.575729 | −6.129398 |
| PCTDC_451 | −2.319706 | −2.740891 | −2.636069 | −1.662029 | −1.269787 |
| PCTDC_452 | −1.103615 | −1.966168 | −1.985103 | −2.362963 | −2.419588 |
| PCTDC_453 | −1.377476 | −3.19715 | −1.681339 | −3.330876 | −2.699991 |
| PCTDC_454 | −2.107279 | −2.543648 | −2.400486 | −2.117301 | −2.781379 |
| PCTDC_455 | 1.084275 | −2.359633 | −1.907869 | −2.50018 | −2.821481 |
| PCTDC_456 | −1.352769 | −3.292715 | −2.280744 | −2.313621 | −2.07801 |
| PCTDC_457 | −3.244624 | −3.680325 | −9.38241 | −4.090302 | −5.999517 |
| PCTDC_458 | −3.411826 | −3.752882 | −9.920869 | −4.132503 | −6.257953 |
| PCTDC_459 | −3.277509 | −3.452338 | −8.875525 | −3.475946 | −5.319915 |
| PCTDC_460 | −3.497402 | −3.646024 | −9.96344 | −4.267687 | −7.248205 |
| PCTDC_461 | −4.164809 | −3.766206 | −15.002592 | −9.621099 | −8.862043 |
| PCTDC_462 | −2.854793 | −3.078645 | −6.668007 | −3.693722 | −6.207499 |
| PCTDC_463 | −2.439668 | −3.133138 | −6.110227 | −3.292491 | −4.770925 |
| PCTDC_464 | −2.221495 | −2.100314 | −3.848471 | −2.619261 | −2.567279 |
| PCTDC_465 | −2.458162 | −2.530385 | −6.778717 | −4.317293 | −3.369215 |
| PCTDC_466 | −1.572988 | −2.917847 | −2.465304 | −2.271055 | −4.42402 |
| PCTDC_467 | −2.167579 | −2.072933 | −3.064006 | −2.583501 | −2.76667 |
| PCTDC_468 | −2.842929 | −1.416433 | −1.290887 | −4.525883 | −3.491836 |
| PCTDC_469 | −1.438455 | −2.230007 | −1.712926 | −1.977794 | −2.058432 |
| PCTDC_470 | −1.927791 | −4.439088 | −3.31788 | −2.961011 | 1.182987 |
| PCTDC_471 | −1.748365 | −4.446304 | −2.649345 | −3.490811 | −4.192489 |
| PCTDC_472 | 1.03894 | 1.027216 | −3.45271 | −2.19805 | −3.567147 |
| PCTDC_473 | −1.171918 | −3.867051 | −1.335986 | −3.011726 | −6.024663 |
| PCTDC_474 | −3.128487 | −5.867784 | −3.845832 | −3.756094 | −4.628043 |
| PCTDC_475 | −2.470888 | −3.857581 | −3.838137 | −3.065844 | −2.988186 |
| PCTDC_476 | −1.222385 | −2.412448 | −2.36894 | −2.051659 | −2.843233 |
| PCTDC_477 | −1.575935 | −3.358423 | −1.957228 | −4.20353 | −5.172051 |
| PCTDC_478 | −1.946568 | −2.538936 | −2.167247 | −1.973462 | −2.095788 |
| PCTDC_479 | −3.227538 | −3.466124 | −8.528288 | −3.727987 | −6.107713 |
| PCTDC_480 | −1.596215 | −2.417819 | −2.582085 | −2.210896 | −2.703929 |
| PCTDC_481 | −3.057556 | −3.262084 | −7.538472 | −3.32997 | −4.996814 |
| PCTDC_482 | −1.332372 | −5.460417 | −2.407066 | −3.417602 | −5.091409 |
| PCTDC_483 | −2.9404 | −3.102393 | −1.634451 | −1.067689 | 1.100071 |
| PCTDC_484 | −1.224315 | −2.444646 | −2.167004 | −2.873597 | −4.347968 |
| PCTDC_485 | −1.956571 | −3.849904 | −2.381093 | −3.594158 | −3.283784 |
| PCTDC_486 | −1.156493 | −2.355411 | −2.087242 | −2.743011 | −2.741241 |
| PCTDC_487 | −1.089697 | −2.300259 | −1.924453 | −2.163176 | −2.667205 |
| PCTDC_488 | −1.21457 | −2.370535 | −2.094783 | −2.922597 | −4.340714 |
| PCTDC_489 | −4.917046 | −8.368398 | −4.67966 | −5.141819 | −3.87472 |
| PCTDC_490 | −2.486346 | −1.86256 | −1.67286 | −1.805379 | −1.350162 |
| PCTDC_491 | −1.42621 | −2.36949 | −1.819114 | −2.279974 | −3.299198 |
| PCTDC_492 | −2.250829 | −13.135315 | −8.658033 | −10.866399 | −11.625659 |
| PCTDC_493 | 1.001932 | −4.354498 | −1.930646 | −2.972657 | −5.828303 |
| PCTDC_494 | −4.708869 | −8.360419 | −5.433639 | −11.695395 | −7.803391 |
| PCTDC_495 | −6.488033 | −15.314687 | −6.099616 | −10.37993 | −5.293031 |
| PCTDC_496 | −1.691502 | −3.558874 | −4.366205 | −3.123582 | −3.897883 |
| PCTDC_497 | −3.464158 | −3.641393 | −9.802439 | −3.830014 | −6.246689 |
| PCTDC_498 | −1.79345 | −2.961699 | −2.436046 | −1.53983 | 1.072635 |
| PCTDC_499 | −3.888875 | −3.158231 | −12.658903 | −3.36037 | −7.937511 |
| PCTDC_500 | −3.521348 | −3.002847 | −10.687489 | −3.434129 | −7.020942 |
| PCTDC_501 | −1.868472 | −3.872467 | −2.818968 | −2.871452 | −4.185874 |
| PCTDC_502 | −2.307466 | 1.134177 | −1.232951 | −1.094307 | −1.509788 |
| PCTDC_503 | −1.379813 | −2.539931 | −1.973483 | −2.806978 | −2.619855 |
| PCTDC_504 | −2.9028 | −2.780434 | −6.381547 | −3.298102 | −5.283781 |
| PCTDC_505 | −4.801282 | −11.344074 | −5.839832 | −10.373031 | −5.292884 |
| PCTDC_506 | −1.191914 | −2.479965 | −2.444037 | −2.647597 | −2.140528 |
| PCTDC_507 | −4.26957 | −3.998628 | −20.232414 | −11.585198 | −12.86114 |
| PCTDC_508 | −1.914974 | −4.588605 | −2.526306 | −4.673424 | −3.409321 |
| PCTDC_509 | −3.287681 | −9.000772 | 4.655367 | −13.595597 | −4.621663 |
| PCTDC_510 | −1.287828 | −3.334848 | −3.086293 | −4.46172 | −3.106186 |
| PCTDC_511 | −1.546756 | −3.090829 | −2.238058 | −2.172862 | −2.05809 |
| PCTDC_512 | −3.542684 | −3.158082 | −12.497798 | −24.719374 | −4.924631 |
| PCTDC_513 | −4.163492 | −4.528132 | −23.560839 | −88.310661 | −9.291848 |
| PCTDC_514 | −3.902461 | −3.665555 | −20.874205 | −74.998177 | −6.763952 |
| PCTDC_515 | −2.058109 | −7.408581 | −2.21213 | −3.995366 | −3.363424 |
| PCTDC_516 | −1.330293 | −2.353537 | −2.324155 | −2.567971 | −2.820413 |
| PCTDC_517 | −2.037686 | −4.844729 | −2.684412 | −2.387803 | −2.425425 |
| PCTDC_518 | −2.044653 | −5.675608 | −2.003303 | −5.242962 | −5.370054 |
| PCTDC_519 | −10.524405 | −26.187635 | −12.124947 | −8.147792 | −19.470036 |
| PCTDC_520 | −2.032907 | −3.105246 | 1.947639 | −3.280555 | −2.722996 |
| PCTDC_521 | −2.104322 | −2.855173 | −4.322774 | −2.842998 | −5.603982 |
| PCTDC_522 | −1.117974 | −3.012714 | −2.165339 | −3.430023 | −4.755522 |
| PCTDC_523 | −2.291559 | −3.577276 | −2.483721 | −2.613158 | −2.39375 |
| PCTDC_524 | −3.104864 | −2.932681 | −5.816575 | −2.131141 | −2.879459 |
| PCTDC_525 | −2.59016 | −2.453159 | −1.29434 | −4.881182 | −2.744209 |
| PCTDC_526 | −1.186716 | −2.263018 | −2.14326 | −2.677976 | −4.123821 |
| PCTDC_527 | −3.758474 | −3.695833 | −11.288051 | −6.075418 | −6.068587 |
| PCTDC_528 | −1.392221 | −2.307213 | −2.355418 | −2.664037 | −1.883761 |
| PCTDC_529 | −3.404735 | −3.16146 | −9.616374 | −3.619349 | −6.840977 |
| PCTDC_530 | −1.291287 | −4.9881 | −2.314329 | −2.677203 | −6.71598 |
| PCTDC_531 | −1.167032 | −4.373553 | −2.174679 | −2.273024 | −6.779548 |
| PCTDC_532 | −1.769813 | −2.953695 | −3.218176 | −2.363033 | −2.318671 |
| PCTDC_533 | −2.571501 | −4.802047 | −3.536669 | −3.118577 | −2.818208 |
| PCTDC_534 | −1.273614 | −1.930238 | −1.540489 | −2.00633 | −2.243512 |
| PCTDC_535 | −1.55068 | −2.843627 | −2.637512 | −3.457897 | −3.254817 |
| PCTDC_536 | −1.305724 | −2.593612 | −2.25954 | −1.919988 | −2.160357 |
| PCTDC_537 | −4.299753 | −3.52406 | −19.381191 | −3.533262 | −10.068835 |
| PCTDC_538 | −1.352274 | −3.180976 | −2.307213 | −3.092135 | −3.120386 |
| PCTDC_539 | −1.645554 | −7.03511 | −6.858676 | −2.140382 | −6.644153 |
| PCTDC_540 | −1.500036 | −3.543043 | −2.433013 | −2.404769 | −1.862214 |
| PCTDC_541 | −2.316446 | −4.198337 | −3.335201 | −3.99356 | −3.080166 |
| PCTDC_542 | −2.981153 | −2.738776 | −6.353964 | −2.783772 | −4.630699 |
| PCTDC_543 | −1.82209 | −3.629715 | −2.379959 | −3.298835 | −3.24351 |
| PCTDC_544 | −3.161205 | −7.419415 | −4.592446 | −6.918106 | −6.446481 |
| PCTDC_545 | −2.349786 | −4.703971 | −3.517354 | −2.734774 | −2.279695 |
| PCTDC_546 | −4.128414 | −5.972315 | −6.127929 | −7.578036 | −5.788638 |
| PCTDC_547 | −1.716096 | −2.021892 | −1.823375 | −1.964153 | −1.837394 |
| PCTDC_548 | −2.282952 | −1.362833 | −2.893267 | −1.174093 | −2.488058 |
| PCTDC_549 | −1.267364 | −2.617886 | −2.139586 | −3.319463 | −2.148385 |
| PCTDC_550 | −3.298179 | −2.67091 | −6.901036 | −3.05029 | −6.368939 |
| PCTDC_551 | −1.616773 | −3.305795 | −1.547398 | −3.197367 | −3.784653 |
| PCTDC_552 | −1.677305 | −3.195515 | −2.50823 | −2.247194 | −2.804085 |
| PCTDC_553 | −1.56604 | −2.19495 | −2.009058 | −1.910682 | −2.605141 |
| PCTDC_554 | −1.314985 | −2.044226 | −2.369961 | −2.089493 | −1.931527 |
| PCTDC_555 | −3.648843 | −7.010541 | −5.203244 | −6.406971 | −5.155677 |
| PCTDC_556 | −4.482344 | −5.974912 | −4.990507 | −4.360383 | −3.330212 |
| PCTDC_557 | −5.09733 | −4.356937 | −15.4079 | −22.830832 | −12.213466 |
| PCTDC_558 | −1.879507 | −3.64272 | −2.36905 | −3.298157 | −3.581275 |
| PCTDC_559 | −1.201338 | −4.644512 | −2.458729 | −1.756415 | −4.717901 |
| PCTDC_560 | −2.377509 | −4.225865 | −3.607449 | −3.226445 | −3.282101 |
| PCTDC_561 | −12.009212 | −30.645575 | −10.47272 | −14.096417 | −9.249123 |
| PCTDC_562 | 1.116945 | −3.024733 | −1.926901 | −1.925363 | −2.04705 |
| PCTDC_563 | −1.095156 | −3.6972 | −2.891324 | −3.374032 | −2.836388 |
| PCTDC_564 | −2.084297 | −4.547463 | −3.041965 | −3.241731 | −3.54576 |
| PCTDC_565 | −1.825188 | −2.484365 | −1.917063 | −1.932943 | −1.893031 |
| PCTDC_566 | −1.18682 | −2.648138 | −1.998425 | −2.664293 | −4.492978 |
| PCTDC_567 | −1.59086 | −2.869549 | −2.669139 | −2.646847 | −2.239221 |
| PCTDC_568 | −1.414268 | −2.792088 | −1.331242 | −3.16828 | −3.233897 |
| PCTDC_569 | −2.230147 | −1.457797 | 1.069658 | −1.722803 | −2.220284 |
| PCTDC_570 | −6.931448 | −44.141563 | −11.76804 | −19.895657 | −37.167835 |
| PCTDC_571 | −1.873176 | −2.435471 | −2.313335 | −3.99035 | −6.524507 |
| PCTDC_572 | −1.225797 | −3.97152 | −1.70684 | −3.714783 | −4.567413 |

### Example 6

Signal P Analysis and TMHMM Analysis

Transcripts that were differentially expressed greater than 2 fold and in 30% or more of the tumors were further analyzed by Signal P and TMHMM analysis to determine if they were membrane associated.

Signal Sequence and TMHMM Analysis

SignalP V2.0 comprises two signal peptide prediction methods, SignalP-NN (based on neural networks, corresponding to SignalP V1.1) and SignalP-HMM (based on a hidden Markov model). For eukaryotic data, SignalP-HMM has been shown to have substantially improved discrimination between signal peptides and uncleaved signal anchors, but it has a slightly lower accuracy in predicting the precise location of the cleavage site (Nielsen and Krogh, In Proceedings of the Sixth International Conference on Intelligent Systems for Molecular Biology, (1998). AAAI Press, Menlo Park, Calif., pp. 122-130). Since in all proteins examined, the predicted site of signal cleavage was exactly the same for both programs only the results of SignalP-HMM program are shown. TMHMM is membrane protein topology prediction program based on a HMM. This program has been shown to correctly predict 97-98% of the transmembrane helices (Krogh et al., J Mol Bio, 305:567-580(2001).). Additionally, TMHMM can discriminate between soluble and membrane proteins with both a greater than 99% specificity and sensitivity.

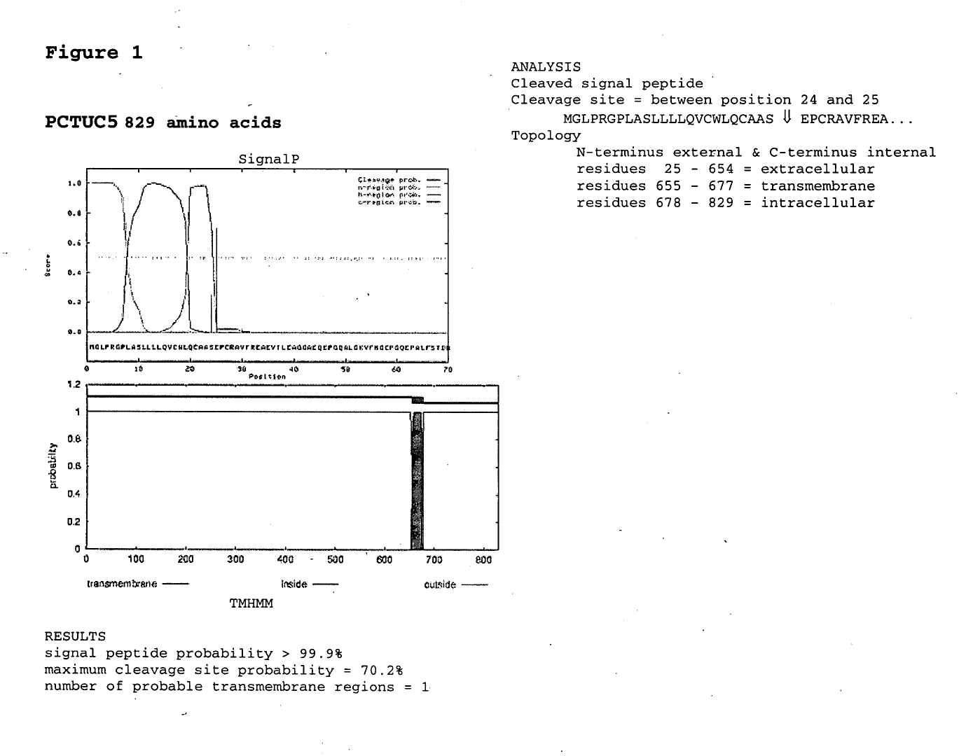

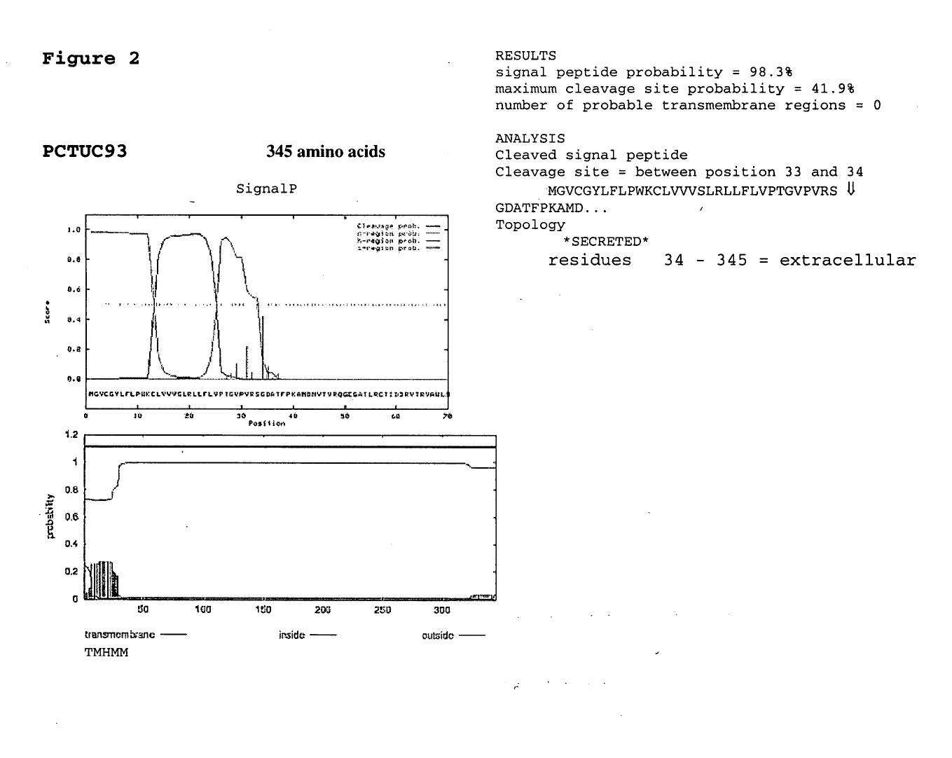

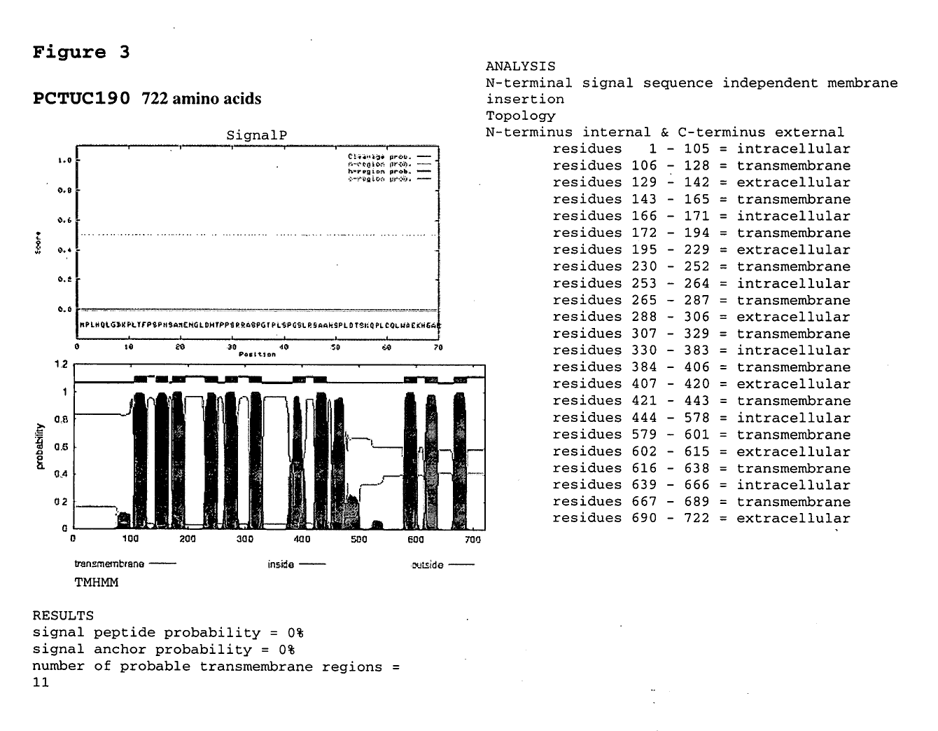

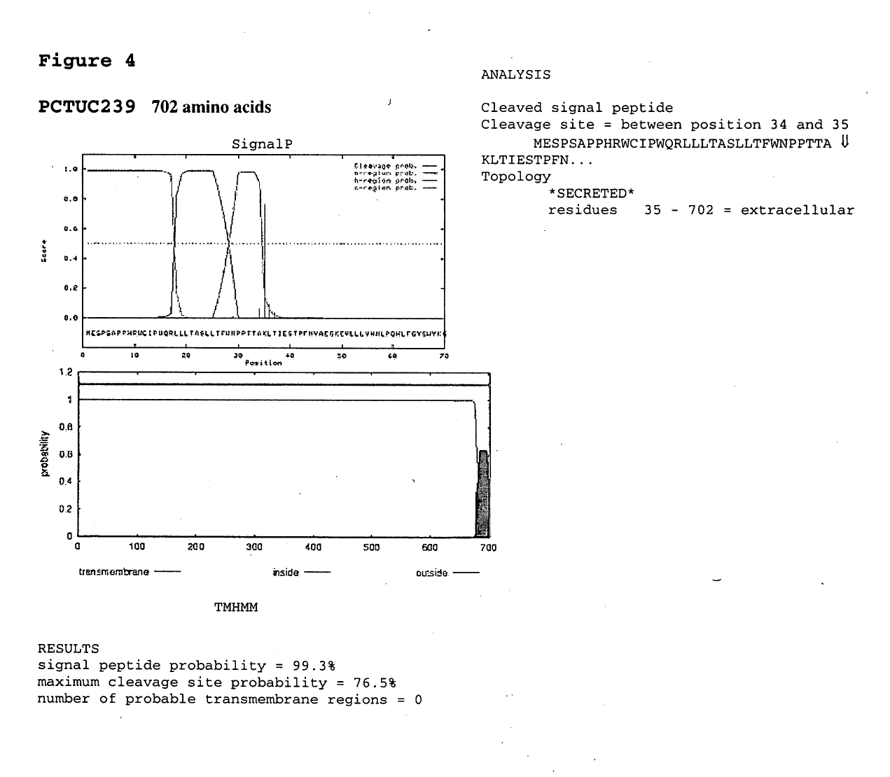

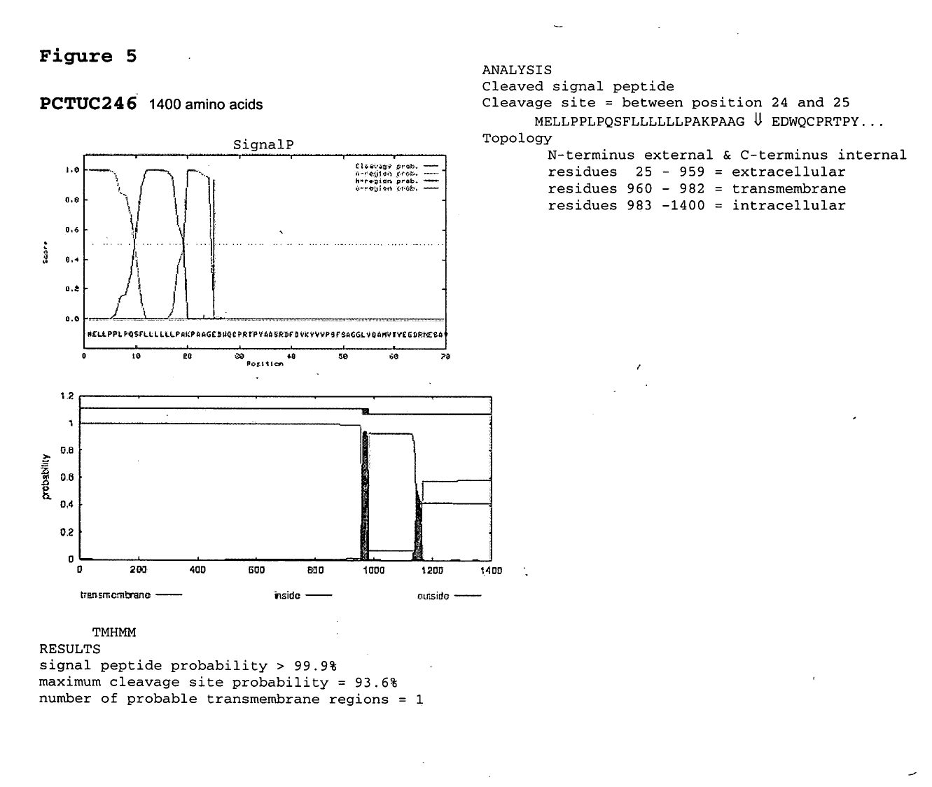

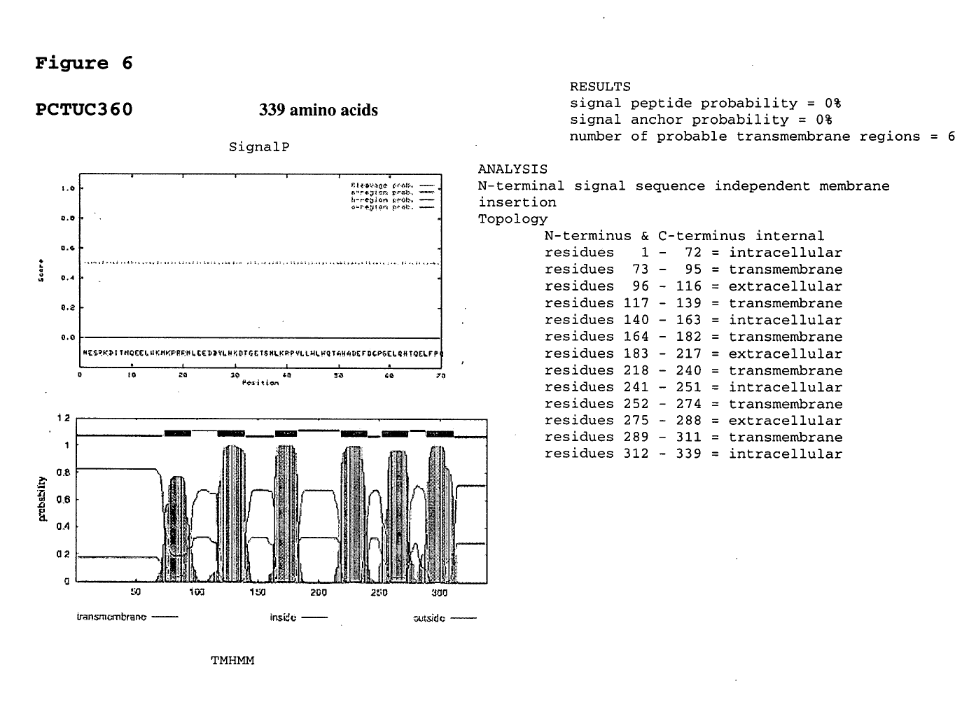

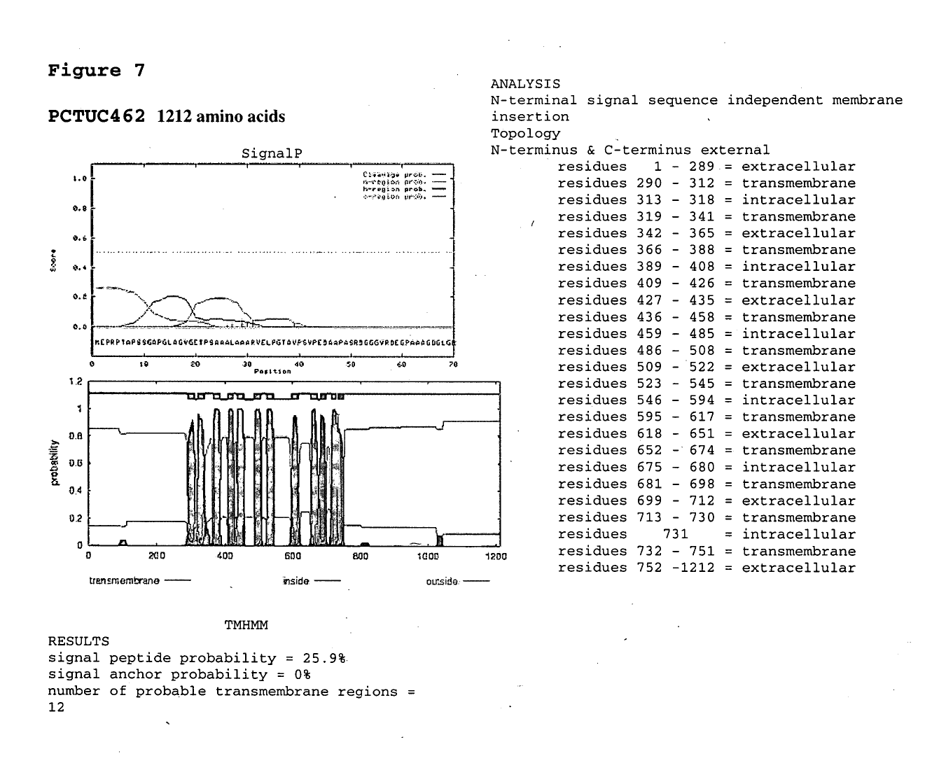

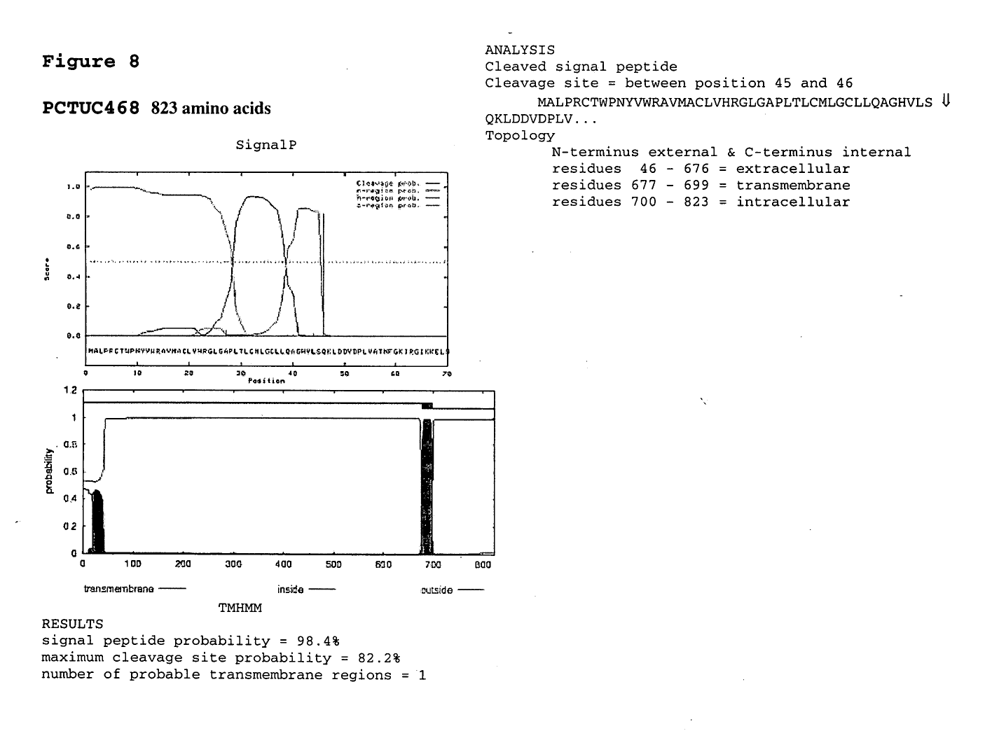

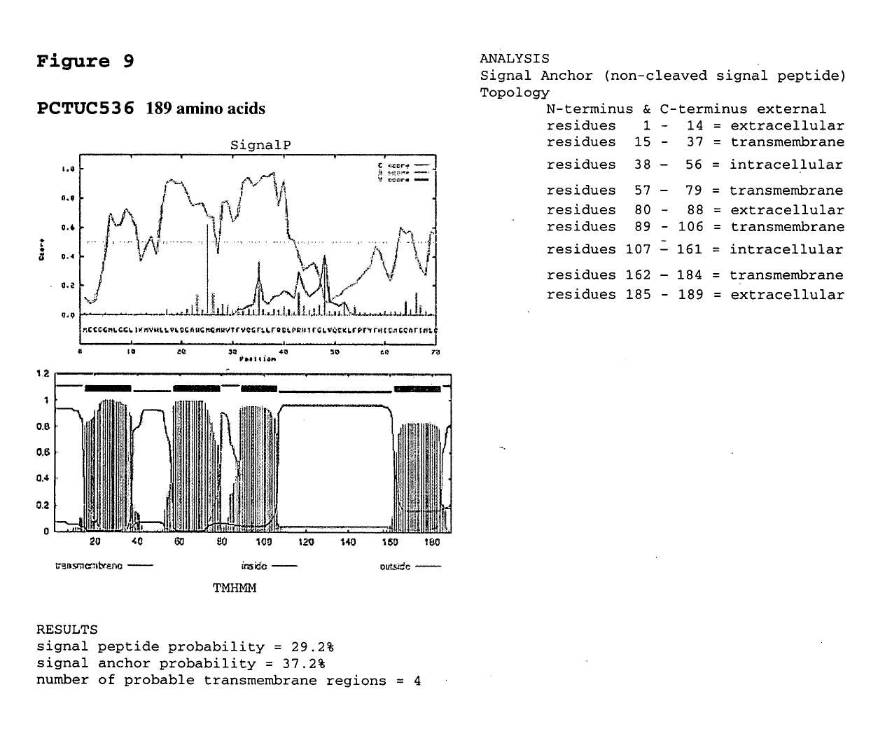

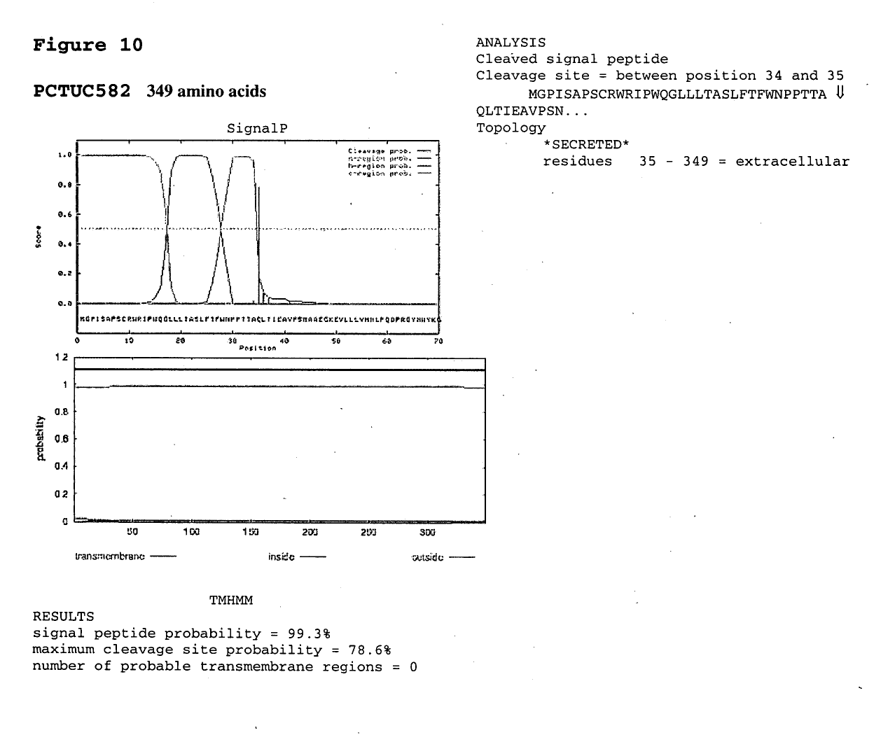

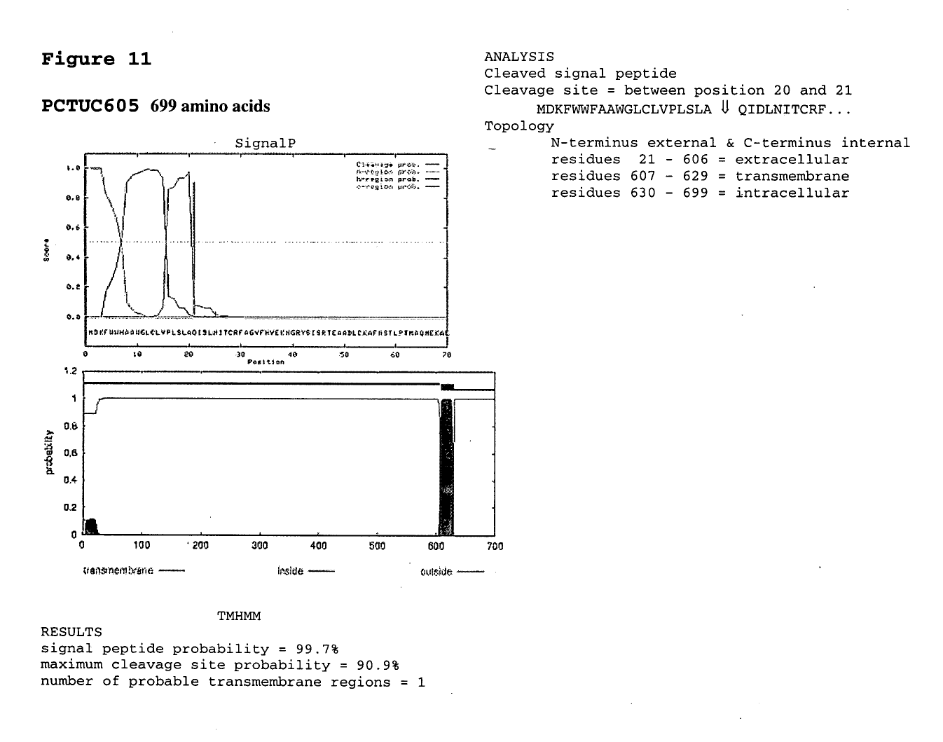

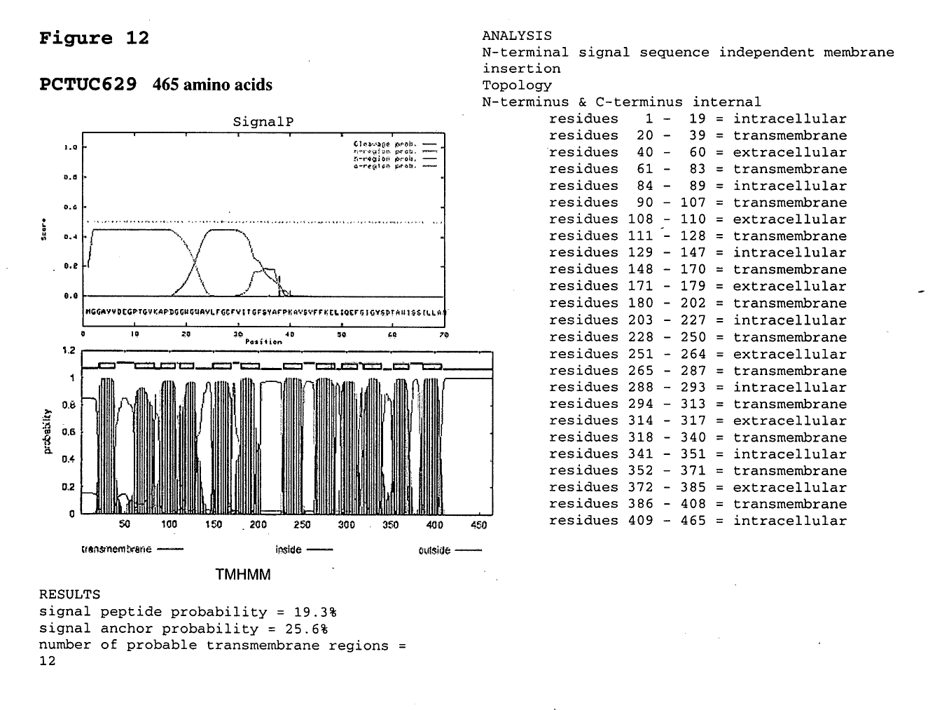

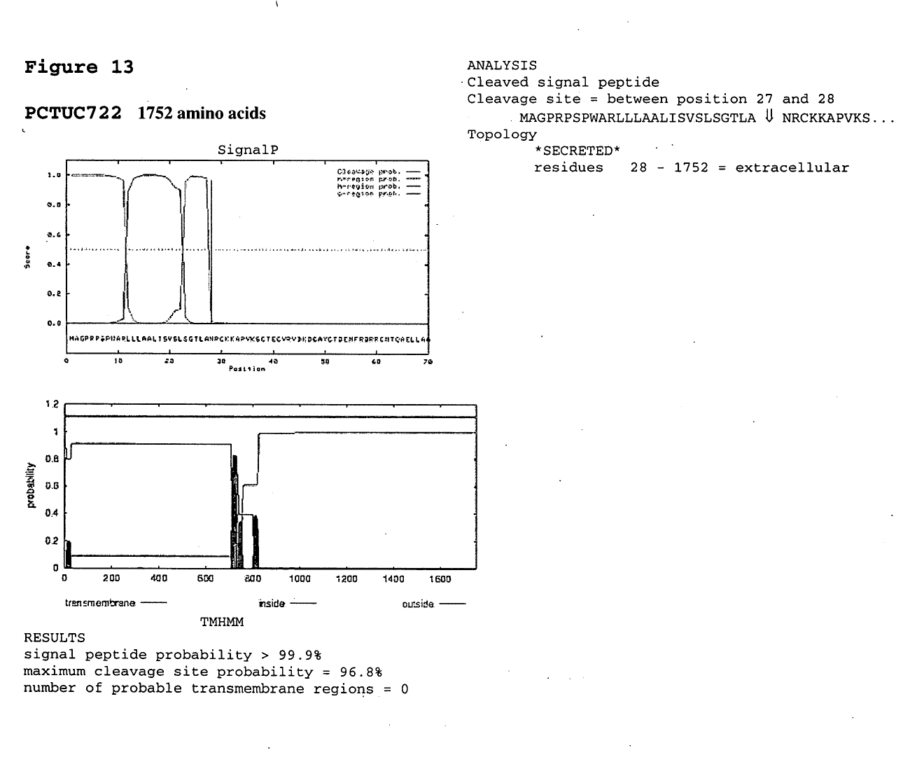

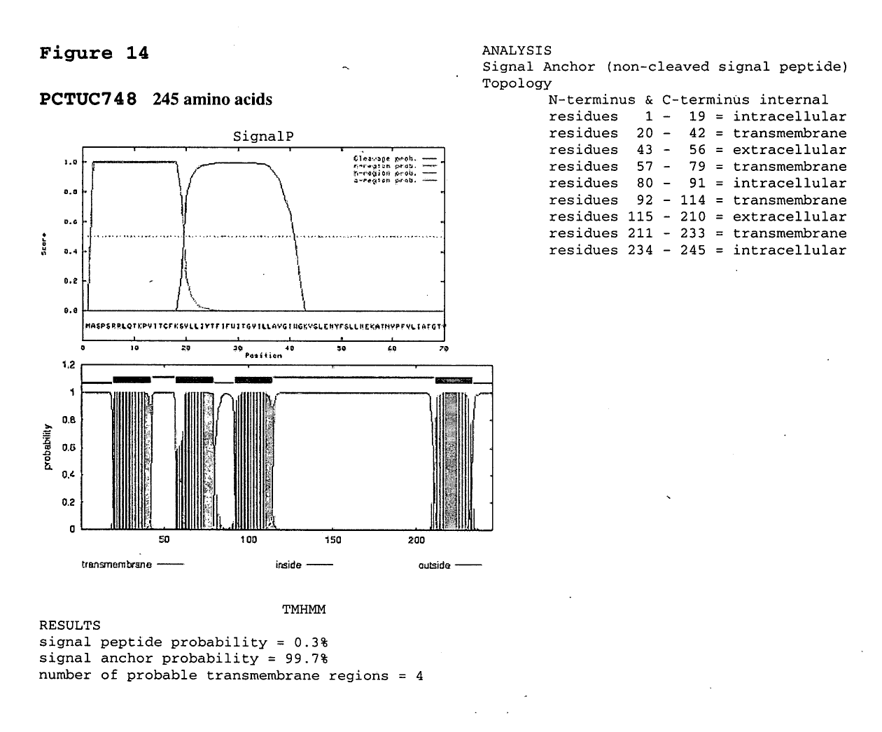

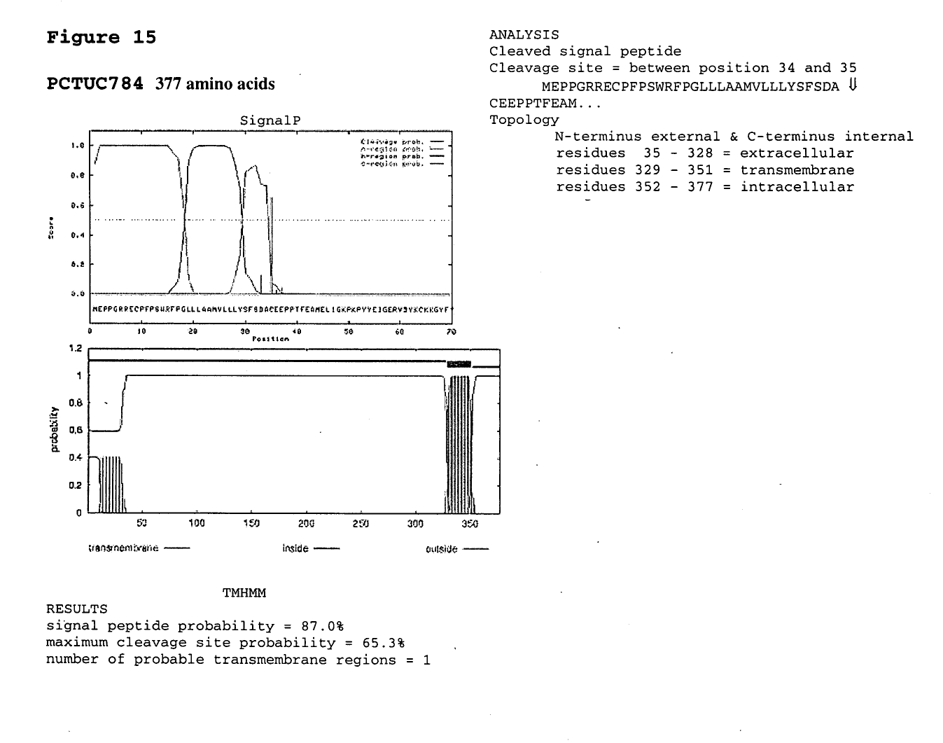

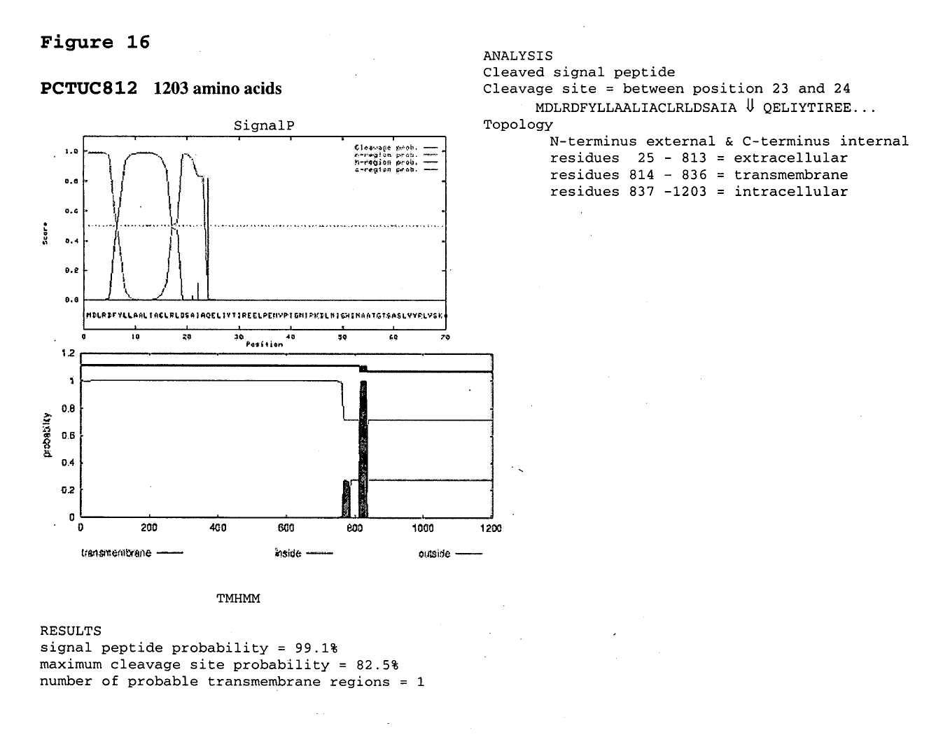

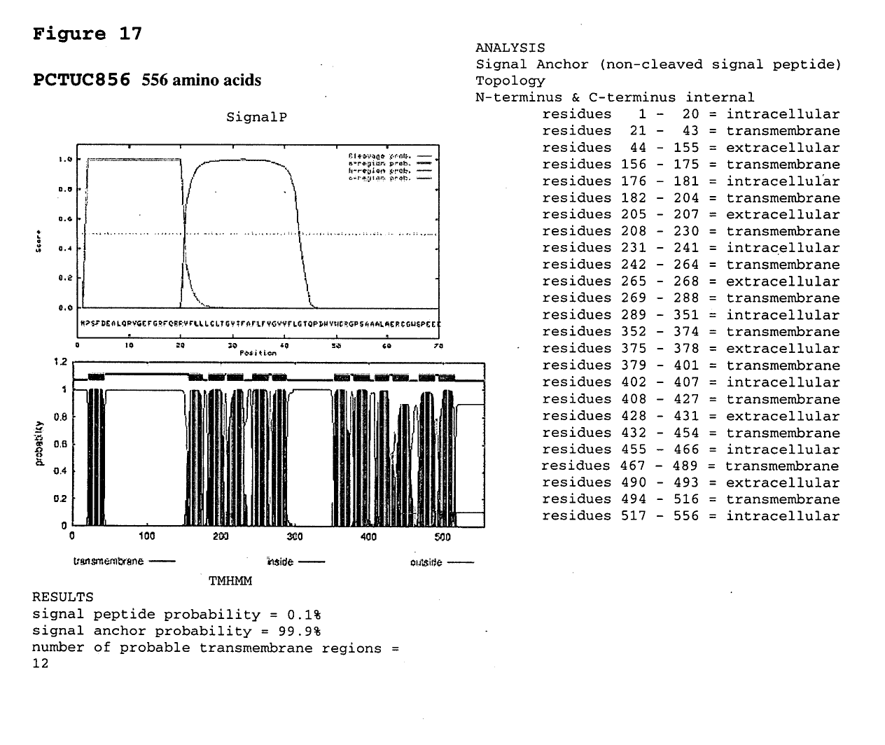

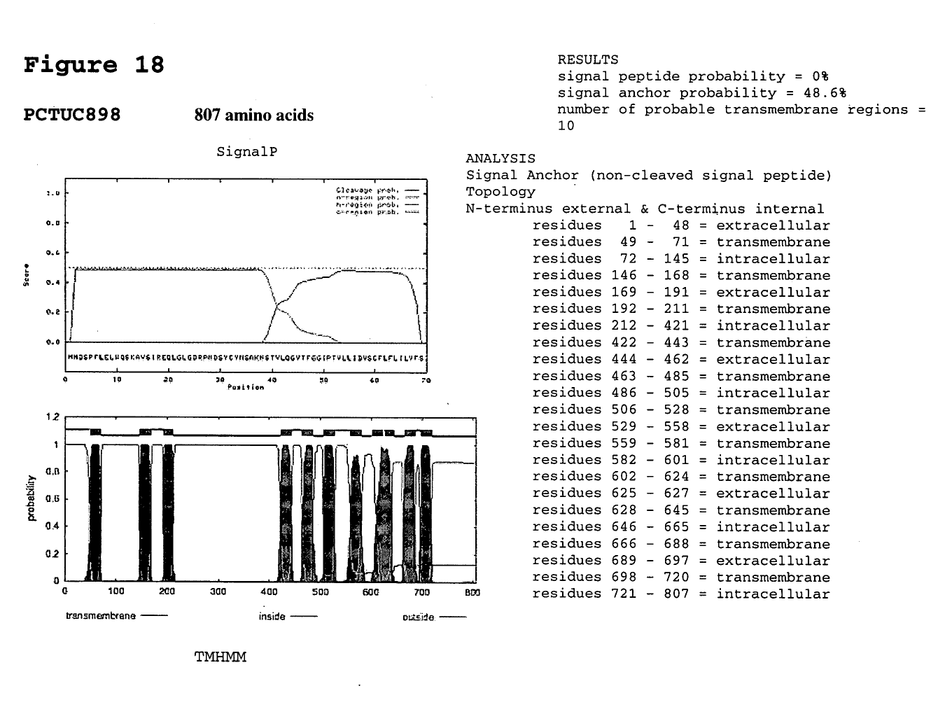

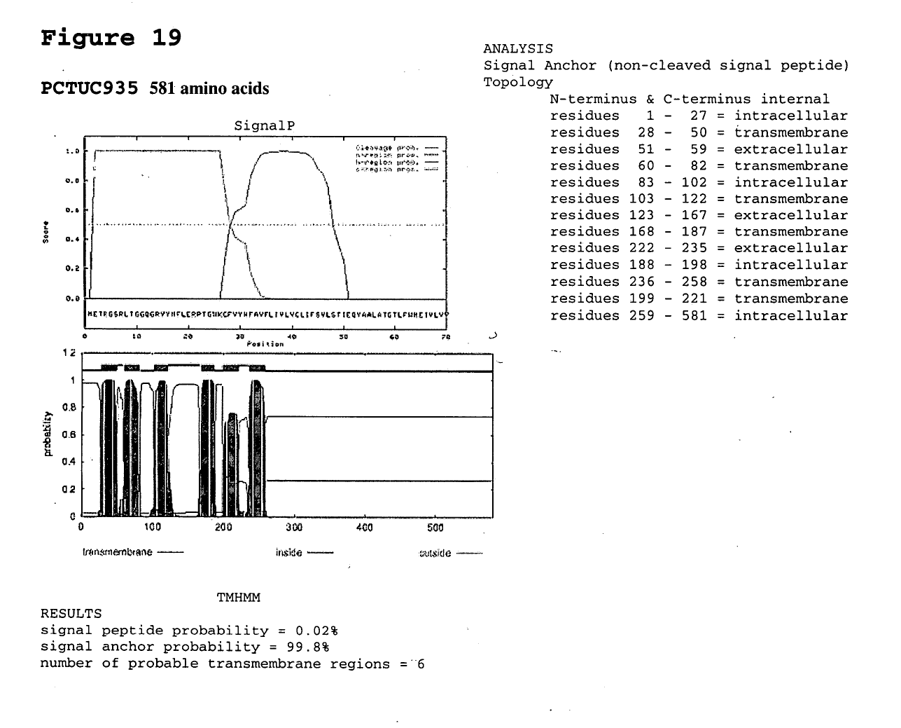

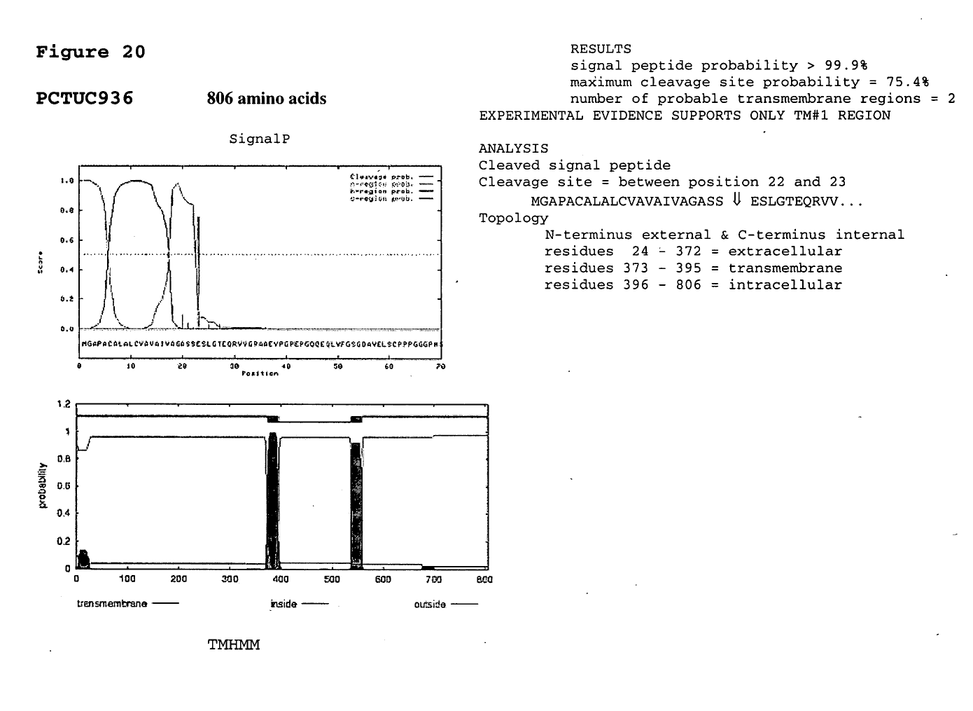

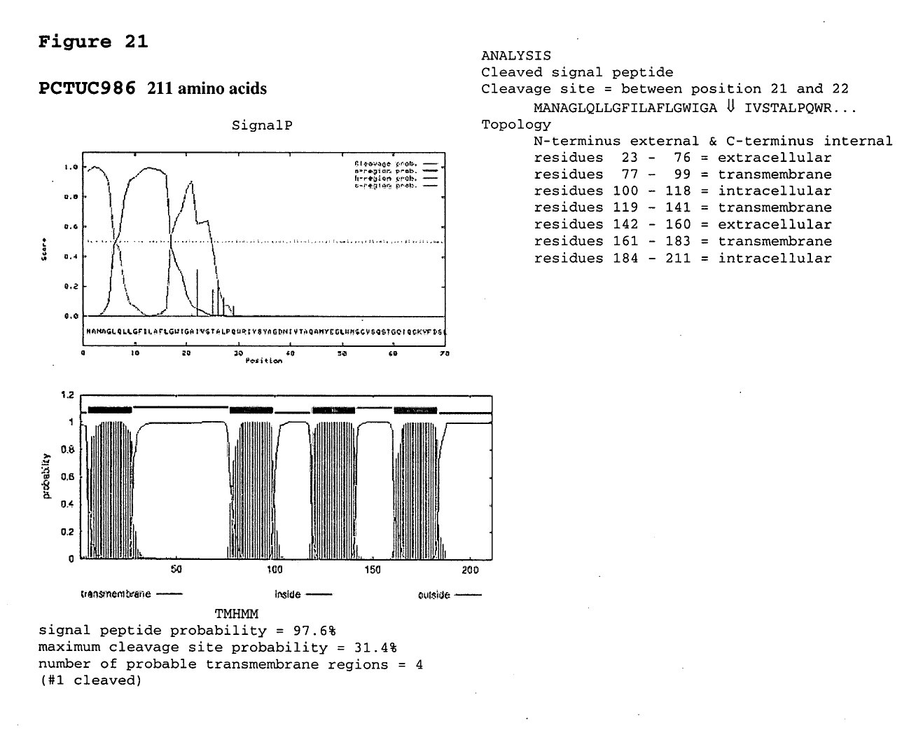

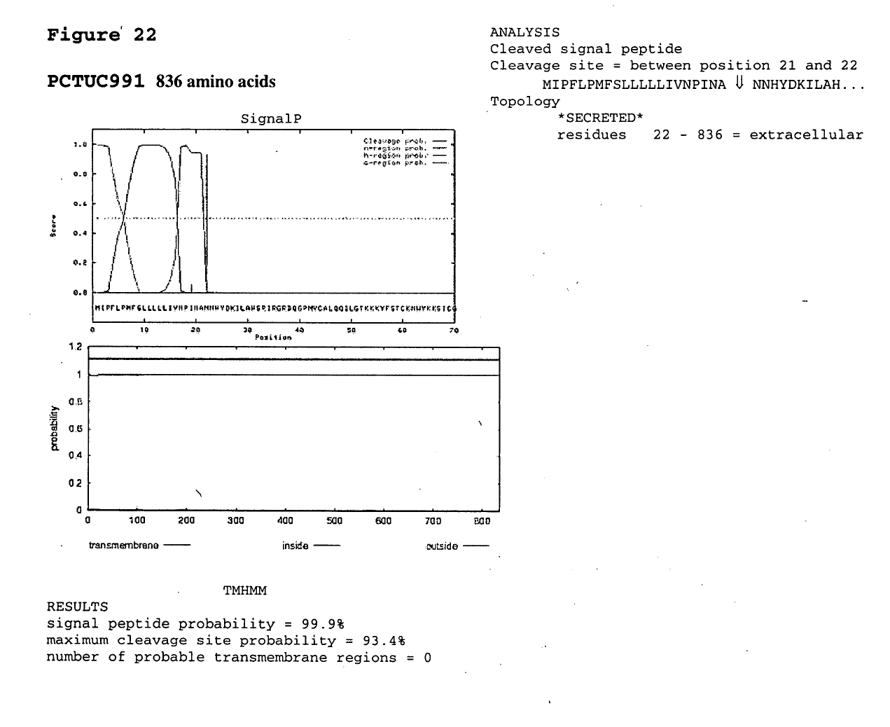

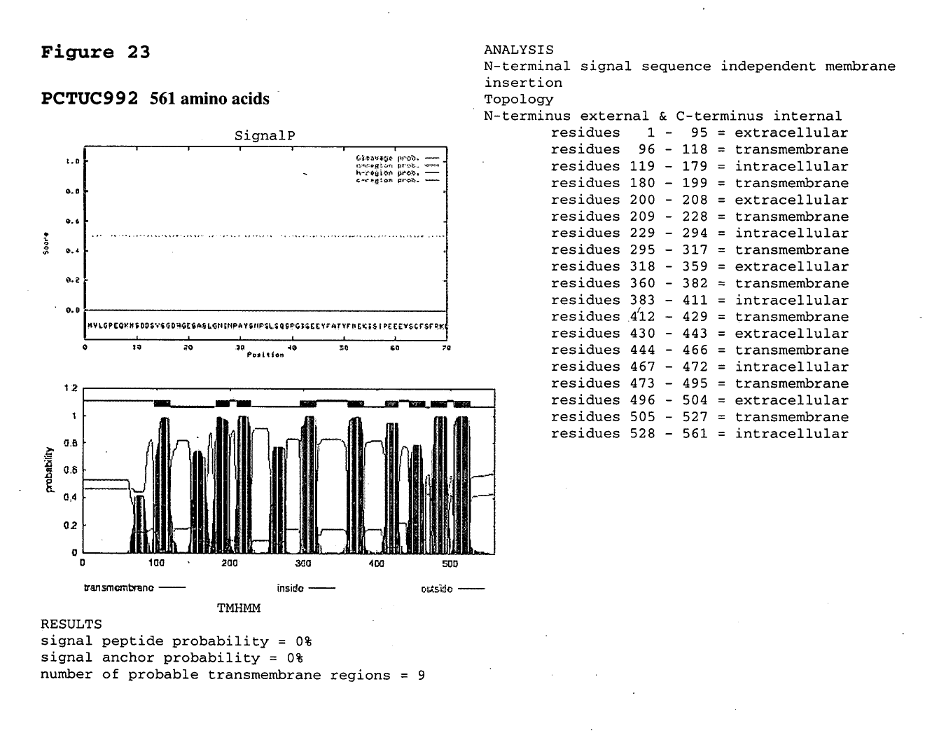

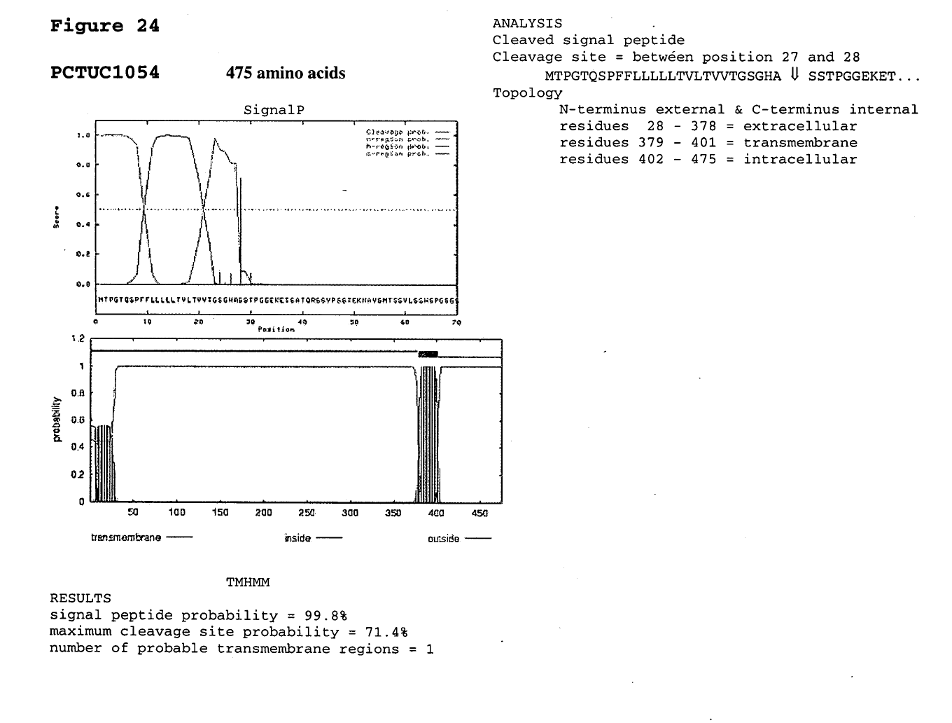

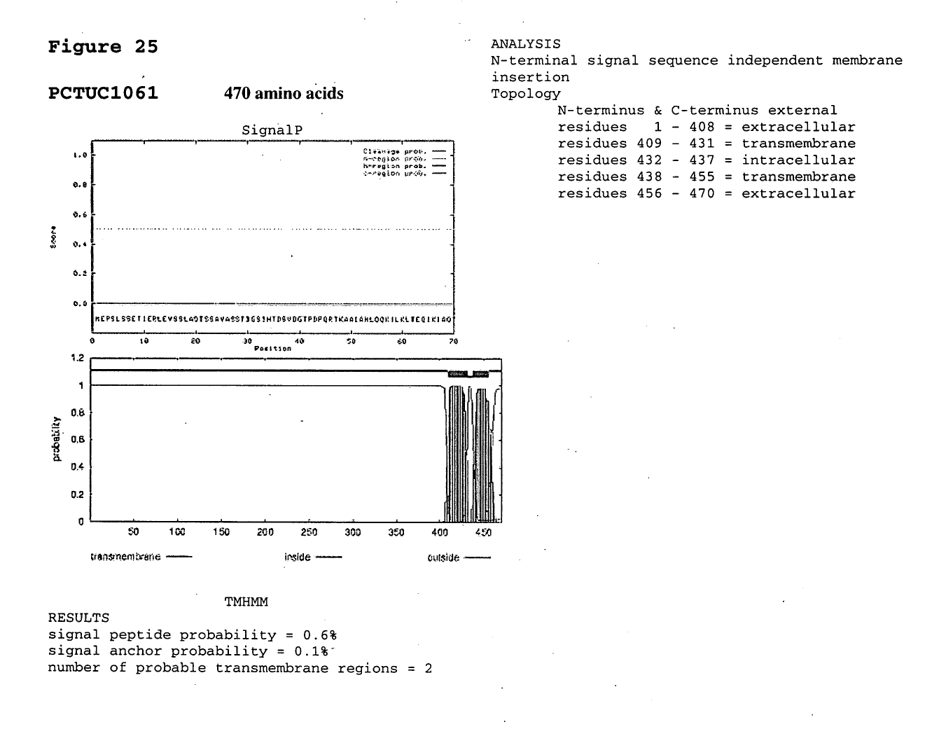

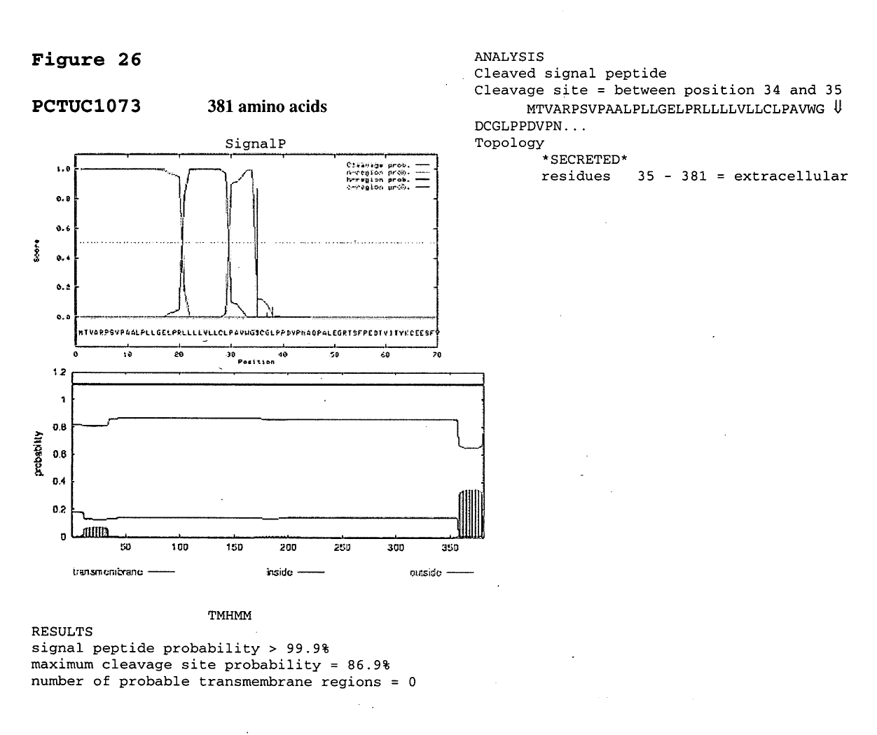

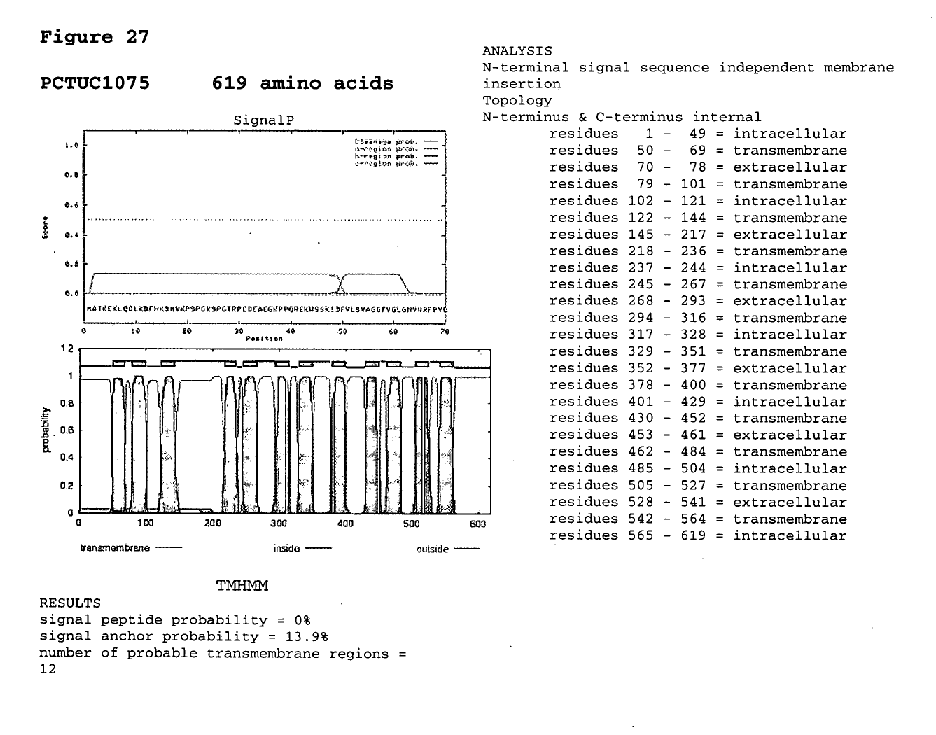

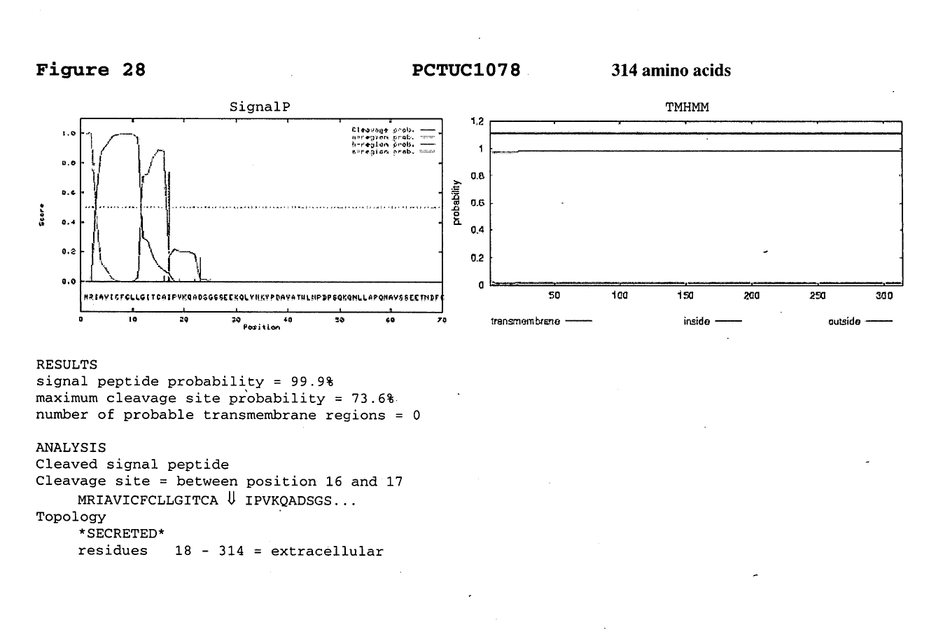

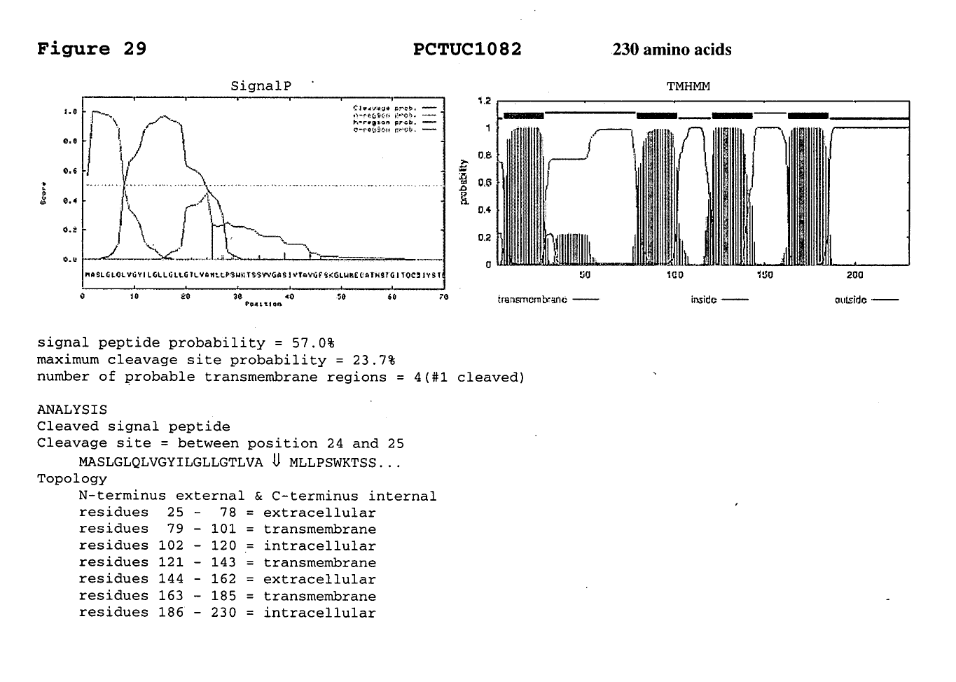

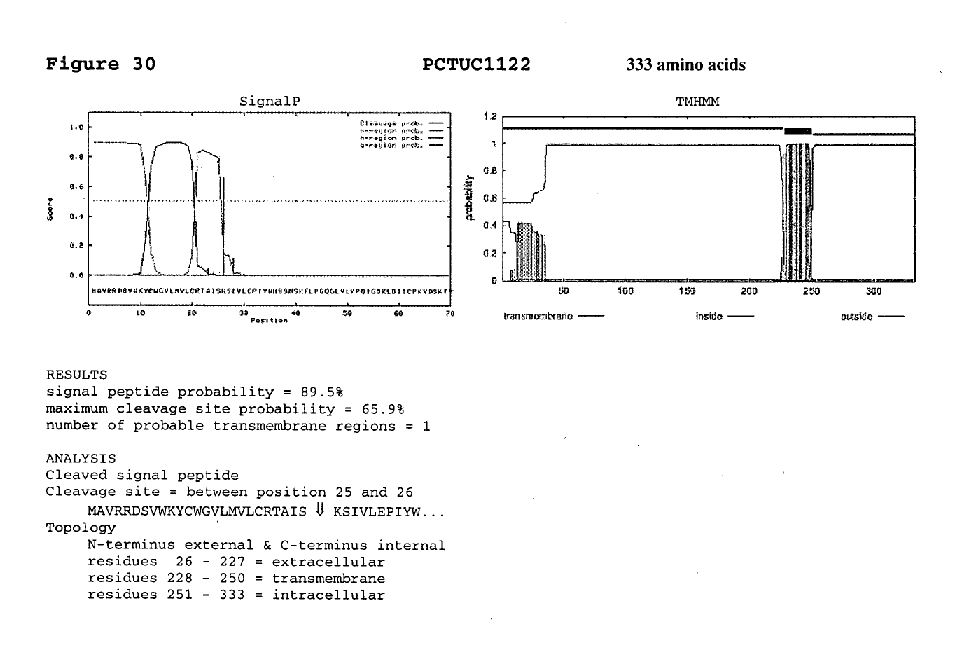

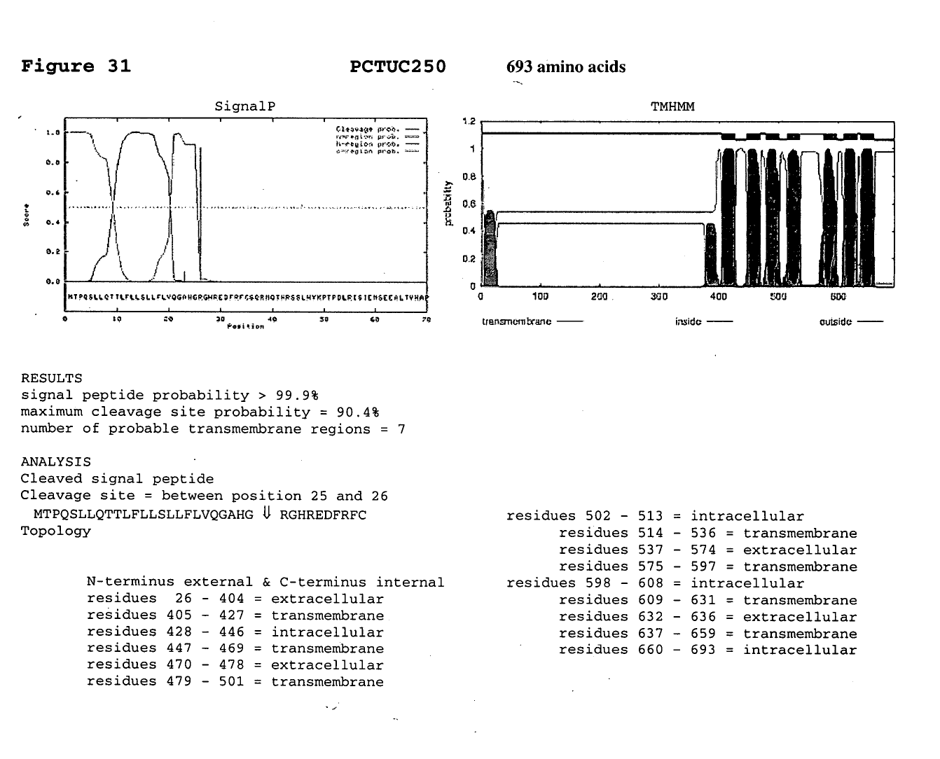

FIGS. 1-31 shows the SignalP and TMHMM analysis of the protein sequences of PCTUC-5 (SEQ ID NO:39), PCTUC-93 (SEQ ID NO:46), PCTUC-190 (SEQ ID NO:57), PCTUC-239 (SEQ ID NO:65), PCTUC-246 (SEQ ID NO:40), PCTUC-360 (SEQ ID NO:53), PCTUC-462 (SEQ ID NO:58), PCTUC-468 (SEQ ID NO:48), PCTUC-536 (SEQ ID NO:3114), PCTUC-582 (SEQ ID NO:64), PCTUC-605 (SEQ ID NO:71), PCTUC-629 (SEQ ID NO:67), PCTUC-722 (SEQ ID NO:61), PCTUC-748 (SEQ ID NO:63), PCTUC-784 (SEQ ID NO:72), PCTUC-812 (SEQ ID NO:66), PCTUC-856 (SEQ ID NO:49), PCTUC-898 (SEQ ID NO:43), PCTUC-935 (SEQ ID NO:70), PCTUC-936 (SEQ ID NO:42), PCTUC-986 (SEQ ID NO:47), PCTUC-991 (SEQ ID NO:75), PCTUC-992 (SEQ ID NO:60), PCTUC-1054 (SEQ ID NO:59), PCTUC-1061 (SEQ ID NO:55), PCTUC-1073 (SEQ ID NO:56), PCTUC-1075 (SEQ ID NO:73), PCTUC-1078 (SEQ ID NO:68), PCTUC-1082 (SEQ ID NO:54), PCTUC-1122 (SEQ ID NO:62), and PCTUC-250 (SEQ ID NO:41) respectively. The predictions of the SignalP and TMHMM analyses for subcellular localization and membrane topology of these peptides are summarized in Table 5.

TABLE 5

| | | Cleaved | | Number of |
|---|---|---|---|---|
| | Product | Signal | | TM |
| Gene | Location | Sequence | Topology | Segments |
| PCTUC5 | plasma | yes | N-terminus | 1 |
| | membrane | | outside & | |
| | | | C-terminus | |
| | | | inside | |
| PCTUC93 | secreted | yes | N-terminus | 0 |
| | | | outside & | |
| | | | C-terminus | |
| | | | outside | |
| PCTUC190 | plasma | NA | N-terminus | 11 |
| | membrane | | inside & | |
| | | | C-terminus | |
| | | | outside | |
| PCTUC239 | secreted | yes | N-terminus | 0 |
| | | | outside & | |
| | | | C-terminus | |
| | | | outside | |
| PCTUC246 | plasma | yes | N-terminus | 1 |
| | membrane | | outside & | |
| | | | C-terminus | |
| | | | inside | |
| PCTCUC250 | Plasma | yes | N-terminus | 7 |
| | membrane | | inside & | |
| | | | C-terminus | |
| | | | inside | |
| PCTUC360 | plasma | NA | N-terminus | 6 |
| | membrane | | inside & | |
| | | | C-terminus | |
| | | | inside | |
| PCTUC462 | plasma | NA | N-terminus | 12 |
| | membrane | | outside & | |
| | | | C-terminus | |
| | | | outside | |
| PCTUC468 | plasma | yes | N-terminus | 1 |
| | membrane | | outside & | |
| | | | C-terminus | |
| | | | inside | |
| PCTUC536 | plasma | no | N-terminus | 4 |
| | membrane | | outside & | |
| | | | C-terminus | |
| | | | outside | |
| PCTUC582 | secreted | yes | N-terminus | 0 |
| | | | outside & | |
| | | | C-terminus | |
| | | | outside | |
| PCTUC605 | plasma | yes | N-terminus | 1 |
| | membrane | | outside & | |
| | | | C-terminus | |
| | | | inside | |
| PCTUC629 | plasma | NA | N-terminus | 12 |
| | membrane | | inside & | |
| | | | C-terminus | |
| | | | inside | |
| PCTUC722 | secreted | yes | N-terminus | 0 |
| | | | outside & | |
| | | | C-terminus | |
| | | | outside | |
| PCTUC748 | plasma | no | N-terminus | 4 |
| | membrane | | inside & | |
| | | | C-terminus | |
| | | | inside | |
| PCTUC784 | plasma | yes | N-terminus | 1 |
| | membrane | | outside & | |
| | | | C-terminus | |
| | | | inside | |
| PCTUC812 | plasma | yes | N-terminus | 1 |
| | membrane | | outside & | |
| | | | C-terminus | |
| | | | inside | |
| PCTUC856 | plasma | no | N-terminus | 12 |
| | membrane | | inside & | |
| | | | C-terminus | |
| | | | inside | |
| PCTUC898 | plasma | no | N-terminus | 10 |
| | membrane | | outside & | |
| | | | C-terminus | |
| | | | inside | |
| PCTUC935 | plasma | no | N-terminus | 6 |
| | membrane | | inside & | |
| | | | C-terminus | |
| | | | inside | |
| PCTUC936 | plasma | yes | N-terminus | 1 |
| | membrane | | outside & | |
| | | | C-terminus | |
| | | | inside | |
| PCTUC986 | plasma | yes | N-terminus | 3 |
| | membrane | | outside & | |
| | | | C-terminus | |
| | | | inside | |
| PCTUC991 | secreted | yes | N-terminus | 0 |
| | | | outside & | |
| | | | C-terminus | |
| | | | outside | |
| PCTUC992 | plasma | NA | N-terminus | 9 |
| | membrane | | outside & | |
| | | | C-terminus | |
| | | | inside | |
| PCTUC1054 | plasma | yes | N-terminus | 1 |
| | membrane | | outside & | |
| | | | C-terminus | |
| | | | inside | |
| PCTUC1061 | plasma | NA | N-terminus | 2 |
| | membrane | | outside & | |
| | | | C-terminus | |
| | | | outside | |
| PCTUC1073 | secreted | yes | N-terminus | 0 |
| | | | outside & | |
| | | | C-terminus | |
| | | | outside | |
| PCTUC1075 | plasma | NA | N-terminus | 12 |
| | membrane | | inside & | |
| | | | C-terminus | |
| | | | inside | |
| PCTUC1078 | secreted | yes | N-terminus | 0 |
| | | | outside & | |
| | | | C-terminus | |
| | | | outside | |
| PCTUC1082 | plasma | yes | N-terminus | 3 |
| | membrane | | inside & | |
| | | | C-terminus | |
| | | | outside | |
| PCTUC1122 | plasma | yes | N-terminus | 1 |
| | membrane | | outside & | |
| | | | C-terminus | |
| | | | inside | |

NA = not applicable (N-terminal signal sequence independent membrane insertion)

### Example 7

SYBR Green PCR Analysis of the Time Dependence of Expression of Cell Surface Associated Genes.

Where possible microarray results were confirmed using the SYBR green procedure. The SYBR green PCR procedure is a way to perform real-time PCR using the SYBR Green 1 Dye. Direct detection of polymerase chain reaction (PCR) product is monitored by measuring the increase in fluorescence caused by the binding of SYBR green dye to double stranded DNA. Gene specific PCR oligonucleotide primer pairs were designed using the Primer Express 1.5 software. (Applied Biosystems, Foster City, Calif.)

The transcripts whose protein products were determined to be associated with the membrane were then further validated by SYBR green (real time PCR) analysis on an additional set of either 6 or 13 colon and 6 breast tumor RNA's and were compared to a pool of normal colon or breast RNA. First one microgram of each mRNA was added to 100 uL of a reverse transcriptase reaction using the ABI Taqman reverse transcription reagents with random hexamers according to the manufacturers protocol (Applied Biosystems, Foster City, Calif.) The thermal cycling conditions included 1 cycle at 25° C. for 10 minutes, 1 cycle at 48° C. for 30 minutes and 1 cycle at 95° C. for 5 minutes. Four hundred microliters of water was then added to the cDNA reaction. The cDNA (10 uL) that was generated was added to a 25 uL SYBR green PCR reaction mixture according to the manufactures protocol. (Applied Biosystems, Foster City, Calif.) The thermal cycling conditions included 1 cycle at 95° C. for ten minutes, 40 cycles at 95° C. for 15 s, annealing at 60° C. for 1 minute. Data is expressed as the fold increase normalized to the same gene using the delta delta CT method for relative quantitation. For comparison data obtained with cDNA from colon and breast tumors were compared to data from pools of normal colon and breast cDNA.

Table 6 shows the SYBR green primers and probe sets used. Table 7 shows the shows the SYBR green in results in the colon tumors (CT) and breast tumors (BT). ND=non-detectable

TABLE 6

| PCTUC | Forward Primer | Reverse Primer |
|---|---|---|
| 5 | CGGAGACAGGCTATGAGTCTGA | TGAAGTCAAACTGCCACATTC |
| | SEQ ID NO: 3012 | SEQ ID NO: 3048 |
| 246 | GCCAGCAACCTACATGAACTTG | TGAGAACTGCGGCTGTTCTG |
| | SEQ ID NO: 3013 | SEQ ID NO: 3049 |
| 250 | CGACATGCTGGGAGATTACATC | AGAGGCTTTGTCACTCAGCAAGA |
| | SEQ ID NO: 3014 | SEQ ID NO: 3050 |
| 936 | TGAGTCTGGGCAGCTGTCC | CTGGACTGCTACCTTTCAAAGCTT |
| | SEQ ID NO: 3015 | SEQ ID NO: 3051 |
| 898 | CATCAGGTTGGAGTGCGTCTT | CGAGGCGATGACATAGTTCACA |
| | SEQ ID NO: 3016 | SEQ ID NO: 3052 |
| 1121 | GGATCAGCCCTGAACTCACT | TTGTCCCTGTCCCTCTCTCT |
| | SEQ ID NO: 3017 | SEQ ID NO: 3053 |
| 1103 | AGACAAGGATGCCGTGGATAA | TGAAGTCCACCTGGGCATCT |
| | SEQ ID NO: 3018 | SEQ ID NO: 3054 |
| 93 | TCAATATAGATGATTGTGCCATCTTCT | CACGTTTATGAGTTGAACTTCTC |
| | SEQ ID NO: 3019 | SEQ ID NO: 3055 |
| 986 | TGCAAAGTCTTTGACTCCTTGCT | TCAAGGCACGGGTTGCTT |
| | SEQ ID NO: 3020 | SEQ ID NO: 3056 |
| 468 | GTCCAAAGAGTTACTTGCAACAGTCT | ATTAGTAAACATTTTGTCATGCAGCAT |
| | SEQ ID NO: 3021 | SEQ ID NO: 3057 |
| 856 | GGCATGGTTTAGGCCCTGTT | TGGCTCTAGGTGTCCACTAAAGG |
| | SEQ ID NO: 3022 | SEQ ID NO: 3058 |
| 536 | CCAAGATGCAGAGGTTGATGAA | TCGTCTCAGGCTTCCTGCTT |
| | SEQ ID NO: 3023 | SEQ ID NO: 3059 |
| 360 | CCGTTTATGGGTAGACATCTTTGG | TGTTGGAGTATACGTGTGGACATG |
| | SEQ ID NO: 3024 | SEQ ID NO: 3060 |
| 1082 | GCCATGCCAGCCTTTCTGT | GCAATGAGCTAAGAGCCAACCT |
| | SEQ ID NO: 3025 | SEQ ID NO: 3061 |
| 1061 | AGCTAGAAGGGCTGGAGAATGC | GAACGTCCTGTTGCGAGTCTT |
| | SEQ ID NO: 3026 | SEQ ID NO: 3062 |
| 1073 | GGTACAAATTATTTGGCTCGACTTC | CACTCTGGCAACGGGTCACT |
| | SEQ ID NO: 3027 | SEQ ID NO: 3063 |
| 190 | TGATGCAATCACACGGGAACT | GAGGTCACAGCCGACTTTAAACC |
| | SEQ ID NO: 3028 | SEQ ID NO: 3064 |
| 462 | CATGGCATGGTTAGAAGCTCTATCT | ACACTCTGATGATTCCCACGAACTA |
| | SEQ ID NO: 3029 | SEQ ID NO: 3065 |
| 1054 | CGTTCTCTCCATTGCTTGTTAGC | CACAGGACAGGGATGGAGAAG |
| | SEQ ID NO: 3030 | SEQ ID NO: 3066 |
| 992 | TCAAGGGAGCCAAGAGCTCTT | TTGACAGTGTGTTTATGTGGAATGTT |
| | SEQ ID NO: 3031 | SEQ ID NO: 3067 |
| 722 | GACAGCAAGGTGCCCTCAGT | GTAGGCGCACACCTTCATCTC |
| | SEQ ID NO: 3032 | SEQ ID NO: 3068 |
| 1122 | TGTCTGCGAAGAAGGCTAGGAG | ATGGACTGAAGCTGTTGTTGCC |
| | SEQ ID NO: 3033 | SEQ ID NO: 3069 |
| 748 | TCAAGATCCGTGCTCGCAGT | GGGATACAGGGTTTCAACGA |
| | SEQ ID NO: 3034 | SEQ ID NO: 3070 |
| 582 | GTTCAGCGTACATCCGGAGACT | TGACCATTTACCCACCACAGGT |
| | SEQ ID NO: 3035 | SEQ ID NO: 3071 |
| 239 | TTGTCATCCGTCTTCTGAC | GTGGGCACCTTTGATTCCT |
| | SEQ ID NO: 3036 | SEQ ID NO: 3072 |
| 812 | CGTAAGCAGTATGGCTCCAA | AGCACCTCCTGCTTGCTTAT |
| | SEQ ID NO: 3037 | SEQ ID NO: 3073 |
| 629 | ACCCAAACTCCACAAAGCCATT | GCCAGGATGAACACGTACATGTA |
| | SEQ ID NO: 3038 | SEQ ID NO: 3074 |
| 1078 | CCCTTCCAAGTAAGTCCAACGA | TGTCAGGTCTGCGAAACTTCTT |
| | SEQ ID NO: 3039 | SEQ ID NO: 3075 |
| 1124 | GACAGTCACAGCAGCCTTGACA | TGAACGGCGTGGATTCAATA |
| | SEQ ID NO: 3040 | SEQ ID NO: 3076 |
| 935 | TGCAGATCCTGAGGATGCTAC | TCCTTCTCAGCCAGGTACACAA |
| | SEQ ID NO: 3041 | SEQ ID NO: 3077 |
| 605 | GTGGAGGACAGAAAGCCAAGTG | GCACCATTTCCTGAGACTTGCT |
| | SEQ ID NO: 3042 | SEQ ID NO: 3078 |
| 784 | TGGCTCTCGGTTTCTCTGCTT | CGCGGAAGACGCTGTTATT |
| | SEQ ID NO: 3043 | SEQ ID NO: 3079 |
| 1075 | TGGCTTGATCAAGGGCCTTA | TGGTCACGTTTCGGTTTCAT |
| | SEQ ID NO: 3044 | SEQ ID NO: 3080 |
| 1125 | AGAAGAGCTGCCAGGAAGTGTT | TCCACATGACCAGACTCTCCA |
| | SEQ ID NO: 3045 | SEQ ID NO: 3081 |
| 991 | TGGAAAACAGCAAACCACCTT | CAGAGCAGATGCCAAGCCTAA |
| | SEQ ID NO: 3046 | SEQ ID NO: 3082 |
| 1126 | TTCTGAGGCATTAAGCCAGCA | TGCATGGAGTTGCTGCTGT |
| | SEQ ID NO: 3047 | SEQ ID NO: 3083 |

TABLE 7

| PCTUC | CT-1 | CT-2 | CT-3 | CT-4 | CT-5 | CT-6 | CT-7 | CT-8 | CT-9 | CT-10 |
|---|---|---|---|---|---|---|---|---|---|---|
| 5 | 49.3 | 0.0 | 67.2 | 12.5 | 83.0 | 74.6 | | | | |
| 246 | 2.6 | 0.0 | 2.3 | 1.0 | 25.7 | 2.7 | | | | |
| 250 | 3.4 | 0.0 | 7.8 | 2.3 | 0.8 | 3.5 | | | | |
| 936 | 252.4 | 5.5 | 3.9 | 2.7 | 89.1 | 5.2 | | | | |
| 898 | 4.8 | 1.5 | 5.3 | 2.7 | 4.9 | 7.1 | | | | |
| 1121 | 4.2 | 6.9 | 5.8 | 2.8 | 24.3 | 12.6 | | | | |
| 1103 | 5.4 | 1.1 | 1.9 | 3.0 | 5.9 | 5.8 | | | | |
| 93 | 0.6 | 0.1 | 1.2 | 0.4 | 0.2 | 0.8 | | | | |
| 986 | 27.8 | 31.3 | 17.5 | 11.0 | 3.5 | 16.6 | | | | |
| 468 | ND | ND | ND | ND | ND | ND | | | | |
| 856 | 3.4 | 5.2 | 1.8 | 1.6 | 4.8 | 5.8 | | | | |
| 536 | 2.2 | 2.5 | 2.9 | 1.7 | 1.7 | 1.9 | 11.0 | 7.5 | 5.7 | 5.2 |
| 360 | 1.2 | 1.4 | 1.7 | 0.4 | 1.6 | 1.2 | | | | |
| 1082 | 1.7 | 2.6 | 65.8 | 0.4 | 16.1 | 1.7 | | | | |
| 1061 | 1.6 | 1.5 | 2.2 | 0.8 | 1.0 | 1.7 | 1.4 | 4.6 | 1.6 | 0.6 |
| 1073 | 3.3 | 4.6 | 1.9 | 0.7 | 0.6 | 1.0 | | | | |
| 190 | 16.9 | 8.0 | 9.8 | 4.7 | 12.4 | 9.6 | | | | |
| 462 | 1.9 | 1.9 | 3.7 | 0.9 | 1.0 | 1.6 | | | | |
| 1054 | ND | ND | ND | ND | ND | ND | | | | |
| 992 | ND | ND | ND | ND | ND | ND | | | | |
| 722 | 1.9 | 3.5 | 2.4 | 0.6 | 1.0 | 1.2 | | | | |
| 1122 | 1.1 | 1.7 | 0.7 | 0.4 | 0.4 | 0.6 | 0.5 | 0.6 | 1.2 | 0.7 |
| 748 | 0.8 | 0.3 | 2.1 | 0.5 | 0.3 | 1.3 | 11.4 | 14.3 | 2.1 | 13.4 |
| 582 | 3.9 | 0.2 | 0.7 | 1.0 | 1.1 | 2.3 | | | | |
| 239 | 19.4 | 0.0 | 7.9 | 37.9 | 7.0 | 8.6 | | | | |
| 812 | 0.3 | 0.0 | 0.2 | 0.1 | 0.3 | 0.3 | | | | |
| 629 | 2.8 | 0.0 | 3.4 | 2.2 | 2.7 | 2.8 | | | | |
| 1078 | 16.0 | 5.6 | 4.0 | 0.7 | 0.2 | 0.9 | | | | |
| 1124 | 4.1 | 0.1 | 2.1 | 1.3 | 0.1 | 0.7 | | | | |
| 935 | 3.2 | 2.3 | 7.7 | 0.9 | 0.2 | 2.0 | | | | |
| 605 | 1.8 | 1.7 | 3.0 | 1.0 | 1.5 | 2.2 | | | | |
| 784 | 1.5 | 0.9 | 2.7 | 0.7 | 0.2 | 0.8 | 2.4 | 5.0 | 3.4 | 2.5 |
| 1075 | 1.7 | 0.4 | 4.4 | 0.5 | 0.2 | 2.0 | | | | |
| 1125 | 1.1 | 0.9 | 2.1 | 0.7 | 0.4 | 1.7 | 2.7 | 3.9 | 3.5 | 5.2 |
| 991 | 1.8 | 1.4 | 1.3 | 0.7 | 0.2 | 0.5 | 3.7 | 10.1 | 5.2 | 11.0 |
| 1126 | 4.0 | 7.8 | 1.7 | 4.5 | 24.2 | 4.8 | | | | |

| PCTUC | CT-11 | CT-12 | CT-13 | BT-1 | BT-2 | BT-3 | BT-4 | BT-5 | BT-6 |
|---|---|---|---|---|---|---|---|---|---|
| 5 | | | | 1.8 | 0.3 | 0.5 | 0.4 | 1.4 | 2.4 |
| 246 | | | | 3.8 | 2.8 | 4.4 | 8.9 | 8.1 | 2.8 |
| 250 | | | | 0.7 | 0.4 | 0.5 | 0.4 | 0.7 | 0.5 |
| 936 | | | | 0.1 | 0.1 | 0.6 | 68.1 | 15.7 | 2.5 |
| 898 | | | | 0.9 | 0.7 | 1.3 | 0.8 | 1.7 | 0.6 |
| 1121 | | | | 2.1 | 0.9 | 0.6 | 5.5 | 4.4 | 0.9 |
| 1103 | | | | 8.1 | 22.5 | 2.9 | 19.4 | 33.6 | 29.3 |
| 93 | | | | 1.0 | 0.7 | 2.1 | 1.3 | 7.9 | 0.4 |
| 986 | | | | 1.1 | 0.1 | 0.5 | 0.1 | 0.2 | 0.7 |
| 468 | | | | ND | ND | ND | ND | ND | ND |
| 856 | | | | 0.8 | 0.3 | 0.1 | 0.1 | 0.3 | 0.7 |
| 536 | 4.8 | 6.9 | 1.0 | 1.8 | 1.4 | 1.9 | 4.0 | 9.3 | 1.4 |
| 360 | | | | 1.6 | 0.7 | 1.7 | 0.6 | 1.0 | 0.0 |
| 1082 | | | | 0.3 | 0.2 | 0.0 | 0.4 | 1.4 | 0.6 |
| 1061 | 0.4 | 1.5 | 1.0 | 0.7 | 0.6 | 1.2 | 1.0 | 1.1 | 0.5 |
| 1073 | | | | 1.0 | 0.8 | 1.9 | 1.1 | 3.4 | 0.7 |
| 190 | | | | 0.6 | 0.4 | 0.2 | 0.7 | 2.3 | 2.3 |
| 462 | | | | 0.4 | 0.2 | 1.5 | 0.3 | 1.1 | 0.1 |
| 1054 | | | | 2.5 | 4.5 | 8.9 | 0.0 | 8.0 | 4.1 |
| 992 | | | | ND | ND | ND | ND | ND | ND |
| 722 | | | | 0.9 | 0.4 | 0.7 | 0.7 | 0.6 | 0.4 |
| 1122 | 0.8 | 0.6 | 0.9 | 0.3 | 0.4 | 0.3 | 0.2 | 0.3 | 0.2 |
| 748 | 1.5 | 15.6 | 3.5 | 0.6 | 0.2 | 0.3 | 0.5 | 0.9 | 0.4 |
| 582 | | | | 12.6 | 28.2 | 12.5 | 3.0 | 4.2 | 3.2 |
| 239 | | | | 2.6 | 68.8 | 11.4 | 217.8 | 9.3 | 4.8 |
| 812 | | | | 0.1 | 0.1 | 0.1 | 0.0 | 0.2 | 0.1 |
| 629 | | | | 2.1 | 2.8 | 3.6 | 5.7 | 3.7 | 1.3 |
| 1078 | | | | 4.1 | 1.7 | 0.6 | 1.3 | 8.2 | 5.1 |
| 1124 | | | | 0.7 | 3.6 | 0.5 | 9.2 | 5.7 | 0.2 |
| 935 | | | | 0.8 | 0.6 | 0.3 | 0.3 | 0.8 | 0.3 |
| 605 | | | | 1.5 | 0.7 | 1.4 | 1.4 | 6.6 | 0.9 |
| 784 | 0.9 | 3.8 | 5.4 | 0.6 | 0.4 | 2.7 | 0.2 | 0.7 | 0.2 |
| 1075 | | | | 1.6 | 2.0 | 2.7 | 0.2 | 1.9 | 0.4 |
| 1125 | 1.0 | 4.0 | 3.4 | 1.2 | 1.5 | 0.6 | 2.4 | 3.2 | 1.7 |
| 991 | 5.1 | 2.3 | 2.7 | 1.6 | 0.6 | 2.3 | 0.4 | 3.5 | 0.6 |
| 1126 | | | | 1.8 | 2.3 | 3.8 | 10.1 | 6.7 | 0.8 |

### Example 8

Gene Expression in E. coli

This example illustrates preparation of an unglycosylated form of polypeptide by recombinant expression in E. coli.

The DNA sequence encoding polypeptide is initially amplified using selected PCR primers. The primers should contain restriction enzyme sites that correspond to the restriction enzyme sites on the selected expression vector. A variety of expression vectors can be employed. An example of a suitable vector is pBR322 (derived from E. coli; see Bolivar et al., Gene, 2:95 (1977)) that contains genes for ampicillin and tetracycline resistance. The vector is digested with restriction enzyme and dephosphorylated. The PCR amplified sequences are then ligated into the vector. The vector will preferably include sequences, which encode for an antibiotic resistance gene, a trp promoter, a polyhis leader (including the first six STII codons, polyhis sequence, and enterokinase cleavage site), the region encoding a protein of the present invention, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture can subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized protein can then be purified using a metal chelating column under conditions that allow tight binding of the protein.

Proteins of the present invention can be expressed in E. coli in a poly-His tagged form, using the following procedure. The DNA encoding a protein of the present invention is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₂)SO₄, 0.71 g sodium citrate-2H₂O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄,) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1 M and 0.02 M, respectively, and the solution is stirred overnight at 4° C. This step results in a denatured protein with all cysteine residues blocked by sulfitolization. The solution is centrifuged at 40,000 rpm in a Beckman Ultracentrifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calblochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 9

Expression of Polypeptides in Mammalian Cells

This example illustrates preparation of a potentially glycosylated form of polypeptide by recombinant expression in mammalian cells.

The vector, pRK5 (see EP 307,247, published Mar. 15, 1989), is employed as the expression vector. Optionally, the DNA is ligated into pRK5 with selected restriction enzymes to allow insertion of the DNA using ligation methods such as described in Sambrook et al, supra., and is designated pRK5-DNA.

In one embodiment, the selected host cells can be NIH3T3 cells, using the vectors and transfection methods described herein for other mammalian cells. Transfected NIH3T3 cells, over-expressing gene encoding a protein of the present invention or expressing antisense, are tested for activity.

In one embodiment, the selected host cells can be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-DNA is mixed with about 1 μg DNA encoding the VA RNA gene (Thimmappaya et al., Cell, 31:543 (1982)) and dissolved in 500 μl of 1 mM Tris-HCl, 0.1 mM EDTA, 0.227 M CaCl₂, To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added, and the cells are incubated for about 5 days.

Approximately 24 hours after transfection, the culture medium is removed and replaced with culture medium (alone) or culture medium containing 200 μCi/ml ³⁵S-cysteine and 200 μCi/ml ³⁵S-methionine. After a 12-hour incubation, the conditioned medium is collected, concentrated on a spin filter, and loaded onto a 15% SDS gel. The processed gel can be dried and exposed to film for a selected period of time to reveal the presence of polypeptide. The cultures containing transfected cells can undergo further incubation (in serum free medium) and the medium is then tested in selected bioassays.

In an alternative technique, a protein of the present invention can be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and reintroduced into the spinner flask containing tissue culture medium, 5 μg/ml bovine insulin, and 0.1 μg/ml bovine transferrin. After about four days, the conditioned media is centrifuged and filtered to remove cells and debris. The sample containing expressed a protein of the present invention can then be concentrated and purified by any selected method, such as dialysis and/or column chromatography.

In another embodiment, a protein of the present invention can be expressed in CHO cells. The pRK5-DNA can be transfected into CHO cells using known reagents such as CaPO₄, or DEAE-dextran. As described above, the cell cultures can be incubated, and the medium replaced with culture medium (alone) or medium containing a radiolabel such as ³⁵S-methionine. After determining the presence of polypeptide, the culture medium can be replaced with serum free medium. Preferably, the cultures are incubated for about 6 days, and then the conditioned medium is harvested. The medium containing the expressed a protein of the present invention can then be concentrated and purified by any selected method.

Epitope-tagged polypeptide can also be expressed in host CHO cells. The DNA can be subcloned out of the pRK5 vector. The subclone insert can undergo PCR to fuse in frame with a selected epitope tag such as a poly-His tag or myc tag such as in pcDNA3.1/myc-his, (Invitrogen, Carlsbad, Calif.). This vector has the neo gene for selection of stable clones using G418 and the human cytomegalovirus promoter operationally linked to the genes of the present invention. Labeling can be performed, as described above, to verify expression. The culture medium containing the expressed poly-His tagged polypeptide can then be concentrated and purified by any selected method, such as by Ni²⁺-chelate affinity chromatography.

Protein of the present invention can also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 gm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media can be employed, a production medium described in U.S. Pat. No. 5,122,469, issued Jun. 16, 1992 can actually be used. A 3L production spinner is seeded at 1.2×106 cells/mL. On day 0, the cell number pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken.

Throughout the production, the pH is adjusted as necessary to keep it at around 7.2. After 10 days, or until the viability dropped below 70%, the cell culture is harvested by centrifugation and filtering through a 0.22 gm filter. The filtrate was either stored at 4° C. or immediately loaded onto columns for purification. For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) that had been equilibrated in 20 mM Na phosphate buffer, pH 6.8. After loading, the column is washed extensively with equilibration buffer before elution with 100 mM citric acid, pH 3.5. The eluted protein is immediately neutralized by collecting one ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Stable expression in CHO cells was also accomplished without fusing the gene of interest to the Fc domain. In this case, a plasmid that expresses the gene of interest, such as pcDNA3.1/myc-his, described above, as a c-myc, poly His fusion, was transfected into approximately 5×10⁵ cells in a 35 mm tissue culture dish. The following day, the cells were dislodged by trypsin/EDTA or other comparable cell dissociation mixture, diluted into 100 ml growth media and plated into ten 100 mm tissue culture plates. The following day, the media was removed and replaced with media containing the appropriate amount of selection drug. In this case, the selection drug is G418 (Geneticin, Life Technologies) added to approximately 800 ug/ml. After approximately 10 days, the cells that did not stably take up the DNA are dead and small colonies of stably transfected cells are visible. These cells can be dissociated either singly, using cloning cylinders (Freshney, 2000), or as a pool and plated in larger tissue culture plates. Expression of the gene of interest can be detected in the individual clones, after expansion, by the techniques detailed above or by other techniques such as Western blotting using antibodies to the protein of interest or to the epitope tags.

In addition, the gene of interest was cloned into pIRES2-eGFP vector (Clonetech). This vector expresses an enhanced green fluorescence protein (eGFP) placed downstream from the internal ribosomal entry site (IRES). Cells are transfected with this vector and selected using G418. Cells that take up the DNA can be identified by fluorescence microscopy by identification of green fluorescent cells. Moreover, cells that express the gene of interest can be isolated or enriched by fluorescence activated cell sorting. Expression of the gene of interest by the GFP expressing cells was accomplished as above

In addition to CHO, HEK293 and HUVEC cells, the genes of interest are expressed in various tumor cell lines using the same vectors and detection techniques described above. Some of the human tumor cell lines used include SW480 (ATCC, CCL-228), HCT-116 (ATCC, CCL-247), DLD-1 (ATCC, CCL-221), LS 174T (ATCC, CCL-188) or HT-29, ATCC, HTB-38).

### Example 10

Gene Expression in Yeast

Recombinant expression of polypeptide in yeast can also be accomplished.

First, yeast expression vectors are constructed for intracellular production or secretion of a protein of the present invention from the ADH2/GAPDH promoter. DNA encoding a protein of the present invention and the promoter is inserted into suitable restriction enzyme sites in the selected plasmid to direct intracellular expression of a protein of the present invention. For secretion, DNA encoding a protein of the present invention can be cloned into the selected plasmid, together with DNA encoding the ADH2/GAPDH promoter, a native signal peptide of a protein of the present invention or other mammalian signal peptide, or, for example, a yeast alpha-factor or invertase secretory signal/leader sequence, and linker sequences (if needed) for expression of a protein of the present invention.

Yeast cells, such as yeast strain AB 110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant polypeptide can subsequently be isolated and purified by removing the yeast cells from the fermentation medium by centrifugation and then concentrating the medium using selected cartridge filters. The concentrate containing polypeptide can further be purified using selected column chromatography resins.

### Example 11

Expression of Polypeptides in Baculovirus-Infected Cells

The following method describes recombinant expression of polypeptide in Baculovirus-infected insect cells.

The DNA sequence coding for polypeptide is fused upstream of an epitope tag contained within a baculovirus expression vector. Such epitope tags include poly-His tags and immunoglobulin tags (like Fc regions of IgG). A variety of plasmids can be employed, including plasmids derived from commercially available plasmids such as pVL 1393 (Novagen). Briefly, the sequence encoding polypeptide or the desired portion of the coding sequence such as the sequence encoding the extracellular domain of a transmembrane protein or the sequence encoding the mature protein if the protein is extracellular is amplified by PCR with primers complementary to the 5′ and 3′ regions. The 5′ primer can incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-His tagged polypeptide can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 01 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 gm filter. A Ni²⁺-NTA agarose column (commercially available from Qiagen) is prepared with a bed volume of 5 mL, washed with 25 mL of water, and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged protein are pooled and dialyzed against loading buffer.

Alternatively, purification of the IgG tagged (or Fc tagged) a protein of the present invention can be performed using known chromatography techniques, including for instance, Protein A or protein G column chromatography.

### Example 12

Preparation of Monoclonal Antibodies that Bind a Polypeptide of the Present Invention

This example illustrates preparation of monoclonal antibodies, which can specifically bind a protein of the present invention.

Techniques for producing the monoclonal antibodies are known in the art and are described, for instance, in Goding, supra. Immunogens that can be employed include purified a protein of the present invention, fusion proteins containing a protein of the present invention, and cells expressing recombinant a protein of the present invention on the cell surface. Selection of the immunogen can be made by the skilled artisan without undue experimentation. Mice, such as Balb/c, are immunized with the immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind footpads. The immunized mice are then boosted 10 to 12 days later with additional immunogen emulsified in the selected adjuvant. Thereafter, for several weeks, the mice can also be boosted with additional immunization injections. Serum samples can be periodically obtained from the mice by retro-orbital bleeding for testing in ELISA assays to detect antibodies.

After a suitable antibody titer has been detected, the animals “positive” for antibodies can be injected with a final intravenous injection of polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells, which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against a protein of the present invention. Determination of “positive” hybridoma cells secreting the desired monoclonal antibodies against polypeptide is within the skill in the art.

The positive hybridoma cells can be injected intraperitoneally into syngeneic Balb/c mice to produce ascites containing the monoclonal antibodies against a protein of the present invention. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 13

Preparation of Antibodies that Bind a Polypeptide of the Present Invention

This example illustrates preparation of polyclonal antibodies, which can specifically bind a protein of the present invention.

Techniques for producing the polyclonal antibodies are known in the art and are described, for instance, in Harlow and Lane, Antibodies: A Laboratory Manual, Cold Spring Harbor Laboratory, (1988). Immunogens that can be employed include purified a protein of the present invention, fusion proteins containing a protein of the present invention, and cells expressing recombinant a protein of the present invention on the cell surface or synthetic peptides derived from a protein of present invention coupled to a carrier protein. Selection of the immunogen can be made by the skilled artisan without undue experimentation. Tables 8-14 show exempliary peptides suitable for polyclonal antibody production. Rabbits, such as New Zealand White, are immunized with the immunogen emulsified in complete Freund's adjuvant and injected subcutaneously or intramuscular in an amount from 50-1000 micrograms. The immunized mice are then boosted 2 to 4 weeks later with additional immunogen emulsified in the selected adjuvant. Thereafter, for several months, the mice can also be boosted with additional immunization injections. Serum samples can be periodically obtained from the ear vein of rabbits for testing in ELISA assays to detect antibodies.

Purification of the polyclonal antibodies from serum can be accomplished using ammonium sulfate precipitation, followed by gel exclusion chromatography. Alternatively, affinity chromatography based upon binding of antibody to protein A, protein G or the immunogen can be employed.

TABLE 8
KIAA 0792 peptides

| | |
|---|---|
| CKIEQALAQTGSVAAAPQEALSN | (SEQ ID NO:3084) |
| CKIELPRDARKETVESHFRDLSN | (SEQ ID NO:3085) |
| CKIEDLLDKDPYSFGRTTIALSN | (SEQ ID NO:3086) |

TABLE 9
GPR-56 peptides

| | |
|---|---|
| CKIERGHREDFRFASQRNQTLSN | (SEQ ID NO:3087) |
| CKIEHAPFPAAHPASRSFPDLSN | (SEQ ID NO:3088) |
| CKIERLQARGGPSPLKSNSDLSN | (SEQ ID NO:3089) |

TABLE 10
P cadherin peptides

| | |
|---|---|
| VTDQNDHKPKFTQ | (SEQ ID NO:3090) |
| DANDNAPMFDPQKY | (SEQ ID NO:3091) |
| DVNEAPVFVPPSK | (SEQ ID NO:3092) |
| DVNDHGPVPEPRQI | (SEQ ID NO:3093) |
| RDWVVAPISVPE | (SEQ ID NO:3094) |
| YTLTIQATDMDGDGSTTTAV | (SEQ ID NO:3095) |

TABLE 11
FGFR3 peptides

| | |
|---|---|
| VENKFGSIRQTYTLD | (SEQ ID NO:3096) |
| GLPANQTAVLGSDVE | (SEQ ID NO:3097) |
| GLPANQTAILGSDVE | (SEQ ID NO:3098) |
| PYVTVLKTAGANTTDK | (SEQ ID NO:3099) |
| PYVTVLKSWISESVEAD | (SEQ ID NO:3100) |
| PYVTVLKSWISEVEAD | (SEQ ID NO:3101) |

TABLE 12
CCK4 peptides

| | |
|---|---|
| KQPSSQDALQGRRALLR | (SEQ ID NO:3102) |
| PAGSIEAQAVLQVLEKLK | (SEQ ID NO:3103) |
| KSLQSKDEQQQLDFRRE | (SEQ ID NO:3104) |

TABLE 13
TM4SF6

| | |
|---|---|
| EIKNSFKNNYEKALKQYN | (SEQ ID NO:3105) |
| DYRDWTDTNYYSEKGFPK | (SEQ ID NO:3106) |
| MASPSRRLQTKPVIT | (SEQ ID NO:3107) |

TABLE 14
IFITM

| | |
|---|---|
| MNHIVQTFSPVNSGQ | (SEQ ID NO:3108) |
| MSHTVQTFFSPVNSG | (SEQ ID NO:3109) |
| EMLKEEQEVAMLGGP | (SEQ ID NO:3110) |
| EMLKEEHEVAVLGGP | (SEQ ID NO:3111) |
| KSRDRKMVGDVTGAQ | (SEQ ID NO:3112) |

### Example 14

Purification of Polypeptides Using Specific Antibodies

Native or recombinant polypeptides can be purified by a variety of standard techniques in the art of protein purification. For example, pro-polypeptide, mature polypeptide, or pre-polypeptide is purified by immunoaffinity chromatography using antibodies specific for the polypeptide of interest. In general, an immunoaffinity column is constructed by covalently coupling the antibody to an activated chromatographic resin. Polyclonal immunoglobulins are prepared from immune sera either by precipitation with ammonium sulfate or by purification on immobilized Protein A (Pharmacia LK.B Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LK.B Biotechnology). The antibody is coupled to the resin, the resin is blocked, and the derivative resin is washed according to the manufacturer's instructions.

Such an immunoaffinity column is utilized in the purification of polypeptide by preparing a fraction from cells containing polypeptide in a soluble form. This preparation is derived by solubilization of the whole cell or of a subcellular fraction obtained via differential centrifugation by the addition of detergent or by other methods well known in the art. Alternatively, soluble polypeptide containing a signal sequence can be secreted in useful quantity into the medium in which the cells are grown.

A soluble polypeptide-containing preparation is passed over the immunoaffinity column, and the column is washed under conditions that allow the preferential absorbance of polypeptide (e.g., high ionic strength buffers in the presence of detergent). Then, the column is eluted under conditions that disrupt antibody/polypeptide binding (e.g., a low pH buffer such as approximately pH 2-3, or a high concentration of a chaotrope such as urea or thiocyanate ion), and polypeptide is collected.

1. An antibody that immunospecifically-binds to p-cadherin or a fragment thereof.

2. The antibody of claim 1 wherein said p-cadherin has the amino acid sequence of SEQ ID NO: 39.

3. The antibody of claim 2, wherein said antibody is a monoclonal antibody.

4. The antibody of claim 2, wherein said antibody is an antibody fragment selected from the group consisting of a FV fragment, a Fab fragment, (Fab)₂ fragment, a single chain antibody.

5. The antibody of claim 4 wherein said antibody is conjugated with at least one polyethylene glycol moiety.

6. The antibody of claim 3 wherein said antibody is an antagonist.

7. The antibody of claim 6 wherein the antibody is a humanized antibody.

8. The antibody of claim 6 wherein the antibody is a human antibody.

9. A method of identifying an agent that binds to p-cadherin comprising:
(a) contacting p-cadherin with said agent; and
(b) determining whether said agent binds to p-cadherin.

10. A method for identifying an agent that modulates the expression or activity of p-cadherin comprising:
(a) providing a cell expressing said polypeptide in an operational manner;
(b) contacting the cell with said agent; and
(c) determining whether the agent modulates expression or activity of said polypeptide;
whereby an alteration in expression or activity of p-cadherin indicates said agent modulates expression or activity of p-cadherin.

11. A method of treating or preventing a cancer-associated disorder, said method comprising administering to a subject in which such treatment or prevention is desired said antibody of claim 1 in an amount sufficient to treat or prevent said cancer-associated disorder in said subject.

12. A method of detecting differentially expressed genes correlated with a cancerous state of a mammalian cell, the method comprising the step of detecting at least one differentially expressed gene product in a test sample derived from a cell suspected of being cancerous, where the gene product is encoded by a sequence of SEQ ID NO:1 wherein detection of differentially expressed product is correlated with a cancerous state of the cell from which the test sample was derived.

13. A method for monitoring the progression of a cancer in a patient, the method comprising:
a) detecting in a patient sample at a first point in time, the expression of a marker, wherein the marker a nucleic acid molecule of SEQ ID NO:1;
b) repeating step a) at a subsequent point in time; and
c) comparing the level of expression detected in steps a) and b), and therefrom monitoring the progression of the cancer.

14. A method of assessing the efficacy of a test compound for inhibiting a cancer in a patient, the method comprising comparing:
a) expression of a marker in a first sample obtained from the patient exposed to the test compound, wherein the marker is selected the nucleic acid molecule of SEQ ID NO:1, and
b) expression of the marker in a second sample obtained from the patient, wherein the sample is not exposed to the test compound, wherein a significantly lower level of expression of the marker in the first sample, relative to the second sample, is an indication that the test compound is efficacious for inhibiting the cancer in the patient.
